SEQUENCE LISTING (41 sequences)

SEQ ID NO: 1 - DNA, 10206 bp, Megathura crenulata
gtagacacagtggtgagaaagaatgtggacagtttgagcagtgacgaggttctagccctcgagaaggctctcgatgatctccagcaagacgacagcaaccagggttatcaggccatagcaggctaccatggtgttccaacaatgtgtgtggacaaacatgaaaaaaatgtggcctgttgtcttcacggtatgccatcctttcccctttggcatcgtctctacgttgtccagttggaaagagcgctgataaggaagaaggcaaccatcagcatcccctactgggattggactagtgagctcacccatcttccagaacttgtgtctcatccactgtttgtcgacactgaggggggcaaggctcatgacaattcttggtaccgagctgacatcactttcctcaacaagaagacaagtcgtgctgtagatgaccgtctttttgaaaaagtacagccaggacaccacactcgcctcatggagggaatccttgacgctctggaacaggacgagttttgcaaatttgagatccagtttgagctggctcacaacgccatacattatctagttgggggcagacacacatattccatgtctcacctcgagtacacgtcatacgatcctttgttctttttacatcacttcaacactgaccgtatctttgctatatggcaacgacttcaacagctcagaggcaaggatcccaacagtgctgactgtgcccataaccttattcacactccaatggaaccgttcgacagagacactaatccactggatctgacaagagagcacgccaaacctgctgatagctttgactacgggcgcctcggctaccagtatgacgacctctccttgaatggcatgagtccagaggaactcaacgtatatctaggagaacgagcagctaaggagcgtacatttgccagcttcatattatctggttttggtggctctgccaatgttgtagtctatgtctgccgacctgctcatgatgaaatcgctgatgatcaatgtatcaaagcaggagatttcttccttcttggcggaccaactgaaatgaagtggggattctacagggcataccactttgatgttactgacagcgttgcttctatagatgatgatggtcatgggcattattacgtgaagtccgagttattcagcgtcaacggaagcgctttgtcaaatgacattcttcgacaacccaccttagtgcaccgaccagctaaaggtcattttgataagcctccagttccagttgctcaagctaacctggctgtcagaaagaatatcaacgatctaactgcagaggaaacatacagcttgaggaaagctatggaaagattccagaatgactcagttgacgggtaccaagctacagtagaattccatgcccttccagcaagatgtccacgcccagatgctaaagatagatttgcatgctgcgttcatggcatggcaaccttcccccactggcatagactctttgtcactcaagttgaagatgctctacttcgtcgcgggtctactatcggtctaccttactgggactggaccatgccaatggatcatctgccagaactggctatgagtgagacctatccccatccagtttctagcgcaatcatggcaaatccattttaccatgccgaagtggcttttgaaaataacgtcacacacagagatacagacaatagactctttgagaaaccgtcttttggacaccacacgcatctgtttgacgctatggtctatgcttttgaacaagaagacttttgcgactttgaagttcagtttgaactcgttcacaataacatacatgcgtgggttggaggaagtgaaaagtactccatgtcttctcttcactacactgcttttgatcctattttctacctccatcactcaaatgttgatcgtctctgggccatttggcaagctcttcaaatcaggaggaacaaaccttacaaggcccattgtgcctggtcacaggaaagggaaccattaaagcctttttcattcagttcacctctcaacaacaacgataaaacctaccaaaactctgttccaactaatgtttacgactatgagggagttttggactatcgatatgatgacctccagtttggtggcatgaccctgtcagaacttgaaaaatatatacacaagcagaaacagcatgacagaagttttgcaggattcttcctttctcatattggaacatcagcaaacgtagacgtccttatcagtgcaaagggccaagaggcccacacgattggaagctttgcagtgcttggtggatctaaggaaatgaaatgggctttcgacagaatgtacaaatatgaaattactgatgctgccaaaaagctgaacattgatgtgaatgatgccactatttctgttaagatcactgatgttgatggaacttccttgtcaccagagctaattccgtctcctgctctcatgtttgaacctgggcatattgttgccaaagactttggccacagcaaaaaaatcaggaagaacgttcattccctgacagctgaagaacaaaattcgttgaggcgagctatggcagatctccaagatgacaaaacacgaggtggattccaacagattgcagcatttcacggagaaccaaaatggtgcccacgccctgaagcggagaagaaatttgcatgctgcgttcacggaatggctgttttccctcactggcacagattgctgacagttcaaggagaaaatgctctgaggaaacatggatttactggtggattgccctattgggactggactcgaccaatgagcgcccttccacattttgttgctgatcctacttacaatgattctgtttccagcctcgaagaagataacccatggtatcatggtcacatagattctgttgggcatgatactacaagagctgtgcgtgatgatctttatcaatctcctggtttcggtcactacacagatattgcaaaacaagtccttctggcctttgagcaggacgatttctgtgattttgaggtacaatttgaaattgcccataatttcatacatgctctagttggtggtaacgaaccatacagtatgtcatctttgaggtatactacatacgatccaatcttcttcttgcaccactccaatacagaccgactttgggccatttggcaagctttgcaaaaataccgggggaaaccatacaacactgcaaactgtgccattgcatccatgagaaaaccacttcagccatttggtcttgatagtgtcataaatccagatgacgaaactcgtgaacattcggttcctttccgagtcttcgactacaagaacaacttcgactatgagtatgagagcctggcatttaatggtctgtctattgcccaactggaccgagagttgcagagaagaaagtcacatgacagagtctttgcaggattccttcttcatgaaattggacagtctgcactcgtgaaattctacgtttgcaaacacaatgtatctgactgtgaccattatgctggagaattctacattttgggagatgaagctgagatgccttggaggtatgaccgtgtgtacaagtacgagataacacagcagctgcacgatttagatctacatgttggagataatttctttcttaaatatgaagcctttgatctgaatggcggaagtcttggtggaagtatcttttctcagccttcggtgattttcgagccaactgcaggttcacaccaggctgatgaatatcgtgaggcagtaacaagcgctagccacataagaaaaaatatccgggacctctcagagggagaaattgagagcatcagatctgctttcctccaaattcaaaaagagggtatatatgaaaacattgcaaagttccatggaaaaccaggactttgtgaatatgatggacatcctgttgcttgttgtgtccatggcatgcccacctttccccactggcacagactgtacgttcttcaggtggagaatgcgctcttagaacgagggtctgcagttgctgttccttactgggactggaccgagaaagctgactctctgccatcattaatcaatgatgcaacttatttcaattcacgatcccagacctttgatcctaatcctttcttcaggggacatatcgccttcgagaatgctgtgacgtccagagatcctcagccagaactatgggacaataaggacttctacgagaatgtcatgctggctcttgagcaagacaacttctgtgactttgagattcagcttgagctgatacacaacgcccttcattctagacttggaggaagggctaaatactccctttcgtctcttgattataccgcatttgatcctgtatttttccttcaccatgcaaacgttgacagaatctgggccatctggcaggacttgcagagatatagaaagaaaccatacaatgaggctgactgcgcagtcaacgagatgcgtaaacctcttcaaccatttaataacccagaacttaacagtgattccatgacgcttaaacacaacctcccacaagacagttttgattatcaaaaccgcttcaggtaccaatatgataaccttcaatttaaccacttcagcatacaaaagctagaccaaactattcaggctagaaaacaacacgacagagtttttgctggctttattcttcacaacattgggacatctgttgttgtagatatttatatttgcgttgaacaaggaggagaacaaaactgcaagacaaaggcgggttccttcacgattctggggggagaaacagaaatgccattccactttgaccgcttgtacaaatttgacataacgtctgctctgcataaacttggtgttcccttggacggacatggattcgacatcaaagttgacgtcagagctgtcaatggatcgcatcttgatccacacatcctcaacgaaccgagtctgctttttgttcctggtgaacattcacatcaaactgcagatgggctttcacaacataatcttgtgcgaaaagaagtaagctctcttacaacactggagaaacattttttgaggaaagctctcaagaacatgcaagcagatgattctccagacggatatcaagctattgcttctttccacgctttgcctcctctttgtccaagtccatctgctgcacatagacacgcttgttgcctccatggtatggctaccttccctcagtggcacagactctacacagttcagttcgaagattctttgaaacgacatggttctattgtcggacttccatattgggattggctgaaaccgcagtctgcactccctgatttggtgacacaggagacatacgagcacctgttttcacacaaaaccttcccaaatccgttcctcaaggcaaatatagaatttgagggagagggagtaacaacagagagggatgttgatgctgaacacctctttgcaaaaggaaatctggtttacaacaactggttttacaatcaggcactatatgcactagaacaagaaaattactgtgactttgaaatacagttcgaaattttgcataatggaattcattcatgggttggaggatcaaagacccattcaataggtcatcttcattacgcatcatacgatccactgttctatatccaccattcgcagacagatcgcatttgggctatctggcaagctttccaggagcacagaggtctttcagggaaggaagcacactgcgccctggagcaaatgaaagaccctctcaaacctttcagctttggaagtccctataatttgaacaaacgcactcaagagttctccaagcctgaagacacatttgattatcaccgattcgggtatgagtatgattccctcgaatttgttggcatgtctgtttcaagtttacataactatataaaacaacaacaggaagctgatagagtcttcgcaggattccttcttaaaggatttggacaatcagcatccgtatcgtttgatatctgcagaccagaccagagttgccaagaagctggatacttctcagttctcggtggaagttcagaaatgccgtggcagtttgacaggctttacaagtacgacattacaaaaacgttgaaagacatgaaactgcgatacgatgacgcatttaccatcaaggttcacataaaggatatagctggagctgagttggacagcgatctgattccaactccttctgttctctttgaagaaggaaagcatgggatcaatgtacgtcacgttggtcgtaatcggattcgtatggaactatctgaactcaccgagagagatctcgccagcctgaaatctgcaatgaggtctctacaagctgacgatggggtgaacggttatcaagccattgcatcattccacggtctcccggcttcttgtcatgatgatgagggacatgagattgcctgttgtatccacggaatgccagtattcccacactggcacaggctttacaccctgcaaatggacatggctctgttatctcacggatctgctgttgctattccatactgggactggaccaaacctatcagcaaactgcctgatctcttcaccagccctgaatattacgatccttggagggatgcagttgtcaataatccatttgctaaaggctacattaaatccgaggacgcttacacggttagggatcctcaggacattttgtaccacttgcaggacgaaacgggaacatctgttttgttagatcaaactcttttagccttagaacagacagatttctgtgattttgaggttcaatttgaggtcgtccataatgctattcactacttggtgggtggtcgacaagtttatgctctttcttctctacactatgcttcatatgacccagccttctttattcatcactcctttgttgacaaaatatgggcagtctggcaagctctgcaaaagaagagaaagcgtccctatcataaagcggattgtgctcttaacatgatgaccaaaccaatgcgaccatttgcacacgatttcaatcacaatggattcacaaaaatgcacgcagtccccaacactctatttgactttcaggaccttttctacacgtatgacaacttagaaattgctggcatgaatgttaatcagttggaagcggaaatcaaccggcgaaaaagccaaacaagagtctttgccgggttccttctacatggcattggaagatcagctgatgtacgattttggatttgcaagacagctgacgactgccacgcatctggcatgatctttatcttaggaggttctaaagagatgcactgggcctatgacaggaactttaaatacgacatcacccaagctttgaaggctcagtccatacaccctgaagatgtgtttgacactgatgctcctttcttcattaaagtggagatccatggtgtaaacaagactgctctcccatcttcagctatcccagcacctactataatctactcagctggtgaaggtcataccgatgatcacggctcagatcatattgctggcagtggagtcaggaaagacgtgacgtctcttaccgcatctgagatagagaacctgaggcatgctctgcaaagcgtgatggatgatgatggacccaatggattccaggcaattgctgcttatcacggaagtcctcccatgtgtcacatgcctgatggtagagacgttgcatgttgtactcatggaatggcatctttccctcactggcacagactgtttgtgaaacagatggaggatgcactggctgcgcatggagctcacattggcataccatactgggattggacaagtgcgtttagtcatctgcctgccctagtgactgaccacgagcacaatcccttccaccacggacatattgctcatcggaatgtggatacatctcgatctccgagagacatgctgttcaatgaccctgaacacgggtcagaatcattcttctatagacaggttctcttggctctagaacagacagacttctgccaatttgaagttcagtttgaaataacacacaatgcaatccactcttggactggaggacatactccatatggaatgtcatcactggaatatacagcatatgatccactcttttatctccaccattccaacactgatcgtatctgggccatctggcaggcactccagaaatacagaggttttcaatacaacgcagctcattgcgatatccaggttctgaaacaacctcttaaaccattcagcgagtccaggaatccaaacccagtcaccagagccaattctagggcagtcgattcatttgattatgagagactcaattatcaatatgacacacttaccttccacggacattctatctcagaacttgatgccatgcttcaagagagaaagaaggaagagagaacatttgcagccttcctgttgcacggatttggcgccagtgctgatgtttcgtttgatgtctgcacacctgatggtcattgtgcctttgctggaaccttcgcggtacttggtggggagctggagatgccctggtcctttgaaagattgttccgttatgatatcacaaaggttctcaagcagatgaatctttactatgattctgagtttcatttcgcgttgaaggttgttggcacagatggaacagaactgccatcggatagtatcaagagcccaacaattgagcatcatcaagcagatcaccagggtcatggtggggagggtggccaacatcctgaacatactggatttgtaaggaaggacatagattctctttccttggatgaagccaatgatctgaaaaatgcactgtacaagctgcagaacgaccaaagcctcagtggatatgaatcaatagctggtttccatggctatccctacctctgtcctgacaacggcgatgacaagtatgcctgctgtgtccatggaatgcctgtatttccacactggcacagacttcttactatccagtttgagagagcactcaaagaacatggttcacatttgggtgttccctattgggattggaccaagtcaatgactgctcttccagctttctttgccgattcttccaataacaaccctttctacaaatatcacattatgaaagctggacatgatacaacgagacagccgaatgatcttctcttcaatcaaccacaggttcatggatatgactatctatactaccttgccctttcaacactagaagaggataactactgtgactttgaggttcagtatgaaaattttcacaatgcagtacatatctggctcgggggaactgaaacctactccatggctactcttgaatattcagcgtttgatccagtctttatgatcttgcattctggtctcgacagactctggatcatttggcaaaagctgcaaaagctcagaaagaaaccatataatgcagccaaatgtgctggccacatgattgatgaacctttgcatccttttgactacgagtctgttaaccaagatggcttcactcgagcaaacgctaaacctacaacagtgtttgacagtcacaggtttgattatcactatgacaacctggatgtacgaggaaagagtattcaggaaataaacgccatcatccacgatttgaggacccaaccaagaatctttgctggttttgttctttctggaatatacacttcagctaatgttaagatatacttaatcagagaaggccatgacgatgaatatgtaggaacatttgcagttttaggaggagccaaggagatgccatgggcctatgagaggattttcaagtatgacattacagatgtcgcccacaaactgaaaatgcaccatgatgatacattcaacttcagacttgaaattcaatcttacagtggcgaaacactaacatcacatcttcctgaacctctcataatccacagaccagccaacaaggactatgatgtcttgattattccattaggaagcggacgcaagcttcctcctaaagtcattgtcaagagaggaacagaaattgagtttcatccagtcgatgacacgatcaacagacctgttgtagatcttggcagctacactgctctctacaactgtgtggtgccacccttcacataccacggatatgagctcgatcatgcctactcccttagggatgggcactactatattgcaggcccaaccaaagacctctgcgccagcggaaatgttagaatacatatccacattgaagacgagtaa

SEQ ID NO: 2 - DNA, 10206 bp, Megathura crenulata
  SEQ ID NO2 is the complementary sequence of the coding sequence of SEQ ID NO1
ttactcgtcttcaatgtggatatgtattctaacatttccgctggcgcagaggtctttggttgggcctgcaatatagtagtgcccatccctaagggagtaggcatgatcgagctcatatccgtggtatgtgaagggtggcaccacacagttgtagagagcagtgtagctgccaagatctacaacaggtctgttgatcgtgtcatcgactggatgaaactcaatttctgttcctctcttgacaatgactttaggaggaagcttgcgtccgcttcctaatggaataatcaagacatcatagtccttgttggctggtctgtggattatgagaggttcaggaagatgtgatgttagtgtttcgccactgtaagattgaatttcaagtctgaagttgaatgtatcatcatggtgcattttcagtttgtgggcgacatctgtaatgtcatacttgaaaatcctctcataggcccatggcatctccttggctcctcctaaaactgcaaatgttcctacatattcatcgtcatggccttctctgattaagtatatcttaacattagctgaagtgtatattccagaaagaacaaaaccagcaaagattcttggttgggtcctcaaatcgtggatgatggcgtttatttcctgaatactctttcctcgtacatccaggttgtcatagtgataatcaaacctgtgactgtcaaacactgttgtaggtttagcgtttgctcgagtgaagccatcttggttaacagactcgtagtcaaaaggatgcaaaggttcatcaatcatgtggccagcacatttggctgcattatatggtttctttctgagcttttgcagcttttgccaaatgatccagagtctgtcgagaccagaatgcaagatcataaagactggatcaaacgctgaatattcaagagtagccatggagtaggtttcagttcccccgagccagatatgtactgcattgtgaaaattttcatactgaacctcaaagtcacagtagttatcctcttctagtgttgaaagggcaaggtagtatagatagtcatatccatgaacctgtggttgattgaagagaagatcattcggctgtctcgttgtatcatgtccagctttcataatgtgatatttgtagaaagggttgttattggaagaatcggcaaagaaagctggaagagcagtcattgacttggtccaatcccaatagggaacacccaaatgtgaaccatgttctttgagtgctctctcaaactggatagtaagaagtctgtgccagtgtggaaatacaggcattccatggacacagcaggcatacttgtcatcgccgttgtcaggacagaggtagggatagccatggaaaccagctattgattcatatccactgaggctttggtcgttctgcagcttgtacagtgcatttttcagatcattggcttcatccaaggaaagagaatctatgtccttccttacaaatccagtatgttcaggatgttggccaccctccccaccatgaccctggtgatctgcttgatgatgctcaattgttgggctcttgatactatccgatggcagttctgttccatctgtgccaacaaccttcaacgcgaaatgaaactcagaatcatagtaaagattcatctgcttgagaacctttgtgatatcataacggaacaatctttcaaaggaccagggcatctccagctccccaccaagtaccgcgaaggttccagcaaaggcacaatgaccatcaggtgtgcagacatcaaacgaaacatcagcactggcgccaaatccgtgcaacaggaaggctgcaaatgttctctcttccttctttctctcttgaagcatggcatcaagttctgagatagaatgtccgtggaaggtaagtgtgtcatattgataattgagtctctcataatcaaatgaatcgactgccctagaattggctctggtgactgggtttggattcctggactcgctgaatggtttaagaggttgtttcagaacctggatatcgcaatgagctgcgttgtattgaaaacctctgtatttctggagtgcctgccagatggcccagatacgatcagtgttggaatggtggagataaaagagtggatcatatgctgtatattccagtgatgacattccatatggagtatgtcctccagtccaagagtggattgcattgtgtgttatttcaaactgaacttcaaattggcagaagtctgtctgttctagagccaagagaacctgtctatagaagaatgattctgacccgtgttcagggtcattgaacagcatgtctctcggagatcgagatgtatccacattccgatgagcaatatgtccgtggtggaagggattgtgctcgtggtcagtcactagggcaggcagatgactaaacgcacttgtccaatcccagtatggtatgccaatgtgagctccatgcgcagccagtgcatcctccatctgtttcacaaacagtctgtgccagtgagggaaagatgccattccatgagtacaacatgcaacgtctctaccatcaggcatgtgacacatgggaggacttccgtgataagcagcaattgcctggaatccattgggtccatcatcatccatcacgctttgcagagcatgcctcaggttctctatctcagatgcggtaagagacgtcacgtctttcctgactccactgccagcaatatgatctgagccgtgatcatcggtatgaccttcaccagctgagtagattatagtaggtgctgggatagctgaagatgggagagcagtcttgtttacaccatggatctccactttaatgaagaaaggagcatcagtgtcaaacacatcttcagggtgtatggactgagccttcaaagcttgggtgatgtcgtatttaaagttcctgtcataggcccagtgcatctctttagaacctcctaagataaagatcatgccagatgcgtggcagtcgtcagctgtcttgcaaatccaaaatcgtacatcagctgatcttccaatgccatgtagaaggaacccggcaaagactcttgtttggctttttcgccggttgatttccgcttccaactgattaacattcatgccagcaatttctaagttgtcatacgtgtagaaaaggtcctgaaagtcaaatagagtgttggggactgcgtgcatttttgtgaatccattgtgattgaaatcgtgtgcaaatggtcgcattggtttggtcatcatgttaagagcacaatccgctttatgatagggacgctttctcttcttttgcagagcttgccagactgcccatattttgtcaacaaaggagtgatgaataaagaaggctgggtcatatgaagcatagtgtagagaagaaagagcataaacttgtcgaccacccaccaagtagtgaatagcattatggacgacctcaaattgaacctcaaaatcacagaaatctgtctgttctaaggctaaaagagtttgatctaacaaaacagatgttcccgtttcgtcctgcaagtggtacaaaatgtcctgaggatccctaaccgtgtaagcgtcctcggatttaatgtagcctttagcaaatggattattgacaactgcatccctccaaggatcgtaatattcagggctggtgaagagatcaggcagtttgctgataggtttggtccagtcccagtatggaatagcaacagcagatccgtgagataacagagccatgtccatttgcagggtgtaaagcctgtgccagtgtgggaatactggcattccgtggatacaacaggcaatctcatgtccctcatcatcatgacaagaagccgggagaccgtggaatgatgcaatggcttgataaccgttcaccccatcgtcagcttgtagagacctcattgcagatttcaggctggcgagatctctctcggtgagttcagatagttccatacgaatccgattacgaccaacgtgacgtacattgatcccatgctttccttcttcaaagagaacagaaggagttggaatcagatcgctgtccaactcagctccagctatatcctttatgtgaaccttgatggtaaatgcgtcatcgtatcgcagtttcatgtctttcaacgtttttgtaatgtcgtacttgtaaagcctgtcaaactgccacggcatttctgaacttccaccgagaactgagaagtatccagcttcttggcaactctggtctggtctgcagatatcaaacgatacggatgctgattgtccaaatcctttaagaaggaatcctgcgaagactctatcagcttcctgttgttgttttatatagttatgtaaacttgaaacagacatgccaacaaattcgagggaatcatactcatacccgaatcggtgataatcaaatgtgtcttcaggcttggagaactcttgagtgcgtttgttcaaattatagggacttccaaagctgaaaggtttgagagggtctttcatttgctccagggcgcagtgtgcttccttccctgaaagacctctgtgctcctggaaagcttgccagatagcccaaatgcgatctgtctgcgaatggtggatatagaacagtggatcgtatgatgcgtaatgaagatgacctattgaatgggtctttgatcctccaacccatgaatgaattccattatgcaaaatttcgaactgtatttcaaagtcacagtaattttcttgttctagtgcatatagtgcctgattgtaaaaccagttgttgtaaaccagatttccttttgcaaagaggtgttcagcatcaacatccctctctgttgttactccctctccctcaaattctatatttgccttgaggaacggatttgggaaggttttgtgtgaaaacaggtgctcgtatgtctcctgtgtcaccaaatcagggagtgcagactgcggtttcagccaatcccaatatggaagtccgacaatagaaccatgtcgtttcaaagaatcttcgaactgaactgtgtagagtctgtgccactgagggaaggtagccataccatggaggcaacaagcgtgtctatgtgcagcagatggacttggacaaagaggaggcaaagcgtggaaagaagcaatagcttgatatccgtctggagaatcatctgcttgcatgttcttgagagctttcctcaaaaaatgtttctccagtgttgtaagagagcttacttcttttcgcacaagattatgttgtgaaagcccatctgcagtttgatgtgaatgttcaccaggaacaaaaagcagactcggttcgttgaggatgtgtggatcaagatgcgatccattgacagctctgacgtcaactttgatgtcgaatccatgtccgtccaagggaacaccaagtttatgcagagcagacgttatgtcaaatttgtacaagcggtcaaagtggaatggcatttctgtttctccccccagaatcgtgaaggaacccgcctttgtcttgcagttttgttctcctccttgttcaacgcaaatataaatatctacaacaacagatgtcccaatgttgtgaagaataaagccagcaaaaactctgtcgtgttgttttctagcctgaatagtttggtctagcttttgtatgctgaagtggttaaattgaaggttatcatattggtacctgaagcggttttgataatcaaaactgtcttgtgggaggttgtgtttaagcgtcatggaatcactgttaagttctgggttattaaatggttgaagaggtttacgcatctcgttgactgcgcagtcagcctcattgtatggtttctttctatatctctgcaagtcctgccagatggcccagattctgtcaacgtttgcatggtgaaggaaaaatacaggatcaaatgcggtataatcaagagacgaaagggagtatttagcccttcctccaagtctagaatgaagggcgttgtgtatcagctcaagctgaatctcaaagtcacagaagttgtcttgctcaagagccagcatgacattctcgtagaagtccttattgtcccatagttctggctgaggatctctggacgtcacagcattctcgaaggcgatatgtcccctgaagaaaggattaggatcaaaggtctgggatcgtgaattgaaataagttgcatcattgattaatgatggcagagagtcagctttctcggtccagtcccagtaaggaacagcaactgcagaccctcgttctaagagcgcattctccacctgaagaacgtacagtctgtgccagtggggaaaggtgggcatgccatggacacaacaagcaacaggatgtccatcatattcacaaagtcctggttttccatggaactttgcaatgttttcatatataccctctttttgaatttggaggaaagcagatctgatgctctcaatttctccctctgagaggtcccggatattttttcttatgtggctagcgcttgttactgcctcacgatattcatcagcctggtgtgaacctgcagttggctcgaaaatcaccgaaggctgagaaaagatacttccaccaagacttccgccattcagatcaaaggcttcatatttaagaaagaaattatctccaacatgtagatctaaatcgtgcagctgctgtgttatctcgtacttgtacacacggtcatacctccaaggcatctcagcttcatctcccaaaatgtagaattctccagcataatggtcacagtcagatacattgtgtttgcaaacgtagaatttcacgagtgcagactgtccaatttcatgaagaaggaatcctgcaaagactctgtcatgtgactttcttctctgcaactctcggtccagttgggcaatagacagaccattaaatgccaggctctcatactcatagtcgaagttgttcttgtagtcgaagactcggaaaggaaccgaatgttcacgagtttcgtcatctggatttatgacactatcaagaccaaatggctgaagtggttttctcatggatgcaatggcacagtttgcagtgttgtatggtttcccccggtatttttgcaaagcttgccaaatggcccaaagtcggtctgtattggagtggtgcaagaagaagattggatcgtatgtagtatacctcaaagatgacatactgtatggttcgttaccaccaactagagcatgtatgaaattatgggcaatttcaaattgtacctcaaaatcacagaaatcgtcctgctcaaaggccagaaggacttgttttgcaatatctgtgtagtgaccgaaaccaggagattgataaagatcatcacgcacagctcttgtagtatcatgcccaacagaatctatgtgaccatgataccatgggttatcttcttcgaggctggaaacagaatcattgtaagtaggatcagcaacaaaatgtggaagggcgctcattggtcgagtccagtcccaatagggcaatccaccagtaaatccatgtttcctcagagcattttctccttgaactgtcagcaatctgtgccagtgagggaaaacagccattccgtgaacgcagcatgcaaatttcttctccgcttcagggcgtgggcaccattttggttctccgtgaaatgctgcaatctgttggaatccacctcgtgttttgtcatcttggagatctgccatagctcgcctcaacgaattttgttcttcagctgtcagggaatgaacgttcttcctgatttttttgctgtggccaaagtctttggcaacaatatgcccaggttcaaacatgagagcaggagacggaattagctctggtgacaaggaagttccatcaacatcagtgatcttaacagaaatagtggcatcattcacatcaatgttcagctttttggcagcatcagtaatttcatatttgtacattctgtcgaaagcccatttcatttccttagatccaccaagcactgcaaagcttccaatcgtgtgggcctcttggccctttgcactgataaggacgtctacgtttgctgatgttccaatatgagaaaggaagaatcctgcaaaacttctgtcatgctgtttctgcttgtgtatatatttttcaagttctgacagggtcatgccaccaaactggaggtcatcatatcgatagtccaaaactccctcatagtcgtaaacattagttggaacagagttttggtaggttttatcgttgttgttgagaggtgaactgaatgaaaaaggctttaatggttccctttcctgtgaccaggcacaatgggccttgtaaggtttgttcctcctgatttgaagagcttgccaaatggcccagagacgatcaacatttgagtgatggaggtagaaaataggatcaaaagcagtgtagtgaagagaagacatggagtacttttcacttcctccaacccacgcatgtatgttattgtgaacgagttcaaactgaacttcaaagtcgcaaaagtcttcttgttcaaaagcatagaccatagcgtcaaacagatgcgtgtggtgtccaaaagacggtttctcaaagagtctattgtctgtatctctgtgtgtgacgttattttcaaaagccacttcggcatggtaaaatggatttgccatgattgcgctagaaactggatggggataggtctcactcatagccagttctggcagatgatccattggcatggtccagtcccagtaaggtagaccgatagtagacccgcgacgaagtagagcatcttcaacttgagtgacaaagagtctatgccagtgggggaaggttgccatgccatgaacgcagcatgcaaatctatctttagcatctgggcgtggacatcttgctggaagggcatggaattctactgtagcttggtacccgtcaactgagtcattctggaatctttccatagctttcctcaagctgtatgtttcctctgcagttagatcgttgatattctttctgacagccaggttagcttgagcaactggaactggaggcttatcaaaatgacctttagctggtcggtgcactaaggtgggttgtcgaagaatgtcatttgacaaagcgcttccgttgacgctgaataactcggacttcacgtaataatgcccatgaccatcatcatctatagaagcaacgctgtcagtaacatcaaagtggtatgccctgtagaatccccacttcatttcagttggtccgccaagaaggaagaaatctcctgctttgatacattgatcatcagcgatttcatcatgagcaggtcggcagacatagactacaacattggcagagccaccaaaaccagataatatgaagctggcaaatgtacgctccttagctgctcgttctcctagatatacgttgagttcctctggactcatgccattcaaggagaggtcgtcatactggtagccgaggcgcccgtagtcaaagctatcagcaggtttggcgtgctctcttgtcagatccagtggattagtgtctctgtcgaacggttccattggagtgtgaataaggttatgggcacagtcagcactgttgggatccttgcctctgagctgttgaagtcgttgccatatagcaaagatacggtcagtgttgaagtgatgtaaaaagaacaaaggatcgtatgacgtgtactcgaggtgagacatggaatatgtgtgtctgcccccaactagataatgtatggcgttgtgagccagctcaaactggatctcaaatttgcaaaactcgtcctgttccagagcgtcaaggattccctccatgaggcgagtgtggtgtcctggctgtactttttcaaaaagacggtcatctacagcacgacttgtcttcttgttgaggaaagtgatgtcagctcggtaccaagaattgtcatgagccttgcccccctcagtgtcgacaaacagtggatgagacacaagttctggaagatgggtgagctcactagtccaatcccagtaggggatgctgatggttgccttcttccttatcagcgctctttccaactggacaacgtagagacgatgccaaaggggaaaggatggcataccgtgaagacaacaggccacatttttttcatgtttgtccacacacattgttggaacaccatggtagcctgctatggcctgataaccctggttgctgtcgtcttgctggagatcatcgagagccttctcgagggctagaacctcgtcactgctcaaactgtccacattctttctcaccactgtgtctac

SEQ ID NO: 3 - protein, 3401 aa, Megathura crenulata
  SEQ ID NO3 is the deduced amino acid sequence of SEQ ID NO1
VDTVVRKNVDSLSSDEVLALEKALDDLQQDDSNQGYQAIAGYHGVPTMCVDKHEKNVACCLHGMPSFPLWHRLYVVQLERALIRKKATISIPYWDWTSELTHLPELVSHPLFVDTEGGKAHDNSWYRADITFLNKKTSRAVDDRLFEKVQPGHHTRLMEGILDALEQDEFCKFEIQFELAHNAIHYLVGGRHTYSMSHLEYTSYDPLFFLHHFNTDRIFAIWQRLQQLRGKDPNSADCAHNLIHTPMEPFDRDTNPLDLTREHAKPADSFDYGRLGYQYDDLSLNGMSPEELNVYLGERAAKERTFASFILSGFGGSANVVVYVCRPAHDEIADDQCIKAGDFFLLGGPTEMKWGFYRAYHFDVTDSVASIDDDGHGHYYVKSELFSVNGSALSNDILRQPTLVHRPAKGHFDKPPVPVAQANLAVRKNINDLTAEETYSLRKAMERFQNDSVDGYQATVEFHALPARCPRPDAKDRFACCVHGMATFPHWHRLFVTQVEDALLRRGSTIGLPYWDWTMPMDHLPELAMSETYPHPVSSAIMANPFYHAEVAFENNVTHRDTDNRLFEKPSFGHHTHLFDAMVYAFEQEDFCDFEVQFELVHNNIHAWVGGSEKYSMSSLHYTAFDPIFYLHHSNVDRLWAIWQALQIRRNKPYKAHCAWSQEREPLKPFSFSSPLNNNDKTYQNSVPTNVYDYEGVLDYRYDDLQFGGMTLSELEKYIHKQKQHDRSFAGFFLSHIGTSANVDVLISAKGQEAHTIGSFAVLGGSKEMKWAFDRMYKYEITDAAKKLNIDVNDATISVKITDVDGTSLSPELIPSPALMFEPGHIVAKDFGHSKKIRKNVHSLTAEEQNSLRRAMADLQDDKTRGGFQQIAAFHGEPKWCPRPEAEKKFACCVHGMAVFPHWHRLLTVQGENALRKHGFTGGLPYWDWTRPMSALPHFVADPTYNDSVSSLEEDNPWYHGHIDSVGHDTTRAVRDDLYQSPGFGHYTDIAKQVLLAFEQDDFCDFEVQFEIAHNFIHALVGGNEPYSMSSLRYTTYDPIFFLHHSNTDRLWAIWQALQKYRGKPYNTANCAIASMRKPLQPFGLDSVINPDDETREHSVPFRVFDYKNNFDYEYESLAFNGLSIAQLDRELQRRKSHDRVFAGFLLHEIGQSALVKFYVCKHNVSDCDHYAGEFYILGDEAEMPWRYDRVYKYEITQQLHDLDLHVGDNFFLKYEAFDLNGGSLGGSIFSQPSVIFEPTAGSHQADEYREAVTSASHIRKNIRDLSEGEIESIRSAFLQIQKEGIYENIAKFHGKPGLCEYDGHPVACCVHGMPTFPHWHRLYVLQVENALLERGSAVAVPYWDWTEKADSLPSLINDATYFNSRSQTFDPNPFFRGHIAFENAVTSRDPQPELWDNKDFYENVMLALEQDNFCDFEIQLELIHNALHSRLGGRAKYSLSSLDYTAFDPVFFLHHANVDRIWAIWQDLQRYRKKPYNEADCAVNEMRKPLQPFNNPELNSDSMTLKHNLPQDSFDYQNRFRYQYDNLQFNHFSIQKLDQTIQARKQHDRVFAGFILHNIGTSVVVDIYICVEQGGEQNCKTKAGSFTILGGETEMPFHFDRLYKFDITSALHKLGVPLDGHGFDIKVDVRAVNGSHLDPHILNEPSLLFVPGEHSHQTADGLSQHNLVRKEVSSLTTLEKHFLRKALKNMQADDSPDGYQAIASFHALPPLCPSPSAAHRHACCLHGMATFPQWHRLYTVQFEDSLKRHGSIVGLPYWDWLKPQSALPDLVTQETYEHLFSHKTFPNPFLKANIEFEGEGVTTERDVDAEHLFAKGNLVYNNWFYNQALYALEQENYCDFEIQFEILHNGIHSWVGGSKTHSIGHLHYASYDPLFYIHHSQTDRIWAIWQAFQEHRGLSGKEAHCALEQMKDPLKPFSFGSPYNLNKRTQEFSKPEDTFDYHRFGYEYDSLEFVGMSVSSLHNYIKQQQEADRVFAGFLLKGFGQSASVSFDICRPDQSCQEAGYFSVLGGSSEMPWQFDRLYKYDITKTLKDMKLRYDDAFTIKVHIKDIAGAELDSDLIPTPSVLFEEGKHGINVRHVGRNRIRMELSELTERDLASLKSAMRSLQADDGVNGYQAIASFHGLPASCHDDEGHEIACCIHGMPVFPHWHRLYTLQMDMALLSHGSAVAIPYWDWTKPISKLPDLFTSPEYYDPWRDAVVNNPFAKGYIKSEDAYTVRDPQDILYHLQDETGTSVLLDQTLLALEQTDFCDFEVQFEVVHNAIHYLVGGRQVYALSSLHYASYDPAFFIHHSFVDKIWAVWQALQKKRKRPYHKADCALNMMTKPMRPFAHDFNHNGFTKMHAVPNTLFDFQDLFYTYDNLEIAGMNVNQLEAEINRRKSQTRVFAGFLLHGIGRSADVRFWICKTADDCHASGMIFILGGSKEMHWAYDRNFKYDITQALKAQSIHPEDVFDTDAPFFIKVEIHGVNKTALPSSAIPAPTIIYSAGEGHTDDHGSDHIAGSGVRKDVTSLTASEIENLRHALQSVMDDDGPNGFQAIAAYHGSPPMCHMPDGRDVACCTHGMASFPHWHRLFVKQMEDALAAHGAHIGIPYWDWTSAFSHLPALVTDHEHNPFHHGHIAHRNVDTSRSPRDMLFNDPEHGSESFFYRQVLLALEQTDFCQFEVQFEITHNAIHSWTGGHTPYGMSSLEYTAYDPLFYLHHSNTDRIWAIWQALQKYRGFQYNAAHCDIQVLKQPLKPFSESRNPNPVTRANSRAVDSFDYERLNYQYDTLTFHGHSISELDAMLQERKKEERTFAAFLLHGFGASADVSFDVCTPDGHCAFAGTFAVLGGELEMPWSFERLFRYDITKVLKQMNLYYDSEFHFALKVVGTDGTELPSDSIKSPTIEHHQADHQGHGGEGGQHPEHTGFVRKDIDSLSLDEANDLKNALYKLQNDQSLSGYESIAGFHGYPYLCPDNGDDKYACCVHGMPVFPHWHRLLTIQFERALKEHGSHLGVPYWDWTKSMTALPAFFADSSNNNPFYKYHIMKAGHDTTRQPNDLLFNQPQVHGYDYLYYLALSTLEEDNYCDFEVQYENFHNAVHIWLGGTETYSMATLEYSAFDPVFMILHSGLDRLWIIWQKLQKLRKKPYNAAKCAGHMIDEPLHPFDYESVNQDGFTRANAKPTTVFDSHRFDYHYDNLDVRGKSIQEINAIIHDLRTQPRIFAGFVLSGIYTSANVKIYLIREGHDDEYVGTFAVLGGAKEMPWAYERIFKYDITDVAHKLKMHHDDTFNFRLEIQSYSGETLTSHLPEPLIIHRPANKDYDVLIIPLGSGRKLPPKVIVKRGTEIEFHPVDDTINRPVVDLGSYTALYNCVVPPFTYHGYELDHAYSLRDGHYYIAGPTKDLCASGNVRIHIHIEDE

SEQ ID NO: 4 - DNA, 57 bp, Megathura crenulata
atgtggaccatcttggctctcctcacggcgaccctcttgtttgagggagccttctcg

SEQ ID NO: 5 - DNA, 57 bp, Megathura crenulata
  SEQ ID NO5 is the complementary sequence of the coding sequence of SEQ ID NO4
cgagaaggctccctcaaacaagagggtcgccgtgaggagagccaagatggtccacat

SEQ ID NO: 6 - protein, 19 aa, Megathura crenulata
  SEQ ID NO6 is the deduced amino acid sequence of SEQ ID NO4
MWTILALLTATLLFEGAFS

SEQ ID NO: 7 - DNA, 10263 bp, Megathura crenulata
atgtggaccatcttggctctcctcacggcgaccctcttgtttgagggagccttctcggtagacacagtggtgagaaagaatgtggacagtttgagcagtgacgaggttctagccctcgagaaggctctcgatgatctccagcaagacgacagcaaccagggttatcaggccatagcaggctaccatggtgttccaacaatgtgtgtggacaaacatgaaaaaaatgtggcctgttgtcttcacggtatgccatcctttcccctttggcatcgtctctacgttgtccagttggaaagagcgctgataaggaagaaggcaaccatcagcatcccctactgggattggactagtgagctcacccatcttccagaacttgtgtctcatccactgtttgtcgacactgaggggggcaaggctcatgacaattcttggtaccgagctgacatcactttcctcaacaagaagacaagtcgtgctgtagatgaccgtctttttgaaaaagtacagccaggacaccacactcgcctcatggagggaatccttgacgctctggaacaggacgagttttgcaaatttgagatccagtttgagctggctcacaacgccatacattatctagttgggggcagacacacatattccatgtctcacctcgagtacacgtcatacgatcctttgttctttttacatcacttcaacactgaccgtatctttgctatatggcaacgacttcaacagctcagaggcaaggatcccaacagtgctgactgtgcccataaccttattcacactccaatggaaccgttcgacagagacactaatccactggatctgacaagagagcacgccaaacctgctgatagctttgactacgggcgcctcggctaccagtatgacgacctctccttgaatggcatgagtccagaggaactcaacgtatatctaggagaacgagcagctaaggagcgtacatttgccagcttcatattatctggttttggtggctctgccaatgttgtagtctatgtctgccgacctgctcatgatgaaatcgctgatgatcaatgtatcaaagcaggagatttcttccttcttggcggaccaactgaaatgaagtggggattctacagggcataccactttgatgttactgacagcgttgcttctatagatgatgatggtcatgggcattattacgtgaagtccgagttattcagcgtcaacggaagcgctttgtcaaatgacattcttcgacaacccaccttagtgcaccgaccagctaaaggtcattttgataagcctccagttccagttgctcaagctaacctggctgtcagaaagaatatcaacgatctaactgcagaggaaacatacagcttgaggaaagctatggaaagattccagaatgactcagttgacgggtaccaagctacagtagaattccatgcccttccagcaagatgtccacgcccagatgctaaagatagatttgcatgctgcgttcatggcatggcaaccttcccccactggcatagactctttgtcactcaagttgaagatgctctacttcgtcgcgggtctactatcggtctaccttactgggactggaccatgccaatggatcatctgccagaactggctatgagtgagacctatccccatccagtttctagcgcaatcatggcaaatccattttaccatgccgaagtggcttttgaaaataacgtcacacacagagatacagacaatagactctttgagaaaccgtcttttggacaccacacgcatctgtttgacgctatggtctatgcttttgaacaagaagacttttgcgactttgaagttcagtttgaactcgttcacaataacatacatgcgtgggttggaggaagtgaaaagtactccatgtcttctcttcactacactgcttttgatcctattttctacctccatcactcaaatgttgatcgtctctgggccatttggcaagctcttcaaatcaggaggaacaaaccttacaaggcccattgtgcctggtcacaggaaagggaaccattaaagcctttttcattcagttcacctctcaacaacaacgataaaacctaccaaaactctgttccaactaatgtttacgactatgagggagttttggactatcgatatgatgacctccagtttggtggcatgaccctgtcagaacttgaaaaatatatacacaagcagaaacagcatgacagaagttttgcaggattcttcctttctcatattggaacatcagcaaacgtagacgtccttatcagtgcaaagggccaagaggcccacacgattggaagctttgcagtgcttggtggatctaaggaaatgaaatgggctttcgacagaatgtacaaatatgaaattactgatgctgccaaaaagctgaacattgatgtgaatgatgccactatttctgttaagatcactgatgttgatggaacttccttgtcaccagagctaattccgtctcctgctctcatgtttgaacctgggcatattgttgccaaagactttggccacagcaaaaaaatcaggaagaacgttcattccctgacagctgaagaacaaaattcgttgaggcgagctatggcagatctccaagatgacaaaacacgaggtggattccaacagattgcagcatttcacggagaaccaaaatggtgcccacgccctgaagcggagaagaaatttgcatgctgcgttcacggaatggctgttttccctcactggcacagattgctgacagttcaaggagaaaatgctctgaggaaacatggatttactggtggattgccctattgggactggactcgaccaatgagcgcccttccacattttgttgctgatcctacttacaatgattctgtttccagcctcgaagaagataacccatggtatcatggtcacatagattctgttgggcatgatactacaagagctgtgcgtgatgatctttatcaatctcctggtttcggtcactacacagatattgcaaaacaagtccttctggcctttgagcaggacgatttctgtgattttgaggtacaatttgaaattgcccataatttcatacatgctctagttggtggtaacgaaccatacagtatgtcatctttgaggtatactacatacgatccaatcttcttcttgcaccactccaatacagaccgactttgggccatttggcaagctttgcaaaaataccgggggaaaccatacaacactgcaaactgtgccattgcatccatgagaaaaccacttcagccatttggtcttgatagtgtcataaatccagatgacgaaactcgtgaacattcggttcctttccgagtcttcgactacaagaacaacttcgactatgagtatgagagcctggcatttaatggtctgtctattgcccaactggaccgagagttgcagagaagaaagtcacatgacagagtctttgcaggattccttcttcatgaaattggacagtctgcactcgtgaaattctacgtttgcaaacacaatgtatctgactgtgaccattatgctggagaattctacattttgggagatgaagctgagatgccttggaggtatgaccgtgtgtacaagtacgagataacacagcagctgcacgatttagatctacatgttggagataatttctttcttaaatatgaagcctttgatctgaatggcggaagtcttggtggaagtatcttttctcagccttcggtgattttcgagccaactgcaggttcacaccaggctgatgaatatcgtgaggcagtaacaagcgctagccacataagaaaaaatatccgggacctctcagagggagaaattgagagcatcagatctgctttcctccaaattcaaaaagagggtatatatgaaaacattgcaaagttccatggaaaaccaggactttgtgaatatgatggacatcctgttgcttgttgtgtccatggcatgcccacctttccccactggcacagactgtacgttcttcaggtggagaatgcgctcttagaacgagggtctgcagttgctgttccttactgggactggaccgagaaagctgactctctgccatcattaatcaatgatgcaacttatttcaattcacgatcccagacctttgatcctaatcctttcttcaggggacatatcgccttcgagaatgctgtgacgtccagagatcctcagccagaactatgggacaataaggacttctacgagaatgtcatgctggctcttgagcaagacaacttctgtgactttgagattcagcttgagctgatacacaacgcccttcattctagacttggaggaagggctaaatactccctttcgtctcttgattataccgcatttgatcctgtatttttccttcaccatgcaaacgttgacagaatctgggccatctggcaggacttgcagagatatagaaagaaaccatacaatgaggctgactgcgcagtcaacgagatgcgtaaacctcttcaaccatttaataacccagaacttaacagtgattccatgacgcttaaacacaacctcccacaagacagttttgattatcaaaaccgcttcaggtaccaatatgataaccttcaatttaaccacttcagcatacaaaagctagaccaaactattcaggctagaaaacaacacgacagagtttttgctggctttattcttcacaacattgggacatctgttgttgtagatatttatatttgcgttgaacaaggaggagaacaaaactgcaagacaaaggcgggttccttcacgattctggggggagaaacagaaatgccattccactttgaccgcttgtacaaatttgacataacgtctgctctgcataaacttggtgttcccttggacggacatggattcgacatcaaagttgacgtcagagctgtcaatggatcgcatcttgatccacacatcctcaacgaaccgagtctgctttttgttcctggtgaacattcacatcaaactgcagatgggctttcacaacataatcttgtgcgaaaagaagtaagctctcttacaacactggagaaacattttttgaggaaagctctcaagaacatgcaagcagatgattctccagacggatatcaagctattgcttctttccacgctttgcctcctctttgtccaagtccatctgctgcacatagacacgcttgttgcctccatggtatggctaccttccctcagtggcacagactctacacagttcagttcgaagattctttgaaacgacatggttctattgtcggacttccatattgggattggctgaaaccgcagtctgcactccctgatttggtgacacaggagacatacgagcacctgttttcacacaaaaccttcccaaatccgttcctcaaggcaaatatagaatttgagggagagggagtaacaacagagagggatgttgatgctgaacacctctttgcaaaaggaaatctggtttacaacaactggttttacaatcaggcactatatgcactagaacaagaaaattactgtgactttgaaatacagttcgaaattttgcataatggaattcattcatgggttggaggatcaaagacccattcaataggtcatcttcattacgcatcatacgatccactgttctatatccaccattcgcagacagatcgcatttgggctatctggcaagctttccaggagcacagaggtctttcagggaaggaagcacactgcgccctggagcaaatgaaagaccctctcaaacctttcagctttggaagtccctataatttgaacaaacgcactcaagagttctccaagcctgaagacacatttgattatcaccgattcgggtatgagtatgattccctcgaatttgttggcatgtctgtttcaagtttacataactatataaaacaacaacaggaagctgatagagtcttcgcaggattccttcttaaaggatttggacaatcagcatccgtatcgtttgatatctgcagaccagaccagagttgccaagaagctggatacttctcagttctcggtggaagttcagaaatgccgtggcagtttgacaggctttacaagtacgacattacaaaaacgttgaaagacatgaaactgcgatacgatgacgcatttaccatcaaggttcacataaaggatatagctggagctgagttggacagcgatctgattccaactccttctgttctctttgaagaaggaaagcatgggatcaatgtacgtcacgttggtcgtaatcggattcgtatggaactatctgaactcaccgagagagatctcgccagcctgaaatctgcaatgaggtctctacaagctgacgatggggtgaacggttatcaagccattgcatcattccacggtctcccggcttcttgtcatgatgatgagggacatgagattgcctgttgtatccacggaatgccagtattcccacactggcacaggctttacaccctgcaaatggacatggctctgttatctcacggatctgctgttgctattccatactgggactggaccaaacctatcagcaaactgcctgatctcttcaccagccctgaatattacgatccttggagggatgcagttgtcaataatccatttgctaaaggctacattaaatccgaggacgcttacacggttagggatcctcaggacattttgtaccacttgcaggacgaaacgggaacatctgttttgttagatcaaactcttttagccttagaacagacagatttctgtgattttgaggttcaatttgaggtcgtccataatgctattcactacttggtgggtggtcgacaagtttatgctctttcttctctacactatgcttcatatgacccagccttctttattcatcactcctttgttgacaaaatatgggcagtctggcaagctctgcaaaagaagagaaagcgtccctatcataaagcggattgtgctcttaacatgatgaccaaaccaatgcgaccatttgcacacgatttcaatcacaatggattcacaaaaatgcacgcagtccccaacactctatttgactttcaggaccttttctacacgtatgacaacttagaaattgctggcatgaatgttaatcagttggaagcggaaatcaaccggcgaaaaagccaaacaagagtctttgccgggttccttctacatggcattggaagatcagctgatgtacgattttggatttgcaagacagctgacgactgccacgcatctggcatgatctttatcttaggaggttctaaagagatgcactgggcctatgacaggaactttaaatacgacatcacccaagctttgaaggctcagtccatacaccctgaagatgtgtttgacactgatgctcctttcttcattaaagtggagatccatggtgtaaacaagactgctctcccatcttcagctatcccagcacctactataatctactcagctggtgaaggtcataccgatgatcacggctcagatcatattgctggcagtggagtcaggaaagacgtgacgtctcttaccgcatctgagatagagaacctgaggcatgctctgcaaagcgtgatggatgatgatggacccaatggattccaggcaattgctgcttatcacggaagtcctcccatgtgtcacatgcctgatggtagagacgttgcatgttgtactcatggaatggcatctttccctcactggcacagactgtttgtgaaacagatggaggatgcactggctgcgcatggagctcacattggcataccatactgggattggacaagtgcgtttagtcatctgcctgccctagtgactgaccacgagcacaatcccttccaccacggacatattgctcatcggaatgtggatacatctcgatctccgagagacatgctgttcaatgaccctgaacacgggtcagaatcattcttctatagacaggttctcttggctctagaacagacagacttctgccaatttgaagttcagtttgaaataacacacaatgcaatccactcttggactggaggacatactccatatggaatgtcatcactggaatatacagcatatgatccactcttttatctccaccattccaacactgatcgtatctgggccatctggcaggcactccagaaatacagaggttttcaatacaacgcagctcattgcgatatccaggttctgaaacaacctcttaaaccattcagcgagtccaggaatccaaacccagtcaccagagccaattctagggcagtcgattcatttgattatgagagactcaattatcaatatgacacacttaccttccacggacattctatctcagaacttgatgccatgcttcaagagagaaagaaggaagagagaacatttgcagccttcctgttgcacggatttggcgccagtgctgatgtttcgtttgatgtctgcacacctgatggtcattgtgcctttgctggaaccttcgcggtacttggtggggagctggagatgccctggtcctttgaaagattgttccgttatgatatcacaaaggttctcaagcagatgaatctttactatgattctgagtttcatttcgcgttgaaggttgttggcacagatggaacagaactgccatcggatagtatcaagagcccaacaattgagcatcatcaagcagatcaccagggtcatggtggggagggtggccaacatcctgaacatactggatttgtaaggaaggacatagattctctttccttggatgaagccaatgatctgaaaaatgcactgtacaagctgcagaacgaccaaagcctcagtggatatgaatcaatagctggtttccatggctatccctacctctgtcctgacaacggcgatgacaagtatgcctgctgtgtccatggaatgcctgtatttccacactggcacagacttcttactatccagtttgagagagcactcaaagaacatggttcacatttgggtgttccctattgggattggaccaagtcaatgactgctcttccagctttctttgccgattcttccaataacaaccctttctacaaatatcacattatgaaagctggacatgatacaacgagacagccgaatgatcttctcttcaatcaaccacaggttcatggatatgactatctatactaccttgccctttcaacactagaagaggataactactgtgactttgaggttcagtatgaaaattttcacaatgcagtacatatctggctcgggggaactgaaacctactccatggctactcttgaatattcagcgtttgatccagtctttatgatcttgcattctggtctcgacagactctggatcatttggcaaaagctgcaaaagctcagaaagaaaccatataatgcagccaaatgtgctggccacatgattgatgaacctttgcatccttttgactacgagtctgttaaccaagatggcttcactcgagcaaacgctaaacctacaacagtgtttgacagtcacaggtttgattatcactatgacaacctggatgtacgaggaaagagtattcaggaaataaacgccatcatccacgatttgaggacccaaccaagaatctttgctggttttgttctttctggaatatacacttcagctaatgttaagatatacttaatcagagaaggccatgacgatgaatatgtaggaacatttgcagttttaggaggagccaaggagatgccatgggcctatgagaggattttcaagtatgacattacagatgtcgcccacaaactgaaaatgcaccatgatgatacattcaacttcagacttgaaattcaatcttacagtggcgaaacactaacatcacatcttcctgaacctctcataatccacagaccagccaacaaggactatgatgtcttgattattccattaggaagcggacgcaagcttcctcctaaagtcattgtcaagagaggaacagaaattgagtttcatccagtcgatgacacgatcaacagacctgttgtagatcttggcagctacactgctctctacaactgtgtggtgccacccttcacataccacggatatgagctcgatcatgcctactcccttagggatgggcactactatattgcaggcccaaccaaagacctctgcgccagcggaaatgttagaatacatatccacattgaagacgagtaa

SEQ ID NO: 8 - DNA, 10263 bp, Megathura crenulata
  SEQ ID NO8 is the complementary sequence of the coding sequence of SEQ ID NO7
ttactcgtcttcaatgtggatatgtattctaacatttccgctggcgcagaggtctttggttgggcctgcaatatagtagtgcccatccctaagggagtaggcatgatcgagctcatatccgtggtatgtgaagggtggcaccacacagttgtagagagcagtgtagctgccaagatctacaacaggtctgttgatcgtgtcatcgactggatgaaactcaatttctgttcctctcttgacaatgactttaggaggaagcttgcgtccgcttcctaatggaataatcaagacatcatagtccttgttggctggtctgtggattatgagaggttcaggaagatgtgatgttagtgtttcgccactgtaagattgaatttcaagtctgaagttgaatgtatcatcatggtgcattttcagtttgtgggcgacatctgtaatgtcatacttgaaaatcctctcataggcccatggcatctccttggctcctcctaaaactgcaaatgttcctacatattcatcgtcatggccttctctgattaagtatatcttaacattagctgaagtgtatattccagaaagaacaaaaccagcaaagattcttggttgggtcctcaaatcgtggatgatggcgtttatttcctgaatactctttcctcgtacatccaggttgtcatagtgataatcaaacctgtgactgtcaaacactgttgtaggtttagcgtttgctcgagtgaagccatcttggttaacagactcgtagtcaaaaggatgcaaaggttcatcaatcatgtggccagcacatttggctgcattatatggtttctttctgagcttttgcagcttttgccaaatgatccagagtctgtcgagaccagaatgcaagatcataaagactggatcaaacgctgaatattcaagagtagccatggagtaggtttcagttcccccgagccagatatgtactgcattgtgaaaattttcatactgaacctcaaagtcacagtagttatcctcttctagtgttgaaagggcaaggtagtatagatagtcatatccatgaacctgtggttgattgaagagaagatcattcggctgtctcgttgtatcatgtccagctttcataatgtgatatttgtagaaagggttgttattggaagaatcggcaaagaaagctggaagagcagtcattgacttggtccaatcccaatagggaacacccaaatgtgaaccatgttctttgagtgctctctcaaactggatagtaagaagtctgtgccagtgtggaaatacaggcattccatggacacagcaggcatacttgtcatcgccgttgtcaggacagaggtagggatagccatggaaaccagctattgattcatatccactgaggctttggtcgttctgcagcttgtacagtgcatttttcagatcattggcttcatccaaggaaagagaatctatgtccttccttacaaatccagtatgttcaggatgttggccaccctccccaccatgaccctggtgatctgcttgatgatgctcaattgttgggctcttgatactatccgatggcagttctgttccatctgtgccaacaaccttcaacgcgaaatgaaactcagaatcatagtaaagattcatctgcttgagaacctttgtgatatcataacggaacaatctttcaaaggaccagggcatctccagctccccaccaagtaccgcgaaggttccagcaaaggcacaatgaccatcaggtgtgcagacatcaaacgaaacatcagcactggcgccaaatccgtgcaacaggaaggctgcaaatgttctctcttccttctttctctcttgaagcatggcatcaagttctgagatagaatgtccgtggaaggtaagtgtgtcatattgataattgagtctctcataatcaaatgaatcgactgccctagaattggctctggtgactgggtttggattcctggactcgctgaatggtttaagaggttgtttcagaacctggatatcgcaatgagctgcgttgtattgaaaacctctgtatttctggagtgcctgccagatggcccagatacgatcagtgttggaatggtggagataaaagagtggatcatatgctgtatattccagtgatgacattccatatggagtatgtcctccagtccaagagtggattgcattgtgtgttatttcaaactgaacttcaaattggcagaagtctgtctgttctagagccaagagaacctgtctatagaagaatgattctgacccgtgttcagggtcattgaacagcatgtctctcggagatcgagatgtatccacattccgatgagcaatatgtccgtggtggaagggattgtgctcgtggtcagtcactagggcaggcagatgactaaacgcacttgtccaatcccagtatggtatgccaatgtgagctccatgcgcagccagtgcatcctccatctgtttcacaaacagtctgtgccagtgagggaaagatgccattccatgagtacaacatgcaacgtctctaccatcaggcatgtgacacatgggaggacttccgtgataagcagcaattgcctggaatccattgggtccatcatcatccatcacgctttgcagagcatgcctcaggttctctatctcagatgcggtaagagacgtcacgtctttcctgactccactgccagcaatatgatctgagccgtgatcatcggtatgaccttcaccagctgagtagattatagtaggtgctgggatagctgaagatgggagagcagtcttgtttacaccatggatctccactttaatgaagaaaggagcatcagtgtcaaacacatcttcagggtgtatggactgagccttcaaagcttgggtgatgtcgtatttaaagttcctgtcataggcccagtgcatctctttagaacctcctaagataaagatcatgccagatgcgtggcagtcgtcagctgtcttgcaaatccaaaatcgtacatcagctgatcttccaatgccatgtagaaggaacccggcaaagactcttgtttggctttttcgccggttgatttccgcttccaactgattaacattcatgccagcaatttctaagttgtcatacgtgtagaaaaggtcctgaaagtcaaatagagtgttggggactgcgtgcatttttgtgaatccattgtgattgaaatcgtgtgcaaatggtcgcattggtttggtcatcatgttaagagcacaatccgctttatgatagggacgctttctcttcttttgcagagcttgccagactgcccatattttgtcaacaaaggagtgatgaataaagaaggctgggtcatatgaagcatagtgtagagaagaaagagcataaacttgtcgaccacccaccaagtagtgaatagcattatggacgacctcaaattgaacctcaaaatcacagaaatctgtctgttctaaggctaaaagagtttgatctaacaaaacagatgttcccgtttcgtcctgcaagtggtacaaaatgtcctgaggatccctaaccgtgtaagcgtcctcggatttaatgtagcctttagcaaatggattattgacaactgcatccctccaaggatcgtaatattcagggctggtgaagagatcaggcagtttgctgataggtttggtccagtcccagtatggaatagcaacagcagatccgtgagataacagagccatgtccatttgcagggtgtaaagcctgtgccagtgtgggaatactggcattccgtggatacaacaggcaatctcatgtccctcatcatcatgacaagaagccgggagaccgtggaatgatgcaatggcttgataaccgttcaccccatcgtcagcttgtagagacctcattgcagatttcaggctggcgagatctctctcggtgagttcagatagttccatacgaatccgattacgaccaacgtgacgtacattgatcccatgctttccttcttcaaagagaacagaaggagttggaatcagatcgctgtccaactcagctccagctatatcctttatgtgaaccttgatggtaaatgcgtcatcgtatcgcagtttcatgtctttcaacgtttttgtaatgtcgtacttgtaaagcctgtcaaactgccacggcatttctgaacttccaccgagaactgagaagtatccagcttcttggcaactctggtctggtctgcagatatcaaacgatacggatgctgattgtccaaatcctttaagaaggaatcctgcgaagactctatcagcttcctgttgttgttttatatagttatgtaaacttgaaacagacatgccaacaaattcgagggaatcatactcatacccgaatcggtgataatcaaatgtgtcttcaggcttggagaactcttgagtgcgtttgttcaaattatagggacttccaaagctgaaaggtttgagagggtctttcatttgctccagggcgcagtgtgcttccttccctgaaagacctctgtgctcctggaaagcttgccagatagcccaaatgcgatctgtctgcgaatggtggatatagaacagtggatcgtatgatgcgtaatgaagatgacctattgaatgggtctttgatcctccaacccatgaatgaattccattatgcaaaatttcgaactgtatttcaaagtcacagtaattttcttgttctagtgcatatagtgcctgattgtaaaaccagttgttgtaaaccagatttccttttgcaaagaggtgttcagcatcaacatccctctctgttgttactccctctccctcaaattctatatttgccttgaggaacggatttgggaaggttttgtgtgaaaacaggtgctcgtatgtctcctgtgtcaccaaatcagggagtgcagactgcggtttcagccaatcccaatatggaagtccgacaatagaaccatgtcgtttcaaagaatcttcgaactgaactgtgtagagtctgtgccactgagggaaggtagccataccatggaggcaacaagcgtgtctatgtgcagcagatggacttggacaaagaggaggcaaagcgtggaaagaagcaatagcttgatatccgtctggagaatcatctgcttgcatgttcttgagagctttcctcaaaaaatgtttctccagtgttgtaagagagcttacttcttttcgcacaagattatgttgtgaaagcccatctgcagtttgatgtgaatgttcaccaggaacaaaaagcagactcggttcgttgaggatgtgtggatcaagatgcgatccattgacagctctgacgtcaactttgatgtcgaatccatgtccgtccaagggaacaccaagtttatgcagagcagacgttatgtcaaatttgtacaagcggtcaaagtggaatggcatttctgtttctccccccagaatcgtgaaggaacccgcctttgtcttgcagttttgttctcctccttgttcaacgcaaatataaatatctacaacaacagatgtcccaatgttgtgaagaataaagccagcaaaaactctgtcgtgttgttttctagcctgaatagtttggtctagcttttgtatgctgaagtggttaaattgaaggttatcatattggtacctgaagcggttttgataatcaaaactgtcttgtgggaggttgtgtttaagcgtcatggaatcactgttaagttctgggttattaaatggttgaagaggtttacgcatctcgttgactgcgcagtcagcctcattgtatggtttctttctatatctctgcaagtcctgccagatggcccagattctgtcaacgtttgcatggtgaaggaaaaatacaggatcaaatgcggtataatcaagagacgaaagggagtatttagcccttcctccaagtctagaatgaagggcgttgtgtatcagctcaagctgaatctcaaagtcacagaagttgtcttgctcaagagccagcatgacattctcgtagaagtccttattgtcccatagttctggctgaggatctctggacgtcacagcattctcgaaggcgatatgtcccctgaagaaaggattaggatcaaaggtctgggatcgtgaattgaaataagttgcatcattgattaatgatggcagagagtcagctttctcggtccagtcccagtaaggaacagcaactgcagaccctcgttctaagagcgcattctccacctgaagaacgtacagtctgtgccagtggggaaaggtgggcatgccatggacacaacaagcaacaggatgtccatcatattcacaaagtcctggttttccatggaactttgcaatgttttcatatataccctctttttgaatttggaggaaagcagatctgatgctctcaatttctccctctgagaggtcccggatattttttcttatgtggctagcgcttgttactgcctcacgatattcatcagcctggtgtgaacctgcagttggctcgaaaatcaccgaaggctgagaaaagatacttccaccaagacttccgccattcagatcaaaggcttcatatttaagaaagaaattatctccaacatgtagatctaaatcgtgcagctgctgtgttatctcgtacttgtacacacggtcatacctccaaggcatctcagcttcatctcccaaaatgtagaattctccagcataatggtcacagtcagatacattgtgtttgcaaacgtagaatttcacgagtgcagactgtccaatttcatgaagaaggaatcctgcaaagactctgtcatgtgactttcttctctgcaactctcggtccagttgggcaatagacagaccattaaatgccaggctctcatactcatagtcgaagttgttcttgtagtcgaagactcggaaaggaaccgaatgttcacgagtttcgtcatctggatttatgacactatcaagaccaaatggctgaagtggttttctcatggatgcaatggcacagtttgcagtgttgtatggtttcccccggtatttttgcaaagcttgccaaatggcccaaagtcggtctgtattggagtggtgcaagaagaagattggatcgtatgtagtatacctcaaagatgacatactgtatggttcgttaccaccaactagagcatgtatgaaattatgggcaatttcaaattgtacctcaaaatcacagaaatcgtcctgctcaaaggccagaaggacttgttttgcaatatctgtgtagtgaccgaaaccaggagattgataaagatcatcacgcacagctcttgtagtatcatgcccaacagaatctatgtgaccatgataccatgggttatcttcttcgaggctggaaacagaatcattgtaagtaggatcagcaacaaaatgtggaagggcgctcattggtcgagtccagtcccaatagggcaatccaccagtaaatccatgtttcctcagagcattttctccttgaactgtcagcaatctgtgccagtgagggaaaacagccattccgtgaacgcagcatgcaaatttcttctccgcttcagggcgtgggcaccattttggttctccgtgaaatgctgcaatctgttggaatccacctcgtgttttgtcatcttggagatctgccatagctcgcctcaacgaattttgttcttcagctgtcagggaatgaacgttcttcctgatttttttgctgtggccaaagtctttggcaacaatatgcccaggttcaaacatgagagcaggagacggaattagctctggtgacaaggaagttccatcaacatcagtgatcttaacagaaatagtggcatcattcacatcaatgttcagctttttggcagcatcagtaatttcatatttgtacattctgtcgaaagcccatttcatttccttagatccaccaagcactgcaaagcttccaatcgtgtgggcctcttggccctttgcactgataaggacgtctacgtttgctgatgttccaatatgagaaaggaagaatcctgcaaaacttctgtcatgctgtttctgcttgtgtatatatttttcaagttctgacagggtcatgccaccaaactggaggtcatcatatcgatagtccaaaactccctcatagtcgtaaacattagttggaacagagttttggtaggttttatcgttgttgttgagaggtgaactgaatgaaaaaggctttaatggttccctttcctgtgaccaggcacaatgggccttgtaaggtttgttcctcctgatttgaagagcttgccaaatggcccagagacgatcaacatttgagtgatggaggtagaaaataggatcaaaagcagtgtagtgaagagaagacatggagtacttttcacttcctccaacccacgcatgtatgttattgtgaacgagttcaaactgaacttcaaagtcgcaaaagtcttcttgttcaaaagcatagaccatagcgtcaaacagatgcgtgtggtgtccaaaagacggtttctcaaagagtctattgtctgtatctctgtgtgtgacgttattttcaaaagccacttcggcatggtaaaatggatttgccatgattgcgctagaaactggatggggataggtctcactcatagccagttctggcagatgatccattggcatggtccagtcccagtaaggtagaccgatagtagacccgcgacgaagtagagcatcttcaacttgagtgacaaagagtctatgccagtgggggaaggttgccatgccatgaacgcagcatgcaaatctatctttagcatctgggcgtggacatcttgctggaagggcatggaattctactgtagcttggtacccgtcaactgagtcattctggaatctttccatagctttcctcaagctgtatgtttcctctgcagttagatcgttgatattctttctgacagccaggttagcttgagcaactggaactggaggcttatcaaaatgacctttagctggtcggtgcactaaggtgggttgtcgaagaatgtcatttgacaaagcgcttccgttgacgctgaataactcggacttcacgtaataatgcccatgaccatcatcatctatagaagcaacgctgtcagtaacatcaaagtggtatgccctgtagaatccccacttcatttcagttggtccgccaagaaggaagaaatctcctgctttgatacattgatcatcagcgatttcatcatgagcaggtcggcagacatagactacaacattggcagagccaccaaaaccagataatatgaagctggcaaatgtacgctccttagctgctcgttctcctagatatacgttgagttcctctggactcatgccattcaaggagaggtcgtcatactggtagccgaggcgcccgtagtcaaagctatcagcaggtttggcgtgctctcttgtcagatccagtggattagtgtctctgtcgaacggttccattggagtgtgaataaggttatgggcacagtcagcactgttgggatccttgcctctgagctgttgaagtcgttgccatatagcaaagatacggtcagtgttgaagtgatgtaaaaagaacaaaggatcgtatgacgtgtactcgaggtgagacatggaatatgtgtgtctgcccccaactagataatgtatggcgttgtgagccagctcaaactggatctcaaatttgcaaaactcgtcctgttccagagcgtcaaggattccctccatgaggcgagtgtggtgtcctggctgtactttttcaaaaagacggtcatctacagcacgacttgtcttcttgttgaggaaagtgatgtcagctcggtaccaagaattgtcatgagccttgcccccctcagtgtcgacaaacagtggatgagacacaagttctggaagatgggtgagctcactagtccaatcccagtaggggatgctgatggttgccttcttccttatcagcgctctttccaactggacaacgtagagacgatgccaaaggggaaaggatggcataccgtgaagacaacaggccacatttttttcatgtttgtccacacacattgttggaacaccatggtagcctgctatggcctgataaccctggttgctgtcgtcttgctggagatcatcgagagccttctcgagggctagaacctcgtcactgctcaaactgtccacattctttctcaccactgtgtctaccgagaaggctccctcaaacaagagggtcgccgtgaggagagccaagatggtccacat

SEQ ID NO: 9 - protein, 3420 aa, Megathura crenulata
  SEQ ID NO9 is the deduced amino acid sequence of SEQ ID NO7
MWTILALLTATLLFEGAFSVDTVVRKNVDSLSSDEVLALEKALDDLQQDDSNQGYQAIAGYHGVPTMCVDKHEKNVACCLHGMPSFPLWHRLYVVQLERALIRKKATISIPYWDWTSELTHLPELVSHPLFVDTEGGKAHDNSWYRADITFLNKKTSRAVDDRLFEKVQPGHHTRLMEGILDALEQDEFCKFEIQFELAHNAIHYLVGGRHTYSMSHLEYTSYDPLFFLHHFNTDRIFAIWQRLQQLRGKDPNSADCAHNLIHTPMEPFDRDTNPLDLTREHAKPADSFDYGRLGYQYDDLSLNGMSPEELNVYLGERAAKERTFASFILSGFGGSANVVVYVCRPAHDEIADDQCIKAGDFFLLGGPTEMKWGFYRAYHFDVTDSVASIDDDGHGHYYVKSELFSVNGSALSNDILRQPTLVHRPAKGHFDKPPVPVAQANLAVRKNINDLTAEETYSLRKAMERFQNDSVDGYQATVEFHALPARCPRPDAKDRFACCVHGMATFPHWHRLFVTQVEDALLRRGSTIGLPYWDWTMPMDHLPELAMSETYPHPVSSAIMANPFYHAEVAFENNVTHRDTDNRLFEKPSFGHHTHLFDAMVYAFEQEDFCDFEVQFELVHNNIHAWVGGSEKYSMSSLHYTAFDPIFYLHHSNVDRLWAIWQALQIRRNKPYKAHCAWSQEREPLKPFSFSSPLNNNDKTYQNSVPTNVYDYEGVLDYRYDDLQFGGMTLSELEKYIHKQKQHDRSFAGFFLSHIGTSANVDVLISAKGQEAHTIGSFAVLGGSKEMKWAFDRMYKYEITDAAKKLNIDVNDATISVKITDVDGTSLSPELIPSPALMFEPGHIVAKDFGHSKKIRKNVHSLTAEEQNSLRRAMADLQDDKTRGGFQQIAAFHGEPKWCPRPEAEKKFACCVHGMAVFPHWHRLLTVQGENALRKHGFTGGLPYWDWTRPMSALPHFVADPTYNDSVSSLEEDNPWYHGHIDSVGHDTTRAVRDDLYQSPGFGHYTDIAKQVLLAFEQDDFCDFEVQFEIAHNFIHALVGGNEPYSMSSLRYTTYDPIFFLHHSNTDRLWAIWQALQKYRGKPYNTANCAIASMRKPLQPFGLDSVINPDDETREHSVPFRVFDYKNNFDYEYESLAFNGLSIAQLDRELQRRKSHDRVFAGFLLHEIGQSALVKFYVCKHNVSDCDHYAGEFYILGDEAEMPWRYDRVYKYEITQQLHDLDLHVGDNFFLKYEAFDLNGGSLGGSIFSQPSVIFEPTAGSHQADEYREAVTSASHIRKNIRDLSEGEIESIRSAFLQIQKEGIYENIAKFHGKPGLCEYDGHPVACCVHGMPTFPHWHRLYVLQVENALLERGSAVAVPYWDWTEKADSLPSLINDATYFNSRSQTFDPNPFFRGHIAFENAVTSRDPQPELWDNKDFYENVMLALEQDNFCDFEIQLELIHNALHSRLGGRAKYSLSSLDYTAFDPVFFLHHANVDRIWAIWQDLQRYRKKPYNEADCAVNEMRKPLQPFNNPELNSDSMTLKHNLPQDSFDYQNRFRYQYDNLQFNHFSIQKLDQTIQARKQHDRVFAGFILHNIGTSVVVDIYICVEQGGEQNCKTKAGSFTILGGETEMPFHFDRLYKFDITSALHKLGVPLDGHGFDIKVDVRAVNGSHLDPHILNEPSLLFVPGEHSHQTADGLSQHNLVRKEVSSLTTLEKHFLRKALKNMQADDSPDGYQAIASFHALPPLCPSPSAAHRHACCLHGMATFPQWHRLYTVQFEDSLKRHGSIVGLPYWDWLKPQSALPDLVTQETYEHLFSHKTFPNPFLKANIEFEGEGVTTERDVDAEHLFAKGNLVYNNWFYNQALYALEQENYCDFEIQFEILHNGIHSWVGGSKTHSIGHLHYASYDPLFYIHHSQTDRIWAIWQAFQEHRGLSGKEAHCALEQMKDPLKPFSFGSPYNLNKRTQEFSKPEDTFDYHRFGYEYDSLEFVGMSVSSLHNYIKQQQEADRVFAGFLLKGFGQSASVSFDICRPDQSCQEAGYFSVLGGSSEMPWQFDRLYKYDITKTLKDMKLRYDDAFTIKVHIKDIAGAELDSDLIPTPSVLFEEGKHGINVRHVGRNRIRMELSELTERDLASLKSAMRSLQADDGVNGYQAIASFHGLPASCHDDEGHEIACCIHGMPVFPHWHRLYTLQMDMALLSHGSAVAIPYWDWTKPISKLPDLFTSPEYYDPWRDAVVNNPFAKGYIKSEDAYTVRDPQDILYHLQDETGTSVLLDQTLLALEQTDFCDFEVQFEVVHNAIHYLVGGRQVYALSSLHYASYDPAFFIHHSFVDKIWAVWQALQKKRKRPYHKADCALNMMTKPMRPFAHDFNHNGFTKMHAVPNTLFDFQDLFYTYDNLEIAGMNVNQLEAEINRRKSQTRVFAGFLLHGIGRSADVRFWICKTADDCHASGMIFILGGSKEMHWAYDRNFKYDITQALKAQSIHPEDVFDTDAPFFIKVEIHGVNKTALPSSAIPAPTIIYSAGEGHTDDHGSDHIAGSGVRKDVTSLTASEIENLRHALQSVMDDDGPNGFQAIAAYHGSPPMCHMPDGRDVACCTHGMASFPHWHRLFVKQMEDALAAHGAHIGIPYWDWTSAFSHLPALVTDHEHNPFHHGHIAHRNVDTSRSPRDMLFNDPEHGSESFFYRQVLLALEQTDFCQFEVQFEITHNAIHSWTGGHTPYGMSSLEYTAYDPLFYLHHSNTDRIWAIWQALQKYRGFQYNAAHCDIQVLKQPLKPFSESRNPNPVTRANSRAVDSFDYERLNYQYDTLTFHGHSISELDAMLQERKKEERTFAAFLLHGFGASADVSFDVCTPDGHCAFAGTFAVLGGELEMPWSFERLFRYDITKVLKQMNLYYDSEFHFALKVVGTDGTELPSDSIKSPTIEHHQADHQGHGGEGGQHPEHTGFVRKDIDSLSLDEANDLKNALYKLQNDQSLSGYESIAGFHGYPYLCPDNGDDKYACCVHGMPVFPHWHRLLTIQFERALKEHGSHLGVPYWDWTKSMTALPAFFADSSNNNPFYKYHIMKAGHDTTRQPNDLLFNQPQVHGYDYLYYLALSTLEEDNYCDFEVQYENFHNAVHIWLGGTETYSMATLEYSAFDPVFMILHSGLDRLWIIWQKLQKLRKKPYNAAKCAGHMIDEPLHPFDYESVNQDGFTRANAKPTTVFDSHRFDYHYDNLDVRGKSIQEINAIIHDLRTQPRIFAGFVLSGIYTSANVKIYLIREGHDDEYVGTFAVLGGAKEMPWAYERIFKYDITDVAHKLKMHHDDTFNFRLEIQSYSGETLTSHLPEPLIIHRPANKDYDVLIIPLGSGRKLPPKVIVKRGTEIEFHPVDDTINRPVVDLGSYTALYNCVVPPFTYHGYELDHAYSLRDGHYYIAGPTKDLCASGNVRIHIHIEDE

SEQ ID NO: 10 - DNA, 10495 bp, Megathura crenulata
gatcaccagccatctggtttgatcatgtggaccatcttggctctcctcacggcgaccctcttgtttgagggagccttctcggtagacacagtggtgagaaagaatgtggacagtttgagcagtgacgaggttctagccctcgagaaggctctcgatgatctccagcaagacgacagcaaccagggttatcaggccatagcaggctaccatggtgttccaacaatgtgtgtggacaaacatgaaaaaaatgtggcctgttgtcttcacggtatgccatcctttcccctttggcatcgtctctacgttgtccagttggaaagagcgctgataaggaagaaggcaaccatcagcatcccctactgggattggactagtgagctcacccatcttccagaacttgtgtctcatccactgtttgtcgacactgaggggggcaaggctcatgacaattcttggtaccgagctgacatcactttcctcaacaagaagacaagtcgtgctgtagatgaccgtctttttgaaaaagtacagccaggacaccacactcgcctcatggagggaatccttgacgctctggaacaggacgagttttgcaaatttgagatccagtttgagctggctcacaacgccatacattatctagttgggggcagacacacatattccatgtctcacctcgagtacacgtcatacgatcctttgttctttttacatcacttcaacactgaccgtatctttgctatatggcaacgacttcaacagctcagaggcaaggatcccaacagtgctgactgtgcccataaccttattcacactccaatggaaccgttcgacagagacactaatccactggatctgacaagagagcacgccaaacctgctgatagctttgactacgggcgcctcggctaccagtatgacgacctctccttgaatggcatgagtccagaggaactcaacgtatatctaggagaacgagcagctaaggagcgtacatttgccagcttcatattatctggttttggtggctctgccaatgttgtagtctatgtctgccgacctgctcatgatgaaatcgctgatgatcaatgtatcaaagcaggagatttcttccttcttggcggaccaactgaaatgaagtggggattctacagggcataccactttgatgttactgacagcgttgcttctatagatgatgatggtcatgggcattattacgtgaagtccgagttattcagcgtcaacggaagcgctttgtcaaatgacattcttcgacaacccaccttagtgcaccgaccagctaaaggtcattttgataagcctccagttccagttgctcaagctaacctggctgtcagaaagaatatcaacgatctaactgcagaggaaacatacagcttgaggaaagctatggaaagattccagaatgactcagttgacgggtaccaagctacagtagaattccatgcccttccagcaagatgtccacgcccagatgctaaagatagatttgcatgctgcgttcatggcatggcaaccttcccccactggcatagactctttgtcactcaagttgaagatgctctacttcgtcgcgggtctactatcggtctaccttactgggactggaccatgccaatggatcatctgccagaactggctatgagtgagacctatccccatccagtttctagcgcaatcatggcaaatccattttaccatgccgaagtggcttttgaaaataacgtcacacacagagatacagacaatagactctttgagaaaccgtcttttggacaccacacgcatctgtttgacgctatggtctatgcttttgaacaagaagacttttgcgactttgaagttcagtttgaactcgttcacaataacatacatgcgtgggttggaggaagtgaaaagtactccatgtcttctcttcactacactgcttttgatcctattttctacctccatcactcaaatgttgatcgtctctgggccatttggcaagctcttcaaatcaggaggaacaaaccttacaaggcccattgtgcctggtcacaggaaagggaaccattaaagcctttttcattcagttcacctctcaacaacaacgataaaacctaccaaaactctgttccaactaatgtttacgactatgagggagttttggactatcgatatgatgacctccagtttggtggcatgaccctgtcagaacttgaaaaatatatacacaagcagaaacagcatgacagaagttttgcaggattcttcctttctcatattggaacatcagcaaacgtagacgtccttatcagtgcaaagggccaagaggcccacacgattggaagctttgcagtgcttggtggatctaaggaaatgaaatgggctttcgacagaatgtacaaatatgaaattactgatgctgccaaaaagctgaacattgatgtgaatgatgccactatttctgttaagatcactgatgttgatggaacttccttgtcaccagagctaattccgtctcctgctctcatgtttgaacctgggcatattgttgccaaagactttggccacagcaaaaaaatcaggaagaacgttcattccctgacagctgaagaacaaaattcgttgaggcgagctatggcagatctccaagatgacaaaacacgaggtggattccaacagattgcagcatttcacggagaaccaaaatggtgcccacgccctgaagcggagaagaaatttgcatgctgcgttcacggaatggctgttttccctcactggcacagattgctgacagttcaaggagaaaatgctctgaggaaacatggatttactggtggattgccctattgggactggactcgaccaatgagcgcccttccacattttgttgctgatcctacttacaatgattctgtttccagcctcgaagaagataacccatggtatcatggtcacatagattctgttgggcatgatactacaagagctgtgcgtgatgatctttatcaatctcctggtttcggtcactacacagatattgcaaaacaagtccttctggcctttgagcaggacgatttctgtgattttgaggtacaatttgaaattgcccataatttcatacatgctctagttggtggtaacgaaccatacagtatgtcatctttgaggtatactacatacgatccaatcttcttcttgcaccactccaatacagaccgactttgggccatttggcaagctttgcaaaaataccgggggaaaccatacaacactgcaaactgtgccattgcatccatgagaaaaccacttcagccatttggtcttgatagtgtcataaatccagatgacgaaactcgtgaacattcggttcctttccgagtcttcgactacaagaacaacttcgactatgagtatgagagcctggcatttaatggtctgtctattgcccaactggaccgagagttgcagagaagaaagtcacatgacagagtctttgcaggattccttcttcatgaaattggacagtctgcactcgtgaaattctacgtttgcaaacacaatgtatctgactgtgaccattatgctggagaattctacattttgggagatgaagctgagatgccttggaggtatgaccgtgtgtacaagtacgagataacacagcagctgcacgatttagatctacatgttggagataatttctttcttaaatatgaagcctttgatctgaatggcggaagtcttggtggaagtatcttttctcagccttcggtgattttcgagccaactgcaggttcacaccaggctgatgaatatcgtgaggcagtaacaagcgctagccacataagaaaaaatatccgggacctctcagagggagaaattgagagcatcagatctgctttcctccaaattcaaaaagagggtatatatgaaaacattgcaaagttccatggaaaaccaggactttgtgaatatgatggacatcctgttgcttgttgtgtccatggcatgcccacctttccccactggcacagactgtacgttcttcaggtggagaatgcgctcttagaacgagggtctgcagttgctgttccttactgggactggaccgagaaagctgactctctgccatcattaatcaatgatgcaacttatttcaattcacgatcccagacctttgatcctaatcctttcttcaggggacatatcgccttcgagaatgctgtgacgtccagagatcctcagccagaactatgggacaataaggacttctacgagaatgtcatgctggctcttgagcaagacaacttctgtgactttgagattcagcttgagctgatacacaacgcccttcattctagacttggaggaagggctaaatactccctttcgtctcttgattataccgcatttgatcctgtatttttccttcaccatgcaaacgttgacagaatctgggccatctggcaggacttgcagagatatagaaagaaaccatacaatgaggctgactgcgcagtcaacgagatgcgtaaacctcttcaaccatttaataacccagaacttaacagtgattccatgacgcttaaacacaacctcccacaagacagttttgattatcaaaaccgcttcaggtaccaatatgataaccttcaatttaaccacttcagcatacaaaagctagaccaaactattcaggctagaaaacaacacgacagagtttttgctggctttattcttcacaacattgggacatctgttgttgtagatatttatatttgcgttgaacaaggaggagaacaaaactgcaagacaaaggcgggttccttcacgattctggggggagaaacagaaatgccattccactttgaccgcttgtacaaatttgacataacgtctgctctgcataaacttggtgttcccttggacggacatggattcgacatcaaagttgacgtcagagctgtcaatggatcgcatcttgatccacacatcctcaacgaaccgagtctgctttttgttcctggtgaacattcacatcaaactgcagatgggctttcacaacataatcttgtgcgaaaagaagtaagctctcttacaacactggagaaacattttttgaggaaagctctcaagaacatgcaagcagatgattctccagacggatatcaagctattgcttctttccacgctttgcctcctctttgtccaagtccatctgctgcacatagacacgcttgttgcctccatggtatggctaccttccctcagtggcacagactctacacagttcagttcgaagattctttgaaacgacatggttctattgtcggacttccatattgggattggctgaaaccgcagtctgcactccctgatttggtgacacaggagacatacgagcacctgttttcacacaaaaccttcccaaatccgttcctcaaggcaaatatagaatttgagggagagggagtaacaacagagagggatgttgatgctgaacacctctttgcaaaaggaaatctggtttacaacaactggttttacaatcaggcactatatgcactagaacaagaaaattactgtgactttgaaatacagttcgaaattttgcataatggaattcattcatgggttggaggatcaaagacccattcaataggtcatcttcattacgcatcatacgatccactgttctatatccaccattcgcagacagatcgcatttgggctatctggcaagctttccaggagcacagaggtctttcagggaaggaagcacactgcgccctggagcaaatgaaagaccctctcaaacctttcagctttggaagtccctataatttgaacaaacgcactcaagagttctccaagcctgaagacacatttgattatcaccgattcgggtatgagtatgattccctcgaatttgttggcatgtctgtttcaagtttacataactatataaaacaacaacaggaagctgatagagtcttcgcaggattccttcttaaaggatttggacaatcagcatccgtatcgtttgatatctgcagaccagaccagagttgccaagaagctggatacttctcagttctcggtggaagttcagaaatgccgtggcagtttgacaggctttacaagtacgacattacaaaaacgttgaaagacatgaaactgcgatacgatgacgcatttaccatcaaggttcacataaaggatatagctggagctgagttggacagcgatctgattccaactccttctgttctctttgaagaaggaaagcatgggatcaatgtacgtcacgttggtcgtaatcggattcgtatggaactatctgaactcaccgagagagatctcgccagcctgaaatctgcaatgaggtctctacaagctgacgatggggtgaacggttatcaagccattgcatcattccacggtctcccggcttcttgtcatgatgatgagggacatgagattgcctgttgtatccacggaatgccagtattcccacactggcacaggctttacaccctgcaaatggacatggctctgttatctcacggatctgctgttgctattccatactgggactggaccaaacctatcagcaaactgcctgatctcttcaccagccctgaatattacgatccttggagggatgcagttgtcaataatccatttgctaaaggctacattaaatccgaggacgcttacacggttagggatcctcaggacattttgtaccacttgcaggacgaaacgggaacatctgttttgttagatcaaactcttttagccttagaacagacagatttctgtgattttgaggttcaatttgaggtcgtccataatgctattcactacttggtgggtggtcgacaagtttatgctctttcttctctacactatgcttcatatgacccagccttctttattcatcactcctttgttgacaaaatatgggcagtctggcaagctctgcaaaagaagagaaagcgtccctatcataaagcggattgtgctcttaacatgatgaccaaaccaatgcgaccatttgcacacgatttcaatcacaatggattcacaaaaatgcacgcagtccccaacactctatttgactttcaggaccttttctacacgtatgacaacttagaaattgctggcatgaatgttaatcagttggaagcggaaatcaaccggcgaaaaagccaaacaagagtctttgccgggttccttctacatggcattggaagatcagctgatgtacgattttggatttgcaagacagctgacgactgccacgcatctggcatgatctttatcttaggaggttctaaagagatgcactgggcctatgacaggaactttaaatacgacatcacccaagctttgaaggctcagtccatacaccctgaagatgtgtttgacactgatgctcctttcttcattaaagtggagatccatggtgtaaacaagactgctctcccatcttcagctatcccagcacctactataatctactcagctggtgaaggtcataccgatgatcacggctcagatcatattgctggcagtggagtcaggaaagacgtgacgtctcttaccgcatctgagatagagaacctgaggcatgctctgcaaagcgtgatggatgatgatggacccaatggattccaggcaattgctgcttatcacggaagtcctcccatgtgtcacatgcctgatggtagagacgttgcatgttgtactcatggaatggcatctttccctcactggcacagactgtttgtgaaacagatggaggatgcactggctgcgcatggagctcacattggcataccatactgggattggacaagtgcgtttagtcatctgcctgccctagtgactgaccacgagcacaatcccttccaccacggacatattgctcatcggaatgtggatacatctcgatctccgagagacatgctgttcaatgaccctgaacacgggtcagaatcattcttctatagacaggttctcttggctctagaacagacagacttctgccaatttgaagttcagtttgaaataacacacaatgcaatccactcttggactggaggacatactccatatggaatgtcatcactggaatatacagcatatgatccactcttttatctccaccattccaacactgatcgtatctgggccatctggcaggcactccagaaatacagaggttttcaatacaacgcagctcattgcgatatccaggttctgaaacaacctcttaaaccattcagcgagtccaggaatccaaacccagtcaccagagccaattctagggcagtcgattcatttgattatgagagactcaattatcaatatgacacacttaccttccacggacattctatctcagaacttgatgccatgcttcaagagagaaagaaggaagagagaacatttgcagccttcctgttgcacggatttggcgccagtgctgatgtttcgtttgatgtctgcacacctgatggtcattgtgcctttgctggaaccttcgcggtacttggtggggagctggagatgccctggtcctttgaaagattgttccgttatgatatcacaaaggttctcaagcagatgaatctttactatgattctgagtttcatttcgcgttgaaggttgttggcacagatggaacagaactgccatcggatagtatcaagagcccaacaattgagcatcatcaagcagatcaccagggtcatggtggggagggtggccaacatcctgaacatactggatttgtaaggaaggacatagattctctttccttggatgaagccaatgatctgaaaaatgcactgtacaagctgcagaacgaccaaagcctcagtggatatgaatcaatagctggtttccatggctatccctacctctgtcctgacaacggcgatgacaagtatgcctgctgtgtccatggaatgcctgtatttccacactggcacagacttcttactatccagtttgagagagcactcaaagaacatggttcacatttgggtgttccctattgggattggaccaagtcaatgactgctcttccagctttctttgccgattcttccaataacaaccctttctacaaatatcacattatgaaagctggacatgatacaacgagacagccgaatgatcttctcttcaatcaaccacaggttcatggatatgactatctatactaccttgccctttcaacactagaagaggataactactgtgactttgaggttcagtatgaaaattttcacaatgcagtacatatctggctcgggggaactgaaacctactccatggctactcttgaatattcagcgtttgatccagtctttatgatcttgcattctggtctcgacagactctggatcatttggcaaaagctgcaaaagctcagaaagaaaccatataatgcagccaaatgtgctggccacatgattgatgaacctttgcatccttttgactacgagtctgttaaccaagatggcttcactcgagcaaacgctaaacctacaacagtgtttgacagtcacaggtttgattatcactatgacaacctggatgtacgaggaaagagtattcaggaaataaacgccatcatccacgatttgaggacccaaccaagaatctttgctggttttgttctttctggaatatacacttcagctaatgttaagatatacttaatcagagaaggccatgacgatgaatatgtaggaacatttgcagttttaggaggagccaaggagatgccatgggcctatgagaggattttcaagtatgacattacagatgtcgcccacaaactgaaaatgcaccatgatgatacattcaacttcagacttgaaattcaatcttacagtggcgaaacactaacatcacatcttcctgaacctctcataatccacagaccagccaacaaggactatgatgtcttgattattccattaggaagcggacgcaagcttcctcctaaagtcattgtcaagagaggaacagaaattgagtttcatccagtcgatgacacgatcaacagacctgttgtagatcttggcagctacactgctctctacaactgtgtggtgccacccttcacataccacggatatgagctcgatcatgcctactcccttagggatgggcactactatattgcaggcccaaccaaagacctctgcgccagcggaaatgttagaatacatatccacattgaagacgagtaactttacagaaataatcatcaacatcagactactttcatttgatggtcatgtccacttcaagatgcttcatccttccaactttgtcagtccaattgatctactgctgactaaccgtttatacttacattgaaacagctgtaactgcttttcagtattcaaaataaatgtttaattttttaaaaaaaaaaaaaaaaaaaaaaaaaaaaaa

SEQ ID NO: 11 - DNA, 10495 bp, Megathura crenulata
  SEQ ID NO11 is the complementary sequence of the sequence of SEQ ID NO10
ttttttttttttttttttttttttttttttaaaaaattaaacatttattttgaatactgaaaagcagttacagctgtttcaatgtaagtataaacggttagtcagcagtagatcaattggactgacaaagttggaaggatgaagcatcttgaagtggacatgaccatcaaatgaaagtagtctgatgttgatgattatttctgtaaagttactcgtcttcaatgtggatatgtattctaacatttccgctggcgcagaggtctttggttgggcctgcaatatagtagtgcccatccctaagggagtaggcatgatcgagctcatatccgtggtatgtgaagggtggcaccacacagttgtagagagcagtgtagctgccaagatctacaacaggtctgttgatcgtgtcatcgactggatgaaactcaatttctgttcctctcttgacaatgactttaggaggaagcttgcgtccgcttcctaatggaataatcaagacatcatagtccttgttggctggtctgtggattatgagaggttcaggaagatgtgatgttagtgtttcgccactgtaagattgaatttcaagtctgaagttgaatgtatcatcatggtgcattttcagtttgtgggcgacatctgtaatgtcatacttgaaaatcctctcataggcccatggcatctccttggctcctcctaaaactgcaaatgttcctacatattcatcgtcatggccttctctgattaagtatatcttaacattagctgaagtgtatattccagaaagaacaaaaccagcaaagattcttggttgggtcctcaaatcgtggatgatggcgtttatttcctgaatactctttcctcgtacatccaggttgtcatagtgataatcaaacctgtgactgtcaaacactgttgtaggtttagcgtttgctcgagtgaagccatcttggttaacagactcgtagtcaaaaggatgcaaaggttcatcaatcatgtggccagcacatttggctgcattatatggtttctttctgagcttttgcagcttttgccaaatgatccagagtctgtcgagaccagaatgcaagatcataaagactggatcaaacgctgaatattcaagagtagccatggagtaggtttcagttcccccgagccagatatgtactgcattgtgaaaattttcatactgaacctcaaagtcacagtagttatcctcttctagtgttgaaagggcaaggtagtatagatagtcatatccatgaacctgtggttgattgaagagaagatcattcggctgtctcgttgtatcatgtccagctttcataatgtgatatttgtagaaagggttgttattggaagaatcggcaaagaaagctggaagagcagtcattgacttggtccaatcccaatagggaacacccaaatgtgaaccatgttctttgagtgctctctcaaactggatagtaagaagtctgtgccagtgtggaaatacaggcattccatggacacagcaggcatacttgtcatcgccgttgtcaggacagaggtagggatagccatggaaaccagctattgattcatatccactgaggctttggtcgttctgcagcttgtacagtgcatttttcagatcattggcttcatccaaggaaagagaatctatgtccttccttacaaatccagtatgttcaggatgttggccaccctccccaccatgaccctggtgatctgcttgatgatgctcaattgttgggctcttgatactatccgatggcagttctgttccatctgtgccaacaaccttcaacgcgaaatgaaactcagaatcatagtaaagattcatctgcttgagaacctttgtgatatcataacggaacaatctttcaaaggaccagggcatctccagctccccaccaagtaccgcgaaggttccagcaaaggcacaatgaccatcaggtgtgcagacatcaaacgaaacatcagcactggcgccaaatccgtgcaacaggaaggctgcaaatgttctctcttccttctttctctcttgaagcatggcatcaagttctgagatagaatgtccgtggaaggtaagtgtgtcatattgataattgagtctctcataatcaaatgaatcgactgccctagaattggctctggtgactgggtttggattcctggactcgctgaatggtttaagaggttgtttcagaacctggatatcgcaatgagctgcgttgtattgaaaacctctgtatttctggagtgcctgccagatggcccagatacgatcagtgttggaatggtggagataaaagagtggatcatatgctgtatattccagtgatgacattccatatggagtatgtcctccagtccaagagtggattgcattgtgtgttatttcaaactgaacttcaaattggcagaagtctgtctgttctagagccaagagaacctgtctatagaagaatgattctgacccgtgttcagggtcattgaacagcatgtctctcggagatcgagatgtatccacattccgatgagcaatatgtccgtggtggaagggattgtgctcgtggtcagtcactagggcaggcagatgactaaacgcacttgtccaatcccagtatggtatgccaatgtgagctccatgcgcagccagtgcatcctccatctgtttcacaaacagtctgtgccagtgagggaaagatgccattccatgagtacaacatgcaacgtctctaccatcaggcatgtgacacatgggaggacttccgtgataagcagcaattgcctggaatccattgggtccatcatcatccatcacgctttgcagagcatgcctcaggttctctatctcagatgcggtaagagacgtcacgtctttcctgactccactgccagcaatatgatctgagccgtgatcatcggtatgaccttcaccagctgagtagattatagtaggtgctgggatagctgaagatgggagagcagtcttgtttacaccatggatctccactttaatgaagaaaggagcatcagtgtcaaacacatcttcagggtgtatggactgagccttcaaagcttgggtgatgtcgtatttaaagttcctgtcataggcccagtgcatctctttagaacctcctaagataaagatcatgccagatgcgtggcagtcgtcagctgtcttgcaaatccaaaatcgtacatcagctgatcttccaatgccatgtagaaggaacccggcaaagactcttgtttggctttttcgccggttgatttccgcttccaactgattaacattcatgccagcaatttctaagttgtcatacgtgtagaaaaggtcctgaaagtcaaatagagtgttggggactgcgtgcatttttgtgaatccattgtgattgaaatcgtgtgcaaatggtcgcattggtttggtcatcatgttaagagcacaatccgctttatgatagggacgctttctcttcttttgcagagcttgccagactgcccatattttgtcaacaaaggagtgatgaataaagaaggctgggtcatatgaagcatagtgtagagaagaaagagcataaacttgtcgaccacccaccaagtagtgaatagcattatggacgacctcaaattgaacctcaaaatcacagaaatctgtctgttctaaggctaaaagagtttgatctaacaaaacagatgttcccgtttcgtcctgcaagtggtacaaaatgtcctgaggatccctaaccgtgtaagcgtcctcggatttaatgtagcctttagcaaatggattattgacaactgcatccctccaaggatcgtaatattcagggctggtgaagagatcaggcagtttgctgataggtttggtccagtcccagtatggaatagcaacagcagatccgtgagataacagagccatgtccatttgcagggtgtaaagcctgtgccagtgtgggaatactggcattccgtggatacaacaggcaatctcatgtccctcatcatcatgacaagaagccgggagaccgtggaatgatgcaatggcttgataaccgttcaccccatcgtcagcttgtagagacctcattgcagatttcaggctggcgagatctctctcggtgagttcagatagttccatacgaatccgattacgaccaacgtgacgtacattgatcccatgctttccttcttcaaagagaacagaaggagttggaatcagatcgctgtccaactcagctccagctatatcctttatgtgaaccttgatggtaaatgcgtcatcgtatcgcagtttcatgtctttcaacgtttttgtaatgtcgtacttgtaaagcctgtcaaactgccacggcatttctgaacttccaccgagaactgagaagtatccagcttcttggcaactctggtctggtctgcagatatcaaacgatacggatgctgattgtccaaatcctttaagaaggaatcctgcgaagactctatcagcttcctgttgttgttttatatagttatgtaaacttgaaacagacatgccaacaaattcgagggaatcatactcatacccgaatcggtgataatcaaatgtgtcttcaggcttggagaactcttgagtgcgtttgttcaaattatagggacttccaaagctgaaaggtttgagagggtctttcatttgctccagggcgcagtgtgcttccttccctgaaagacctctgtgctcctggaaagcttgccagatagcccaaatgcgatctgtctgcgaatggtggatatagaacagtggatcgtatgatgcgtaatgaagatgacctattgaatgggtctttgatcctccaacccatgaatgaattccattatgcaaaatttcgaactgtatttcaaagtcacagtaattttcttgttctagtgcatatagtgcctgattgtaaaaccagttgttgtaaaccagatttccttttgcaaagaggtgttcagcatcaacatccctctctgttgttactccctctccctcaaattctatatttgccttgaggaacggatttgggaaggttttgtgtgaaaacaggtgctcgtatgtctcctgtgtcaccaaatcagggagtgcagactgcggtttcagccaatcccaatatggaagtccgacaatagaaccatgtcgtttcaaagaatcttcgaactgaactgtgtagagtctgtgccactgagggaaggtagccataccatggaggcaacaagcgtgtctatgtgcagcagatggacttggacaaagaggaggcaaagcgtggaaagaagcaatagcttgatatccgtctggagaatcatctgcttgcatgttcttgagagctttcctcaaaaaatgtttctccagtgttgtaagagagcttacttcttttcgcacaagattatgttgtgaaagcccatctgcagtttgatgtgaatgttcaccaggaacaaaaagcagactcggttcgttgaggatgtgtggatcaagatgcgatccattgacagctctgacgtcaactttgatgtcgaatccatgtccgtccaagggaacaccaagtttatgcagagcagacgttatgtcaaatttgtacaagcggtcaaagtggaatggcatttctgtttctccccccagaatcgtgaaggaacccgcctttgtcttgcagttttgttctcctccttgttcaacgcaaatataaatatctacaacaacagatgtcccaatgttgtgaagaataaagccagcaaaaactctgtcgtgttgttttctagcctgaatagtttggtctagcttttgtatgctgaagtggttaaattgaaggttatcatattggtacctgaagcggttttgataatcaaaactgtcttgtgggaggttgtgtttaagcgtcatggaatcactgttaagttctgggttattaaatggttgaagaggtttacgcatctcgttgactgcgcagtcagcctcattgtatggtttctttctatatctctgcaagtcctgccagatggcccagattctgtcaacgtttgcatggtgaaggaaaaatacaggatcaaatgcggtataatcaagagacgaaagggagtatttagcccttcctccaagtctagaatgaagggcgttgtgtatcagctcaagctgaatctcaaagtcacagaagttgtcttgctcaagagccagcatgacattctcgtagaagtccttattgtcccatagttctggctgaggatctctggacgtcacagcattctcgaaggcgatatgtcccctgaagaaaggattaggatcaaaggtctgggatcgtgaattgaaataagttgcatcattgattaatgatggcagagagtcagctttctcggtccagtcccagtaaggaacagcaactgcagaccctcgttctaagagcgcattctccacctgaagaacgtacagtctgtgccagtggggaaaggtgggcatgccatggacacaacaagcaacaggatgtccatcatattcacaaagtcctggttttccatggaactttgcaatgttttcatatataccctctttttgaatttggaggaaagcagatctgatgctctcaatttctccctctgagaggtcccggatattttttcttatgtggctagcgcttgttactgcctcacgatattcatcagcctggtgtgaacctgcagttggctcgaaaatcaccgaaggctgagaaaagatacttccaccaagacttccgccattcagatcaaaggcttcatatttaagaaagaaattatctccaacatgtagatctaaatcgtgcagctgctgtgttatctcgtacttgtacacacggtcatacctccaaggcatctcagcttcatctcccaaaatgtagaattctccagcataatggtcacagtcagatacattgtgtttgcaaacgtagaatttcacgagtgcagactgtccaatttcatgaagaaggaatcctgcaaagactctgtcatgtgactttcttctctgcaactctcggtccagttgggcaatagacagaccattaaatgccaggctctcatactcatagtcgaagttgttcttgtagtcgaagactcggaaaggaaccgaatgttcacgagtttcgtcatctggatttatgacactatcaagaccaaatggctgaagtggttttctcatggatgcaatggcacagtttgcagtgttgtatggtttcccccggtatttttgcaaagcttgccaaatggcccaaagtcggtctgtattggagtggtgcaagaagaagattggatcgtatgtagtatacctcaaagatgacatactgtatggttcgttaccaccaactagagcatgtatgaaattatgggcaatttcaaattgtacctcaaaatcacagaaatcgtcctgctcaaaggccagaaggacttgttttgcaatatctgtgtagtgaccgaaaccaggagattgataaagatcatcacgcacagctcttgtagtatcatgcccaacagaatctatgtgaccatgataccatgggttatcttcttcgaggctggaaacagaatcattgtaagtaggatcagcaacaaaatgtggaagggcgctcattggtcgagtccagtcccaatagggcaatccaccagtaaatccatgtttcctcagagcattttctccttgaactgtcagcaatctgtgccagtgagggaaaacagccattccgtgaacgcagcatgcaaatttcttctccgcttcagggcgtgggcaccattttggttctccgtgaaatgctgcaatctgttggaatccacctcgtgttttgtcatcttggagatctgccatagctcgcctcaacgaattttgttcttcagctgtcagggaatgaacgttcttcctgatttttttgctgtggccaaagtctttggcaacaatatgcccaggttcaaacatgagagcaggagacggaattagctctggtgacaaggaagttccatcaacatcagtgatcttaacagaaatagtggcatcattcacatcaatgttcagctttttggcagcatcagtaatttcatatttgtacattctgtcgaaagcccatttcatttccttagatccaccaagcactgcaaagcttccaatcgtgtgggcctcttggccctttgcactgataaggacgtctacgtttgctgatgttccaatatgagaaaggaagaatcctgcaaaacttctgtcatgctgtttctgcttgtgtatatatttttcaagttctgacagggtcatgccaccaaactggaggtcatcatatcgatagtccaaaactccctcatagtcgtaaacattagttggaacagagttttggtaggttttatcgttgttgttgagaggtgaactgaatgaaaaaggctttaatggttccctttcctgtgaccaggcacaatgggccttgtaaggtttgttcctcctgatttgaagagcttgccaaatggcccagagacgatcaacatttgagtgatggaggtagaaaataggatcaaaagcagtgtagtgaagagaagacatggagtacttttcacttcctccaacccacgcatgtatgttattgtgaacgagttcaaactgaacttcaaagtcgcaaaagtcttcttgttcaaaagcatagaccatagcgtcaaacagatgcgtgtggtgtccaaaagacggtttctcaaagagtctattgtctgtatctctgtgtgtgacgttattttcaaaagccacttcggcatggtaaaatggatttgccatgattgcgctagaaactggatggggataggtctcactcatagccagttctggcagatgatccattggcatggtccagtcccagtaaggtagaccgatagtagacccgcgacgaagtagagcatcttcaacttgagtgacaaagagtctatgccagtgggggaaggttgccatgccatgaacgcagcatgcaaatctatctttagcatctgggcgtggacatcttgctggaagggcatggaattctactgtagcttggtacccgtcaactgagtcattctggaatctttccatagctttcctcaagctgtatgtttcctctgcagttagatcgttgatattctttctgacagccaggttagcttgagcaactggaactggaggcttatcaaaatgacctttagctggtcggtgcactaaggtgggttgtcgaagaatgtcatttgacaaagcgcttccgttgacgctgaataactcggacttcacgtaataatgcccatgaccatcatcatctatagaagcaacgctgtcagtaacatcaaagtggtatgccctgtagaatccccacttcatttcagttggtccgccaagaaggaagaaatctcctgctttgatacattgatcatcagcgatttcatcatgagcaggtcggcagacatagactacaacattggcagagccaccaaaaccagataatatgaagctggcaaatgtacgctccttagctgctcgttctcctagatatacgttgagttcctctggactcatgccattcaaggagaggtcgtcatactggtagccgaggcgcccgtagtcaaagctatcagcaggtttggcgtgctctcttgtcagatccagtggattagtgtctctgtcgaacggttccattggagtgtgaataaggttatgggcacagtcagcactgttgggatccttgcctctgagctgttgaagtcgttgccatatagcaaagatacggtcagtgttgaagtgatgtaaaaagaacaaaggatcgtatgacgtgtactcgaggtgagacatggaatatgtgtgtctgcccccaactagataatgtatggcgttgtgagccagctcaaactggatctcaaatttgcaaaactcgtcctgttccagagcgtcaaggattccctccatgaggcgagtgtggtgtcctggctgtactttttcaaaaagacggtcatctacagcacgacttgtcttcttgttgaggaaagtgatgtcagctcggtaccaagaattgtcatgagccttgcccccctcagtgtcgacaaacagtggatgagacacaagttctggaagatgggtgagctcactagtccaatcccagtaggggatgctgatggttgccttcttccttatcagcgctctttccaactggacaacgtagagacgatgccaaaggggaaaggatggcataccgtgaagacaacaggccacatttttttcatgtttgtccacacacattgttggaacaccatggtagcctgctatggcctgataaccctggttgctgtcgtcttgctggagatcatcgagagccttctcgagggctagaacctcgtcactgctcaaactgtccacattctttctcaccactgtgtctaccgagaaggctccctcaaacaagagggtcgccgtgaggagagccaagatggtccacatgatcaaaccagatggctggtgatc

SEQ ID NO: 12 - protein, 3420 aa, Megathura crenulata; identical to SEQ ID NO: 9
  SEQ ID NO12 is the deduced amino acid sequence of the coding region of SEQ ID NO10

SEQ ID NO: 13 - DNA, 15 bp, Artificial
  Primer 12 klh2cSns-lessdeg71
ttyccwcaytggcay

SEQ ID NO: 14 - DNA, 15 bp, Artificial
  Primer 11 klh2cSns-deg71
ttyccncaytggcay

SEQ ID NO: 15 - DNA, 15 bp, Artificial
  Primer 30 klh2cAnti-lessdeg223
ttgccaaatdggcca

SEQ ID NO: 16 - DNA, 16 bp, Artificial
  Primer 15 klh2cSns-deg96
cntaytgggaytggac

SEQ ID NO: 17 - DNA, 17 bp, Artificial
  Primer 53 Klh2cAnti-deg354
gggatytcngcytcrtc

SEQ ID NO: 18 - DNA, 31 bp, Artificial
  5′ RACE Primer 1
ccatagcgtcaaacagatgcgtgtggtgtcc

SEQ ID NO: 19 - DNA, 28 bp, Artificial
  5′ RACE Primer 2
gcaaagcttgccaaatggcccaaagtcg

SEQ ID NO: 20 - DNA, 23 bp, Artificial
  3′ RACE Primer 1
gagcgcccttccacattttgttg

SEQ ID NO: 21 - DNA, 23 bp, Artificial
  3′ RACE Primer 2
agcgcccttccacattttgttgc

SEQ ID NO: 22 - DNA, 10197 bp, Megathura crenulata
gagaacctggtcagaaagagtgtggagcatctgactcaggaagagaccctggacctgcaggctgccctgcgtgagctgcagatggactcatccagcattggtttccagaaaatagctgctgctcatggcgcgcccgcatcttgtgtgcataaggacacttctatagcttgctgtattcacggcatgcctactttcccgcactggcacagggcgtacgtagttcacatggagcgagctctgcagacgaagaggcgtacttctgggatgccatactgggactggacagaacccatcacccagttgccctctctcgctgcggaccctgtttacatcgattcccagggcggaaaggctcacaccaactactggtacagagggaacattgactttctagacaagaaaaccaatcgtgcggtcgacgaccgtctttttgagaaggtgaagccaggccagcacacccatcttatggagagtgttttggacgcactggagcaggatgaattctgtaaattcgaaatccagttcgagttagcacataatgcgattcactacctggtcggtggaaagcatgattactctatggcaaacctcgagtacaccgcctacgatccgatcttctttcttcaccactccaacgttgacagaattttcgccatctggcagaggctccaagagctccgaaacaaagaccccaaagctatggattgtgcccaagaattgttacatcagaaaatggaacctttcagttgggaggacaatgacataccactaacaaatgaacattccactccagccgatctctttgactatcgtcaacttcactacgactatgacacgttgaatcttaatggtatgactccagaggaactgaaaacctatttggatgagcggtcttcaagggcaagagccttcgcaagcttccgtctgaaaggatttggaggttcagccaatgtctttgtgtacgtctgcatccccgacgacaacgatcgtaatgatgaccactgtgagaaggccggtgatttcttcgttctgggcggaccaagcgagatgaaatggcagttctacaggccatacttgtttgacttgtccgacactgttcacaagatgggaatgaagttggatggacactataccgtcaaggcagagcttttcagtgtcaatggtacagctcttcccgatgatttgttacctcatccagttgttgttcatcatccggagaagggtttcactgatcctccggtgaagcatcaccaaagcgccaatctcctcgttagaaagaacatcaatgatctcacccgtgaagaagtgttgaatctgagagaggcgtttcataaattccaagaagatcgctccgtcgacggttatcaagccacagctgagtatcatggtcttccggccagatgccctcgccctgatgccaaggacagatatgcttgttgtgtccatggaatgccaatcttccctcactggcacaggctttttgtcacacaagttgaagatgctttagtaggccgtggagctaccattggtatcccatactgggactggactgaacccatgacacacattccgggtctggcaggaaacgaaacttatgtggattctcatggtgcatcccacacaaatccttttcatagttcagtgattgcatttgaagaaaatgctccccacaccaaaagacaaatagatcaaagactctttaaacccgctacctttggacaccacacagacctgttcaaccagattttgtatgcctttgaacaagaagattactgtgactttgaagtccaatttgagattacccataacacgattcacgcttggacaggaggaagcgaacatttctcaatgtcgtccctacattacacagctttcgatcctttgttttactttcaccattctaacgttgatcgtctttgggccgtttggcaagccttacagatgagacggcataaaccctacagggcccactgcgccatatctctggaacatatgcatctgaaaccattcgccttttcatctccccttaacaataacgaaaagactcatgccaatgccatgccaaacaagatctacgactatgaaaatgtcctccattacacatacgaagatttaacatttggaggcatctctctggaaaacatagaaaagatgatccacgaaaaccagcaagaagacagaatatatgccggttttctcctggctggcatacgtacttcagcaaatgttgatatcttcattaaaactaccgattccgtgcaacataaggctggaacatttgcagtgctcggtggaagcaaggaaatgaagtggggatttgatcgcgttttcaagtttgacatcacgcacgttttgaaagatctcgatctcactgctgatggcgatttcgaagttactgttgacatcactgaagtcgatggaactaaacttgcatccagtcttattccacatgcttctgtcattcgtgagcatgcacgtgttaaatttgacaaagtgccaaggagtcgtcttattcgaaaaaatgtagaccgtttgagccccgaggagatgaatgaacttcgtaaagccctagccttactgaaagaggacaaaagtgccggtggatttcagcagcttggtgcattccatggggagccaaaatggtgtcctagtcccgaagcatctaaaaaatttgcctgctgtgttcacggcatgtctgtgttccctcactggcatcgactgttgacggttcagagtgaaaatgctttgagacgacatggctacgatggagctttgccgtactgggattggacctctcctcttaatcaccttcccgaactggcagatcatgagaagtacgtcgaccctgaagatggggtagagaagcataacccttggttcgatggtcatatagatacagtcgacaaaacaacaacaagaagtgttcagaataaactcttcgaacagcctgagtttggtcattatacaagcattgccaaacaagtactgctagcgttggaacaggacaatttctgtgactttgaaatccaatatgagattgcccataactacatccatgcacttgtaggaggcgctcagccttatggtatggcatcgcttcgctacactgcttttgatccactattctacttgcatcactctaatacagatcgtatatgggcaatatggcaggctttacagaagtacagaggaaaaccgtacaacgttgctaactgtgctgttacatcgatgagagaacctttgcaaccatttggcctctctgccaatatcaacacagaccatgtaaccaaggagcattcagtgccattcaacgtttttgattacaagaccaatttcaattatgaatatgacactttggaatttaacggtctctcaatctctcagttgaataaaaagctcgaagcgataaagagccaagacaggttctttgcaggcttcctgttatctggtttcaagaaatcatctcttgttaaattcaatatttgcaccgatagcagcaactgtcaccccgctggagagttttaccttctgggtgatgaaaacgagatgccatgggcatacgatagagtcttcaaatatgacataaccgaaaaactccacgatctaaagctgcatgcagaagaccacttctacattgactatgaagtatttgaccttaaaccagcaagcctgggaaaagatttgttcaagcagccttcagtcattcatgaaccaagaataggtcaccatgaaggcgaagtatatcaagctgaagtaacttctgccaaccgtattcgaaaaaacattgaaaatctgagccttggtgaactcgaaagtctgagagctgccttcctggaaattgaaaacgatggaacttacgaatcaatagctaaattccatggtagccctggtttgtgccagttaaatggtaaccccatctcttgttgtgtccatggcatgccaactttccctcactggcacagactgtacgtggttgtcgttgagaatgccctcctgaaaaaaggatcatctgtagctgttccctattgggactggacaaaacgaatcgaacatttacctcacctgatttcagacgccacttactacaattccaggcaacatcactatgagacaaacccattccatcatggcaaaatcacacacgagaatgaaatcactactagggatcccaaggacagcctcttccattcagactacttttacgagcaggtcctttacgccttggagcaggataacttctgtgatttcgagattcagttggagatattacacaatgcattgcattctttacttggtggcaaaggtaaatattccatgtcaaaccttgattacgctgcttttgatcctgtgttcttccttcatcacgcaacgactgacagaatctgggcaatctggcaagaccttcagaggttccgaaaacggccataccgagaagcgaattgcgctatccaattgatgcacacgccactccagccgtttgataagagcgacaacaatgacgaggcaacgaaaacgcatgccactccacatgatggttttgaatatcaaaacagctttggttatgcttacgataatctggaactgaatcactactcgattcctcagcttgatcacatgctgcaagaaagaaaaaggcatgacagagtattcgctggcttcctccttcacaatattggaacatctgccgatgtccatgtatttgtatgtctcccaactggggaacacacgaaggactgcagtcatgaggctggtatgttctccatcttaggcggtcaaacggagatgtcctttgtatttgacagactttacaaacttgacataactaaagccttgaaaaagaacggtgtgcacctgcaaggggatttcgatctggaaattgagattacggctgtgaatggatctcatctagacagtcatgtcatccactctcccactatactgtttgaggccggaacagattctgcccacacagatgatggacacactgaaccagtgatgattcgcaaagatatcacacaattggacaagcgtcaacaactgtcactggtgaaagccctcgagtccatgaaagccgaccattcatctgatgggttccaggcaatcgcttccttccatgctcttcctcctctttgtccatcaccagctgcttcaaagaggtttgcgtgctgcgtccatggcatggcaacgttcccacaatggcaccgtctgtacacagtccaattccaagattctctcagaaaacatggtgcagtcgttggacttccgtactgggactggaccctacctcgttctgaattaccagagctcctgaccgtctcaactattcatgacccggagacaggcagagatataccaaatccatttattggttctaaaatagagtttgaaggagaaaacgtacatactaaaagagatatcaatagggatcgtctcttccagggatcaacaaaaacacatcataactggtttattgagcaagcactgcttgctcttgaacaaaccaactactgcgacttcgaggttcagtttgaaattatgcataatggtgttcatacctgggttggaggcaaggagccctatggaattggccatctgcattatgcttcctatgatccacttttctacatccatcactcccaaactgatcgtatttgggctatatggcaatcgttgcagcgtttcagaggactttctggatctgaggctaactgtgctgtaaatctcatgaaaactcctctgaagcctttcagctttggagcaccatataatcttaatgatcacacgcatgatttctcaaagcctgaagatacattcgactaccaaaagtttggatacatatatgacactctggaatttgcagggtggtcaattcgtggcattgaccatattgtccgtaacaggcaggaacattcaagggtctttgccggattcttgcttgaaggatttggcacctctgccactgtcgatttccaggtctgtcgcacagcgggagactgtgaagatgcagggtacttcaccgtgttgggaggtgaaaaagaaatgccttgggcctttgatcggctttacaagtacgacataacagaaaccttagacaagatgaaccttcgacatgacgaaatcttccagattgaagtaaccattacatcctacgatggaactgtactcgatagtggccttattcccacaccgtcaatcatctatgatcctgctcatcatgatattagttcgcaccacctgtcgctcaacaaggttcgtcatgatctgagtacactgagtgagcgagatattggaagccttaaatatgctttgagcagcttgcaggcagatacctcagcagatggttttgctgccattgcatccttccatggtctgcctgccaaatgtaatgacagccacaataacgaggtggcatgctgtatccatggaatgcctacattcccccactggcacagactctacaccctccaatttgagcaagctctaagaagacatggctctagtgtagcagtaccctactgggactggacaaagccaatacataatattccacatctgttcacagacaaagaatactacgatgtctggagaaataaagtaatgccaaatccatttgcccgagggtatgtcccctcacacgatacatacacggtaagagacgtccaagaaggcctgttccacctgacatcaacgggtgaacactcagcgcttctgaatcaagctcttttggcgctggaacagcacgactactgcgattttgcagtccagtttgaagtcatgcacaacacaatccattacctagtgggaggacctcaagtctattctttgtcatcccttcattatgcttcatatgatccgatcttcttcatacaccactcctttgtagacaaggtttgggctgtctggcaggctcttcaagaaaagagaggccttccatcagaccgtgctgactgcgctgttagtctcatgactcagaacatgaggcctttccatcacgaaattaaccataaccagttcaccaagaaacatgcagttccaaatgatgttttcaagtacgaactcctgggttacagatacgacaatctggaaatcggtggcatgaatttgcatgaaattgaaaaggaaatcaaagacaaacagcaccatgtgagagtgtttgcagggttcctccttcacggaattagaacctcagctgatgtccaattccagatttgtaaaacatcagaagattgtcaccatggaggccaaatcttcgttcttggggggactaaagagatggcctgggcttataaccgtttattcaagtacgatattacccatgctcttcatgacgcacacatcactccagaagacgtattccatccctctgaaccattcttcatcaaggtgtcagtgacagccgtcaacggaacagttcttccggcttcaatcctgcatgcaccaaccattatctatgaacctggtctcgaccatcacgaagatcatcattcttcttctatggctggacatggtgtcagaaaggaaatcaacacacttaccactgcagaggtggacaatctcaaagatgccatgagagccgtcatggcagaccacggtccaaatggataccaggctatagcagcgttccatggaaacccaccaatgtgccctatgccagatggaaagaattactcgtgttgtacacatggcatggctactttcccccactggcacagactgtacacaaaacagatggaagatgccttgaccgcccatggtgccagagtcggccttccttactgggactggacaactgcctttacagctttgccaacttttgtcacagatgaagaggacaatcctttccatcatggtcacatagactatttgggagtggatacaactcggtcgccccgagacaagttgttcaatgatccagagcgaggatcagaatcgttcttctacaggcaggttctcttggctttggagcagacagatttttgtcagtttgaagtccagttcgagattacccacaatgccgtccattcttggactggtggactgaccccctacggaatgtccactctggagtacacgacctatgatcctcttttctggcttcatcatgccaatactgaccgaatatgggctatttggcaagctcttcaagaatacagaggtctcccatacgaccatgccaactgcgaaatccaggcaatgaagaggcccctacggccattctctgatcctattaatcacaatgcgttcactcattctaacgcaaaaccaacagacgttttcgagtacagtagattcaacttccagtatgataacttacggtttcatggtatgacaataaaaaagttggagcatgagcttgagaaacagaaggaagaagacaggacattcgctgccttcctgctccacggcattaaaaaaagtgctgatgtaagctttgacgtgtgcaaccatgatggagaatgccactttgcaggtaccttcgctattcttggaggcgagcacgagatgccttggtcctttgacagacttttccgatatgacatcacccaagttcttaagcaaatgcacctggagtatgattctgatttcacgttccatatgagaattattgacacttcgggtaaacaactgccatcagacctgatcaagatgccaacggttgaacacagcccaggaggaaaacatcatgagaaacatcatgaagatcaccatgaagacatacttgttcgtaaaaacattcactctctgtcacaccatgaagccgaagaattgagagatgcattgtacaagcttcaaaacgatgaaagtcacggtggatatgaacatattgctggattccatggttatcctaacctctgtcctgaaaaaggggatgaaaagtatccttgctgtgttcatggtatgtctatattcccccactggcacaggctccacacgatacagtttgaaagagctctgaagaaacatggttcccatctcggtattccttactgggattggactcagacgatatcgtctcttcctaccttctttgcagattctggtaataataatccattcttcaaataccatatcagaagcataaatcaagacactgttcgagacgttaatgaagcgatattccaacaaacgaagtttggagaattttcgtccatattttacctggccttgcaggctctagaagaggataattattgcgattttgaggtgcagtacgagatacttcacaatgaggtgcatgccttgattggtggagccgagaagtattccatgtccacgctggaatattcagccttcgatccatacttcatgatccaccacgcgtctttggacaagatctggataatctggcaggagcttcagaaacgccgggttaagcctgcacatgctggttcgtgtgccggagacattatgcacgttccacttcatcctttcaattatgaaagcgtcaacaacgatgactttaccagagagaattctctccccaacgcggtcgttgacagtcatagattcaactataaatatgacaatcttaatcttcacggccacaatatagaggagcttgaggaggttctccgcagcctgagacttaaatctcgtgttttcgcaggatttgttctgtccggaatccgcacaactgcagttgtgaaagtgtatatcaaaagcggaaccgattccgacgacgaatatgccggttcgtttgtcatccttggaggagccaaagagatgccttgggcatatgagaggttgtatagatttgacattacagaaactgtacacaatcttaaccttacggatgatcatgttaaattcaggtttgacttaaagaaatatgatcatactgaattggatgcatcagtactgccagctcctataatcgttcgacgtccaaataatgcggtttttgatatcattgagattccaatagggaaagacgtgaatcttccaccgaaggtggtcgtcaagagaggcactaaaatcatgttcatgtctgttgatgaagccgttaccactcccatgctgaacctgggtagttatacagcaatgttcaaatgtaaagtgccaccattcagcttccatgcttttgagcttgggaaaatgtattctgtcgaatctggtgactatttcatgacggcatcaacaactgaattatgcaacgataataacttaaggatacatgtccatgtggatgacgaatag

SEQ ID NO: 23 - DNA, 10197 bp, Megathura crenulata
  SEQ ID NO23 is the complementary sequence of the coding sequence of SEQ ID NO22
ctattcgtcatccacatggacatgtatccttaagttattatcgttgcataattcagttgttgatgccgtcatgaaatagtcaccagattcgacagaatacattttcccaagctcaaaagcatggaagctgaatggtggcactttacatttgaacattgctgtataactacccaggttcagcatgggagtggtaacggcttcatcaacagacatgaacatgattttagtgcctctcttgacgaccaccttcggtggaagattcacgtctttccctattggaatctcaatgatatcaaaaaccgcattatttggacgtcgaacgattataggagctggcagtactgatgcatccaattcagtatgatcatatttctttaagtcaaacctgaatttaacatgatcatccgtaaggttaagattgtgtacagtttctgtaatgtcaaatctatacaacctctcatatgcccaaggcatctctttggctcctccaaggatgacaaacgaaccggcatattcgtcgtcggaatcggttccgcttttgatatacactttcacaactgcagttgtgcggattccggacagaacaaatcctgcgaaaacacgagatttaagtctcaggctgcggagaacctcctcaagctcctctatattgtggccgtgaagattaagattgtcatatttatagttgaatctatgactgtcaacgaccgcgttggggagagaattctctctggtaaagtcatcgttgttgacgctttcataattgaaaggatgaagtggaacgtgcataatgtctccggcacacgaaccagcatgtgcaggcttaacccggcgtttctgaagctcctgccagattatccagatcttgtccaaagacgcgtggtggatcatgaagtatggatcgaaggctgaatattccagcgtggacatggaatacttctcggctccaccaatcaaggcatgcacctcattgtgaagtatctcgtactgcacctcaaaatcgcaataattatcctcttctagagcctgcaaggccaggtaaaatatggacgaaaattctccaaacttcgtttgttggaatatcgcttcattaacgtctcgaacagtgtcttgatttatgcttctgatatggtatttgaagaatggattattattaccagaatctgcaaagaaggtaggaagagacgatatcgtctgagtccaatcccagtaaggaataccgagatgggaaccatgtttcttcagagctctttcaaactgtatcgtgtggagcctgtgccagtgggggaatatagacataccatgaacacagcaaggatacttttcatcccctttttcaggacagaggttaggataaccatggaatccagcaatatgttcatatccaccgtgactttcatcgttttgaagcttgtacaatgcatctctcaattcttcggcttcatggtgtgacagagagtgaatgtttttacgaacaagtatgtcttcatggtgatcttcatgatgtttctcatgatgttttcctcctgggctgtgttcaaccgttggcatcttgatcaggtctgatggcagttgtttacccgaagtgtcaataattctcatatggaacgtgaaatcagaatcatactccaggtgcatttgcttaagaacttgggtgatgtcatatcggaaaagtctgtcaaaggaccaaggcatctcgtgctcgcctccaagaatagcgaaggtacctgcaaagtggcattctccatcatggttgcacacgtcaaagcttacatcagcactttttttaatgccgtggagcaggaaggcagcgaatgtcctgtcttcttccttctgtttctcaagctcatgctccaacttttttattgtcataccatgaaaccgtaagttatcatactggaagttgaatctactgtactcgaaaacgtctgttggttttgcgttagaatgagtgaacgcattgtgattaataggatcagagaatggccgtaggggcctcttcattgcctggatttcgcagttggcatggtcgtatgggagacctctgtattcttgaagagcttgccaaatagcccatattcggtcagtattggcatgatgaagccagaaaagaggatcataggtcgtgtactccagagtggacattccgtagggggtcagtccaccagtccaagaatggacggcattgtgggtaatctcgaactggacttcaaactgacaaaaatctgtctgctccaaagccaagagaacctgcctgtagaagaacgattctgatcctcgctctggatcattgaacaacttgtctcggggcgaccgagttgtatccactcccaaatagtctatgtgaccatgatggaaaggattgtcctcttcatctgtgacaaaagttggcaaagctgtaaaggcagttgtccagtcccagtaaggaaggccgactctggcaccatgggcggtcaaggcatcttccatctgttttgtgtacagtctgtgccagtgggggaaagtagccatgccatgtgtacaacacgagtaattctttccatctggcatagggcacattggtgggtttccatggaacgctgctatagcctggtatccatttggaccgtggtctgccatgacggctctcatggcatctttgagattgtccacctctgcagtggtaagtgtgttgatttcctttctgacaccatgtccagccatagaagaagaatgatgatcttcgtgatggtcgagaccaggttcatagataatggttggtgcatgcaggattgaagccggaagaactgttccgttgacggctgtcactgacaccttgatgaagaatggttcagagggatggaatacgtcttctggagtgatgtgtgcgtcatgaagagcatgggtaatatcgtacttgaataaacggttataagcccaggccatctctttagtccccccaagaacgaagatttggcctccatggtgacaatcttctgatgttttacaaatctggaattggacatcagctgaggttctaattccgtgaaggaggaaccctgcaaacactctcacatggtgctgtttgtctttgatttccttttcaatttcatgcaaattcatgccaccgatttccagattgtcgtatctgtaacccaggagttcgtacttgaaaacatcatttggaactgcatgtttcttggtgaactggttatggttaatttcgtgatggaaaggcctcatgttctgagtcatgagactaacagcgcagtcagcacggtctgatggaaggcctctcttttcttgaagagcctgccagacagcccaaaccttgtctacaaaggagtggtgtatgaagaagatcggatcatatgaagcataatgaagggatgacaaagaatagacttgaggtcctcccactaggtaatggattgtgttgtgcatgacttcaaactggactgcaaaatcgcagtagtcgtgctgttccagcgccaaaagagcttgattcagaagcgctgagtgttcacccgttgatgtcaggtggaacaggccttcttggacgtctcttaccgtgtatgtatcgtgtgaggggacataccctcgggcaaatggatttggcattactttatttctccagacatcgtagtattctttgtctgtgaacagatgtggaatattatgtattggctttgtccagtcccagtagggtactgctacactagagccatgtcttcttagagcttgctcaaattggagggtgtagagtctgtgccagtgggggaatgtaggcattccatggatacagcatgccacctcgttattgtggctgtcattacatttggcaggcagaccatggaaggatgcaatggcagcaaaaccatctgctgaggtatctgcctgcaagctgctcaaagcatatttaaggcttccaatatctcgctcactcagtgtactcagatcatgacgaaccttgttgagcgacaggtggtgcgaactaatatcatgatgagcaggatcatagatgattgacggtgtgggaataaggccactatcgagtacagttccatcgtaggatgtaatggttacttcaatctggaagatttcgtcatgtcgaaggttcatcttgtctaaggtttctgttatgtcgtacttgtaaagccgatcaaaggcccaaggcatttctttttcacctcccaacacggtgaagtaccctgcatcttcacagtctcccgctgtgcgacagacctggaaatcgacagtggcagaggtgccaaatccttcaagcaagaatccggcaaagacccttgaatgttcctgcctgttacggacaatatggtcaatgccacgaattgaccaccctgcaaattccagagtgtcatatatgtatccaaacttttggtagtcgaatgtatcttcaggctttgagaaatcatgcgtgtgatcattaagattatatggtgctccaaagctgaaaggcttcagaggagttttcatgagatttacagcacagttagcctcagatccagaaagtcctctgaaacgctgcaacgattgccatatagcccaaatacgatcagtttgggagtgatggatgtagaaaagtggatcataggaagcataatgcagatggccaattccatagggctccttgcctccaacccaggtatgaacaccattatgcataatttcaaactgaacctcgaagtcgcagtagttggtttgttcaagagcaagcagtgcttgctcaataaaccagttatgatgtgtttttgttgatccctggaagagacgatccctattgatatctcttttagtatgtacgttttctccttcaaactctattttagaaccaataaatggatttggtatatctctgcctgtctccgggtcatgaatagttgagacggtcaggagctctggtaattcagaacgaggtagggtccagtcccagtacggaagtccaacgactgcaccatgttttctgagagaatcttggaattggactgtgtacagacggtgccattgtgggaacgttgccatgccatggacgcagcacgcaaacctctttgaagcagctggtgatggacaaagaggaggaagagcatggaaggaagcgattgcctggaacccatcagatgaatggtcggctttcatggactcgagggctttcaccagtgacagttgttgacgcttgtccaattgtgtgatatctttgcgaatcatcactggttcagtgtgtccatcatctgtgtgggcagaatctgttccggcctcaaacagtatagtgggagagtggatgacatgactgtctagatgagatccattcacagccgtaatctcaatttccagatcgaaatccccttgcaggtgcacaccgttctttttcaaggctttagttatgtcaagtttgtaaagtctgtcaaatacaaaggacatctccgtttgaccgcctaagatggagaacataccagcctcatgactgcagtccttcgtgtgttccccagttgggagacatacaaatacatggacatcggcagatgttccaatattgtgaaggaggaagccagcgaatactctgtcatgcctttttctttcttgcagcatgtgatcaagctgaggaatcgagtagtgattcagttccagattatcgtaagcataaccaaagctgttttgatattcaaaaccatcatgtggagtggcatgcgttttcgttgcctcgtcattgttgtcgctcttatcaaacggctggagtggcgtgtgcatcaattggatagcgcaattcgcttctcggtatggccgttttcggaacctctgaaggtcttgccagattgcccagattctgtcagtcgttgcgtgatgaaggaagaacacaggatcaaaagcagcgtaatcaaggtttgacatggaatatttacctttgccaccaagtaaagaatgcaatgcattgtgtaatatctccaactgaatctcgaaatcacagaagttatcctgctccaaggcgtaaaggacctgctcgtaaaagtagtctgaatggaagaggctgtccttgggatccctagtagtgatttcattctcgtgtgtgattttgccatgatggaatgggtttgtctcatagtgatgttgcctggaattgtagtaagtggcgtctgaaatcaggtgaggtaaatgttcgattcgttttgtccagtcccaatagggaacagctacagatgatccttttttcaggagggcattctcaacgacaaccacgtacagtctgtgccagtgagggaaagttggcatgccatggacacaacaagagatggggttaccatttaactggcacaaaccagggctaccatggaatttagctattgattcgtaagttccatcgttttcaatttccaggaaggcagctctcagactttcgagttcaccaaggctcagattttcaatgttttttcgaatacggttggcagaagttacttcagcttgatatacttcgccttcatggtgacctattcttggttcatgaatgactgaaggctgcttgaacaaatcttttcccaggcttgctggtttaaggtcaaatacttcatagtcaatgtagaagtggtcttctgcatgcagctttagatcgtggagtttttcggttatgtcatatttgaagactctatcgtatgcccatggcatctcgttttcatcacccagaaggtaaaactctccagcggggtgacagttgctgctatcggtgcaaatattgaatttaacaagagatgatttcttgaaaccagataacaggaagcctgcaaagaacctgtcttggctctttatcgcttcgagctttttattcaactgagagattgagagaccgttaaattccaaagtgtcatattcataattgaaattggtcttgtaatcaaaaacgttgaatggcactgaatgctccttggttacatggtctgtgttgatattggcagagaggccaaatggttgcaaaggttctctcatcgatgtaacagcacagttagcaacgttgtacggttttcctctgtacttctgtaaagcctgccatattgcccatatacgatctgtattagagtgatgcaagtagaatagtggatcaaaagcagtgtagcgaagcgatgccataccataaggctgagcgcctcctacaagtgcatggatgtagttatgggcaatctcatattggatttcaaagtcacagaaattgtcctgttccaacgctagcagtacttgtttggcaatgcttgtataatgaccaaactcaggctgttcgaagagtttattctgaacacttcttgttgttgttttgtcgactgtatctatatgaccatcgaaccaagggttatgcttctctaccccatcttcagggtcgacgtacttctcatgatctgccagttcgggaaggtgattaagaggagaggtccaatcccagtacggcaaagctccatcgtagccatgtcgtctcaaagcattttcactctgaaccgtcaacagtcgatgccagtgagggaacacagacatgccgtgaacacagcaggcaaattttttagatgcttcgggactaggacaccattttggctccccatggaatgcaccaagctgctgaaatccaccggcacttttgtcctctttcagtaaggctagggctttacgaagttcattcatctcctcggggctcaaacggtctacattttttcgaataagacgactccttggcactttgtcaaatttaacacgtgcatgctcacgaatgacagaagcatgtggaataagactggatgcaagtttagttccatcgacttcagtgatgtcaacagtaacttcgaaatcgccatcagcagtgagatcgagatctttcaaaacgtgcgtgatgtcaaacttgaaaacgcgatcaaatccccacttcatttccttgcttccaccgagcactgcaaatgttccagccttatgttgcacggaatcggtagttttaatgaagatatcaacatttgctgaagtacgtatgccagccaggagaaaaccggcatatattctgtcttcttgctggttttcgtggatcatcttttctatgttttccagagagatgcctccaaatgttaaatcttcgtatgtgtaatggaggacattttcatagtcgtagatcttgtttggcatggcattggcatgagtcttttcgttattgttaaggggagatgaaaaggcgaatggtttcagatgcatatgttccagagatatggcgcagtgggccctgtagggtttatgccgtctcatctgtaaggcttgccaaacggcccaaagacgatcaacgttagaatggtgaaagtaaaacaaaggatcgaaagctgtgtaatgtagggacgacattgagaaatgttcgcttcctcctgtccaagcgtgaatcgtgttatgggtaatctcaaattggacttcaaagtcacagtaatcttcttgttcaaaggcatacaaaatctggttgaacaggtctgtgtggtgtccaaaggtagcgggtttaaagagtctttgatctatttgtcttttggtgtggggagcattttcttcaaatgcaatcactgaactatgaaaaggatttgtgtgggatgcaccatgagaatccacataagtttcgtttcctgccagacccggaatgtgtgtcatgggttcagtccagtcccagtatgggataccaatggtagctccacggcctactaaagcatcttcaacttgtgtgacaaaaagcctgtgccagtgagggaagattggcattccatggacacaacaagcatatctgtccttggcatcagggcgagggcatctggccggaagaccatgatactcagctgtggcttgataaccgtcgacggagcgatcttcttggaatttatgaaacgcctctctcagattcaacacttcttcacgggtgagatcattgatgttctttctaacgaggagattggcgctttggtgatgcttcaccggaggatcagtgaaacccttctccggatgatgaacaacaactggatgaggtaacaaatcatcgggaagagctgtaccattgacactgaaaagctctgccttgacggtatagtgtccatccaacttcattcccatcttgtgaacagtgtcggacaagtcaaacaagtatggcctgtagaactgccatttcatctcgcttggtccgcccagaacgaagaaatcaccggccttctcacagtggtcatcattacgatcgttgtcgtcggggatgcagacgtacacaaagacattggctgaacctccaaatcctttcagacggaagcttgcgaaggctcttgcccttgaagaccgctcatccaaataggttttcagttcctctggagtcataccattaagattcaacgtgtcatagtcgtagtgaagttgacgatagtcaaagagatcggctggagtggaatgttcatttgttagtggtatgtcattgtcctcccaactgaaaggttccattttctgatgtaacaattcttgggcacaatccatagctttggggtctttgtttcggagctcttggagcctctgccagatggcgaaaattctgtcaacgttggagtggtgaagaaagaagatcggatcgtaggcggtgtactcgaggtttgccatagagtaatcatgctttccaccgaccaggtagtgaatcgcattatgtgctaactcgaactggatttcgaatttacagaattcatcctgctccagtgcgtccaaaacactctccataagatgggtgtgctggcctggcttcaccttctcaaaaagacggtcgtcgaccgcacgattggttttcttgtctagaaagtcaatgttccctctgtaccagtagttggtgtgagcctttccgccctgggaatcgatgtaaacagggtccgcagcgagagagggcaactgggtgatgggttctgtccagtcccagtatggcatcccagaagtacgcctcttcgtctgcagagctcgctccatgtgaactacgtacgccctgtgccagtgcgggaaagtaggcatgccgtgaatacagcaagctatagaagtgtccttatgcacacaagatgcgggcgcgccatgagcagcagctattttctggaaaccaatgctggatgagtccatctgcagctcacgcagggcagcctgcaggtccagggtctcttcctgagtcagatgctccacactctttctgaccaggttctc

SEQ ID NO: 24 - protein, 3398 aa, Megathura crenulata
  SEQ ID NO24 is the deduced amino acid sequence of SEQ ID NO22
ENLVRKSVEHLTQEETLDLQAALRELQMDSSSIGFQKIAAAHGAPASCVHKDTSIACCIHGMPTFPHWHRAYVVHMERALQTKRRTSGMPYWDWTEPITQLPSLAADPVYIDSQGGKAHTNYWYRGNIDFLDKKTNRAVDDRLFEKVKPGQHTHLMESVLDALEQDEFCKFEIQFELAHNAIHYLVGGKHDYSMANLEYTAYDPIFFLHHSNVDRIFAIWQRLQELRNKDPKAMDCAQELLHQKMEPFSWEDNDIPLTNEHSTPADLFDYRQLHYDYDTLNLNGMTPEELKTYLDERSSRARAFASFRLKGFGGSANVFVYVCIPDDNDRNDDHCEKAGDFFVLGGPSEMKWQFYRPYLFDLSDTVHKMGMKLDGHYTVKAELFSVNGTALPDDLLPHPVVVHHPEKGFTDPPVKHHQSANLLVRKNINDLTREEVLNLREAFHKFQEDRSVDGYQATAEYHGLPARCPRPDAKDRYACCVHGMPIFPHWHRLFVTQVEDALVGRGATIGIPYWDWTEPMTHIPGLAGNETYVDSHGASHTNPFHSSVIAFEENAPHTKRQIDQRLFKPATFGHHTDLFNQILYAFEQEDYCDFEVQFEITHNTIHAWTGGSEHFSMSSLHYTAFDPLFYFHHSNVDRLWAVWQALQMRRHKPYRAHCAISLEHMHLKPFAFSSPLNNNEKTHANAMPNKIYDYENVLHYTYEDLTFGGISLENIEKMIHENQQEDRIYAGFLLAGIRTSANVDIFIKTTDSVQHKAGTFAVLGGSKEMKWGFDRVFKFDITHVLKDLDLTADGDFEVTVDITEVDGTKLASSLIPHASVIREHARVKFDKVPRSRLIRKNVDRLSPEEMNELRKALALLKEDKSAGGFQQLGAFHGEPKWCPSPEASKKFACCVHGMSVFPHWHRLLTVQSENALRRHGYDGALPYWDWTSPLNHLPELADHEKYVDPEDGVEKHNPWFDGHIDTVDKTTTRSVQNKLFEQPEFGHYTSIAKQVLLALEQDNFCDFEIQYEIAHNYIHALVGGAQPYGMASLRYTAFDPLFYLHHSNTDRIWAIWQALQKYRGKPYNVANCAVTSMREPLQPFGLSANINTDHVTKEHSVPFNVFDYKTNFNYEYDTLEFNGLSISQLNKKLEAIKSQDRFFAGFLLSGFKKSSLVKFNICTDSSNCHPAGEFYLLGDENEMPWAYDRVFKYDITEKLHDLKLHAEDHFYIDYEVFDLKPASLGKDLFKQPSVIHEPRIGHHEGEVYQAEVTSANRIRKNIENLSLGELESLRAAFLEIENDGTYESIAKFHGSPGLCQLNGNPISCCVHGMPTFPHWHRLYVVVVENALLKKGSSVAVPYWDWTKRIEHLPHLISDATYYNSRQHHYETNPFHHGKITHENEITTRDPKDSLFHSDYFYEQVLYALEQDNFCDFEIQLEILHNALHSLLGGKGKYSMSNLDYAAFDPVFFLHHATTDRIWAIWQDLQRFRKRPYREANCAIQLMHTPLQPFDKSDNNDEATKTHATPHDGFEYQNSFGYAYDNLELNHYSIPQLDHMLQERKRHDRVFAGFLLHNIGTSADVHVFVCLPTGEHTKDCSHEAGMFSILGGQTEMSFVFDRLYKLDITKALKKNGVHLQGDFDLEIEITAVNGSHLDSHVIHSPTILFEAGTDSAHTDDGHTEPVMIRKDITQLDKRQQLSLVKALESMKADHSSDGFQAIASFHALPPLCPSPAASKRFACCVHGMATFPQWHRLYTVQFQDSLRKHGAVVGLPYWDWTLPRSELPELLTVSTIHDPETGRDIPNPFIGSKIEFEGENVHTKRDINRDRLFQGSTKTHHNWFIEQALLALEQTNYCDFEVQFEIMHNGVHTWVGGKEPYGIGHLHYASYDPLFYIHHSQTDRIWAIWQSLQRFRGLSGSEANCAVNLMKTPLKPFSFGAPYNLNDHTHDFSKPEDTFDYQKFGYIYDTLEFAGWSIRGIDHIVRNRQEHSRVFAGFLLEGFGTSATVDFQVCRTAGDCEDAGYFTVLGGEKEMPWAFDRLYKYDITETLDKMNLRHDEIFQIEVTITSYDGTVLDSGLIPTPSIIYDPAHHDISSHHLSLNKVRHDLSTLSERDIGSLKYALSSLQADTSADGFAAIASFHGLPAKCNDSHNNEVACCIHGMPTFPHWHRLYTLQFEQALRRHGSSVAVPYWDWTKPIHNIPHLFTDKEYYDVWRNKVMPNPFARGYVPSHDTYTVRDVQEGLFHLTSTGEHSALLNQALLALEQHDYCDFAVQFEVMHNTIHYLVGGPQVYSLSSLHYASYDPIFFIHHSFVDKVWAVWQALQEKRGLPSDRADCAVSLMTQNMRPFHHEINHNQFTKKHAVPNDVFKYELLGYRYDNLEIGGMNLHEIEKEIKDKQHHVRVFAGFLLHGIRTSADVQFQICKTSEDCHHGGQIFVLGGTKEMAWAYNRLFKYDITHALHDAHITPEDVFHPSEPFFIKVSVTAVNGTVLPASILHAPTIIYEPGLDHHEDHHSSSMAGHGVRKEINTLTTAEVDNLKDAMRAVMADHGPNGYQAIAAFHGNPPMCPMPDGKNYSCCTHGMATFPHWHRLYTKQMEDALTAHGARVGLPYWDWTTAFTALPTFVTDEEDNPFHHGHIDYLGVDTTRSPRDKLFNDPERGSESFFYRQVLLALEQTDFCQFEVQFEITHNAVHSWTGGLTPYGMSTLEYTTYDPLFWLHHANTDRIWAIWQALQEYRGLPYDHANCEIQAMKRPLRPFSDPINHNAFTHSNAKPTDVFEYSRFNFQYDNLRFHGMTIKKLEHELEKQKEEDRTFAAFLLHGIKKSADVSFDVCNHDGECHFAGTFAILGGEHEMPWSFDRLFRYDITQVLKQMHLEYDSDFTFHMRIIDTSGKQLPSDLIKMPTVEHSPGGKHHEKHHEDHHEDILVRKNIHSLSHHEAEELRDALYKLQNDESHGGYEHIAGFHGYPNLCPEKGDEKYPCCVHGMSIFPHWHRLHTIQFERALKKHGSHLGIPYWDWTQTISSLPTFFADSGNNNPFFKYHIRSINQDTVRDVNEAIFQQTKFGEFSSIFYLALQALEEDNYCDFEVQYEILHNEVHALIGGAEKYSMSTLEYSAFDPYFMIHHASLDKIWIIWQELQKRRVKPAHAGSCAGDIMHVPLHPFNYESVNNDDFTRENSLPNAVVDSHRFNYKYDNLNLHGHNIEELEEVLRSLRLKSRVFAGFVLSGIRTTAVVKVYIKSGTDSDDEYAGSFVILGGAKEMPWAYERLYRFDITETVHNLNLTDDHVKFRFDLKKYDHTELDASVLPAPIIVRRPNNAVFDIIEIPIGKDVNLPPKVVVKRGTKIMFMSVDEAVTTPMLNLGSYTAMFKCKVPPFSFHAFELGKMYSVESGDYFMTASTTELCNDNNLRIHVHVDDE

SEQ ID NO: 25 - DNA, 48 bp, Megathura crenulata
atgctgtcggtcaggttgcttatagtcgtgttggctttggccaatgca

SEQ ID NO: 26 - DNA, 48 bp, Megathura crenulata
  SEQ ID NO26 is the complementary sequence of the coding sequence of SEQ ID NO25
tgcattggccaaagccaacacgactataagcaacctgaccgacagcat

SEQ ID NO: 27 - protein, 16 aa, Megathura crenulata
  SEQ ID NO27 is the deduced amino acid sequence of SEQ ID NO25
MLSVRLLIVVLALANA

SEQ ID NO: 28 - DNA, 10245 bp, Megathura crenulata
atgctgtcggtcaggttgcttatagtcgtgttggctttggccaatgcagagaacctggtcagaaagagtgtggagcatctgactcaggaagagaccctggacctgcaggctgccctgcgtgagctgcagatggactcatccagcattggtttccagaaaatagctgctgctcatggcgcgcccgcatcttgtgtgcataaggacacttctatagcttgctgtattcacggcatgcctactttcccgcactggcacagggcgtacgtagttcacatggagcgagctctgcagacgaagaggcgtacttctgggatgccatactgggactggacagaacccatcacccagttgccctctctcgctgcggaccctgtttacatcgattcccagggcggaaaggctcacaccaactactggtacagagggaacattgactttctagacaagaaaaccaatcgtgcggtcgacgaccgtctttttgagaaggtgaagccaggccagcacacccatcttatggagagtgttttggacgcactggagcaggatgaattctgtaaattcgaaatccagttcgagttagcacataatgcgattcactacctggtcggtggaaagcatgattactctatggcaaacctcgagtacaccgcctacgatccgatcttctttcttcaccactccaacgttgacagaattttcgccatctggcagaggctccaagagctccgaaacaaagaccccaaagctatggattgtgcccaagaattgttacatcagaaaatggaacctttcagttgggaggacaatgacataccactaacaaatgaacattccactccagccgatctctttgactatcgtcaacttcactacgactatgacacgttgaatcttaatggtatgactccagaggaactgaaaacctatttggatgagcggtcttcaagggcaagagccttcgcaagcttccgtctgaaaggatttggaggttcagccaatgtctttgtgtacgtctgcatccccgacgacaacgatcgtaatgatgaccactgtgagaaggccggtgatttcttcgttctgggcggaccaagcgagatgaaatggcagttctacaggccatacttgtttgacttgtccgacactgttcacaagatgggaatgaagttggatggacactataccgtcaaggcagagcttttcagtgtcaatggtacagctcttcccgatgatttgttacctcatccagttgttgttcatcatccggagaagggtttcactgatcctccggtgaagcatcaccaaagcgccaatctcctcgttagaaagaacatcaatgatctcacccgtgaagaagtgttgaatctgagagaggcgtttcataaattccaagaagatcgctccgtcgacggttatcaagccacagctgagtatcatggtcttccggccagatgccctcgccctgatgccaaggacagatatgcttgttgtgtccatggaatgccaatcttccctcactggcacaggctttttgtcacacaagttgaagatgctttagtaggccgtggagctaccattggtatcccatactgggactggactgaacccatgacacacattccgggtctggcaggaaacgaaacttatgtggattctcatggtgcatcccacacaaatccttttcatagttcagtgattgcatttgaagaaaatgctccccacaccaaaagacaaatagatcaaagactctttaaacccgctacctttggacaccacacagacctgttcaaccagattttgtatgcctttgaacaagaagattactgtgactttgaagtccaatttgagattacccataacacgattcacgcttggacaggaggaagcgaacatttctcaatgtcgtccctacattacacagctttcgatcctttgttttactttcaccattctaacgttgatcgtctttgggccgtttggcaagccttacagatgagacggcataaaccctacagggcccactgcgccatatctctggaacatatgcatctgaaaccattcgccttttcatctccccttaacaataacgaaaagactcatgccaatgccatgccaaacaagatctacgactatgaaaatgtcctccattacacatacgaagatttaacatttggaggcatctctctggaaaacatagaaaagatgatccacgaaaaccagcaagaagacagaatatatgccggttttctcctggctggcatacgtacttcagcaaatgttgatatcttcattaaaactaccgattccgtgcaacataaggctggaacatttgcagtgctcggtggaagcaaggaaatgaagtggggatttgatcgcgttttcaagtttgacatcacgcacgttttgaaagatctcgatctcactgctgatggcgatttcgaagttactgttgacatcactgaagtcgatggaactaaacttgcatccagtcttattccacatgcttctgtcattcgtgagcatgcacgtgttaaatttgacaaagtgccaaggagtcgtcttattcgaaaaaatgtagaccgtttgagccccgaggagatgaatgaacttcgtaaagccctagccttactgaaagaggacaaaagtgccggtggatttcagcagcttggtgcattccatggggagccaaaatggtgtcctagtcccgaagcatctaaaaaatttgcctgctgtgttcacggcatgtctgtgttccctcactggcatcgactgttgacggttcagagtgaaaatgctttgagacgacatggctacgatggagctttgccgtactgggattggacctctcctcttaatcaccttcccgaactggcagatcatgagaagtacgtcgaccctgaagatggggtagagaagcataacccttggttcgatggtcatatagatacagtcgacaaaacaacaacaagaagtgttcagaataaactcttcgaacagcctgagtttggtcattatacaagcattgccaaacaagtactgctagcgttggaacaggacaatttctgtgactttgaaatccaatatgagattgcccataactacatccatgcacttgtaggaggcgctcagccttatggtatggcatcgcttcgctacactgcttttgatccactattctacttgcatcactctaatacagatcgtatatgggcaatatggcaggctttacagaagtacagaggaaaaccgtacaacgttgctaactgtgctgttacatcgatgagagaacctttgcaaccatttggcctctctgccaatatcaacacagaccatgtaaccaaggagcattcagtgccattcaacgtttttgattacaagaccaatttcaattatgaatatgacactttggaatttaacggtctctcaatctctcagttgaataaaaagctcgaagcgataaagagccaagacaggttctttgcaggcttcctgttatctggtttcaagaaatcatctcttgttaaattcaatatttgcaccgatagcagcaactgtcaccccgctggagagttttaccttctgggtgatgaaaacgagatgccatgggcatacgatagagtcttcaaatatgacataaccgaaaaactccacgatctaaagctgcatgcagaagaccacttctacattgactatgaagtatttgaccttaaaccagcaagcctgggaaaagatttgttcaagcagccttcagtcattcatgaaccaagaataggtcaccatgaaggcgaagtatatcaagctgaagtaacttctgccaaccgtattcgaaaaaacattgaaaatctgagccttggtgaactcgaaagtctgagagctgccttcctggaaattgaaaacgatggaacttacgaatcaatagctaaattccatggtagccctggtttgtgccagttaaatggtaaccccatctcttgttgtgtccatggcatgccaactttccctcactggcacagactgtacgtggttgtcgttgagaatgccctcctgaaaaaaggatcatctgtagctgttccctattgggactggacaaaacgaatcgaacatttacctcacctgatttcagacgccacttactacaattccaggcaacatcactatgagacaaacccattccatcatggcaaaatcacacacgagaatgaaatcactactagggatcccaaggacagcctcttccattcagactacttttacgagcaggtcctttacgccttggagcaggataacttctgtgatttcgagattcagttggagatattacacaatgcattgcattctttacttggtggcaaaggtaaatattccatgtcaaaccttgattacgctgcttttgatcctgtgttcttccttcatcacgcaacgactgacagaatctgggcaatctggcaagaccttcagaggttccgaaaacggccataccgagaagcgaattgcgctatccaattgatgcacacgccactccagccgtttgataagagcgacaacaatgacgaggcaacgaaaacgcatgccactccacatgatggttttgaatatcaaaacagctttggttatgcttacgataatctggaactgaatcactactcgattcctcagcttgatcacatgctgcaagaaagaaaaaggcatgacagagtattcgctggcttcctccttcacaatattggaacatctgccgatgtccatgtatttgtatgtctcccaactggggaacacacgaaggactgcagtcatgaggctggtatgttctccatcttaggcggtcaaacggagatgtcctttgtatttgacagactttacaaacttgacataactaaagccttgaaaaagaacggtgtgcacctgcaaggggatttcgatctggaaattgagattacggctgtgaatggatctcatctagacagtcatgtcatccactctcccactatactgtttgaggccggaacagattctgcccacacagatgatggacacactgaaccagtgatgattcgcaaagatatcacacaattggacaagcgtcaacaactgtcactggtgaaagccctcgagtccatgaaagccgaccattcatctgatgggttccaggcaatcgcttccttccatgctcttcctcctctttgtccatcaccagctgcttcaaagaggtttgcgtgctgcgtccatggcatggcaacgttcccacaatggcaccgtctgtacacagtccaattccaagattctctcagaaaacatggtgcagtcgttggacttccgtactgggactggaccctacctcgttctgaattaccagagctcctgaccgtctcaactattcatgacccggagacaggcagagatataccaaatccatttattggttctaaaatagagtttgaaggagaaaacgtacatactaaaagagatatcaatagggatcgtctcttccagggatcaacaaaaacacatcataactggtttattgagcaagcactgcttgctcttgaacaaaccaactactgcgacttcgaggttcagtttgaaattatgcataatggtgttcatacctgggttggaggcaaggagccctatggaattggccatctgcattatgcttcctatgatccacttttctacatccatcactcccaaactgatcgtatttgggctatatggcaatcgttgcagcgtttcagaggactttctggatctgaggctaactgtgctgtaaatctcatgaaaactcctctgaagcctttcagctttggagcaccatataatcttaatgatcacacgcatgatttctcaaagcctgaagatacattcgactaccaaaagtttggatacatatatgacactctggaatttgcagggtggtcaattcgtggcattgaccatattgtccgtaacaggcaggaacattcaagggtctttgccggattcttgcttgaaggatttggcacctctgccactgtcgatttccaggtctgtcgcacagcgggagactgtgaagatgcagggtacttcaccgtgttgggaggtgaaaaagaaatgccttgggcctttgatcggctttacaagtacgacataacagaaaccttagacaagatgaaccttcgacatgacgaaatcttccagattgaagtaaccattacatcctacgatggaactgtactcgatagtggccttattcccacaccgtcaatcatctatgatcctgctcatcatgatattagttcgcaccacctgtcgctcaacaaggttcgtcatgatctgagtacactgagtgagcgagatattggaagccttaaatatgctttgagcagcttgcaggcagatacctcagcagatggttttgctgccattgcatccttccatggtctgcctgccaaatgtaatgacagccacaataacgaggtggcatgctgtatccatggaatgcctacattcccccactggcacagactctacaccctccaatttgagcaagctctaagaagacatggctctagtgtagcagtaccctactgggactggacaaagccaatacataatattccacatctgttcacagacaaagaatactacgatgtctggagaaataaagtaatgccaaatccatttgcccgagggtatgtcccctcacacgatacatacacggtaagagacgtccaagaaggcctgttccacctgacatcaacgggtgaacactcagcgcttctgaatcaagctcttttggcgctggaacagcacgactactgcgattttgcagtccagtttgaagtcatgcacaacacaatccattacctagtgggaggacctcaagtctattctttgtcatcccttcattatgcttcatatgatccgatcttcttcatacaccactcctttgtagacaaggtttgggctgtctggcaggctcttcaagaaaagagaggccttccatcagaccgtgctgactgcgctgttagtctcatgactcagaacatgaggcctttccatcacgaaattaaccataaccagttcaccaagaaacatgcagttccaaatgatgttttcaagtacgaactcctgggttacagatacgacaatctggaaatcggtggcatgaatttgcatgaaattgaaaaggaaatcaaagacaaacagcaccatgtgagagtgtttgcagggttcctccttcacggaattagaacctcagctgatgtccaattccagatttgtaaaacatcagaagattgtcaccatggaggccaaatcttcgttcttggggggactaaagagatggcctgggcttataaccgtttattcaagtacgatattacccatgctcttcatgacgcacacatcactccagaagacgtattccatccctctgaaccattcttcatcaaggtgtcagtgacagccgtcaacggaacagttcttccggcttcaatcctgcatgcaccaaccattatctatgaacctggtctcgaccatcacgaagatcatcattcttcttctatggctggacatggtgtcagaaaggaaatcaacacacttaccactgcagaggtggacaatctcaaagatgccatgagagccgtcatggcagaccacggtccaaatggataccaggctatagcagcgttccatggaaacccaccaatgtgccctatgccagatggaaagaattactcgtgttgtacacatggcatggctactttcccccactggcacagactgtacacaaaacagatggaagatgccttgaccgcccatggtgccagagtcggccttccttactgggactggacaactgcctttacagctttgccaacttttgtcacagatgaagaggacaatcctttccatcatggtcacatagactatttgggagtggatacaactcggtcgccccgagacaagttgttcaatgatccagagcgaggatcagaatcgttcttctacaggcaggttctcttggctttggagcagacagatttttgtcagtttgaagtccagttcgagattacccacaatgccgtccattcttggactggtggactgaccccctacggaatgtccactctggagtacacgacctatgatcctcttttctggcttcatcatgccaatactgaccgaatatgggctatttggcaagctcttcaagaatacagaggtctcccatacgaccatgccaactgcgaaatccaggcaatgaagaggcccctacggccattctctgatcctattaatcacaatgcgttcactcattctaacgcaaaaccaacagacgttttcgagtacagtagattcaacttccagtatgataacttacggtttcatggtatgacaataaaaaagttggagcatgagcttgagaaacagaaggaagaagacaggacattcgctgccttcctgctccacggcattaaaaaaagtgctgatgtaagctttgacgtgtgcaaccatgatggagaatgccactttgcaggtaccttcgctattcttggaggcgagcacgagatgccttggtcctttgacagacttttccgatatgacatcacccaagttcttaagcaaatgcacctggagtatgattctgatttcacgttccatatgagaattattgacacttcgggtaaacaactgccatcagacctgatcaagatgccaacggttgaacacagcccaggaggaaaacatcatgagaaacatcatgaagatcaccatgaagacatacttgttcgtaaaaacattcactctctgtcacaccatgaagccgaagaattgagagatgcattgtacaagcttcaaaacgatgaaagtcacggtggatatgaacatattgctggattccatggttatcctaacctctgtcctgaaaaaggggatgaaaagtatccttgctgtgttcatggtatgtctatattcccccactggcacaggctccacacgatacagtttgaaagagctctgaagaaacatggttcccatctcggtattccttactgggattggactcagacgatatcgtctcttcctaccttctttgcagattctggtaataataatccattcttcaaataccatatcagaagcataaatcaagacactgttcgagacgttaatgaagcgatattccaacaaacgaagtttggagaattttcgtccatattttacctggccttgcaggctctagaagaggataattattgcgattttgaggtgcagtacgagatacttcacaatgaggtgcatgccttgattggtggagccgagaagtattccatgtccacgctggaatattcagccttcgatccatacttcatgatccaccacgcgtctttggacaagatctggataatctggcaggagcttcagaaacgccgggttaagcctgcacatgctggttcgtgtgccggagacattatgcacgttccacttcatcctttcaattatgaaagcgtcaacaacgatgactttaccagagagaattctctccccaacgcggtcgttgacagtcatagattcaactataaatatgacaatcttaatcttcacggccacaatatagaggagcttgaggaggttctccgcagcctgagacttaaatctcgtgttttcgcaggatttgttctgtccggaatccgcacaactgcagttgtgaaagtgtatatcaaaagcggaaccgattccgacgacgaatatgccggttcgtttgtcatccttggaggagccaaagagatgccttgggcatatgagaggttgtatagatttgacattacagaaactgtacacaatcttaaccttacggatgatcatgttaaattcaggtttgacttaaagaaatatgatcatactgaattggatgcatcagtactgccagctcctataatcgttcgacgtccaaataatgcggtttttgatatcattgagattccaatagggaaagacgtgaatcttccaccgaaggtggtcgtcaagagaggcactaaaatcatgttcatgtctgttgatgaagccgttaccactcccatgctgaacctgggtagttatacagcaatgttcaaatgtaaagtgccaccattcagcttccatgcttttgagcttgggaaaatgtattctgtcgaatctggtgactatttcatgacggcatcaacaactgaattatgcaacgataataacttaaggatacatgtccatgtggatgacgaatag

SEQ ID NO: 29 - DNA, 10245 bp, Megathura crenulata
  SEQ ID NO29 is the complementary sequence of the coding sequence of SEQ ID NO28
ctattcgtcatccacatggacatgtatccttaagttattatcgttgcataattcagttgttgatgccgtcatgaaatagtcaccagattcgacagaatacattttcccaagctcaaaagcatggaagctgaatggtggcactttacatttgaacattgctgtataactacccaggttcagcatgggagtggtaacggcttcatcaacagacatgaacatgattttagtgcctctcttgacgaccaccttcggtggaagattcacgtctttccctattggaatctcaatgatatcaaaaaccgcattatttggacgtcgaacgattataggagctggcagtactgatgcatccaattcagtatgatcatatttctttaagtcaaacctgaatttaacatgatcatccgtaaggttaagattgtgtacagtttctgtaatgtcaaatctatacaacctctcatatgcccaaggcatctctttggctcctccaaggatgacaaacgaaccggcatattcgtcgtcggaatcggttccgcttttgatatacactttcacaactgcagttgtgcggattccggacagaacaaatcctgcgaaaacacgagatttaagtctcaggctgcggagaacctcctcaagctcctctatattgtggccgtgaagattaagattgtcatatttatagttgaatctatgactgtcaacgaccgcgttggggagagaattctctctggtaaagtcatcgttgttgacgctttcataattgaaaggatgaagtggaacgtgcataatgtctccggcacacgaaccagcatgtgcaggcttaacccggcgtttctgaagctcctgccagattatccagatcttgtccaaagacgcgtggtggatcatgaagtatggatcgaaggctgaatattccagcgtggacatggaatacttctcggctccaccaatcaaggcatgcacctcattgtgaagtatctcgtactgcacctcaaaatcgcaataattatcctcttctagagcctgcaaggccaggtaaaatatggacgaaaattctccaaacttcgtttgttggaatatcgcttcattaacgtctcgaacagtgtcttgatttatgcttctgatatggtatttgaagaatggattattattaccagaatctgcaaagaaggtaggaagagacgatatcgtctgagtccaatcccagtaaggaataccgagatgggaaccatgtttcttcagagctctttcaaactgtatcgtgtggagcctgtgccagtgggggaatatagacataccatgaacacagcaaggatacttttcatcccctttttcaggacagaggttaggataaccatggaatccagcaatatgttcatatccaccgtgactttcatcgttttgaagcttgtacaatgcatctctcaattcttcggcttcatggtgtgacagagagtgaatgtttttacgaacaagtatgtcttcatggtgatcttcatgatgtttctcatgatgttttcctcctgggctgtgttcaaccgttggcatcttgatcaggtctgatggcagttgtttacccgaagtgtcaataattctcatatggaacgtgaaatcagaatcatactccaggtgcatttgcttaagaacttgggtgatgtcatatcggaaaagtctgtcaaaggaccaaggcatctcgtgctcgcctccaagaatagcgaaggtacctgcaaagtggcattctccatcatggttgcacacgtcaaagcttacatcagcactttttttaatgccgtggagcaggaaggcagcgaatgtcctgtcttcttccttctgtttctcaagctcatgctccaacttttttattgtcataccatgaaaccgtaagttatcatactggaagttgaatctactgtactcgaaaacgtctgttggttttgcgttagaatgagtgaacgcattgtgattaataggatcagagaatggccgtaggggcctcttcattgcctggatttcgcagttggcatggtcgtatgggagacctctgtattcttgaagagcttgccaaatagcccatattcggtcagtattggcatgatgaagccagaaaagaggatcataggtcgtgtactccagagtggacattccgtagggggtcagtccaccagtccaagaatggacggcattgtgggtaatctcgaactggacttcaaactgacaaaaatctgtctgctccaaagccaagagaacctgcctgtagaagaacgattctgatcctcgctctggatcattgaacaacttgtctcggggcgaccgagttgtatccactcccaaatagtctatgtgaccatgatggaaaggattgtcctcttcatctgtgacaaaagttggcaaagctgtaaaggcagttgtccagtcccagtaaggaaggccgactctggcaccatgggcggtcaaggcatcttccatctgttttgtgtacagtctgtgccagtgggggaaagtagccatgccatgtgtacaacacgagtaattctttccatctggcatagggcacattggtgggtttccatggaacgctgctatagcctggtatccatttggaccgtggtctgccatgacggctctcatggcatctttgagattgtccacctctgcagtggtaagtgtgttgatttcctttctgacaccatgtccagccatagaagaagaatgatgatcttcgtgatggtcgagaccaggttcatagataatggttggtgcatgcaggattgaagccggaagaactgttccgttgacggctgtcactgacaccttgatgaagaatggttcagagggatggaatacgtcttctggagtgatgtgtgcgtcatgaagagcatgggtaatatcgtacttgaataaacggttataagcccaggccatctctttagtccccccaagaacgaagatttggcctccatggtgacaatcttctgatgttttacaaatctggaattggacatcagctgaggttctaattccgtgaaggaggaaccctgcaaacactctcacatggtgctgtttgtctttgatttccttttcaatttcatgcaaattcatgccaccgatttccagattgtcgtatctgtaacccaggagttcgtacttgaaaacatcatttggaactgcatgtttcttggtgaactggttatggttaatttcgtgatggaaaggcctcatgttctgagtcatgagactaacagcgcagtcagcacggtctgatggaaggcctctcttttcttgaagagcctgccagacagcccaaaccttgtctacaaaggagtggtgtatgaagaagatcggatcatatgaagcataatgaagggatgacaaagaatagacttgaggtcctcccactaggtaatggattgtgttgtgcatgacttcaaactggactgcaaaatcgcagtagtcgtgctgttccagcgccaaaagagcttgattcagaagcgctgagtgttcacccgttgatgtcaggtggaacaggccttcttggacgtctcttaccgtgtatgtatcgtgtgaggggacataccctcgggcaaatggatttggcattactttatttctccagacatcgtagtattctttgtctgtgaacagatgtggaatattatgtattggctttgtccagtcccagtagggtactgctacactagagccatgtcttcttagagcttgctcaaattggagggtgtagagtctgtgccagtgggggaatgtaggcattccatggatacagcatgccacctcgttattgtggctgtcattacatttggcaggcagaccatggaaggatgcaatggcagcaaaaccatctgctgaggtatctgcctgcaagctgctcaaagcatatttaaggcttccaatatctcgctcactcagtgtactcagatcatgacgaaccttgttgagcgacaggtggtgcgaactaatatcatgatgagcaggatcatagatgattgacggtgtgggaataaggccactatcgagtacagttccatcgtaggatgtaatggttacttcaatctggaagatttcgtcatgtcgaaggttcatcttgtctaaggtttctgttatgtcgtacttgtaaagccgatcaaaggcccaaggcatttctttttcacctcccaacacggtgaagtaccctgcatcttcacagtctcccgctgtgcgacagacctggaaatcgacagtggcagaggtgccaaatccttcaagcaagaatccggcaaagacccttgaatgttcctgcctgttacggacaatatggtcaatgccacgaattgaccaccctgcaaattccagagtgtcatatatgtatccaaacttttggtagtcgaatgtatcttcaggctttgagaaatcatgcgtgtgatcattaagattatatggtgctccaaagctgaaaggcttcagaggagttttcatgagatttacagcacagttagcctcagatccagaaagtcctctgaaacgctgcaacgattgccatatagcccaaatacgatcagtttgggagtgatggatgtagaaaagtggatcataggaagcataatgcagatggccaattccatagggctccttgcctccaacccaggtatgaacaccattatgcataatttcaaactgaacctcgaagtcgcagtagttggtttgttcaagagcaagcagtgcttgctcaataaaccagttatgatgtgtttttgttgatccctggaagagacgatccctattgatatctcttttagtatgtacgttttctccttcaaactctattttagaaccaataaatggatttggtatatctctgcctgtctccgggtcatgaatagttgagacggtcaggagctctggtaattcagaacgaggtagggtccagtcccagtacggaagtccaacgactgcaccatgttttctgagagaatcttggaattggactgtgtacagacggtgccattgtgggaacgttgccatgccatggacgcagcacgcaaacctctttgaagcagctggtgatggacaaagaggaggaagagcatggaaggaagcgattgcctggaacccatcagatgaatggtcggctttcatggactcgagggctttcaccagtgacagttgttgacgcttgtccaattgtgtgatatctttgcgaatcatcactggttcagtgtgtccatcatctgtgtgggcagaatctgttccggcctcaaacagtatagtgggagagtggatgacatgactgtctagatgagatccattcacagccgtaatctcaatttccagatcgaaatccccttgcaggtgcacaccgttctttttcaaggctttagttatgtcaagtttgtaaagtctgtcaaatacaaaggacatctccgtttgaccgcctaagatggagaacataccagcctcatgactgcagtccttcgtgtgttccccagttgggagacatacaaatacatggacatcggcagatgttccaatattgtgaaggaggaagccagcgaatactctgtcatgcctttttctttcttgcagcatgtgatcaagctgaggaatcgagtagtgattcagttccagattatcgtaagcataaccaaagctgttttgatattcaaaaccatcatgtggagtggcatgcgttttcgttgcctcgtcattgttgtcgctcttatcaaacggctggagtggcgtgtgcatcaattggatagcgcaattcgcttctcggtatggccgttttcggaacctctgaaggtcttgccagattgcccagattctgtcagtcgttgcgtgatgaaggaagaacacaggatcaaaagcagcgtaatcaaggtttgacatggaatatttacctttgccaccaagtaaagaatgcaatgcattgtgtaatatctccaactgaatctcgaaatcacagaagttatcctgctccaaggcgtaaaggacctgctcgtaaaagtagtctgaatggaagaggctgtccttgggatccctagtagtgatttcattctcgtgtgtgattttgccatgatggaatgggtttgtctcatagtgatgttgcctggaattgtagtaagtggcgtctgaaatcaggtgaggtaaatgttcgattcgttttgtccagtcccaatagggaacagctacagatgatccttttttcaggagggcattctcaacgacaaccacgtacagtctgtgccagtgagggaaagttggcatgccatggacacaacaagagatggggttaccatttaactggcacaaaccagggctaccatggaatttagctattgattcgtaagttccatcgttttcaatttccaggaaggcagctctcagactttcgagttcaccaaggctcagattttcaatgttttttcgaatacggttggcagaagttacttcagcttgatatacttcgccttcatggtgacctattcttggttcatgaatgactgaaggctgcttgaacaaatcttttcccaggcttgctggtttaaggtcaaatacttcatagtcaatgtagaagtggtcttctgcatgcagctttagatcgtggagtttttcggttatgtcatatttgaagactctatcgtatgcccatggcatctcgttttcatcacccagaaggtaaaactctccagcggggtgacagttgctgctatcggtgcaaatattgaatttaacaagagatgatttcttgaaaccagataacaggaagcctgcaaagaacctgtcttggctctttatcgcttcgagctttttattcaactgagagattgagagaccgttaaattccaaagtgtcatattcataattgaaattggtcttgtaatcaaaaacgttgaatggcactgaatgctccttggttacatggtctgtgttgatattggcagagaggccaaatggttgcaaaggttctctcatcgatgtaacagcacagttagcaacgttgtacggttttcctctgtacttctgtaaagcctgccatattgcccatatacgatctgtattagagtgatgcaagtagaatagtggatcaaaagcagtgtagcgaagcgatgccataccataaggctgagcgcctcctacaagtgcatggatgtagttatgggcaatctcatattggatttcaaagtcacagaaattgtcctgttccaacgctagcagtacttgtttggcaatgcttgtataatgaccaaactcaggctgttcgaagagtttattctgaacacttcttgttgttgttttgtcgactgtatctatatgaccatcgaaccaagggttatgcttctctaccccatcttcagggtcgacgtacttctcatgatctgccagttcgggaaggtgattaagaggagaggtccaatcccagtacggcaaagctccatcgtagccatgtcgtctcaaagcattttcactctgaaccgtcaacagtcgatgccagtgagggaacacagacatgccgtgaacacagcaggcaaattttttagatgcttcgggactaggacaccattttggctccccatggaatgcaccaagctgctgaaatccaccggcacttttgtcctctttcagtaaggctagggctttacgaagttcattcatctcctcggggctcaaacggtctacattttttcgaataagacgactccttggcactttgtcaaatttaacacgtgcatgctcacgaatgacagaagcatgtggaataagactggatgcaagtttagttccatcgacttcagtgatgtcaacagtaacttcgaaatcgccatcagcagtgagatcgagatctttcaaaacgtgcgtgatgtcaaacttgaaaacgcgatcaaatccccacttcatttccttgcttccaccgagcactgcaaatgttccagccttatgttgcacggaatcggtagttttaatgaagatatcaacatttgctgaagtacgtatgccagccaggagaaaaccggcatatattctgtcttcttgctggttttcgtggatcatcttttctatgttttccagagagatgcctccaaatgttaaatcttcgtatgtgtaatggaggacattttcatagtcgtagatcttgtttggcatggcattggcatgagtcttttcgttattgttaaggggagatgaaaaggcgaatggtttcagatgcatatgttccagagatatggcgcagtgggccctgtagggtttatgccgtctcatctgtaaggcttgccaaacggcccaaagacgatcaacgttagaatggtgaaagtaaaacaaaggatcgaaagctgtgtaatgtagggacgacattgagaaatgttcgcttcctcctgtccaagcgtgaatcgtgttatgggtaatctcaaattggacttcaaagtcacagtaatcttcttgttcaaaggcatacaaaatctggttgaacaggtctgtgtggtgtccaaaggtagcgggtttaaagagtctttgatctatttgtcttttggtgtggggagcattttcttcaaatgcaatcactgaactatgaaaaggatttgtgtgggatgcaccatgagaatccacataagtttcgtttcctgccagacccggaatgtgtgtcatgggttcagtccagtcccagtatgggataccaatggtagctccacggcctactaaagcatcttcaacttgtgtgacaaaaagcctgtgccagtgagggaagattggcattccatggacacaacaagcatatctgtccttggcatcagggcgagggcatctggccggaagaccatgatactcagctgtggcttgataaccgtcgacggagcgatcttcttggaatttatgaaacgcctctctcagattcaacacttcttcacgggtgagatcattgatgttctttctaacgaggagattggcgctttggtgatgcttcaccggaggatcagtgaaacccttctccggatgatgaacaacaactggatgaggtaacaaatcatcgggaagagctgtaccattgacactgaaaagctctgccttgacggtatagtgtccatccaacttcattcccatcttgtgaacagtgtcggacaagtcaaacaagtatggcctgtagaactgccatttcatctcgcttggtccgcccagaacgaagaaatcaccggccttctcacagtggtcatcattacgatcgttgtcgtcggggatgcagacgtacacaaagacattggctgaacctccaaatcctttcagacggaagcttgcgaaggctcttgcccttgaagaccgctcatccaaataggttttcagttcctctggagtcataccattaagattcaacgtgtcatagtcgtagtgaagttgacgatagtcaaagagatcggctggagtggaatgttcatttgttagtggtatgtcattgtcctcccaactgaaaggttccattttctgatgtaacaattcttgggcacaatccatagctttggggtctttgtttcggagctcttggagcctctgccagatggcgaaaattctgtcaacgttggagtggtgaagaaagaagatcggatcgtaggcggtgtactcgaggtttgccatagagtaatcatgctttccaccgaccaggtagtgaatcgcattatgtgctaactcgaactggatttcgaatttacagaattcatcctgctccagtgcgtccaaaacactctccataagatgggtgtgctggcctggcttcaccttctcaaaaagacggtcgtcgaccgcacgattggttttcttgtctagaaagtcaatgttccctctgtaccagtagttggtgtgagcctttccgccctgggaatcgatgtaaacagggtccgcagcgagagagggcaactgggtgatgggttctgtccagtcccagtatggcatcccagaagtacgcctcttcgtctgcagagctcgctccatgtgaactacgtacgccctgtgccagtgcgggaaagtaggcatgccgtgaatacagcaagctatagaagtgtccttatgcacacaagatgcgggcgcgccatgagcagcagctattttctggaaaccaatgctggatgagtccatctgcagctcacgcagggcagcctgcaggtccagggtctcttcctgagtcagatgctccacactctttctgaccaggttctctgcattggccaaagccaacacgactataagcaacctgaccgacagcat

SEQ ID NO: 30 - protein, 3414 aa, Megathura crenulata
  SEQ ID NO30 is the deduced amino acid sequence of SEQ ID NO28
MLSVRLLIVVLALANAENLVRKSVEHLTQEETLDLQAALRELQMDSSSIGFQKIAAAHGAPASCVHKDTSIACCIHGMPTFPHWHRAYVVHMERALQTKRRTSGMPYWDWTEPITQLPSLAADPVYIDSQGGKAHTNYWYRGNIDFLDKKTNRAVDDRLFEKVKPGQHTHLMESVLDALEQDEFCKFEIQFELAHNAIHYLVGGKHDYSMANLEYTAYDPIFFLHHSNVDRIFAIWQRLQELRNKDPKAMDCAQELLHQKMEPFSWEDNDIPLTNEHSTPADLFDYRQLHYDYDTLNLNGMTPEELKTYLDERSSRARAFASFRLKGFGGSANVFVYVCIPDDNDRNDDHCEKAGDFFVLGGPSEMKWQFYRPYLFDLSDTVHKMGMKLDGHYTVKAELFSVNGTALPDDLLPHPVVVHHPEKGFTDPPVKHHQSANLLVRKNINDLTREEVLNLREAFHKFQEDRSVDGYQATAEYHGLPARCPRPDAKDRYACCVHGMPIFPHWHRLFVTQVEDALVGRGATIGIPYWDWTEPMTHIPGLAGNETYVDSHGASHTNPFHSSVIAFEENAPHTKRQIDQRLFKPATFGHHTDLFNQILYAFEQEDYCDFEVQFEITHNTIHAWTGGSEHFSMSSLHYTAFDPLFYFHHSNVDRLWAVWQALQMRRHKPYRAHCAISLEHMHLKPFAFSSPLNNNEKTHANAMPNKIYDYENVLHYTYEDLTFGGISLENIEKMIHENQQEDRIYAGFLLAGIRTSANVDIFIKTTDSVQHKAGTFAVLGGSKEMKWGFDRVFKFDITHVLKDLDLTADGDFEVTVDITEVDGTKLASSLIPHASVIREHARVKFDKVPRSRLIRKNVDRLSPEEMNELRKALALLKEDKSAGGFQQLGAFHGEPKWCPSPEASKKFACCVHGMSVFPHWHRLLTVQSENALRRHGYDGALPYWDWTSPLNHLPELADHEKYVDPEDGVEKHNPWFDGHIDTVDKTTTRSVQNKLFEQPEFGHYTSIAKQVLLALEQDNFCDFEIQYEIAHNYIHALVGGAQPYGMASLRYTAFDPLFYLHHSNTDRIWAIWQALQKYRGKPYNVANCAVTSMREPLQPFGLSANINTDHVTKEHSVPFNVFDYKTNFNYEYDTLEFNGLSISQLNKKLEAIKSQDRFFAGFLLSGFKKSSLVKFNICTDSSNCHPAGEFYLLGDENEMPWAYDRVFKYDITEKLHDLKLHAEDHFYIDYEVFDLKPASLGKDLFKQPSVIHEPRIGHHEGEVYQAEVTSANRIRKNIENLSLGELESLRAAFLEIENDGTYESIAKFHGSPGLCQLNGNPISCCVHGMPTFPHWHRLYVVVVENALLKKGSSVAVPYWDWTKRIEHLPHLISDATYYNSRQHHYETNPFHHGKITHENEITTRDPKDSLFHSDYFYEQVLYALEQDNFCDFEIQLEILHNALHSLLGGKGKYSMSNLDYAAFDPVFFLHHATTDRIWAIWQDLQRFRKRPYREANCAIQLMHTPLQPFDKSDNNDEATKTHATPHDGFEYQNSFGYAYDNLELNHYSIPQLDHMLQERKRHDRVFAGFLLHNIGTSADVHVFVCLPTGEHTKDCSHEAGMFSILGGQTEMSFVFDRLYKLDITKALKKNGVHLQGDFDLEIEITAVNGSHLDSHVIHSPTILFEAGTDSAHTDDGHTEPVMIRKDITQLDKRQQLSLVKALESMKADHSSDGFQAIASFHALPPLCPSPAASKRFACCVHGMATFPQWHRLYTVQFQDSLRKHGAVVGLPYWDWTLPRSELPELLTVSTIHDPETGRDIPNPFIGSKIEFEGENVHTKRDINRDRLFQGSTKTHHNWFIEQALLALEQTNYCDFEVQFEIMHNGVHTWVGGKEPYGIGHLHYASYDPLFYIHHSQTDRIWAIWQSLQRFRGLSGSEANCAVNLMKTPLKPFSFGAPYNLNDHTHDFSKPEDTFDYQKFGYIYDTLEFAGWSIRGIDHIVRNRQEHSRVFAGFLLEGFGTSATVDFQVCRTAGDCEDAGYFTVLGGEKEMPWAFDRLYKYDITETLDKMNLRHDEIFQIEVTITSYDGTVLDSGLIPTPSIIYDPAHHDISSHHLSLNKVRHDLSTLSERDIGSLKYALSSLQADTSADGFAAIASFHGLPAKCNDSHNNEVACCIHGMPTFPHWHRLYTLQFEQALRRHGSSVAVPYWDWTKPIHNIPHLFTDKEYYDVWRNKVMPNPFARGYVPSHDTYTVRDVQEGLFHLTSTGEHSALLNQALLALEQHDYCDFAVQFEVMHNTIHYLVGGPQVYSLSSLHYASYDPIFFIHHSFVDKVWAVWQALQEKRGLPSDRADCAVSLMTQNMRPFHHEINHNQFTKKHAVPNDVFKYELLGYRYDNLEIGGMNLHEIEKEIKDKQHHVRVFAGFLLHGIRTSADVQFQICKTSEDCHHGGQIFVLGGTKEMAWAYNRLFKYDITHALHDAHITPEDVFHPSEPFFIKVSVTAVNGTVLPASILHAPTIIYEPGLDHHEDHHSSSMAGHGVRKEINTLTTAEVDNLKDAMRAVMADHGPNGYQAIAAFHGNPPMCPMPDGKNYSCCTHGMATFPHWHRLYTKQMEDALTAHGARVGLPYWDWTTAFTALPTFVTDEEDNPFHHGHIDYLGVDTTRSPRDKLFNDPERGSESFFYRQVLLALEQTDFCQFEVQFEITHNAVHSWTGGLTPYGMSTLEYTTYDPLFWLHHANTDRIWAIWQALQEYRGLPYDHANCEIQAMKRPLRPFSDPINHNAFTHSNAKPTDVFEYSRFNFQYDNLRFHGMTIKKLEHELEKQKEEDRTFAAFLLHGIKKSADVSFDVCNHDGECHFAGTFAILGGEHEMPWSFDRLFRYDITQVLKQMHLEYDSDFTFHMRIIDTSGKQLPSDLIKMPTVEHSPGGKHHEKHHEDHHEDILVRKNIHSLSHHEAEELRDALYKLQNDESHGGYEHIAGFHGYPNLCPEKGDEKYPCCVHGMSIFPHWHRLHTIQFERALKKHGSHLGIPYWDWTQTISSLPTFFADSGNNNPFFKYHIRSINQDTVRDVNEAIFQQTKFGEFSSIFYLALQALEEDNYCDFEVQYEILHNEVHALIGGAEKYSMSTLEYSAFDPYFMIHHASLDKIWIIWQELQKRRVKPAHAGSCAGDIMHVPLHPFNYESVNNDDFTRENSLPNAVVDSHRFNYKYDNLNLHGHNIEELEEVLRSLRLKSRVFAGFVLSGIRTTAVVKVYIKSGTDSDDEYAGSFVILGGAKEMPWAYERLYRFDITETVHNLNLTDDHVKFRFDLKKYDHTELDASVLPAPIIVRRPNNAVFDIIEIPIGKDVNLPPKVVVKRGTKIMFMSVDEAVTTPMLNLGSYTAMFKCKVPPFSFHAFELGKMYSVESGDYFMTASTTELCNDNNLRIHVHVDDE

SEQ ID NO: 31 - DNA, 10949 bp, Megathura crenulata
ctggcgatttggagaggcgcaatgctgtcggtcaggttgcttatagtcgtgttggctttggccaatgcagagaacctggtcagaaagagtgtggagcatctgactcaggaagagaccctggacctgcaggctgccctgcgtgagctgcagatggactcatccagcattggtttccagaaaatagctgctgctcatggcgcgcccgcatcttgtgtgcataaggacacttctatagcttgctgtattcacggcatgcctactttcccgcactggcacagggcgtacgtagttcacatggagcgagctctgcagacgaagaggcgtacttctgggatgccatactgggactggacagaacccatcacccagttgccctctctcgctgcggaccctgtttacatcgattcccagggcggaaaggctcacaccaactactggtacagagggaacattgactttctagacaagaaaaccaatcgtgcggtcgacgaccgtctttttgagaaggtgaagccaggccagcacacccatcttatggagagtgttttggacgcactggagcaggatgaattctgtaaattcgaaatccagttcgagttagcacataatgcgattcactacctggtcggtggaaagcatgattactctatggcaaacctcgagtacaccgcctacgatccgatcttctttcttcaccactccaacgttgacagaattttcgccatctggcagaggctccaagagctccgaaacaaagaccccaaagctatggattgtgcccaagaattgttacatcagaaaatggaacctttcagttgggaggacaatgacataccactaacaaatgaacattccactccagccgatctctttgactatcgtcaacttcactacgactatgacacgttgaatcttaatggtatgactccagaggaactgaaaacctatttggatgagcggtcttcaagggcaagagccttcgcaagcttccgtctgaaaggatttggaggttcagccaatgtctttgtgtacgtctgcatccccgacgacaacgatcgtaatgatgaccactgtgagaaggccggtgatttcttcgttctgggcggaccaagcgagatgaaatggcagttctacaggccatacttgtttgacttgtccgacactgttcacaagatgggaatgaagttggatggacactataccgtcaaggcagagcttttcagtgtcaatggtacagctcttcccgatgatttgttacctcatccagttgttgttcatcatccggagaagggtttcactgatcctccggtgaagcatcaccaaagcgccaatctcctcgttagaaagaacatcaatgatctcacccgtgaagaagtgttgaatctgagagaggcgtttcataaattccaagaagatcgctccgtcgacggttatcaagccacagctgagtatcatggtcttccggccagatgccctcgccctgatgccaaggacagatatgcttgttgtgtccatggaatgccaatcttccctcactggcacaggctttttgtcacacaagttgaagatgctttagtaggccgtggagctaccattggtatcccatactgggactggactgaacccatgacacacattccgggtctggcaggaaacgaaacttatgtggattctcatggtgcatcccacacaaatccttttcatagttcagtgattgcatttgaagaaaatgctccccacaccaaaagacaaatagatcaaagactctttaaacccgctacctttggacaccacacagacctgttcaaccagattttgtatgcctttgaacaagaagattactgtgactttgaagtccaatttgagattacccataacacgattcacgcttggacaggaggaagcgaacatttctcaatgtcgtccctacattacacagctttcgatcctttgttttactttcaccattctaacgttgatcgtctttgggccgtttggcaagccttacagatgagacggcataaaccctacagggcccactgcgccatatctctggaacatatgcatctgaaaccattcgccttttcatctccccttaacaataacgaaaagactcatgccaatgccatgccaaacaagatctacgactatgaaaatgtcctccattacacatacgaagatttaacatttggaggcatctctctggaaaacatagaaaagatgatccacgaaaaccagcaagaagacagaatatatgccggttttctcctggctggcatacgtacttcagcaaatgttgatatcttcattaaaactaccgattccgtgcaacataaggctggaacatttgcagtgctcggtggaagcaaggaaatgaagtggggatttgatcgcgttttcaagtttgacatcacgcacgttttgaaagatctcgatctcactgctgatggcgatttcgaagttactgttgacatcactgaagtcgatggaactaaacttgcatccagtcttattccacatgcttctgtcattcgtgagcatgcacgtgttaaatttgacaaagtgccaaggagtcgtcttattcgaaaaaatgtagaccgtttgagccccgaggagatgaatgaacttcgtaaagccctagccttactgaaagaggacaaaagtgccggtggatttcagcagcttggtgcattccatggggagccaaaatggtgtcctagtcccgaagcatctaaaaaatttgcctgctgtgttcacggcatgtctgtgttccctcactggcatcgactgttgacggttcagagtgaaaatgctttgagacgacatggctacgatggagctttgccgtactgggattggacctctcctcttaatcaccttcccgaactggcagatcatgagaagtacgtcgaccctgaagatggggtagagaagcataacccttggttcgatggtcatatagatacagtcgacaaaacaacaacaagaagtgttcagaataaactcttcgaacagcctgagtttggtcattatacaagcattgccaaacaagtactgctagcgttggaacaggacaatttctgtgactttgaaatccaatatgagattgcccataactacatccatgcacttgtaggaggcgctcagccttatggtatggcatcgcttcgctacactgcttttgatccactattctacttgcatcactctaatacagatcgtatatgggcaatatggcaggctttacagaagtacagaggaaaaccgtacaacgttgctaactgtgctgttacatcgatgagagaacctttgcaaccatttggcctctctgccaatatcaacacagaccatgtaaccaaggagcattcagtgccattcaacgtttttgattacaagaccaatttcaattatgaatatgacactttggaatttaacggtctctcaatctctcagttgaataaaaagctcgaagcgataaagagccaagacaggttctttgcaggcttcctgttatctggtttcaagaaatcatctcttgttaaattcaatatttgcaccgatagcagcaactgtcaccccgctggagagttttaccttctgggtgatgaaaacgagatgccatgggcatacgatagagtcttcaaatatgacataaccgaaaaactccacgatctaaagctgcatgcagaagaccacttctacattgactatgaagtatttgaccttaaaccagcaagcctgggaaaagatttgttcaagcagccttcagtcattcatgaaccaagaataggtcaccatgaaggcgaagtatatcaagctgaagtaacttctgccaaccgtattcgaaaaaacattgaaaatctgagccttggtgaactcgaaagtctgagagctgccttcctggaaattgaaaacgatggaacttacgaatcaatagctaaattccatggtagccctggtttgtgccagttaaatggtaaccccatctcttgttgtgtccatggcatgccaactttccctcactggcacagactgtacgtggttgtcgttgagaatgccctcctgaaaaaaggatcatctgtagctgttccctattgggactggacaaaacgaatcgaacatttacctcacctgatttcagacgccacttactacaattccaggcaacatcactatgagacaaacccattccatcatggcaaaatcacacacgagaatgaaatcactactagggatcccaaggacagcctcttccattcagactacttttacgagcaggtcctttacgccttggagcaggataacttctgtgatttcgagattcagttggagatattacacaatgcattgcattctttacttggtggcaaaggtaaatattccatgtcaaaccttgattacgctgcttttgatcctgtgttcttccttcatcacgcaacgactgacagaatctgggcaatctggcaagaccttcagaggttccgaaaacggccataccgagaagcgaattgcgctatccaattgatgcacacgccactccagccgtttgataagagcgacaacaatgacgaggcaacgaaaacgcatgccactccacatgatggttttgaatatcaaaacagctttggttatgcttacgataatctggaactgaatcactactcgattcctcagcttgatcacatgctgcaagaaagaaaaaggcatgacagagtattcgctggcttcctccttcacaatattggaacatctgccgatgtccatgtatttgtatgtctcccaactggggaacacacgaaggactgcagtcatgaggctggtatgttctccatcttaggcggtcaaacggagatgtcctttgtatttgacagactttacaaacttgacataactaaagccttgaaaaagaacggtgtgcacctgcaaggggatttcgatctggaaattgagattacggctgtgaatggatctcatctagacagtcatgtcatccactctcccactatactgtttgaggccggaacagattctgcccacacagatgatggacacactgaaccagtgatgattcgcaaagatatcacacaattggacaagcgtcaacaactgtcactggtgaaagccctcgagtccatgaaagccgaccattcatctgatgggttccaggcaatcgcttccttccatgctcttcctcctctttgtccatcaccagctgcttcaaagaggtttgcgtgctgcgtccatggcatggcaacgttcccacaatggcaccgtctgtacacagtccaattccaagattctctcagaaaacatggtgcagtcgttggacttccgtactgggactggaccctacctcgttctgaattaccagagctcctgaccgtctcaactattcatgacccggagacaggcagagatataccaaatccatttattggttctaaaatagagtttgaaggagaaaacgtacatactaaaagagatatcaatagggatcgtctcttccagggatcaacaaaaacacatcataactggtttattgagcaagcactgcttgctcttgaacaaaccaactactgcgacttcgaggttcagtttgaaattatgcataatggtgttcatacctgggttggaggcaaggagccctatggaattggccatctgcattatgcttcctatgatccacttttctacatccatcactcccaaactgatcgtatttgggctatatggcaatcgttgcagcgtttcagaggactttctggatctgaggctaactgtgctgtaaatctcatgaaaactcctctgaagcctttcagctttggagcaccatataatcttaatgatcacacgcatgatttctcaaagcctgaagatacattcgactaccaaaagtttggatacatatatgacactctggaatttgcagggtggtcaattcgtggcattgaccatattgtccgtaacaggcaggaacattcaagggtctttgccggattcttgcttgaaggatttggcacctctgccactgtcgatttccaggtctgtcgcacagcgggagactgtgaagatgcagggtacttcaccgtgttgggaggtgaaaaagaaatgccttgggcctttgatcggctttacaagtacgacataacagaaaccttagacaagatgaaccttcgacatgacgaaatcttccagattgaagtaaccattacatcctacgatggaactgtactcgatagtggccttattcccacaccgtcaatcatctatgatcctgctcatcatgatattagttcgcaccacctgtcgctcaacaaggttcgtcatgatctgagtacactgagtgagcgagatattggaagccttaaatatgctttgagcagcttgcaggcagatacctcagcagatggttttgctgccattgcatccttccatggtctgcctgccaaatgtaatgacagccacaataacgaggtggcatgctgtatccatggaatgcctacattcccccactggcacagactctacaccctccaatttgagcaagctctaagaagacatggctctagtgtagcagtaccctactgggactggacaaagccaatacataatattccacatctgttcacagacaaagaatactacgatgtctggagaaataaagtaatgccaaatccatttgcccgagggtatgtcccctcacacgatacatacacggtaagagacgtccaagaaggcctgttccacctgacatcaacgggtgaacactcagcgcttctgaatcaagctcttttggcgctggaacagcacgactactgcgattttgcagtccagtttgaagtcatgcacaacacaatccattacctagtgggaggacctcaagtctattctttgtcatcccttcattatgcttcatatgatccgatcttcttcatacaccactcctttgtagacaaggtttgggctgtctggcaggctcttcaagaaaagagaggccttccatcagaccgtgctgactgcgctgttagtctcatgactcagaacatgaggcctttccatcacgaaattaaccataaccagttcaccaagaaacatgcagttccaaatgatgttttcaagtacgaactcctgggttacagatacgacaatctggaaatcggtggcatgaatttgcatgaaattgaaaaggaaatcaaagacaaacagcaccatgtgagagtgtttgcagggttcctccttcacggaattagaacctcagctgatgtccaattccagatttgtaaaacatcagaagattgtcaccatggaggccaaatcttcgttcttggggggactaaagagatggcctgggcttataaccgtttattcaagtacgatattacccatgctcttcatgacgcacacatcactccagaagacgtattccatccctctgaaccattcttcatcaaggtgtcagtgacagccgtcaacggaacagttcttccggcttcaatcctgcatgcaccaaccattatctatgaacctggtctcgaccatcacgaagatcatcattcttcttctatggctggacatggtgtcagaaaggaaatcaacacacttaccactgcagaggtggacaatctcaaagatgccatgagagccgtcatggcagaccacggtccaaatggataccaggctatagcagcgttccatggaaacccaccaatgtgccctatgccagatggaaagaattactcgtgttgtacacatggcatggctactttcccccactggcacagactgtacacaaaacagatggaagatgccttgaccgcccatggtgccagagtcggccttccttactgggactggacaactgcctttacagctttgccaacttttgtcacagatgaagaggacaatcctttccatcatggtcacatagactatttgggagtggatacaactcggtcgccccgagacaagttgttcaatgatccagagcgaggatcagaatcgttcttctacaggcaggttctcttggctttggagcagacagatttttgtcagtttgaagtccagttcgagattacccacaatgccgtccattcttggactggtggactgaccccctacggaatgtccactctggagtacacgacctatgatcctcttttctggcttcatcatgccaatactgaccgaatatgggctatttggcaagctcttcaagaatacagaggtctcccatacgaccatgccaactgcgaaatccaggcaatgaagaggcccctacggccattctctgatcctattaatcacaatgcgttcactcattctaacgcaaaaccaacagacgttttcgagtacagtagattcaacttccagtatgataacttacggtttcatggtatgacaataaaaaagttggagcatgagcttgagaaacagaaggaagaagacaggacattcgctgccttcctgctccacggcattaaaaaaagtgctgatgtaagctttgacgtgtgcaaccatgatggagaatgccactttgcaggtaccttcgctattcttggaggcgagcacgagatgccttggtcctttgacagacttttccgatatgacatcacccaagttcttaagcaaatgcacctggagtatgattctgatttcacgttccatatgagaattattgacacttcgggtaaacaactgccatcagacctgatcaagatgccaacggttgaacacagcccaggaggaaaacatcatgagaaacatcatgaagatcaccatgaagacatacttgttcgtaaaaacattcactctctgtcacaccatgaagccgaagaattgagagatgcattgtacaagcttcaaaacgatgaaagtcacggtggatatgaacatattgctggattccatggttatcctaacctctgtcctgaaaaaggggatgaaaagtatccttgctgtgttcatggtatgtctatattcccccactggcacaggctccacacgatacagtttgaaagagctctgaagaaacatggttcccatctcggtattccttactgggattggactcagacgatatcgtctcttcctaccttctttgcagattctggtaataataatccattcttcaaataccatatcagaagcataaatcaagacactgttcgagacgttaatgaagcgatattccaacaaacgaagtttggagaattttcgtccatattttacctggccttgcaggctctagaagaggataattattgcgattttgaggtgcagtacgagatacttcacaatgaggtgcatgccttgattggtggagccgagaagtattccatgtccacgctggaatattcagccttcgatccatacttcatgatccaccacgcgtctttggacaagatctggataatctggcaggagcttcagaaacgccgggttaagcctgcacatgctggttcgtgtgccggagacattatgcacgttccacttcatcctttcaattatgaaagcgtcaacaacgatgactttaccagagagaattctctccccaacgcggtcgttgacagtcatagattcaactataaatatgacaatcttaatcttcacggccacaatatagaggagcttgaggaggttctccgcagcctgagacttaaatctcgtgttttcgcaggatttgttctgtccggaatccgcacaactgcagttgtgaaagtgtatatcaaaagcggaaccgattccgacgacgaatatgccggttcgtttgtcatccttggaggagccaaagagatgccttgggcatatgagaggttgtatagatttgacattacagaaactgtacacaatcttaaccttacggatgatcatgttaaattcaggtttgacttaaagaaatatgatcatactgaattggatgcatcagtactgccagctcctataatcgttcgacgtccaaataatgcggtttttgatatcattgagattccaatagggaaagacgtgaatcttccaccgaaggtggtcgtcaagagaggcactaaaatcatgttcatgtctgttgatgaagccgttaccactcccatgctgaacctgggtagttatacagcaatgttcaaatgtaaagtgccaccattcagcttccatgcttttgagcttgggaaaatgtattctgtcgaatctggtgactatttcatgacggcatcaacaactgaattatgcaacgataataacttaaggatacatgtccatgtggatgacgaatagacaaaggtattctgatgcgttgaggatgtttctgaaacgtttggaatatagtatagtatagtagatgagaaggaatgggctcctgttatcaaactcataataattattttgatagccagatttcctgttttgttaatgaattacgaagacagtctatgttcttttcactgtaagacaggtcttacgttagtccctttctaaagaggtaacattggcagctgagccgtggggaggtggtgcggctgaaatcccctcatttttacgggatggggtttaaccctccccctcccacttttgaaaacgaacgtaaattttactcattcgaaatagctcagagcagatcttagacgatctacgaccccagaccttcccattcggtaatcgcttccgtcgcccatggataagatagttaattgcaagaacgacttctctcatttggttttaagaagaaaagaatattccccaaagtaaaagtatttgtatgagtgttaactcgccattggcctgagagatggctgttaattgaaattaaaatattgtaaatatttgtaacttcgagttaaatggaatcttcgtgtaatgaattttttcctgcaagacattgtatctcatgcagctcatatttataattgtgtcaataaaatgtgtcatttgcaaaaaaaaaaaaaaaaaaaaaaaaaaaa

SEQ ID NO: 32 - DNA, 10949 bp, Megathura crenulata
  SEQ ID NO32 is the complementary sequence of the sequence of SEQ ID NO31
ttttttttttttttttttttttttttttgcaaatgacacattttattgacacaattataaatatgagctgcatgagatacaatgtcttgcaggaaaaaattcattacacgaagattccatttaactcgaagttacaaatatttacaatattttaatttcaattaacagccatctctcaggccaatggcgagttaacactcatacaaatacttttactttggggaatattcttttcttcttaaaaccaaatgagagaagtcgttcttgcaattaactatcttatccatgggcgacggaagcgattaccgaatgggaaggtctggggtcgtagatcgtctaagatctgctctgagctatttcgaatgagtaaaatttacgttcgttttcaaaagtgggagggggagggttaaaccccatcccgtaaaaatgaggggatttcagccgcaccacctccccacggctcagctgccaatgttacctctttagaaagggactaacgtaagacctgtcttacagtgaaaagaacatagactgtcttcgtaattcattaacaaaacaggaaatctggctatcaaaataattattatgagtttgataacaggagcccattccttctcatctactatactatactatattccaaacgtttcagaaacatcctcaacgcatcagaatacctttgtctattcgtcatccacatggacatgtatccttaagttattatcgttgcataattcagttgttgatgccgtcatgaaatagtcaccagattcgacagaatacattttcccaagctcaaaagcatggaagctgaatggtggcactttacatttgaacattgctgtataactacccaggttcagcatgggagtggtaacggcttcatcaacagacatgaacatgattttagtgcctctcttgacgaccaccttcggtggaagattcacgtctttccctattggaatctcaatgatatcaaaaaccgcattatttggacgtcgaacgattataggagctggcagtactgatgcatccaattcagtatgatcatatttctttaagtcaaacctgaatttaacatgatcatccgtaaggttaagattgtgtacagtttctgtaatgtcaaatctatacaacctctcatatgcccaaggcatctctttggctcctccaaggatgacaaacgaaccggcatattcgtcgtcggaatcggttccgcttttgatatacactttcacaactgcagttgtgcggattccggacagaacaaatcctgcgaaaacacgagatttaagtctcaggctgcggagaacctcctcaagctcctctatattgtggccgtgaagattaagattgtcatatttatagttgaatctatgactgtcaacgaccgcgttggggagagaattctctctggtaaagtcatcgttgttgacgctttcataattgaaaggatgaagtggaacgtgcataatgtctccggcacacgaaccagcatgtgcaggcttaacccggcgtttctgaagctcctgccagattatccagatcttgtccaaagacgcgtggtggatcatgaagtatggatcgaaggctgaatattccagcgtggacatggaatacttctcggctccaccaatcaaggcatgcacctcattgtgaagtatctcgtactgcacctcaaaatcgcaataattatcctcttctagagcctgcaaggccaggtaaaatatggacgaaaattctccaaacttcgtttgttggaatatcgcttcattaacgtctcgaacagtgtcttgatttatgcttctgatatggtatttgaagaatggattattattaccagaatctgcaaagaaggtaggaagagacgatatcgtctgagtccaatcccagtaaggaataccgagatgggaaccatgtttcttcagagctctttcaaactgtatcgtgtggagcctgtgccagtgggggaatatagacataccatgaacacagcaaggatacttttcatcccctttttcaggacagaggttaggataaccatggaatccagcaatatgttcatatccaccgtgactttcatcgttttgaagcttgtacaatgcatctctcaattcttcggcttcatggtgtgacagagagtgaatgtttttacgaacaagtatgtcttcatggtgatcttcatgatgtttctcatgatgttttcctcctgggctgtgttcaaccgttggcatcttgatcaggtctgatggcagttgtttacccgaagtgtcaataattctcatatggaacgtgaaatcagaatcatactccaggtgcatttgcttaagaacttgggtgatgtcatatcggaaaagtctgtcaaaggaccaaggcatctcgtgctcgcctccaagaatagcgaaggtacctgcaaagtggcattctccatcatggttgcacacgtcaaagcttacatcagcactttttttaatgccgtggagcaggaaggcagcgaatgtcctgtcttcttccttctgtttctcaagctcatgctccaacttttttattgtcataccatgaaaccgtaagttatcatactggaagttgaatctactgtactcgaaaacgtctgttggttttgcgttagaatgagtgaacgcattgtgattaataggatcagagaatggccgtaggggcctcttcattgcctggatttcgcagttggcatggtcgtatgggagacctctgtattcttgaagagcttgccaaatagcccatattcggtcagtattggcatgatgaagccagaaaagaggatcataggtcgtgtactccagagtggacattccgtagggggtcagtccaccagtccaagaatggacggcattgtgggtaatctcgaactggacttcaaactgacaaaaatctgtctgctccaaagccaagagaacctgcctgtagaagaacgattctgatcctcgctctggatcattgaacaacttgtctcggggcgaccgagttgtatccactcccaaatagtctatgtgaccatgatggaaaggattgtcctcttcatctgtgacaaaagttggcaaagctgtaaaggcagttgtccagtcccagtaaggaaggccgactctggcaccatgggcggtcaaggcatcttccatctgttttgtgtacagtctgtgccagtgggggaaagtagccatgccatgtgtacaacacgagtaattctttccatctggcatagggcacattggtgggtttccatggaacgctgctatagcctggtatccatttggaccgtggtctgccatgacggctctcatggcatctttgagattgtccacctctgcagtggtaagtgtgttgatttcctttctgacaccatgtccagccatagaagaagaatgatgatcttcgtgatggtcgagaccaggttcatagataatggttggtgcatgcaggattgaagccggaagaactgttccgttgacggctgtcactgacaccttgatgaagaatggttcagagggatggaatacgtcttctggagtgatgtgtgcgtcatgaagagcatgggtaatatcgtacttgaataaacggttataagcccaggccatctctttagtccccccaagaacgaagatttggcctccatggtgacaatcttctgatgttttacaaatctggaattggacatcagctgaggttctaattccgtgaaggaggaaccctgcaaacactctcacatggtgctgtttgtctttgatttccttttcaatttcatgcaaattcatgccaccgatttccagattgtcgtatctgtaacccaggagttcgtacttgaaaacatcatttggaactgcatgtttcttggtgaactggttatggttaatttcgtgatggaaaggcctcatgttctgagtcatgagactaacagcgcagtcagcacggtctgatggaaggcctctcttttcttgaagagcctgccagacagcccaaaccttgtctacaaaggagtggtgtatgaagaagatcggatcatatgaagcataatgaagggatgacaaagaatagacttgaggtcctcccactaggtaatggattgtgttgtgcatgacttcaaactggactgcaaaatcgcagtagtcgtgctgttccagcgccaaaagagcttgattcagaagcgctgagtgttcacccgttgatgtcaggtggaacaggccttcttggacgtctcttaccgtgtatgtatcgtgtgaggggacataccctcgggcaaatggatttggcattactttatttctccagacatcgtagtattctttgtctgtgaacagatgtggaatattatgtattggctttgtccagtcccagtagggtactgctacactagagccatgtcttcttagagcttgctcaaattggagggtgtagagtctgtgccagtgggggaatgtaggcattccatggatacagcatgccacctcgttattgtggctgtcattacatttggcaggcagaccatggaaggatgcaatggcagcaaaaccatctgctgaggtatctgcctgcaagctgctcaaagcatatttaaggcttccaatatctcgctcactcagtgtactcagatcatgacgaaccttgttgagcgacaggtggtgcgaactaatatcatgatgagcaggatcatagatgattgacggtgtgggaataaggccactatcgagtacagttccatcgtaggatgtaatggttacttcaatctggaagatttcgtcatgtcgaaggttcatcttgtctaaggtttctgttatgtcgtacttgtaaagccgatcaaaggcccaaggcatttctttttcacctcccaacacggtgaagtaccctgcatcttcacagtctcccgctgtgcgacagacctggaaatcgacagtggcagaggtgccaaatccttcaagcaagaatccggcaaagacccttgaatgttcctgcctgttacggacaatatggtcaatgccacgaattgaccaccctgcaaattccagagtgtcatatatgtatccaaacttttggtagtcgaatgtatcttcaggctttgagaaatcatgcgtgtgatcattaagattatatggtgctccaaagctgaaaggcttcagaggagttttcatgagatttacagcacagttagcctcagatccagaaagtcctctgaaacgctgcaacgattgccatatagcccaaatacgatcagtttgggagtgatggatgtagaaaagtggatcataggaagcataatgcagatggccaattccatagggctccttgcctccaacccaggtatgaacaccattatgcataatttcaaactgaacctcgaagtcgcagtagttggtttgttcaagagcaagcagtgcttgctcaataaaccagttatgatgtgtttttgttgatccctggaagagacgatccctattgatatctcttttagtatgtacgttttctccttcaaactctattttagaaccaataaatggatttggtatatctctgcctgtctccgggtcatgaatagttgagacggtcaggagctctggtaattcagaacgaggtagggtccagtcccagtacggaagtccaacgactgcaccatgttttctgagagaatcttggaattggactgtgtacagacggtgccattgtgggaacgttgccatgccatggacgcagcacgcaaacctctttgaagcagctggtgatggacaaagaggaggaagagcatggaaggaagcgattgcctggaacccatcagatgaatggtcggctttcatggactcgagggctttcaccagtgacagttgttgacgcttgtccaattgtgtgatatctttgcgaatcatcactggttcagtgtgtccatcatctgtgtgggcagaatctgttccggcctcaaacagtatagtgggagagtggatgacatgactgtctagatgagatccattcacagccgtaatctcaatttccagatcgaaatccccttgcaggtgcacaccgttctttttcaaggctttagttatgtcaagtttgtaaagtctgtcaaatacaaaggacatctccgtttgaccgcctaagatggagaacataccagcctcatgactgcagtccttcgtgtgttccccagttgggagacatacaaatacatggacatcggcagatgttccaatattgtgaaggaggaagccagcgaatactctgtcatgcctttttctttcttgcagcatgtgatcaagctgaggaatcgagtagtgattcagttccagattatcgtaagcataaccaaagctgttttgatattcaaaaccatcatgtggagtggcatgcgttttcgttgcctcgtcattgttgtcgctcttatcaaacggctggagtggcgtgtgcatcaattggatagcgcaattcgcttctcggtatggccgttttcggaacctctgaaggtcttgccagattgcccagattctgtcagtcgttgcgtgatgaaggaagaacacaggatcaaaagcagcgtaatcaaggtttgacatggaatatttacctttgccaccaagtaaagaatgcaatgcattgtgtaatatctccaactgaatctcgaaatcacagaagttatcctgctccaaggcgtaaaggacctgctcgtaaaagtagtctgaatggaagaggctgtccttgggatccctagtagtgatttcattctcgtgtgtgattttgccatgatggaatgggtttgtctcatagtgatgttgcctggaattgtagtaagtggcgtctgaaatcaggtgaggtaaatgttcgattcgttttgtccagtcccaatagggaacagctacagatgatccttttttcaggagggcattctcaacgacaaccacgtacagtctgtgccagtgagggaaagttggcatgccatggacacaacaagagatggggttaccatttaactggcacaaaccagggctaccatggaatttagctattgattcgtaagttccatcgttttcaatttccaggaaggcagctctcagactttcgagttcaccaaggctcagattttcaatgttttttcgaatacggttggcagaagttacttcagcttgatatacttcgccttcatggtgacctattcttggttcatgaatgactgaaggctgcttgaacaaatcttttcccaggcttgctggtttaaggtcaaatacttcatagtcaatgtagaagtggtcttctgcatgcagctttagatcgtggagtttttcggttatgtcatatttgaagactctatcgtatgcccatggcatctcgttttcatcacccagaaggtaaaactctccagcggggtgacagttgctgctatcggtgcaaatattgaatttaacaagagatgatttcttgaaaccagataacaggaagcctgcaaagaacctgtcttggctctttatcgcttcgagctttttattcaactgagagattgagagaccgttaaattccaaagtgtcatattcataattgaaattggtcttgtaatcaaaaacgttgaatggcactgaatgctccttggttacatggtctgtgttgatattggcagagaggccaaatggttgcaaaggttctctcatcgatgtaacagcacagttagcaacgttgtacggttttcctctgtacttctgtaaagcctgccatattgcccatatacgatctgtattagagtgatgcaagtagaatagtggatcaaaagcagtgtagcgaagcgatgccataccataaggctgagcgcctcctacaagtgcatggatgtagttatgggcaatctcatattggatttcaaagtcacagaaattgtcctgttccaacgctagcagtacttgtttggcaatgcttgtataatgaccaaactcaggctgttcgaagagtttattctgaacacttcttgttgttgttttgtcgactgtatctatatgaccatcgaaccaagggttatgcttctctaccccatcttcagggtcgacgtacttctcatgatctgccagttcgggaaggtgattaagaggagaggtccaatcccagtacggcaaagctccatcgtagccatgtcgtctcaaagcattttcactctgaaccgtcaacagtcgatgccagtgagggaacacagacatgccgtgaacacagcaggcaaattttttagatgcttcgggactaggacaccattttggctccccatggaatgcaccaagctgctgaaatccaccggcacttttgtcctctttcagtaaggctagggctttacgaagttcattcatctcctcggggctcaaacggtctacattttttcgaataagacgactccttggcactttgtcaaatttaacacgtgcatgctcacgaatgacagaagcatgtggaataagactggatgcaagtttagttccatcgacttcagtgatgtcaacagtaacttcgaaatcgccatcagcagtgagatcgagatctttcaaaacgtgcgtgatgtcaaacttgaaaacgcgatcaaatccccacttcatttccttgcttccaccgagcactgcaaatgttccagccttatgttgcacggaatcggtagttttaatgaagatatcaacatttgctgaagtacgtatgccagccaggagaaaaccggcatatattctgtcttcttgctggttttcgtggatcatcttttctatgttttccagagagatgcctccaaatgttaaatcttcgtatgtgtaatggaggacattttcatagtcgtagatcttgtttggcatggcattggcatgagtcttttcgttattgttaaggggagatgaaaaggcgaatggtttcagatgcatatgttccagagatatggcgcagtgggccctgtagggtttatgccgtctcatctgtaaggcttgccaaacggcccaaagacgatcaacgttagaatggtgaaagtaaaacaaaggatcgaaagctgtgtaatgtagggacgacattgagaaatgttcgcttcctcctgtccaagcgtgaatcgtgttatgggtaatctcaaattggacttcaaagtcacagtaatcttcttgttcaaaggcatacaaaatctggttgaacaggtctgtgtggtgtccaaaggtagcgggtttaaagagtctttgatctatttgtcttttggtgtggggagcattttcttcaaatgcaatcactgaactatgaaaaggatttgtgtgggatgcaccatgagaatccacataagtttcgtttcctgccagacccggaatgtgtgtcatgggttcagtccagtcccagtatgggataccaatggtagctccacggcctactaaagcatcttcaacttgtgtgacaaaaagcctgtgccagtgagggaagattggcattccatggacacaacaagcatatctgtccttggcatcagggcgagggcatctggccggaagaccatgatactcagctgtggcttgataaccgtcgacggagcgatcttcttggaatttatgaaacgcctctctcagattcaacacttcttcacgggtgagatcattgatgttctttctaacgaggagattggcgctttggtgatgcttcaccggaggatcagtgaaacccttctccggatgatgaacaacaactggatgaggtaacaaatcatcgggaagagctgtaccattgacactgaaaagctctgccttgacggtatagtgtccatccaacttcattcccatcttgtgaacagtgtcggacaagtcaaacaagtatggcctgtagaactgccatttcatctcgcttggtccgcccagaacgaagaaatcaccggccttctcacagtggtcatcattacgatcgttgtcgtcggggatgcagacgtacacaaagacattggctgaacctccaaatcctttcagacggaagcttgcgaaggctcttgcccttgaagaccgctcatccaaataggttttcagttcctctggagtcataccattaagattcaacgtgtcatagtcgtagtgaagttgacgatagtcaaagagatcggctggagtggaatgttcatttgttagtggtatgtcattgtcctcccaactgaaaggttccattttctgatgtaacaattcttgggcacaatccatagctttggggtctttgtttcggagctcttggagcctctgccagatggcgaaaattctgtcaacgttggagtggtgaagaaagaagatcggatcgtaggcggtgtactcgaggtttgccatagagtaatcatgctttccaccgaccaggtagtgaatcgcattatgtgctaactcgaactggatttcgaatttacagaattcatcctgctccagtgcgtccaaaacactctccataagatgggtgtgctggcctggcttcaccttctcaaaaagacggtcgtcgaccgcacgattggttttcttgtctagaaagtcaatgttccctctgtaccagtagttggtgtgagcctttccgccctgggaatcgatgtaaacagggtccgcagcgagagagggcaactgggtgatgggttctgtccagtcccagtatggcatcccagaagtacgcctcttcgtctgcagagctcgctccatgtgaactacgtacgccctgtgccagtgcgggaaagtaggcatgccgtgaatacagcaagctatagaagtgtccttatgcacacaagatgcgggcgcgccatgagcagcagctattttctggaaaccaatgctggatgagtccatctgcagctcacgcagggcagcctgcaggtccagggtctcttcctgagtcagatgctccacactctttctgaccaggttctctgcattggccaaagccaacacgactataagcaacctgaccgacagcattgcgcctctccaaatcgccag

SEQ ID NO: 33 - protein, 3414 aa, Megathura crenulata; identical to SEQ ID NO: 30
  SEQ ID NO33 is the deduced amino acid sequence of the coding region of SEQ ID NO31

SEQ ID NO: 34 - DNA, 28 bp, Artificial
  5′ RACE Primer 1
cggggtgacagttgctgctatcggtgca

SEQ ID NO: 35 - DNA, 24 bp, Artificial
  3′ RACE Primer 2
gccttgaccgcccatggtgccaga

SEQ ID NO: 36 - DNA, 27 bp, Artificial
  PCR Primer 3
ggggcgaccgagttgtatccactccca

SEQ ID NO: 37 - DNA, 27 bp, Artificial
  PCR Primer 4
ggctggaacatttgcagtgctcggtgg

SEQ ID NO: 38 - DNA, 49 bp, Artificial
  KLH2 Primer 1 (forward) 5′ to 3′
aggctacgcgttcgaaggagatagaaccatgtggaccatcttggctctc

SEQ ID NO: 39 - DNA, 38 bp, Artificial
  KLH2 Primer 2 (reverse) 5′ to 3′
ctggtgcggccgccttactcgtcttcaatgtggatatg

SEQ ID NO: 40 - DNA, 28 bp, Artificial
  KLH1 Primer 1 (forward) 5′ to 3′
atgctgtcggtcaggttgcttatagtcg

SEQ ID NO: 41 - DNA, 29 bp, Artificial
  KLH1 Primer 2 (reverse) 5′ to 3′
ctattcgtcatccacatggacatgtatcc

(19) United States
(12) Patent Application Publication
(10) Pub. No.: US 2004/0241695 A1
(43) Pub. Date: Dec. 2, 2004

(54) Hemocyanin nucleic acids and polypeptides and related vectors fusion protein conjugates cells and methods of use

(76) Inventors: Mark J Frey, Roscoe, IL (US); Elizabeth A Strachan, Rockford, IL (US)

Correspondence Address: LEYDIG VOIT & MAYER, LTD, TWO PRUDENTIAL PLAZA, SUITE 4900, 180 NORTH STETSON AVENUE, CHICAGO, IL, 60601-6780, US

(21) Appl. No.: 10/488,824

(22) PCT Filed: Sep. 6, 2002
(86) PCT No.: PCT/US02/28581

(30) Foreign Application Priority Data
Sep. 6, 2001 (US) 60317678
Dec. 21, 2001 (US) 60345418

(51) Int. Cl. C12Q 1/68; C07H 21/04; C12P 21/04; C07K 14/435

(52) U.S. Cl. 435/6; 435/69.6; 435/320.1; 435/325; 530/400; 536/23.5

(57) ABSTRACT

An isolated or purified nucleic acid molecule consisting essentially of a nucleotide sequence encoding KLH or a fragment thereof, as well as an isolated or purified nucleic acid molecule consisting essentially of a nucleotide sequence complementary thereto; a vector comprising such a nucleic acid, optionally as part of an encoded fusion protein when the nucleic acid encodes KLH or a fragment thereof; a cell comprising and expressing an above-described nucleic acid, optionally in the form of a vector; an isolated or purified polypeptide molecule consisting essentially of an amino acid sequence encoding KLH-1, KLH-2, or a fragment of either of the foregoing, as well as a conjugate or fusion protein comprising the same and a therapeutically or prophylactically active agent, and related compositions; a method of treating cancer, pathogenic infection, and high blood pressure in a mammal; methods of stimulating an immune response in a mammal; a method of identifying an immunogenic portion of KLH, and an isolated or purified nucleic acid molecule consisting essentially of a nucleotide sequence encoding the leader sequence of KLH-1 or KLH-2, optionally coupled 5′ to an isolated or purified nucleic acid molecule encoding a polypeptide or protein, in which case the nucleic acid is optionally in the form of a vector, as well as a eukaryotic cell comprising and expressing same.

### TECHNICAL FIELD OF THE INVENTION

The present invention relates to an isolated or purified nucleic acid molecule consisting essentially of a nucleotide sequence encoding keyhole limpet hemocyanin (KLH) and related nucleic acid molecules, vectors, cells, polypeptides, conjugates, fusion proteins and compositions; methods of treating cancer, pathogenic infection, and high blood pressure; methods of stimulating an immune response; and an isolated or purified nucleic acid molecule consisting essentially of the KLH leader sequence, optionally fused to an isolated or purified nucleic acid molecule encoding a polypeptide or protein, and related vectors and cells.

### BACKGROUND OF THE INVENTION

KLH is a copper-containing respiratory protein in the marine gastropod Megathura crenulata. There are two isoforms of KLH, namely KLH-1 and KLH-2. KLH is clinically used as an immunotherapeutic agent, specifically in the treatment of bladder cancer (Jurincic et al., J. Urol. 139: 723-726 (1988); and Swerdlow et al., J. Urol. 151: 1718-1722 (1994)). KLH is believed to be potentially useful in the treatment of other carcinomas, such as the epithelial-derived adenocarcinomas, when used as a carrier for mucin-like epitopes and carcinoma ganglioside (Harris et al., Micron 30: 597-623 (1999)). KLH is also widely used as a hapten carrier and as a generalized vaccine component, such as in a vaccine for AIDS (Naylor et al., Int. J. Immunopharmacol. 13 Suppl. 1:117-127 (1991)) and for the prophylaxis of cocaine abuse (Bagasra et al., Immunopharmacology 23(3):173-179 (1992)). It is also used in the diagnosis of Schistosomiasis (Grzych et al., J. Exp. Med. 165: 865-878 (1987); Markl et al., Naturwissenschaften 78: 30-31 (1991); and Wishahi et al., J. Urol. 153: 926-928 (1995)) and in drug assays (Harris et al., supra). Other areas of use include the testing of immune competency (DeKruyff et al., J. Immunol. 15: 2578-2587 (1995); Helling et al., Cancer Res. 54: 197-203 (1994); and Jennemann et al., J. Biochem. (Tokyo) 115: 1047-1052 (1994)), the assessment of stress and the research of inflammatory conditions (Harris et al., supra).

KLH is currently isolated from Megathura crenulata maintained under mariculture conditions, or from naturally occurring organisms in the ocean. Maintaining and or obtaining M. crenulata under such conditions is difficult, costly and/or unreliable. In an effort to overcome these obstacles, Markl et al. (WO 00/55192 and WO 01/14536) attempted to obtain the complete coding sequence of the KLH gene but was unsuccessful. Thus, it is an object of the present invention to enable the recombinant production of KLH. This and other objects and advantages of the present invention, as well as additional inventive features, will become apparent upon reading the detailed description of the invention provided herein.

### BRIEF DESCRIPTION OF THE INVENTION

The present invention provides an isolated or purified nucleic acid molecule consisting essentially of a nucleotide sequence encoding KLH-2 or a fragment thereof comprising at least 1,080 contiguous nucleotides. Similarly provided is an isolated or purified nucleic acid molecule consisting essentially of a nucleotide sequence that is complementary to a nucleotide sequence encoding KLH-2 or a fragment thereof comprising at least 1,080 contiguous nucleotides.

The present invention provides an isolated or purified nucleic acid molecule consisting essentially of a nucleotide sequence encoding KLH-1 or a fragment thereof comprising at least 2,300 contiguous nucleotides. Similarly provided is an isolated or purified nucleic acid molecule consisting essentially of a nucleotide sequence that is complementary to a nucleotide sequence encoding KLH-1 or a fragment thereof comprising at least 2,300 contiguous nucleotides.

In view of the above, the present invention further provides a vector comprising such an isolated or purified nucleic acid. When the isolated or purified nucleic acid molecule consists essentially of a nucleotide sequence encoding KLH or a fragment thereof, the nucleotide sequence is optionally part of an encoded fusion protein.

Also in view of the above, the present invention provides a cell comprising and expressing an above-described isolated or purified nucleic acid molecule. The isolated or purified nucleic acid molecule is optionally in the form of a vector.

Further provided by the present invention is an isolated or purified polypeptide molecule consisting essentially of an amino acid sequence encoding KLH-2 or at least 360 contiguous amino acids of KLH-2, either one of which is optionally glycosylated, amidated, carboxylated, phosphorylated, esterified, N-acylated or converted into an acid addition salt and/or dimerized or polymerized. The isolated or purified polypeptide molecule is essentially free from KLH-1.

Further provided by the present invention is an isolated or purified polypeptide molecule consisting essentially of an amino acid sequence encoding KLH-1 or at least 770 contiguous amino acids of KLH-1, either one of which is optionally glycosylated, amidated, carboxylated, phosphorylated, esterified, N-acylated or converted into an acid addition salt and/or dimerized or polymerized. The isolated or purified polypeptide molecule is essentially free from KLH-2.

In view of the above-described isolated or purified polypeptide molecule, the present invention further provides a conjugate or fusion protein comprising the isolated or purified polypeptide molecule or fragment thereof and a therapeutically or prophylactically active agent. The active agent can be an immunogen, such as an antigen of a pathogen or an antigen of a cancer. Alternatively, the active agent can be hemocyanin β-adrenergic receptor peptide.

Also, in view of the above-described isolated or purified polypeptide molecule, the present invention provides a composition comprising the isolated or purified polypeptide molecule, optionally in the form of a conjugate or a fusion protein comprising a therapeutically or prophylactically active agent, and an excipient or adjuvant.

Still further provided by the present invention is a method of treating cancer prophylactically or therapeutically in a mammal. The method comprises administering to the mammal an effective amount of:

(a) an isolated or purified nucleic acid molecule encoding a fusion protein comprising (i) KLH or an immunogenic fragment thereof, and (ii) an antigen of the cancer, wherein the isolated or purified nucleic acid molecule is optionally in the form of a vector,

(b) a fusion protein comprising (i) KLH or an immunogenic fragment thereof, and (ii) an antigen of the cancer,

(c) a conjugate comprising (i) (a) KLH-2 or an immunogenic fragment thereof comprising at least 360 contiguous amino acids, wherein the KLH-2 is essentially free from KLH-1, or (b) KLH-1 or an immunogenic fragment thereof comprising at least 770 contiguous amino acids, wherein the KLH-1 is essentially free from KLH-2, and (ii) an antigen of the cancer or an anti-cancer agent, or

(d) a composition comprising (i) (a) KLH-2 or an immunogenic fragment thereof comprising at least 360 contiguous amino acids, wherein the KLH-2 is essentially free from KLH-1, or (b) KLH-1 or an immunogenic fragment thereof comprising at least 770 contiguous amino acids, wherein the KLH-1 is essentially free from KLH-2, and (ii) an antigen of the cancer and/or an anti-cancer agent, whereupon the mammal is treated for the cancer prophylactically or therapeutically.

Yet still further provided by the present invention is a method of treating a mammal prophylactically or therapeutically for a pathogenic infection. The method comprises administering to the mammal an effective amount of:

(a) an isolated or purified nucleic acid molecule encoding a fusion protein comprising (i) KLH or an immunogenic fragment thereof, and (ii) an antigen of the pathogen, wherein the isolated or purified nucleic acid molecule is optionally in the form of a vector,

(b) a fusion protein comprising (i) KLH or an immunogenic fragment thereof, and (ii) an antigen of the pathogen,

(c) a conjugate comprising (i) (a) KLH-2 or an immunogenic fragment thereof comprising at least 360 contiguous amino acids, wherein the KLH-2 is essentially free from KLH-1, or (b) KLH-1 or an immunogenic fragment thereof comprising at least 770 contiguous amino acids, wherein the KLH-1 is essentially free from KLH-2, and (ii) an antigen of the pathogen or an anti-pathogen agent, or

(d) a composition comprising (i) (a) KLH-2 or an immunogenic fragment thereof comprising at least 360 contiguous amino acids, wherein the KLH-2 is essentially free from KLH-1, or (b) KLH-1 or an immunogenic fragment thereof comprising at least 770 contiguous amino acids, wherein the KLH-1 is essentially free from KLH-2, and (ii) an antigen of the pathogen and/or an anti-pathogen agent, whereupon the mammal is treated for the pathogenic infection prophylactically or therapeutically.

Methods of stimulating an immune response in a mammal are also provided by the present invention. In one embodiment, the method comprises administering to the mammal an effective amount of:

(a) an isolated or purified nucleic acid molecule consisting essentially of a nucleotide sequence encoding KLH-2 or an immunogenic fragment thereof comprising at least 1,080 contiguous nucleotides, wherein the isolated or purified nucleic acid molecule is optionally in the form of a vector,

(b) an isolated or purified nucleic acid molecule consisting essentially of a nucleotide sequence encoding KLH-1 or an immunogenic fragment thereof comprising at least 2,300 contiguous nucleotides, wherein the isolated or purified nucleic acid molecule is optionally in the form of a vector,

(c) an isolated or purified polypeptide molecule consisting essentially of an amino acid sequence encoding KLH-2 or an immunogenic fragment thereof comprising at least 360 contiguous amino acids, wherein the KLH-2 is essentially free from KLH-1, or

(d) an isolated or purified polypeptide molecule consisting essentially of an amino acid sequence encoding KLH-1 or an immunogenic fragment thereof comprising at least 770 contiguous amino acids, wherein the KLH-1 is essentially free from KLH-2,

whereupon an immune response in the mammal is stimulated. In another embodiment, the method comprises stimulating an immune response to an antigen in a mammal by administering to the mammal an effective amount of dendritic cells which have been previously isolated from the mammal and treated in vitro with:

(a) a fusion protein comprising (i) KLH or an immunogenic fragment thereof, and (ii) an antigen,

(b) a conjugate comprising (i) (a) KLH-2 or an immunogenic fragment thereof comprising at least 360 contiguous amino acids, wherein the KLH-2 is essentially free from KLH-1, or (b) KLH-1 or an immunogenic fragment thereof comprising at least 770 contiguous amino acids, wherein the KLH-1 is essentially free from KLH-2, and (ii) an antigen, or

(c) a composition comprising (i) (a) KLH-2 or an immunogenic fragment thereof comprising at least 360 contiguous amino acids, wherein the KLH-2 is essentially free from KLH-1, or (b) KLH-1 or an immunogenic fragment thereof comprising at least 770 contiguous amino acids, wherein the KLH-1 is essentially free from KLH-2, and (ii) an antigen,

whereupon an immune response to the antigen is induced in the mammal.

In addition, a method of treating high blood pressure in a mammal is provided by the present invention. The method comprises administering to the mammal an effective amount of:

(a) an isolated or purified nucleic acid molecule encoding a fusion protein comprising (i) KLH or an immunogenic fragment thereof, and (ii) hemocyanin β-adrenergic receptor peptide, wherein the isolated or purified nucleic acid molecule is optionally in the form of a vector,

(b) a fusion protein comprising (i) KLH or an immunogenic fragment thereof, and (ii) hemocyanin β-adrenergic receptor peptide,

(c) a conjugate comprising (i) (a) KLH-2 or an immunogenic fragment thereof comprising at least 360 contiguous amino acids, wherein the KLH-2 is essentially free from KLH-1, or (b) KLH-1 or an immunogenic fragment thereof comprising at least 770 contiguous amino acids, wherein the KLH-1 is essentially free from KLH-2, and (ii) hemocyanin β-adrenergic receptor peptide, or

(d) a composition comprising (i) (a) KLH-2 or an immunogenic fragment thereof comprising at least 360 contiguous amino acids, wherein the KLH-2 is essentially free from KLH-1, or (b) KLH-1 or an immunogenic fragment thereof comprising at least 770 amino acids, wherein the KLH-1 is essentially free from KLH-2, and (ii) hemocyanin β-adrenergic receptor peptide, whereupon the mammal is treated for high blood pressure prophylactically or therapeutically.

A method of identifying an immunogenic portion of KLH is also provided. The method comprises:

(i) contacting antigen-presenting cells with KLH or a portion thereof,

(ii) harvesting the antigen-presenting cells that have been contacted with KLH or a portion thereof,

(iii) purifying complexes of major histocompatibility complex (MHC) and peptidic fragments derived from KLH from the harvested antigen-presenting cells,

(iv) isolating the peptidic fragments from the purified complexes, and

(v) analyzing the peptidic fragments to determine which fragments were bound to MHC, whereupon an immunogenic portion of KLH is identified.

An isolated or purified nucleic acid molecule consisting essentially of the nucleotide sequence encoding the leader sequence of KLH is also provided. An isolated or purified nucleic acid molecule consisting essentially of the aforementioned isolated or purified nucleic acid molecule coupled 5′ to an isolated or purified nucleic acid molecule encoding a polypeptide or protein, optionally in the form of a vector, is also provided. Upon expression in a eukaryotic cell, secretion of the encoded polypeptide or protein by the eukaryotic cell results. Thus, a eukaryotic cell comprising and expressing such an isolated or purified nucleic acid molecule is also provided.

### BRIEF DESCRIPTION OF THE FIGURES

FIG. 1 represents the nucleotide (SEQ ID NO: 1 (coding); SEQ ID NO: 2 (complementary)) and deduced amino acid (SEQ ID NO: 3) sequences of KLH-2.

FIG. 2 represents the nucleotide (SEQ ID NO: 4 (coding); SEQ ID NO: 5 (complementary)) and deduced amino acid (SEQ ID NO: 6) sequences of the leader sequence of KLH-2.

FIG. 3 represents the nucleotide (SEQ ID NO: 7 (coding); SEQ ID NO: 8 (complementary)) and deduced amino acid (SEQ ID NO: 9) sequences of KLH-2 with the leader sequence.

FIG. 4 represents the nucleotide (SEQ ID NO: 10 (coding); SEQ ID NO: 11 (complementary)) and deduced amino acid (SEQ ID NO: 12) sequences of the 5′ untranslated region, the coding region, and the 3′ untranslated region of KLH-2.

FIG. 5 represents the nucleotide (SEQ ID NO: 22 (coding); SEQ ID NO: 23 (complementary)) and deduced amino acid (SEQ ID NO: 24) sequences of KLH-1.

FIG. 6 represents the nucleotide (SEQ ID NO: 25 (coding); SEQ ID NO: 26 (complementary)) and deduced amino acid (SEQ ID NO: 27) sequences of the leader sequence of KLH-1.

FIG. 7 represents the nucleotide (SEQ ID NO: 28 (coding); SEQ ID NO: 29 (complementary)) and deduced amino acid (SEQ ID NO: 30) sequences of KLH-1 with the leader sequence.

FIG. 8 represents the nucleotide (SEQ ID NO: 31 (coding); SEQ ID NO: 32 (complementary)) and deduced amino acid (SEQ ID NO: 33) sequences of the 5′ untranslated region, the coding region, and the 3′ untranslated region of KLH-1.

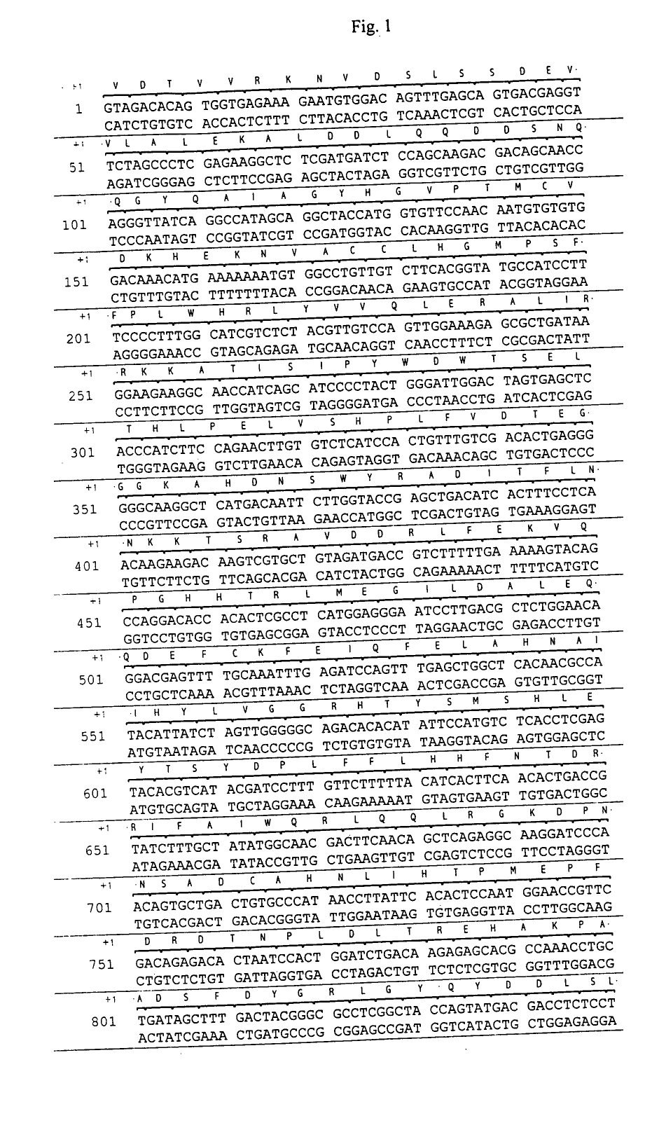

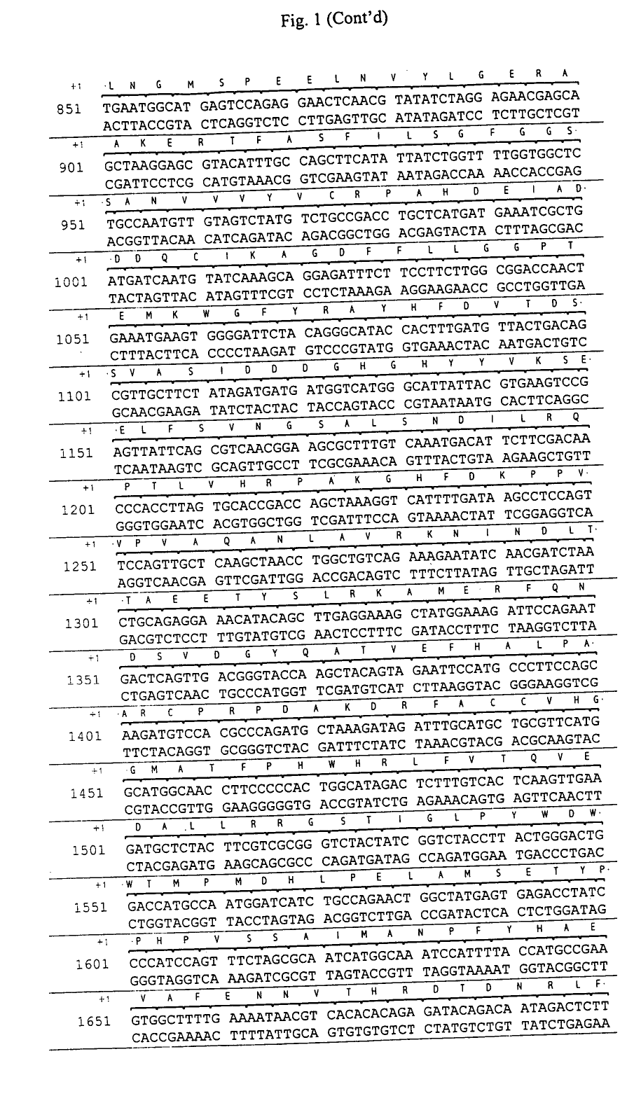

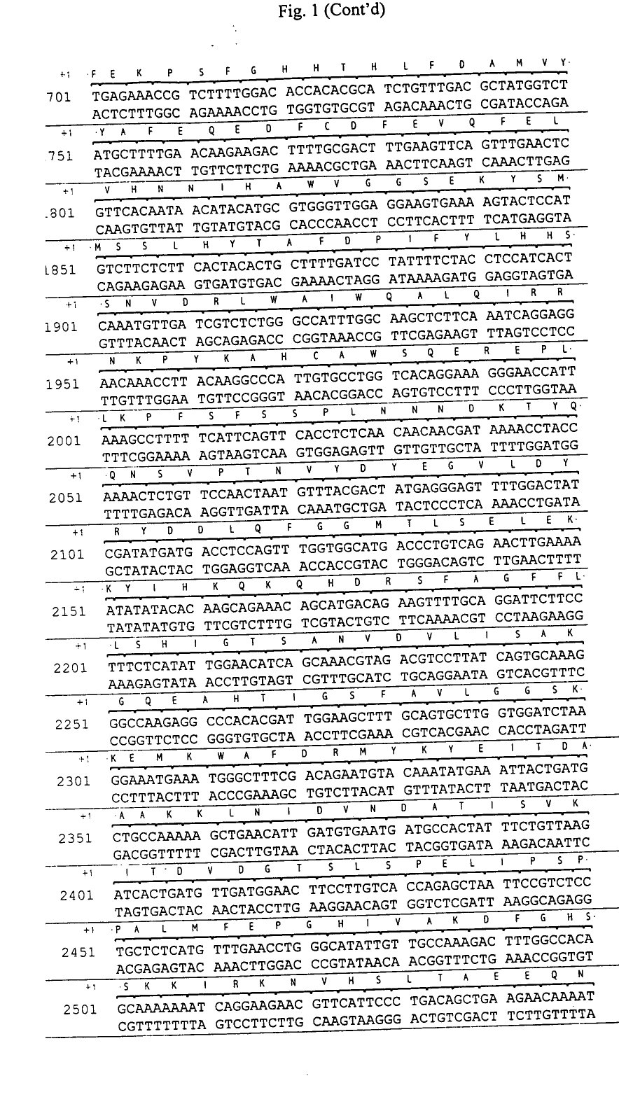

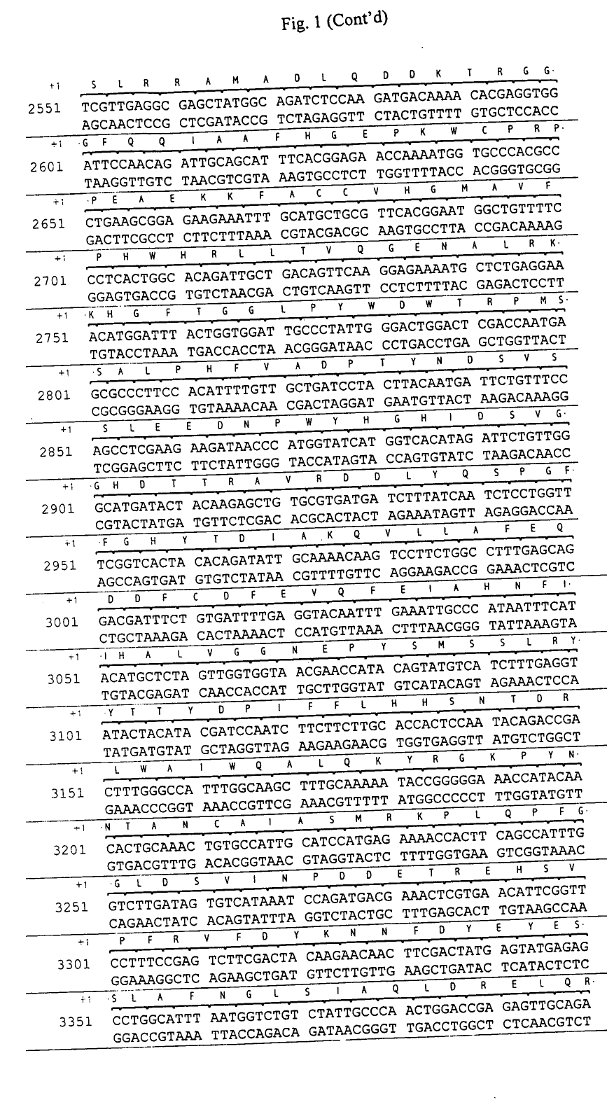

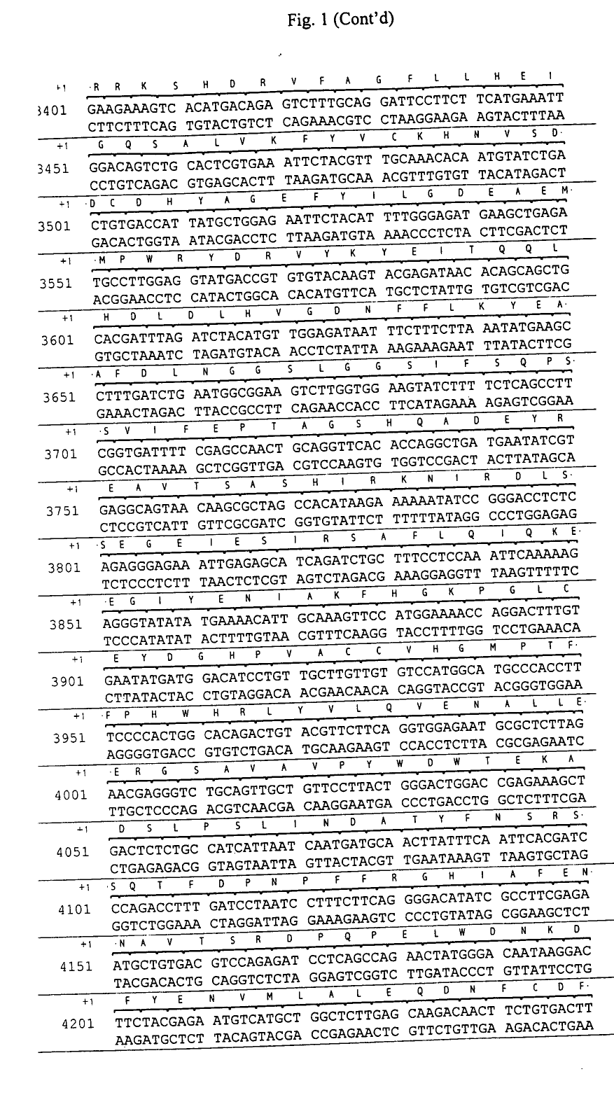

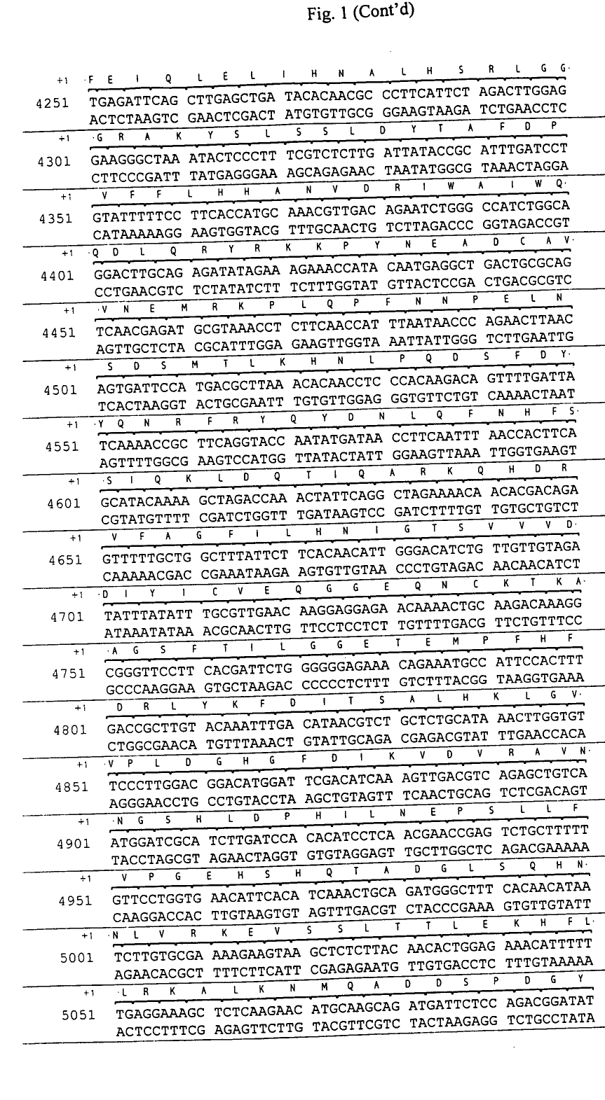

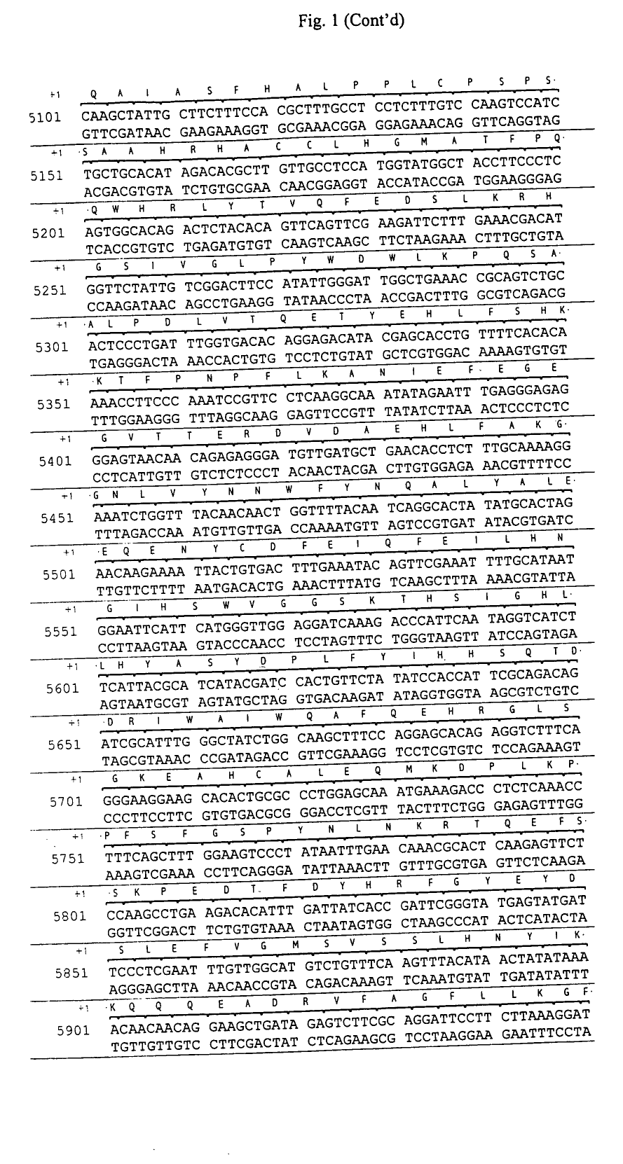

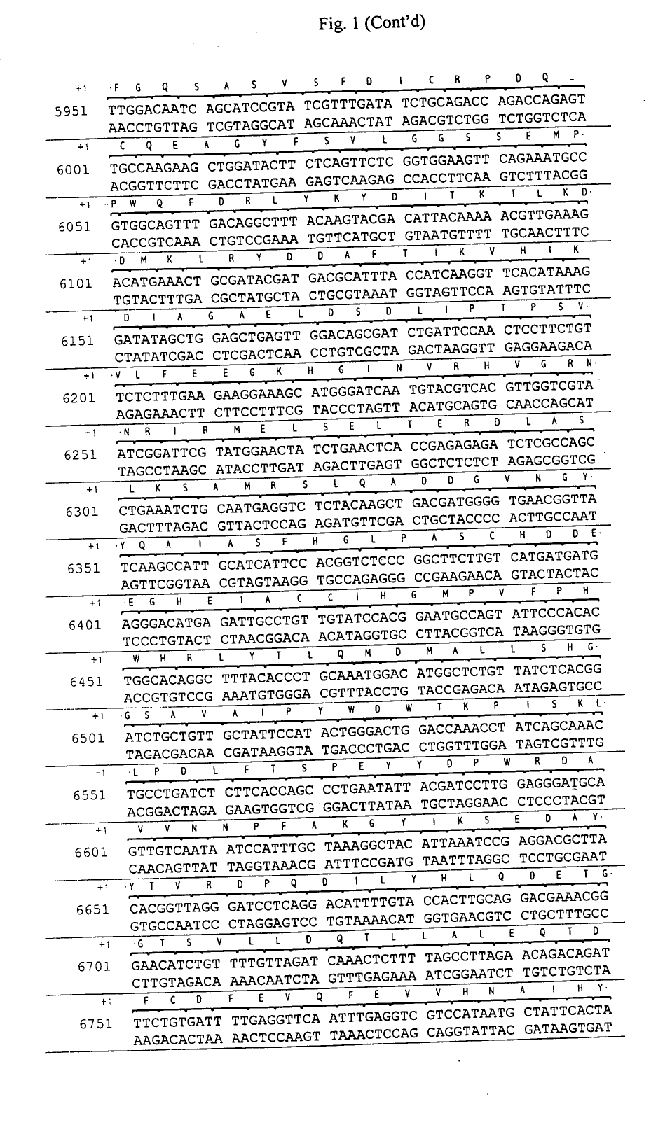

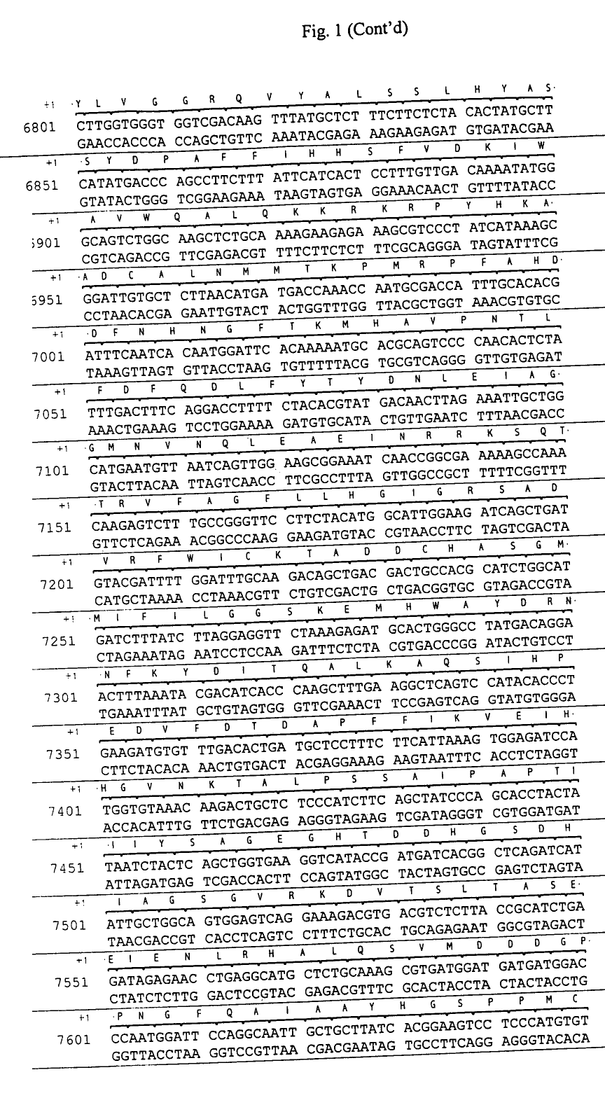

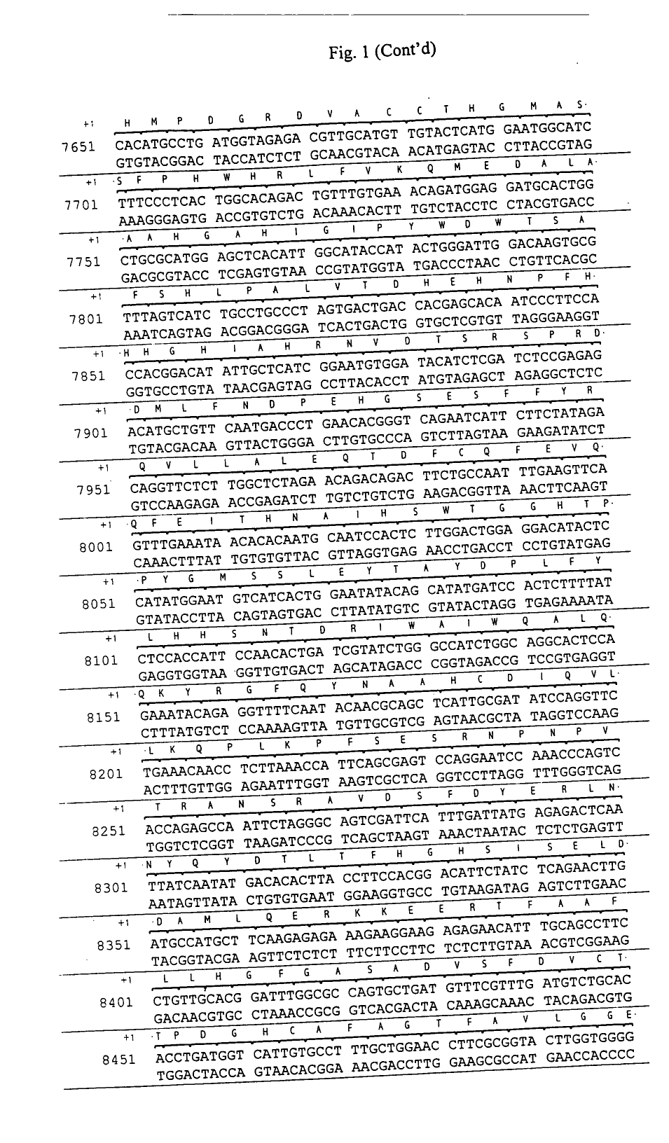

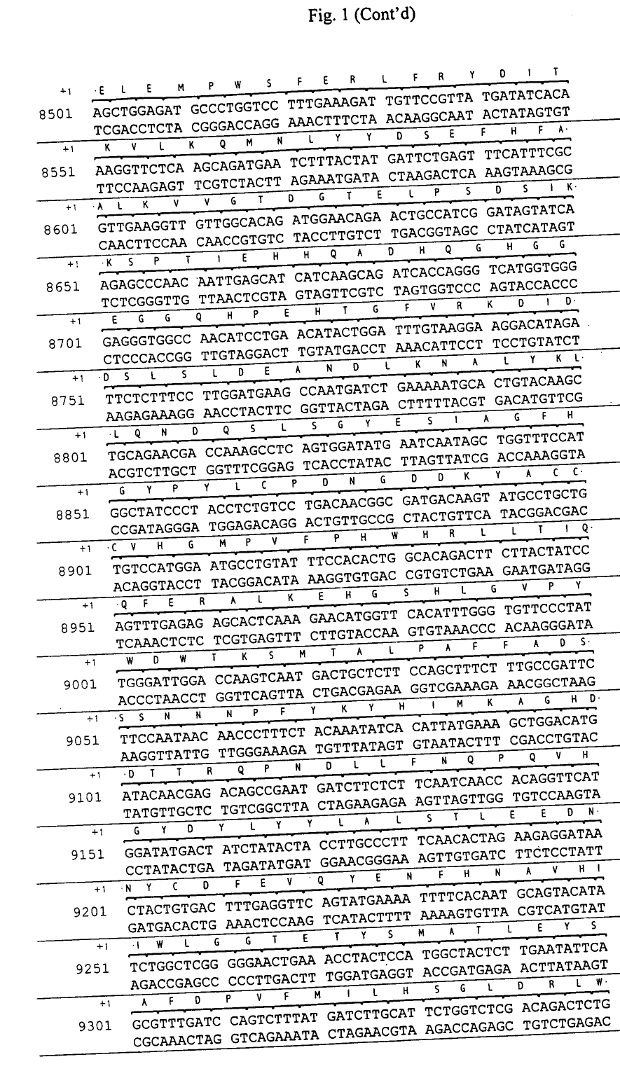

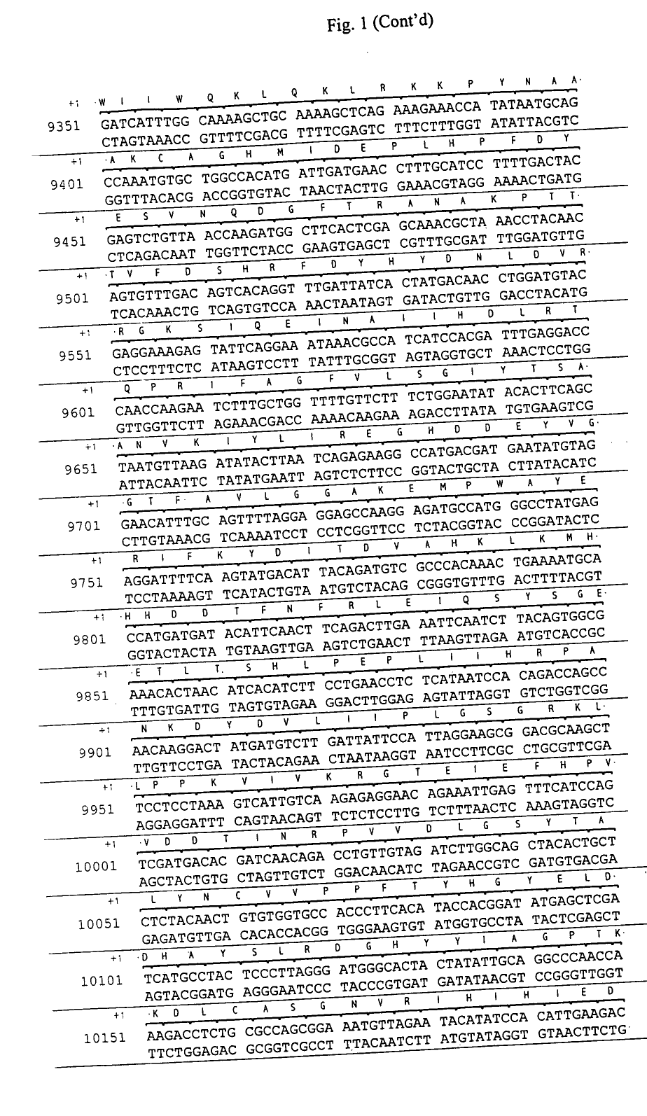

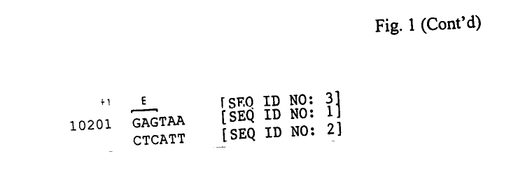

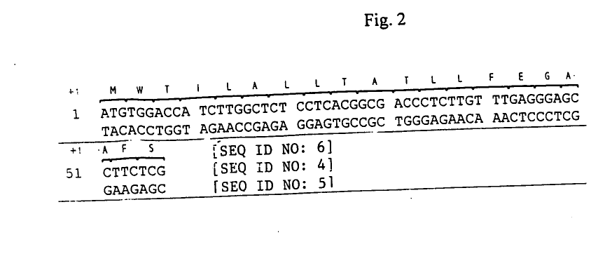

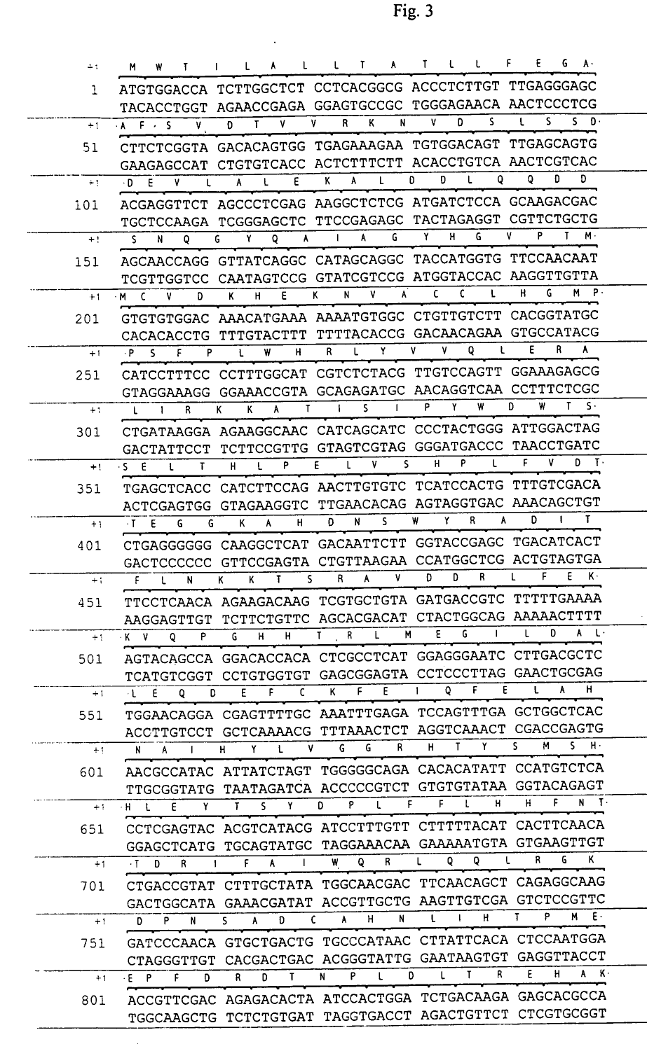

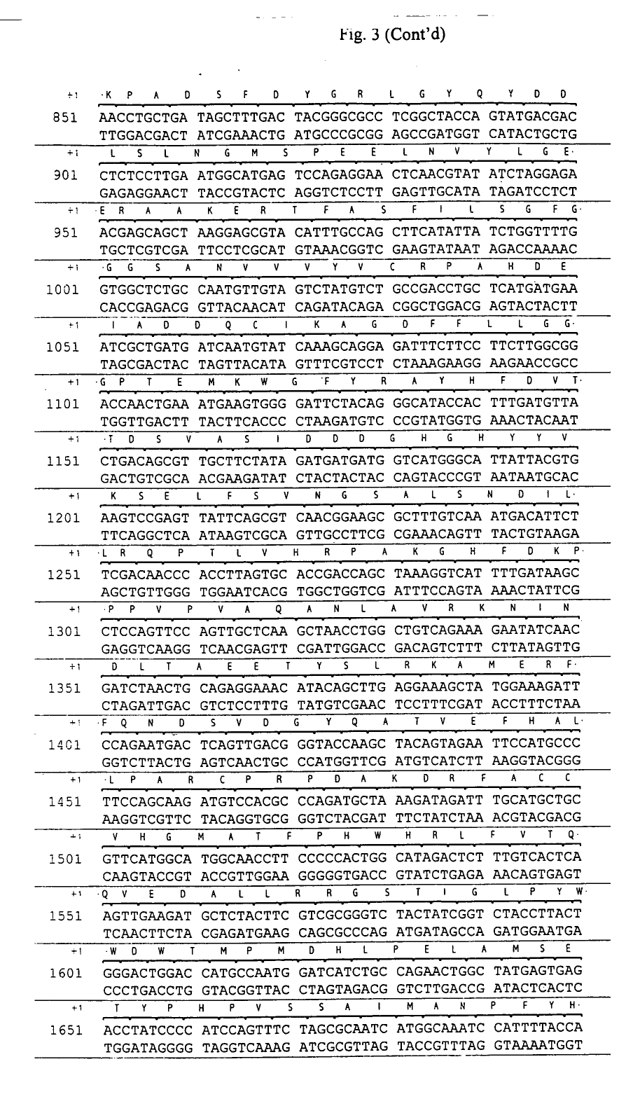

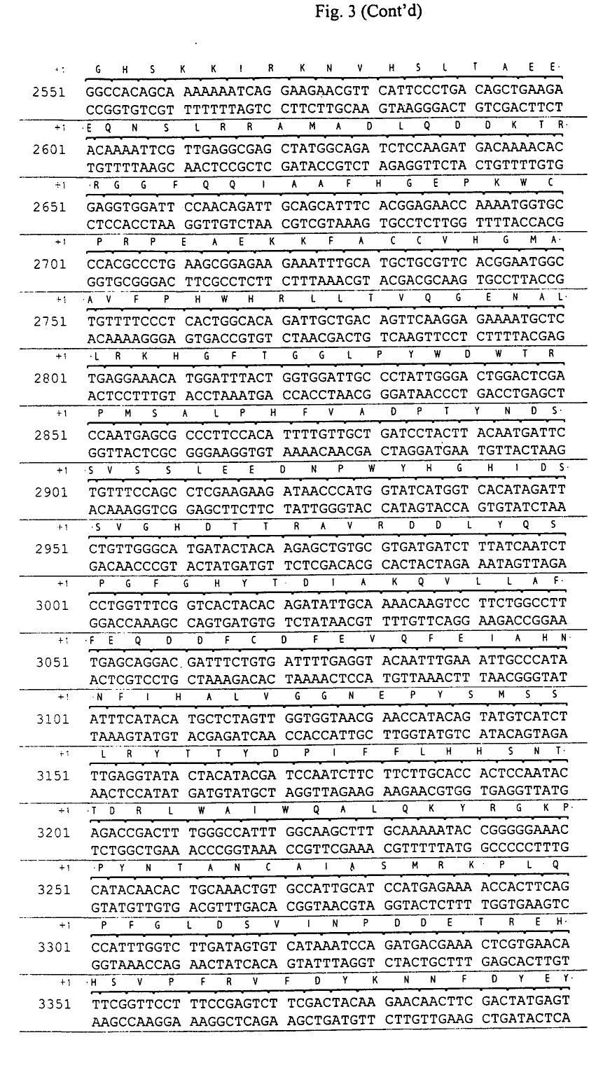

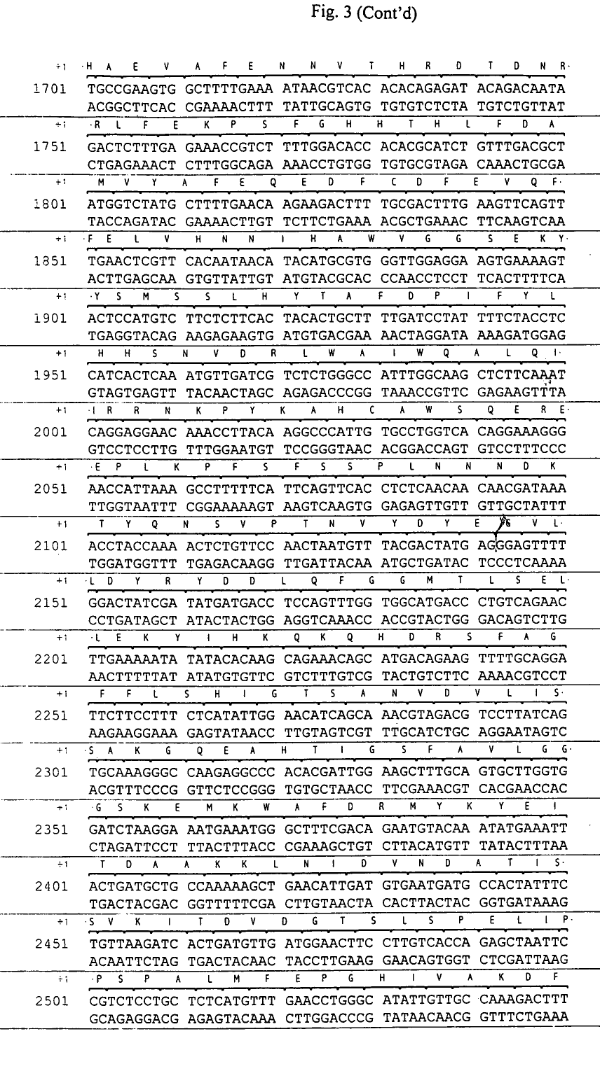

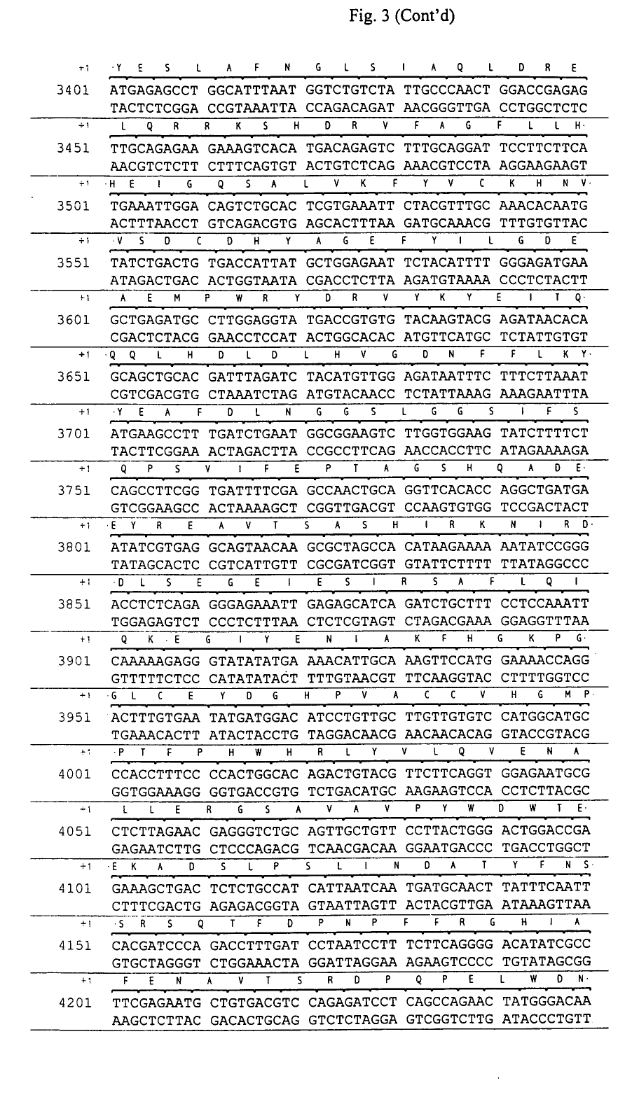

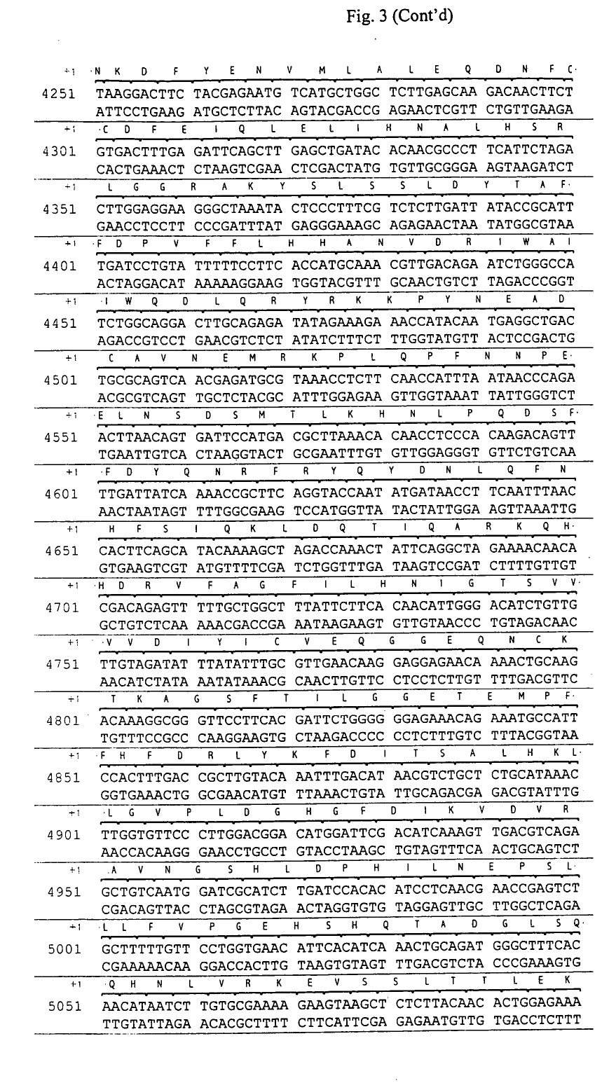

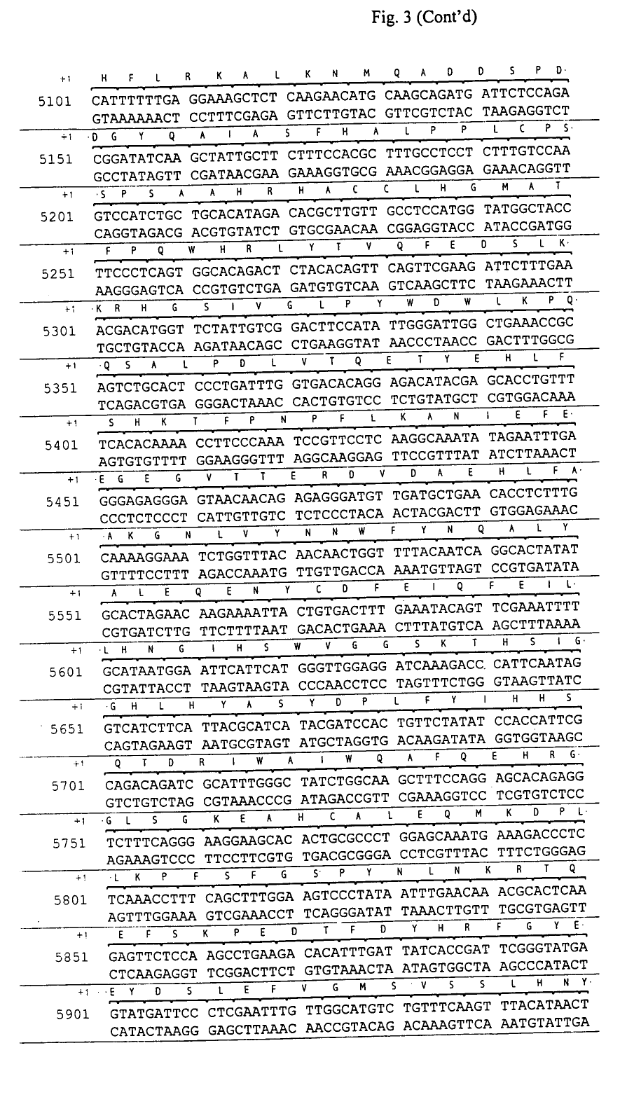

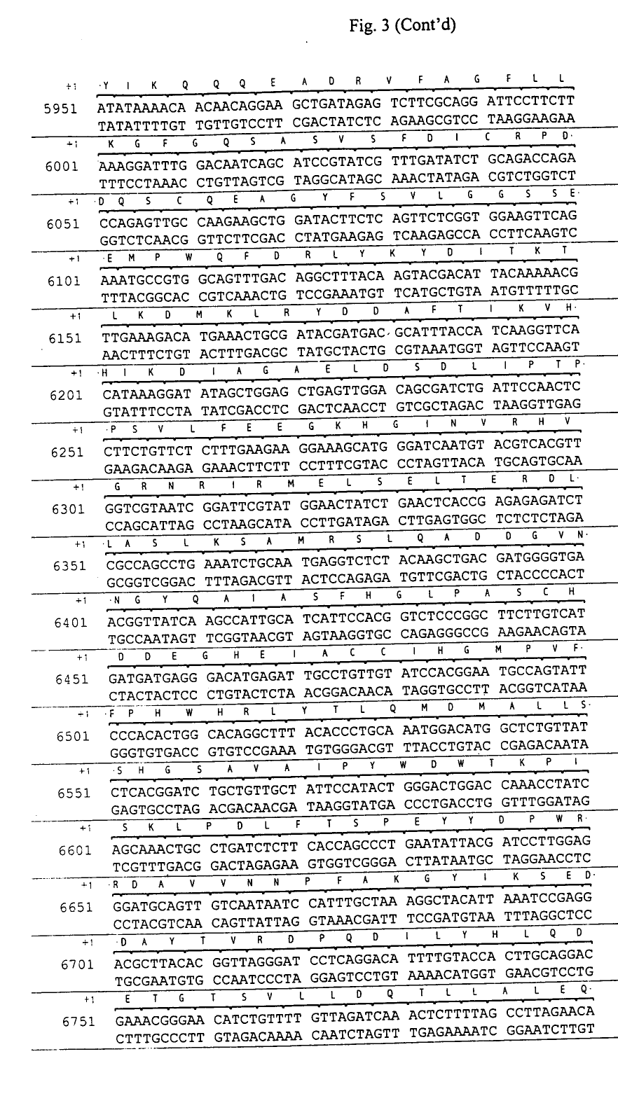

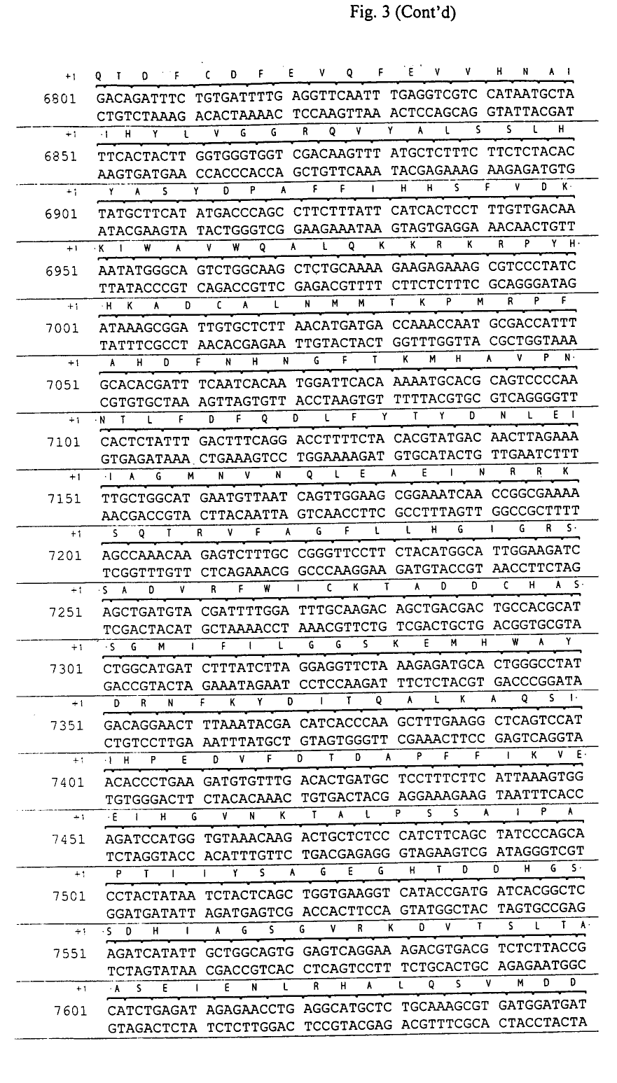

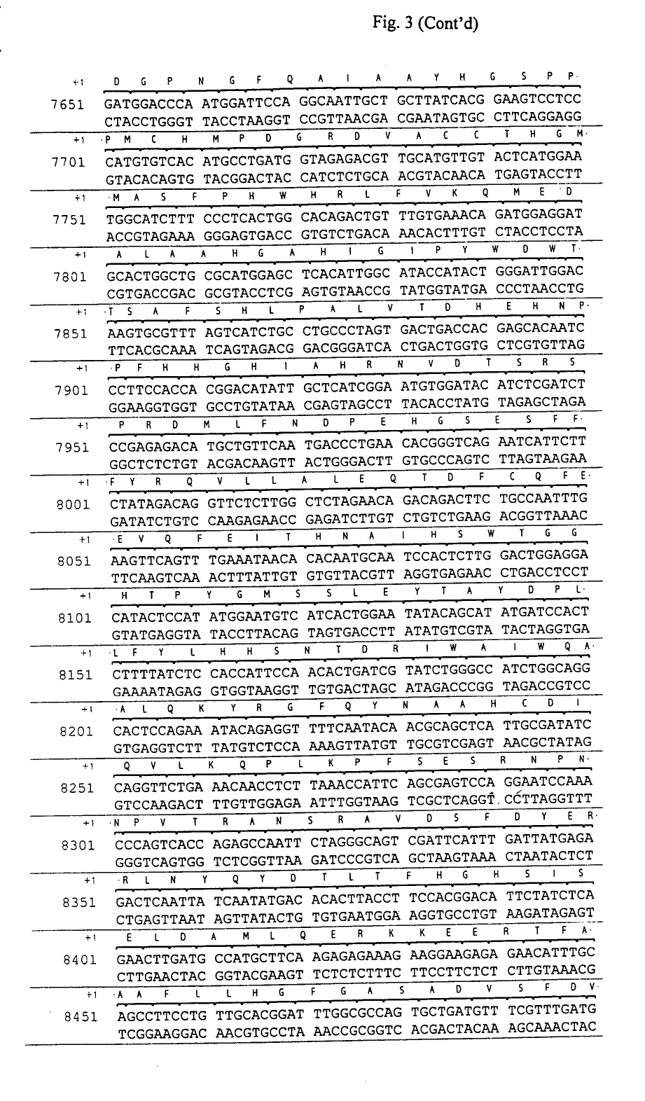

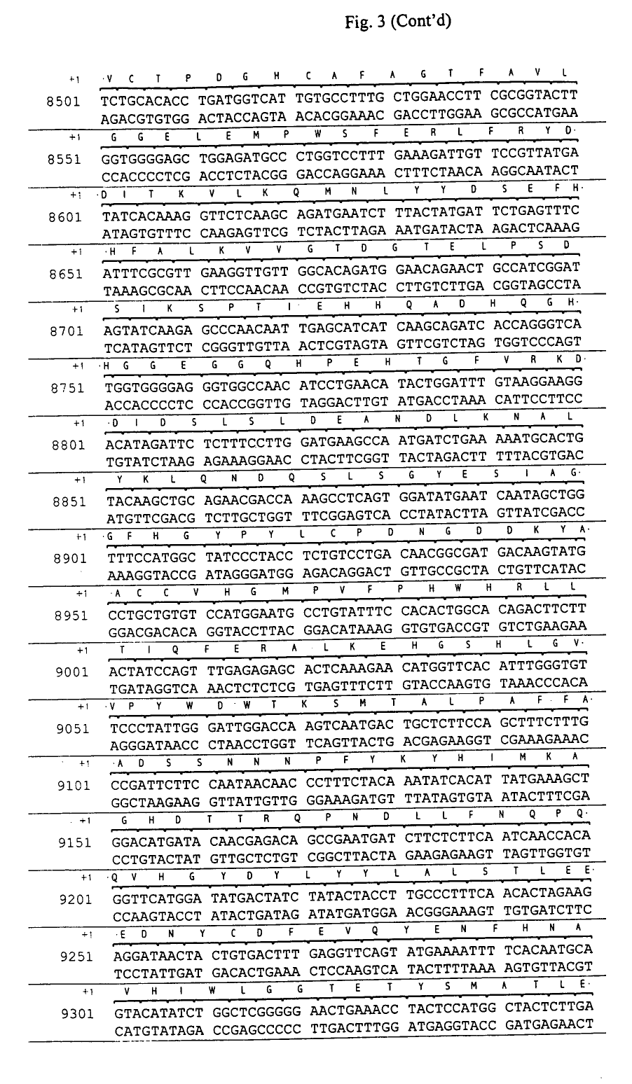

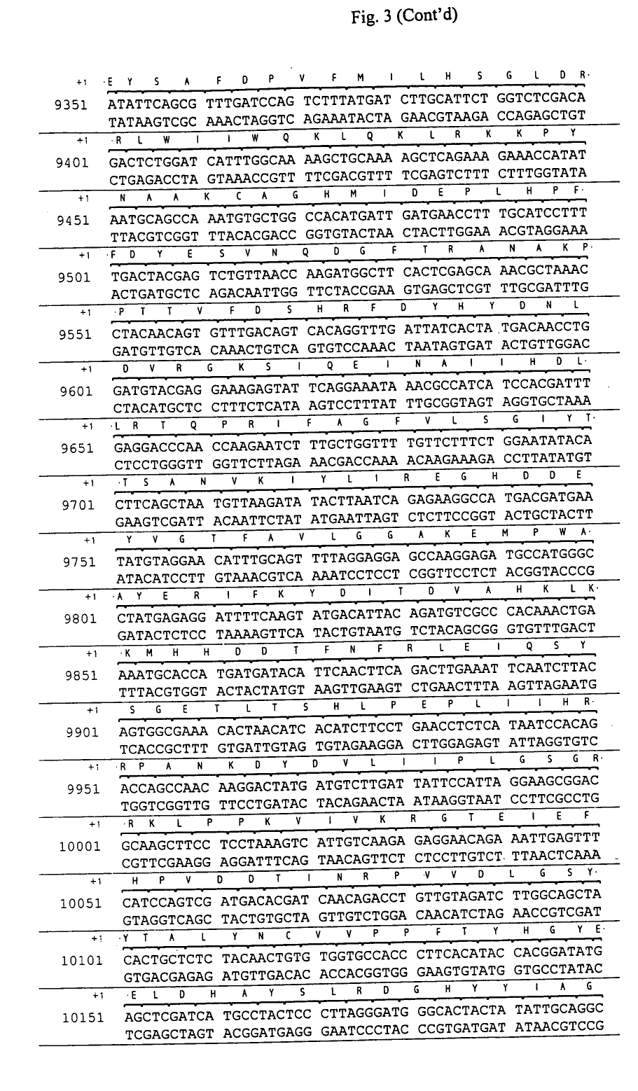

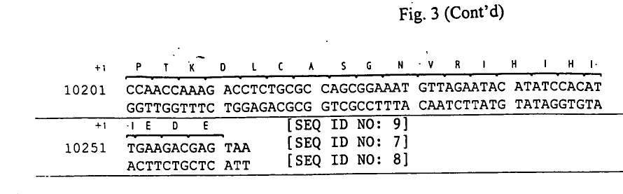

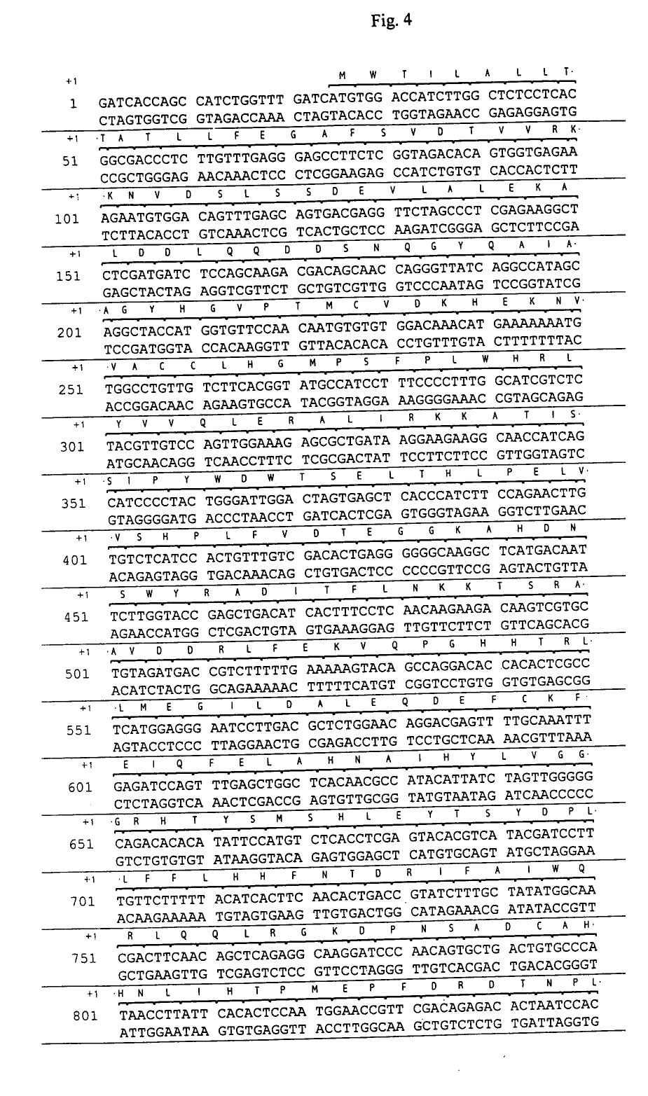

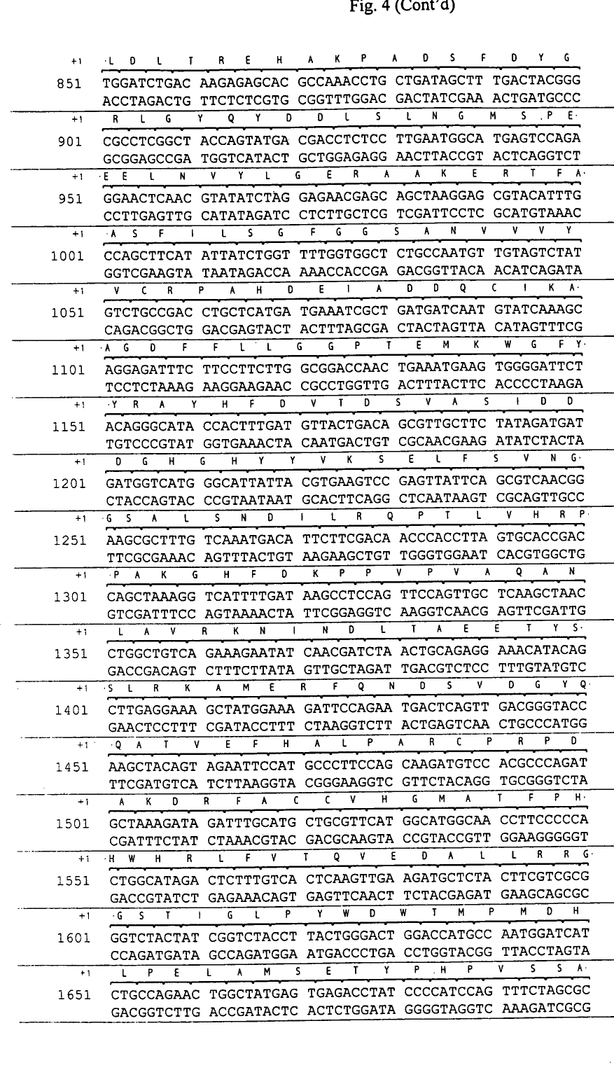

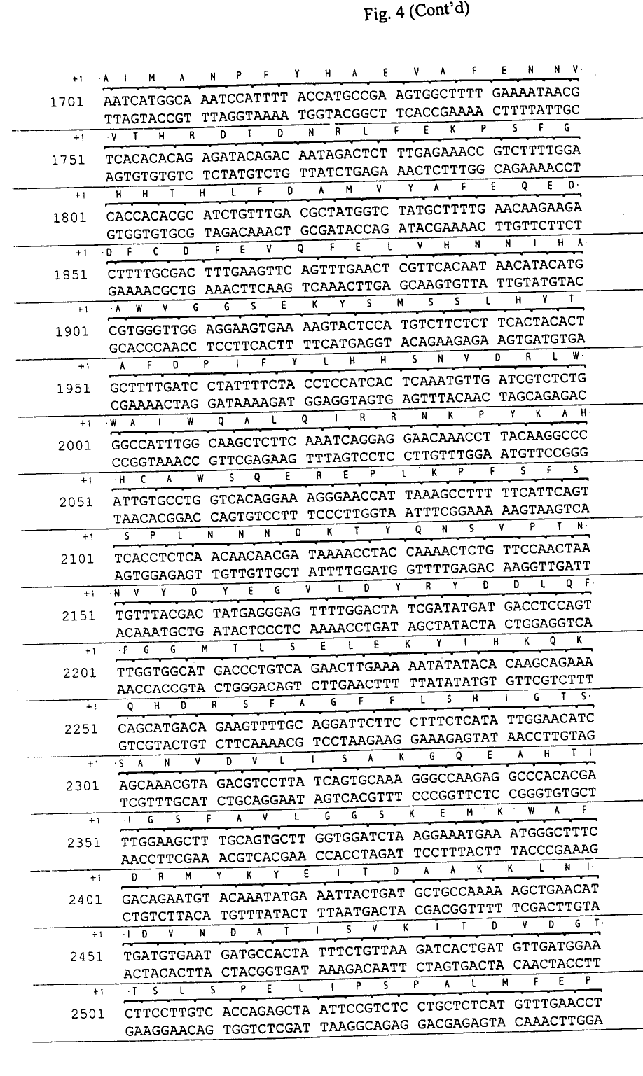

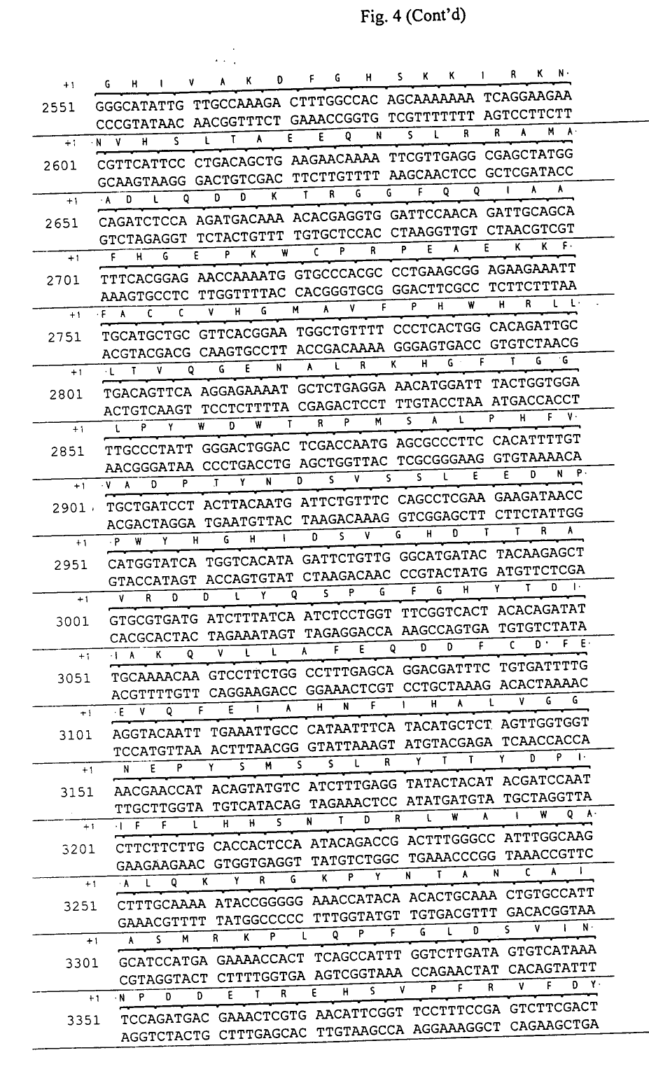

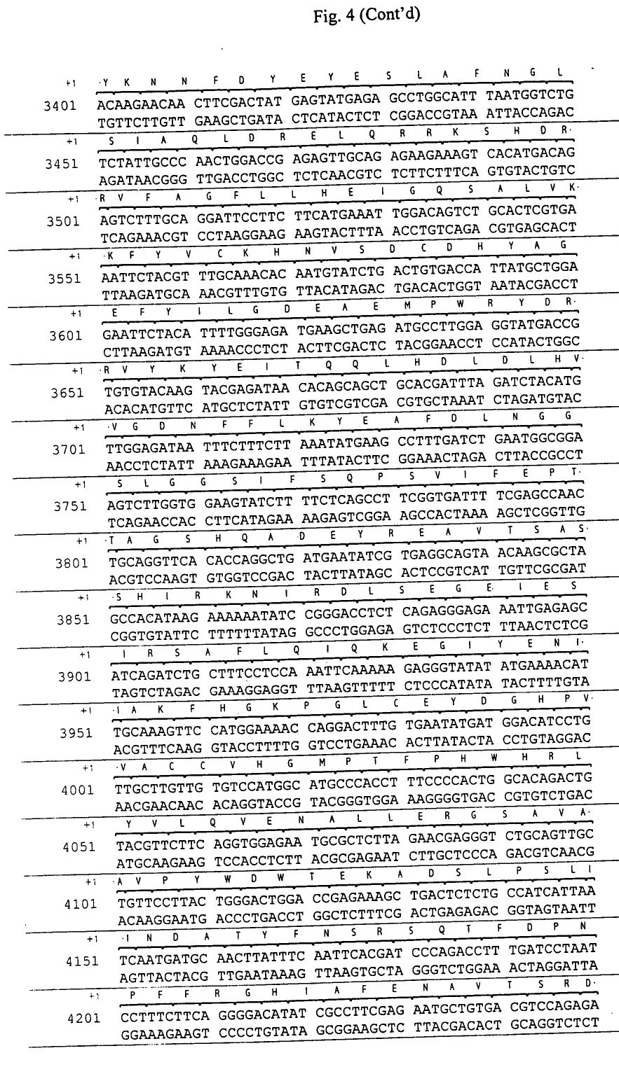

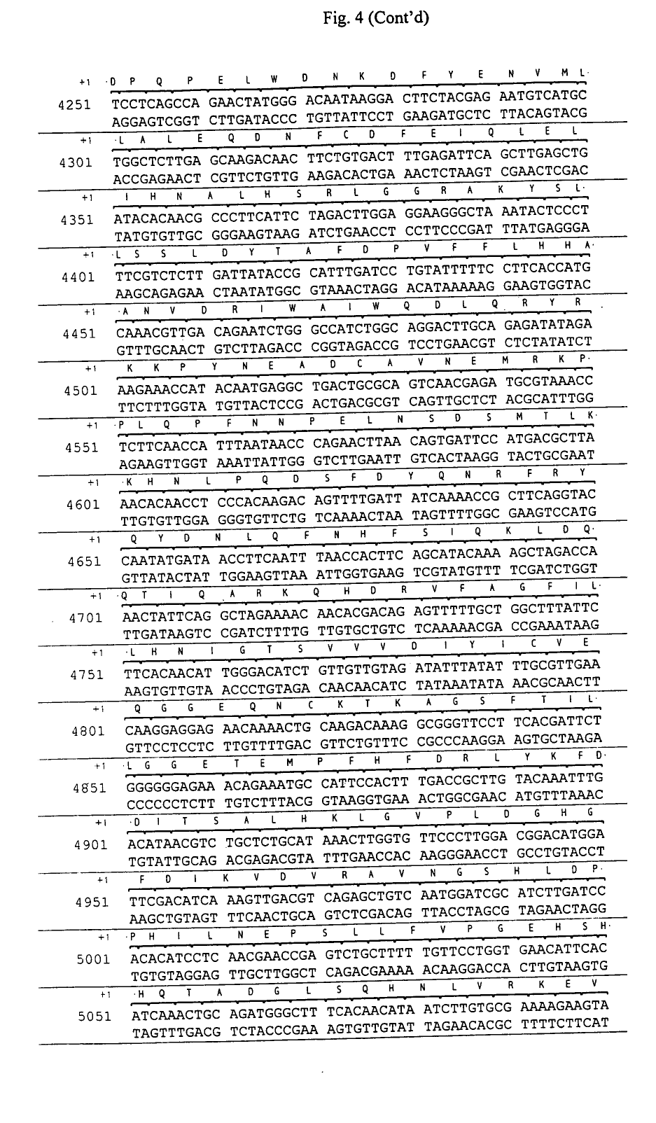

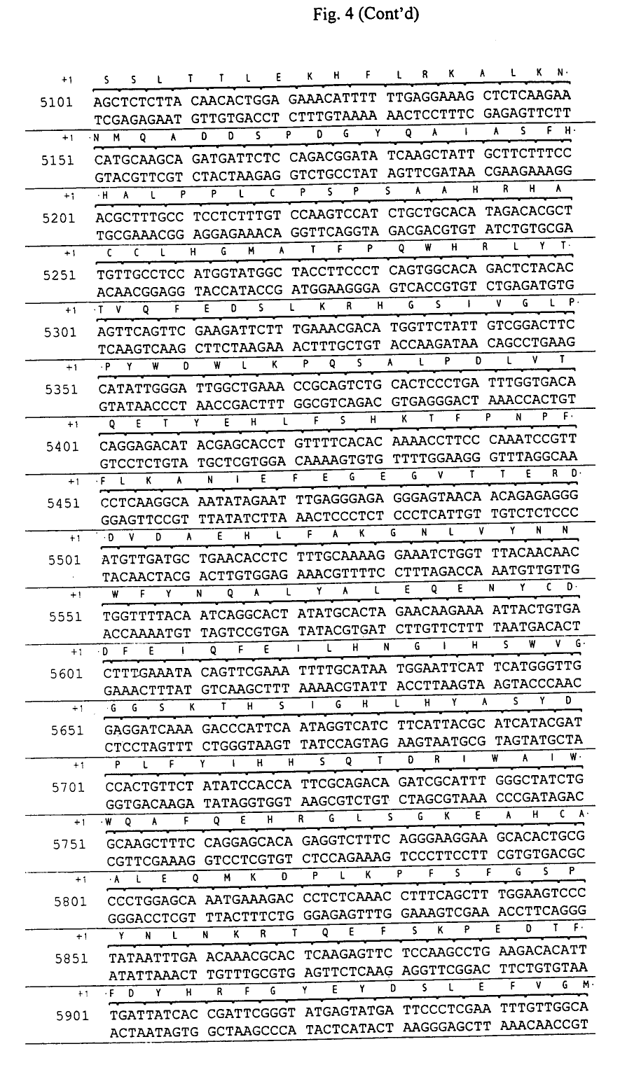

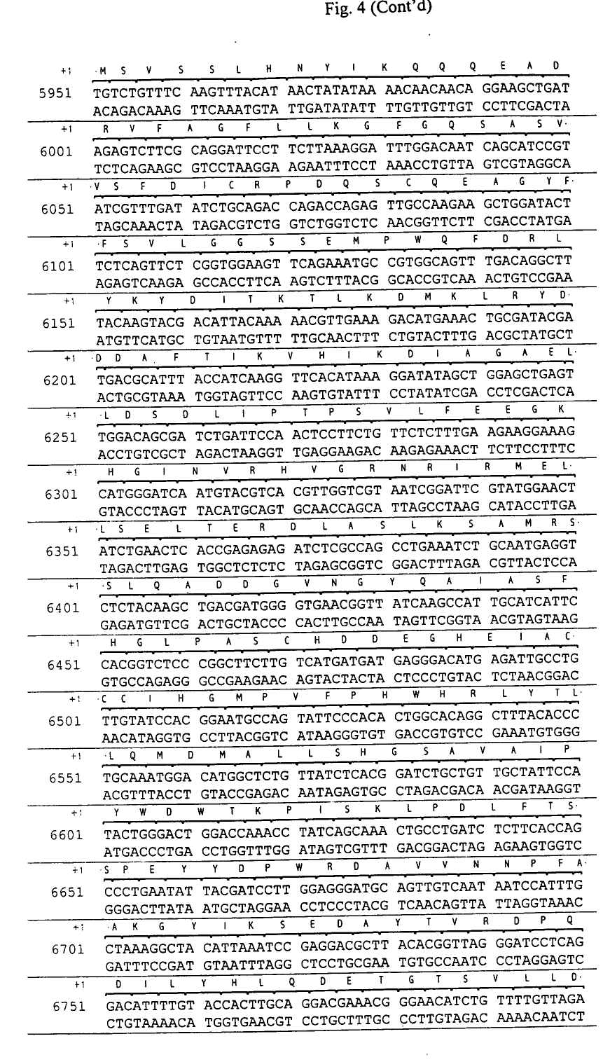

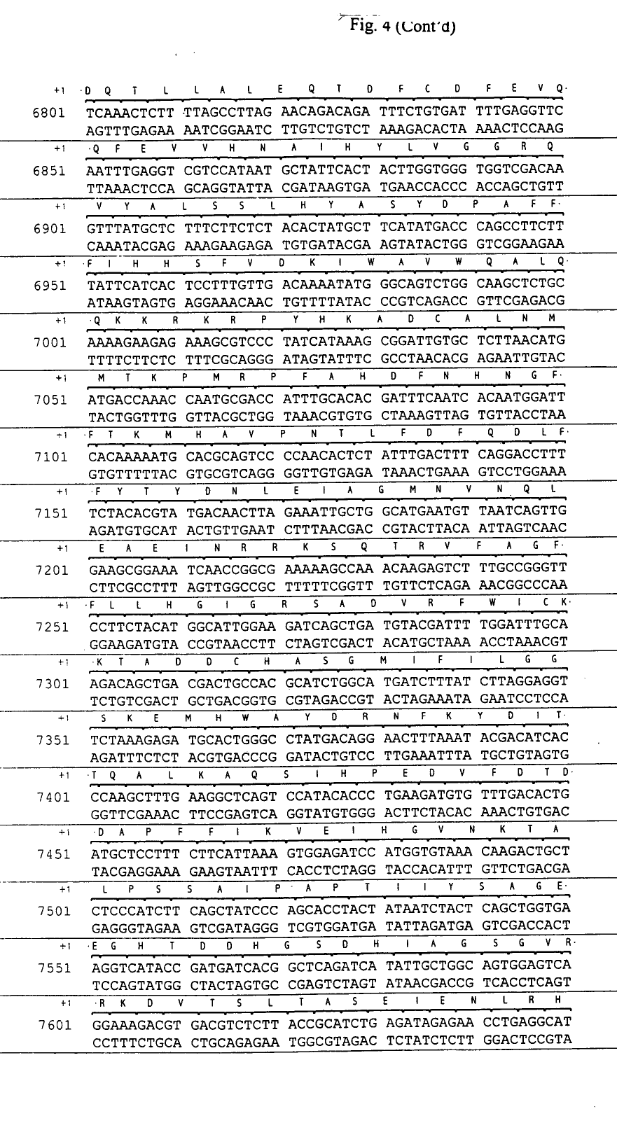

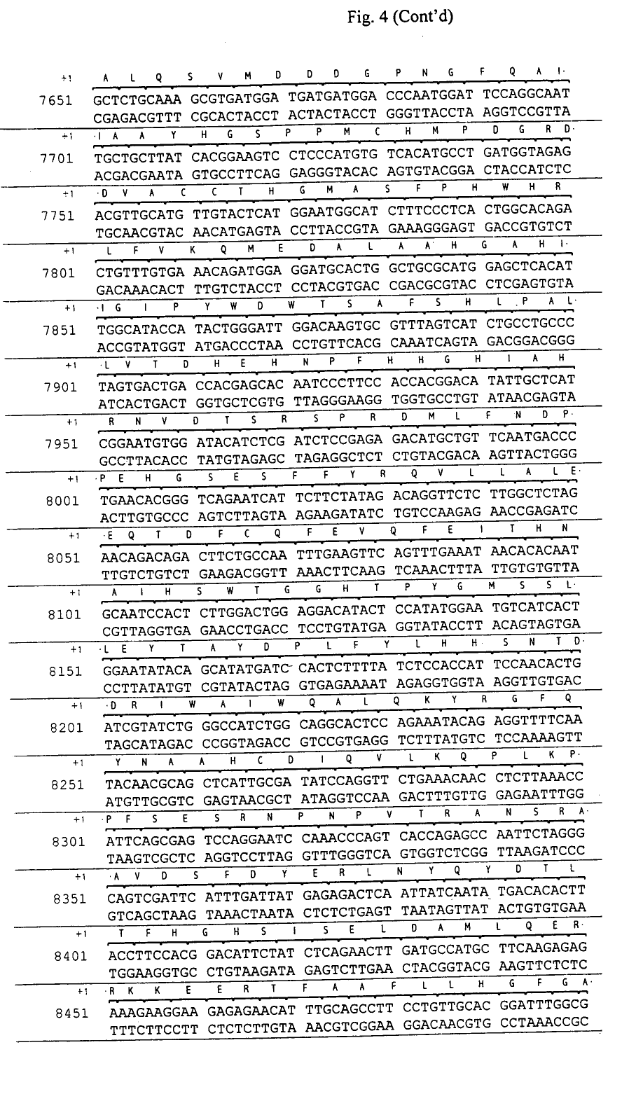

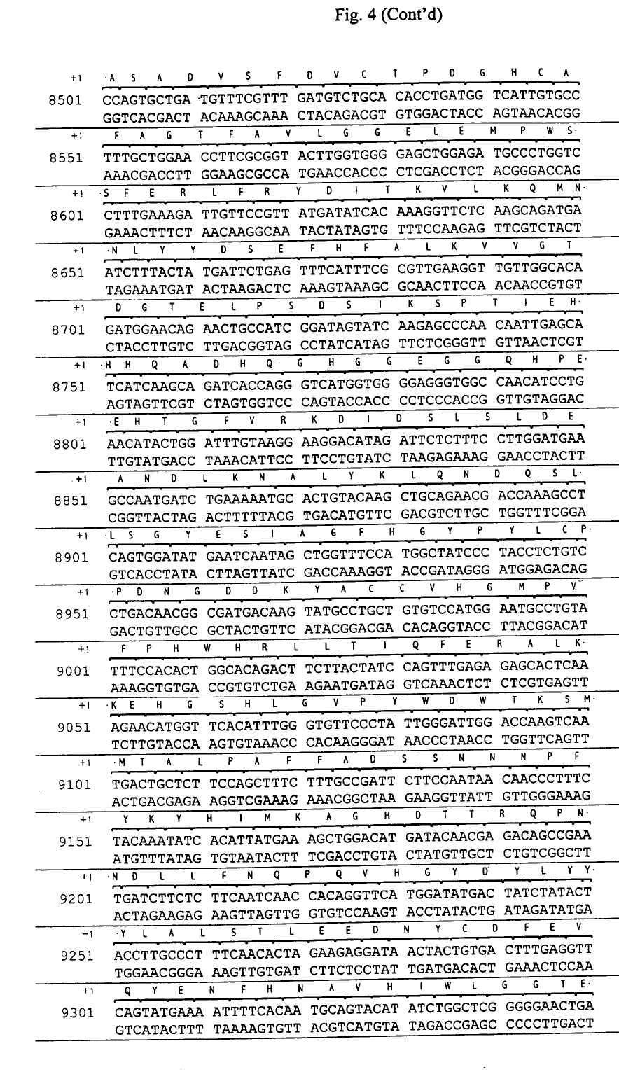

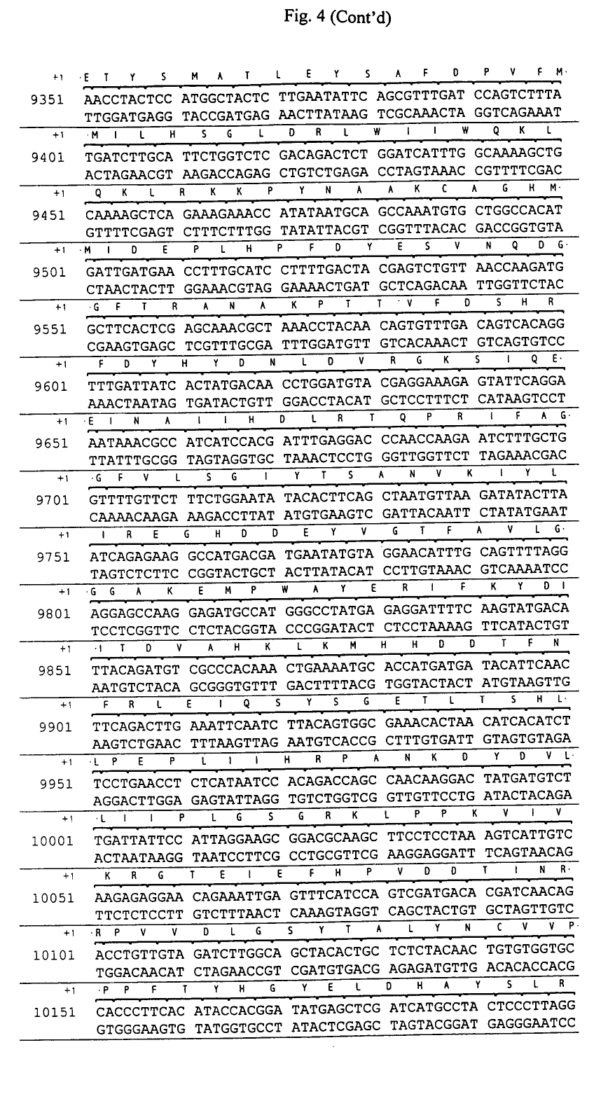

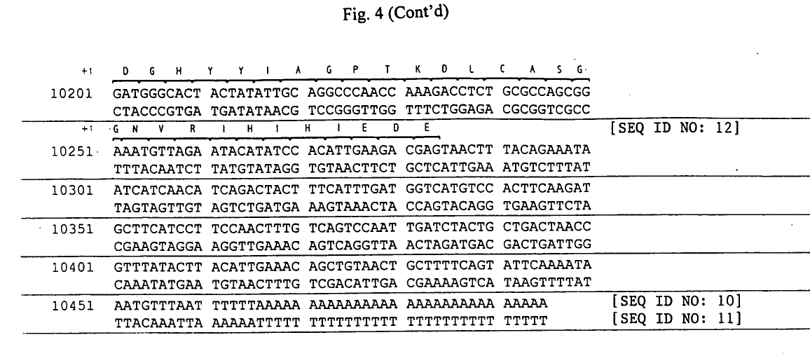

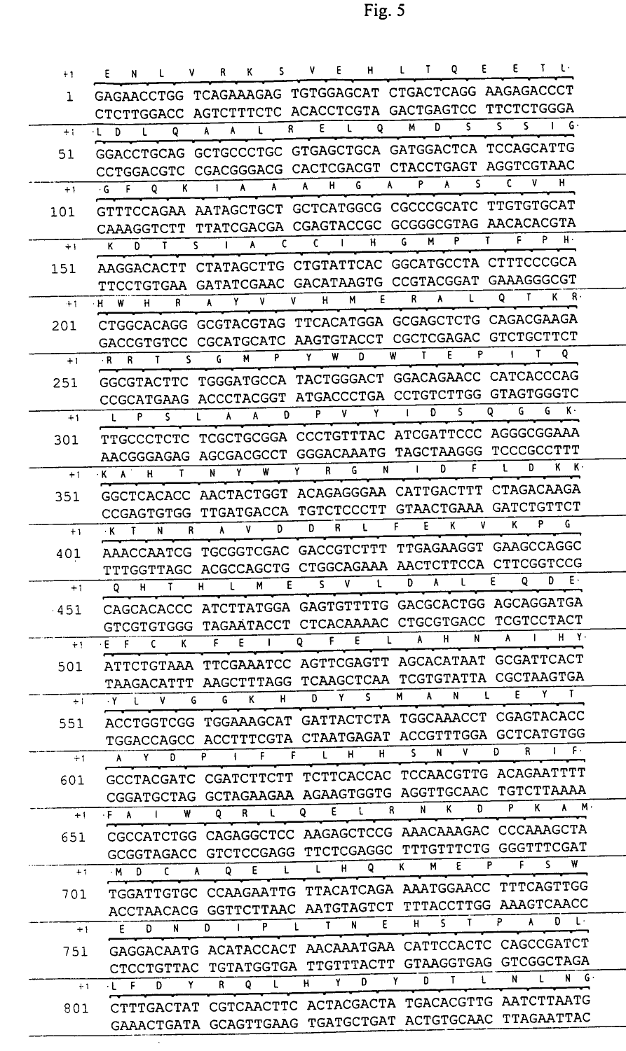

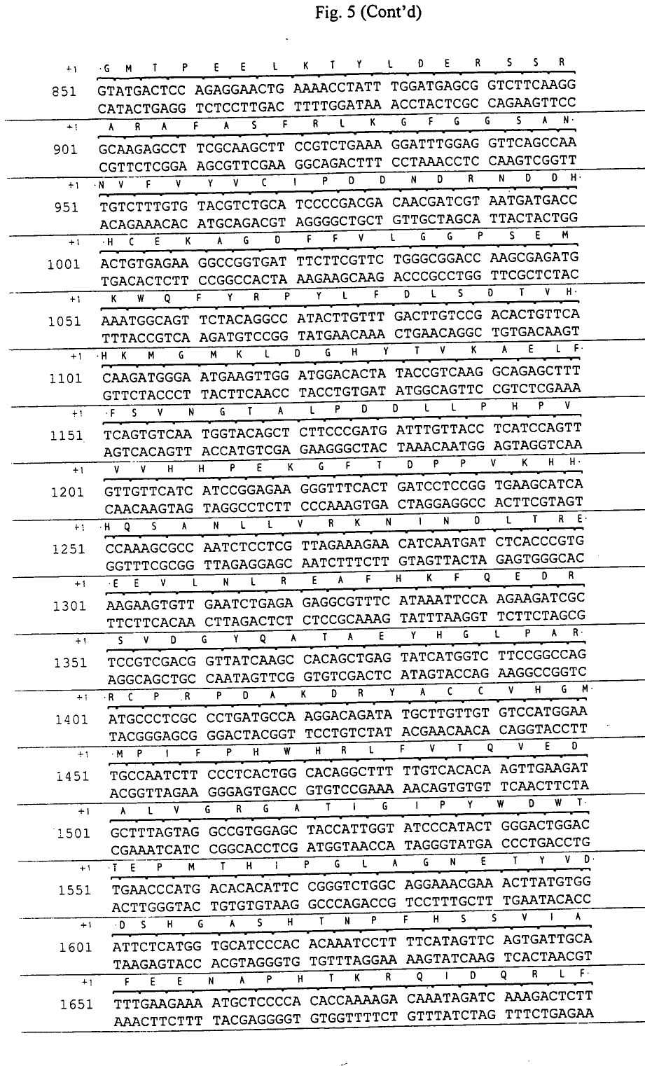

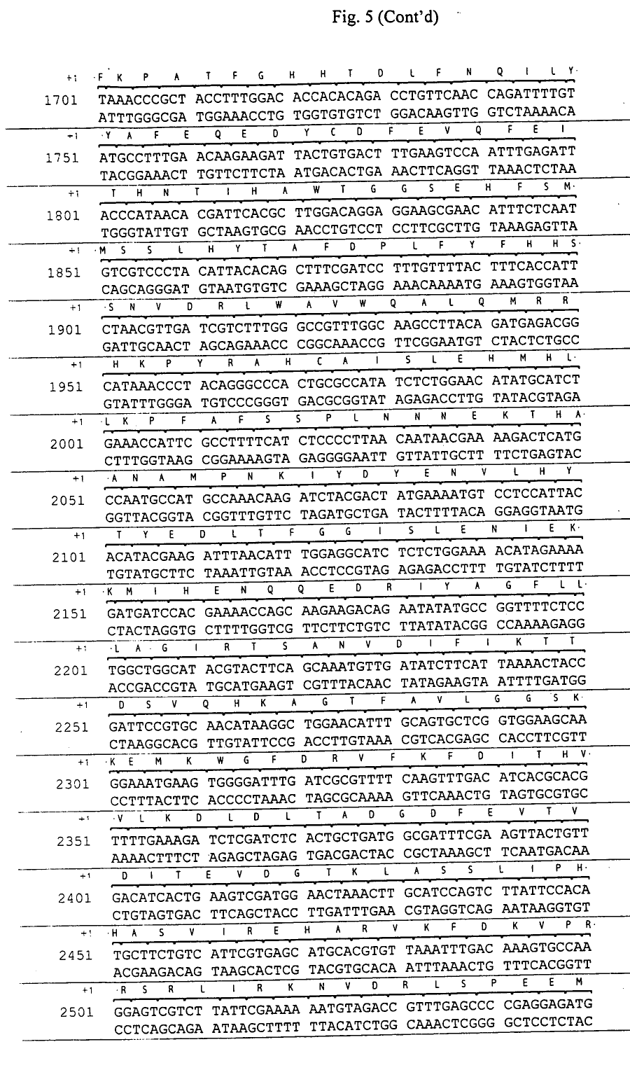

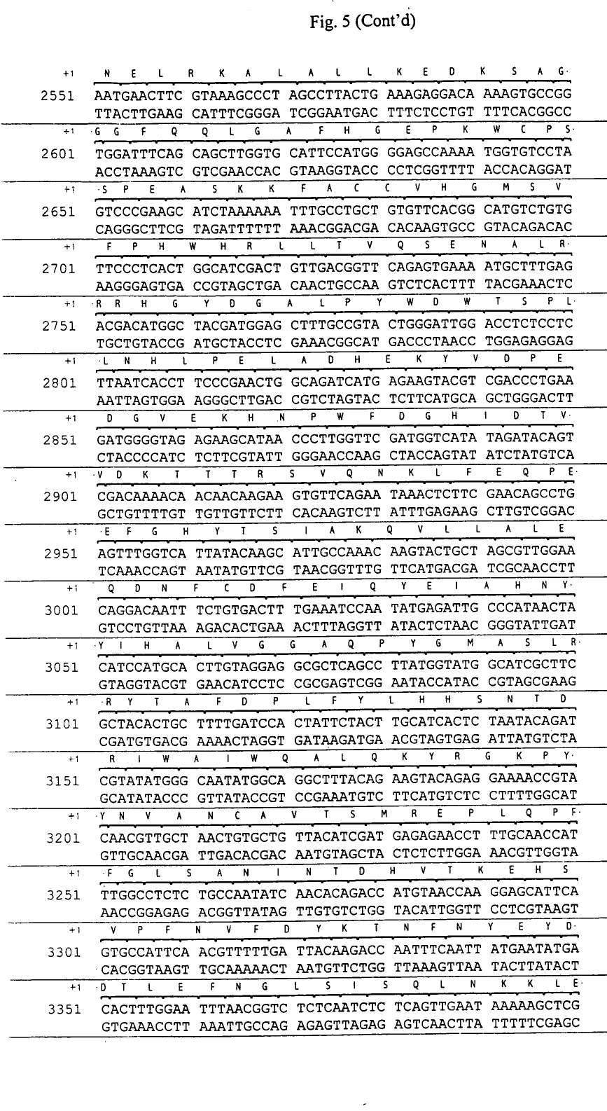

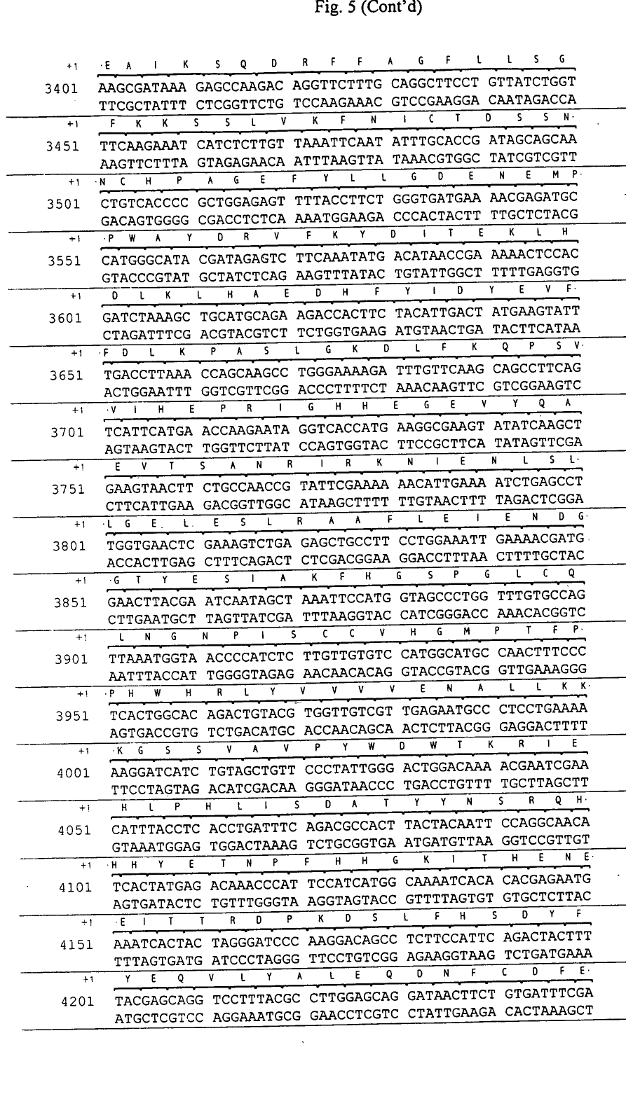

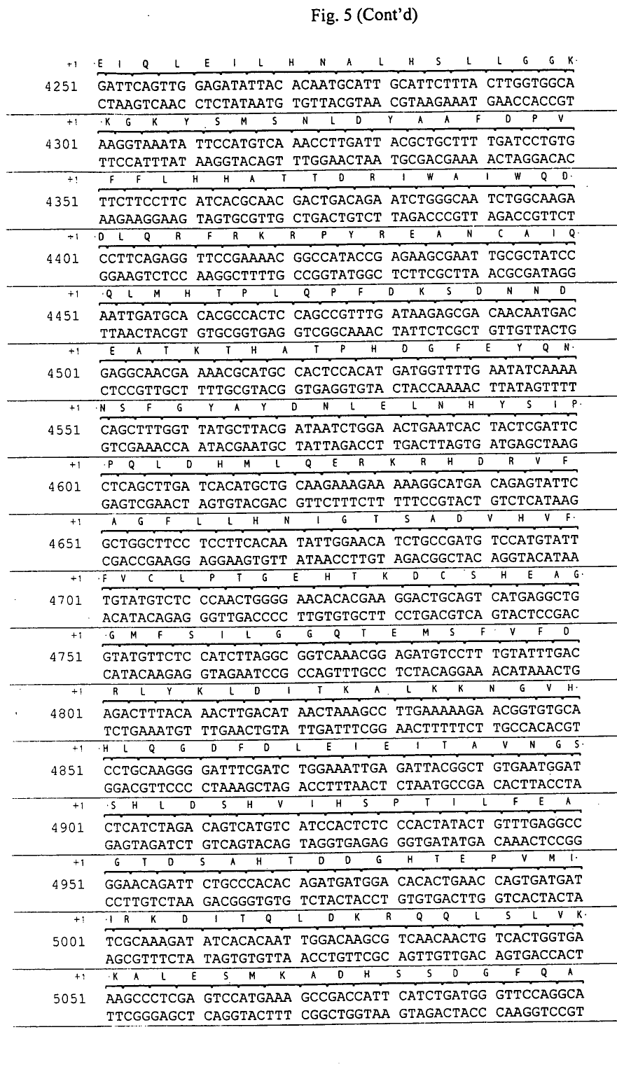

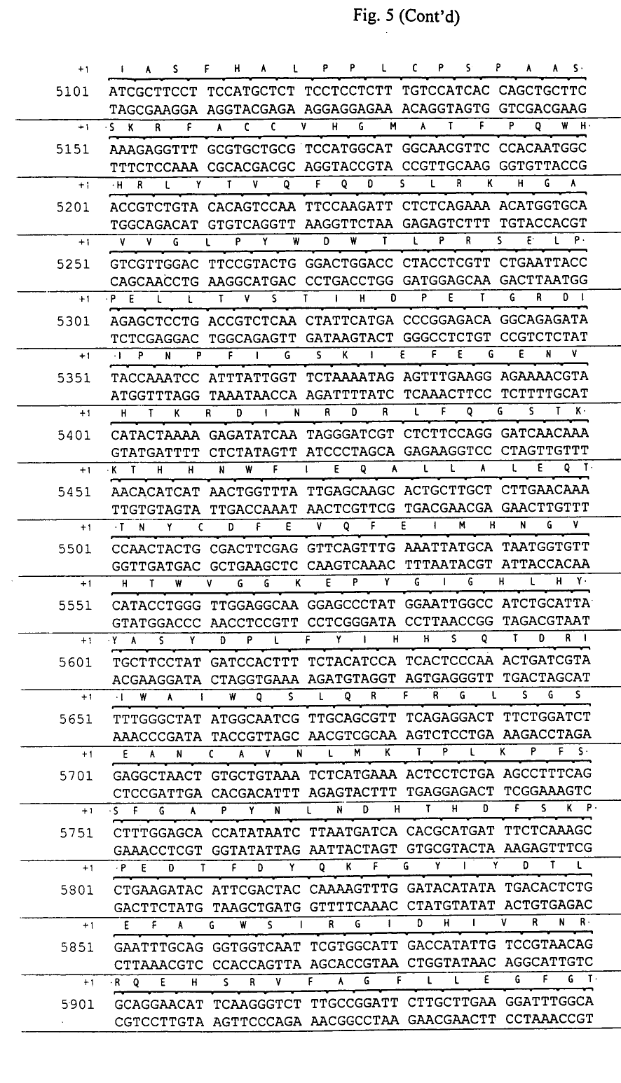

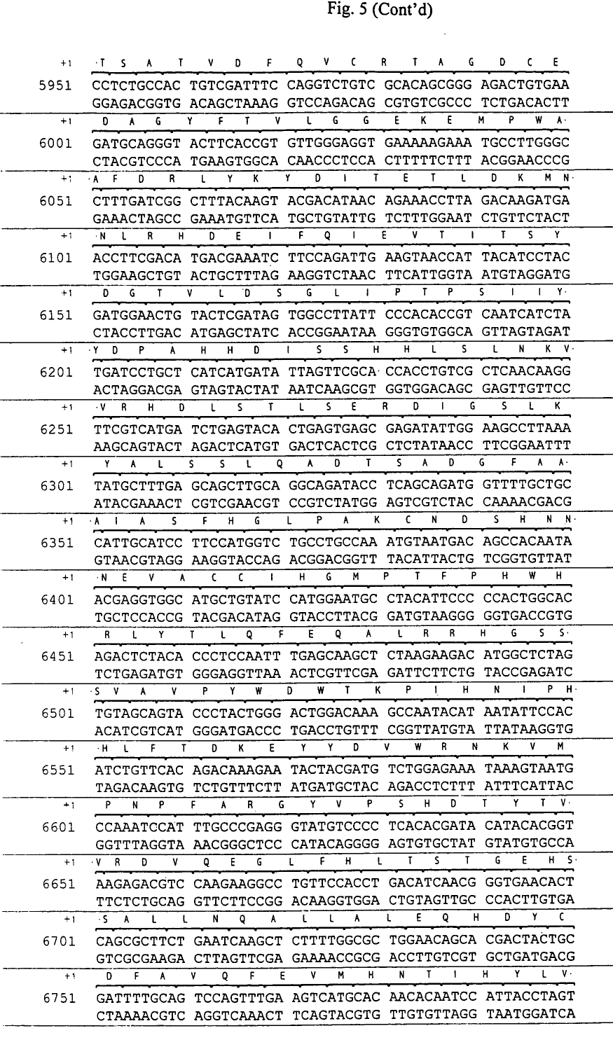

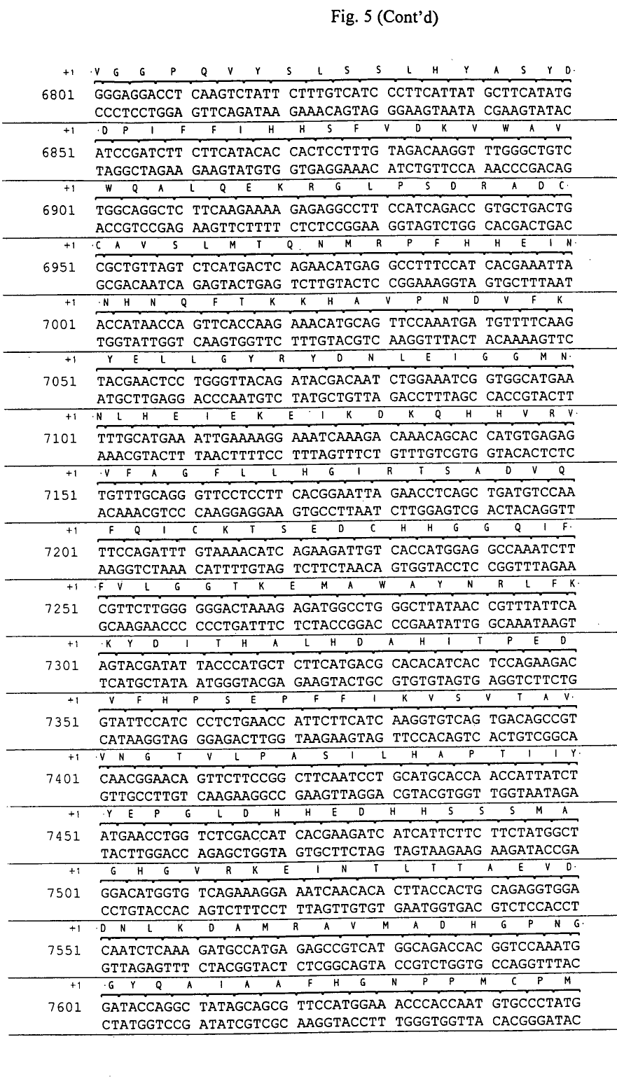

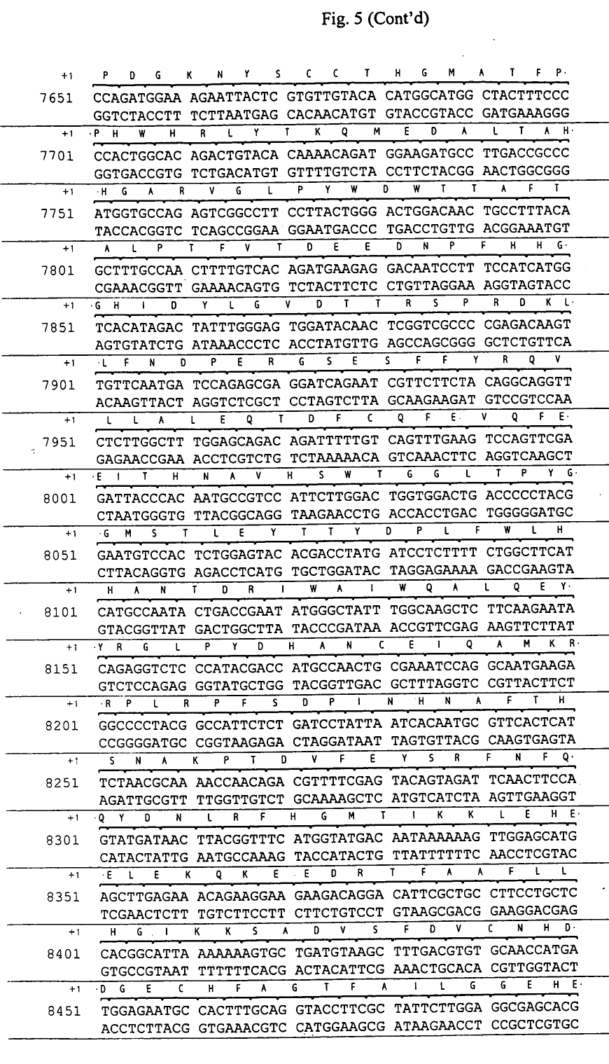

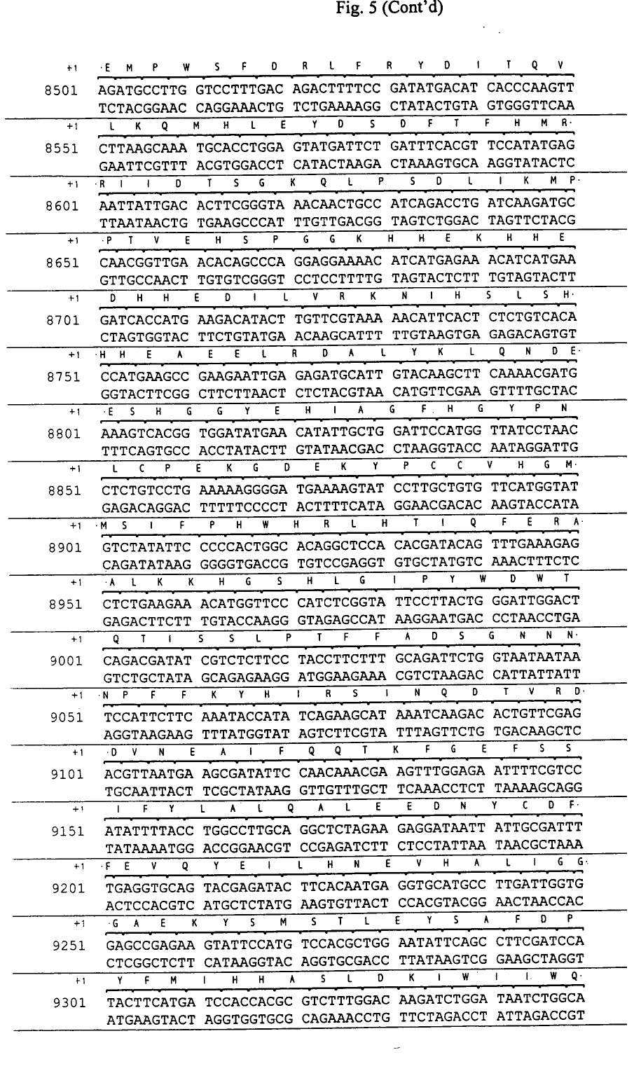

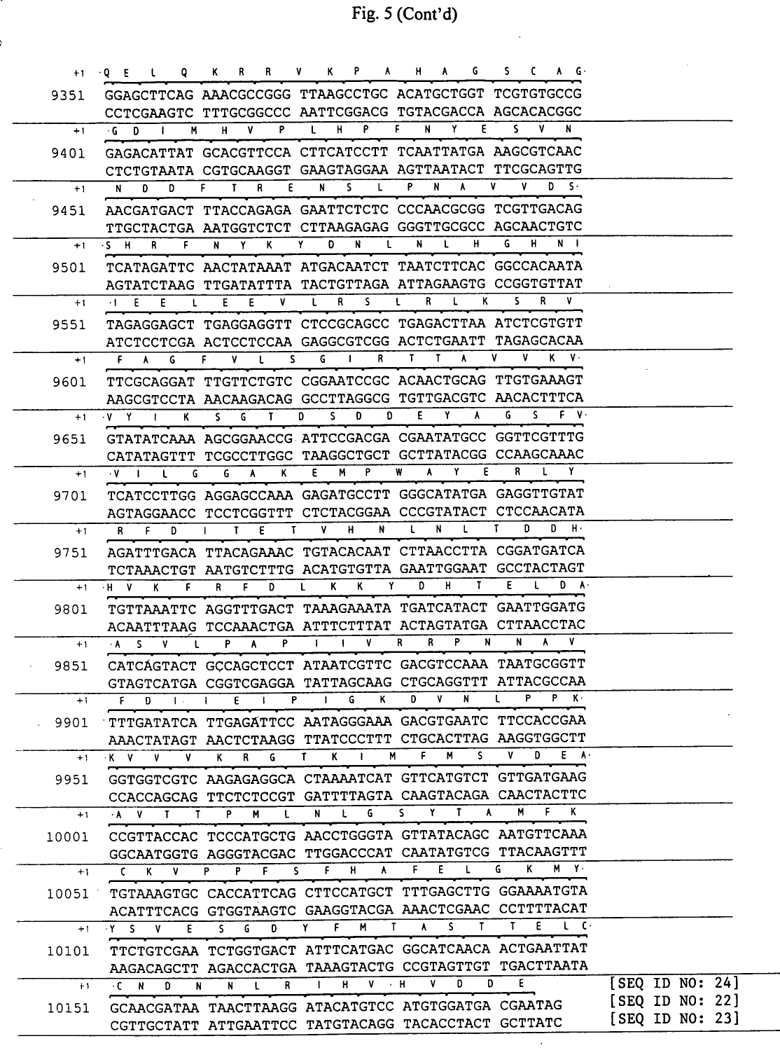

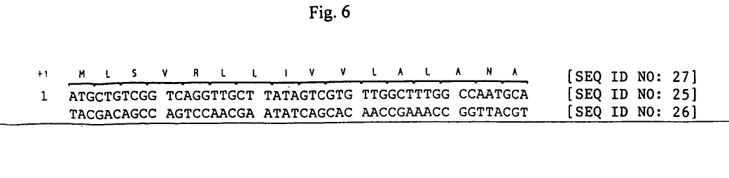

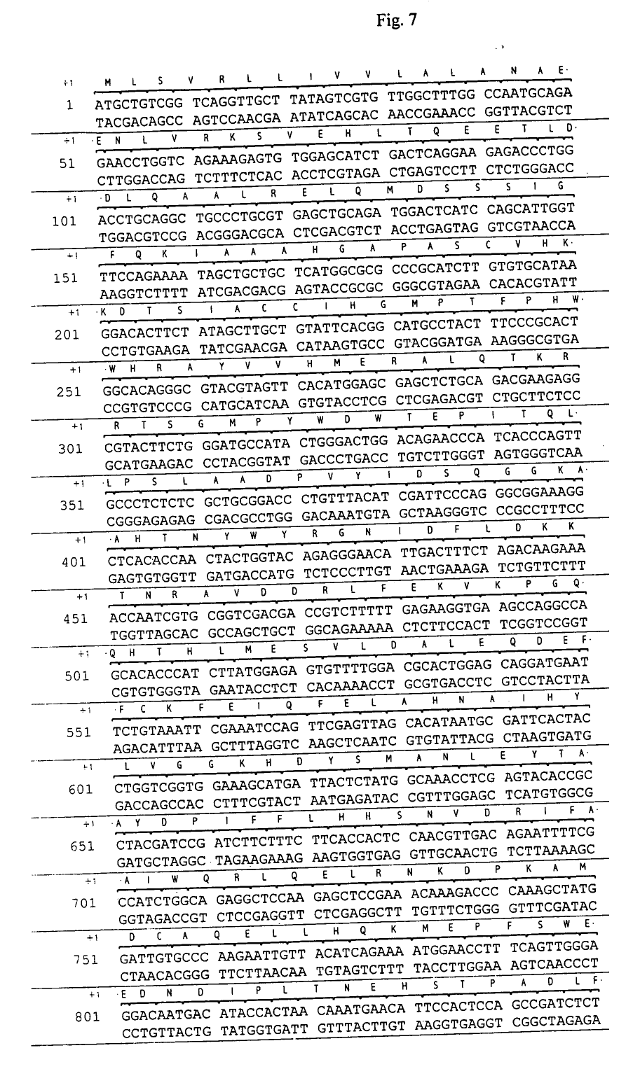

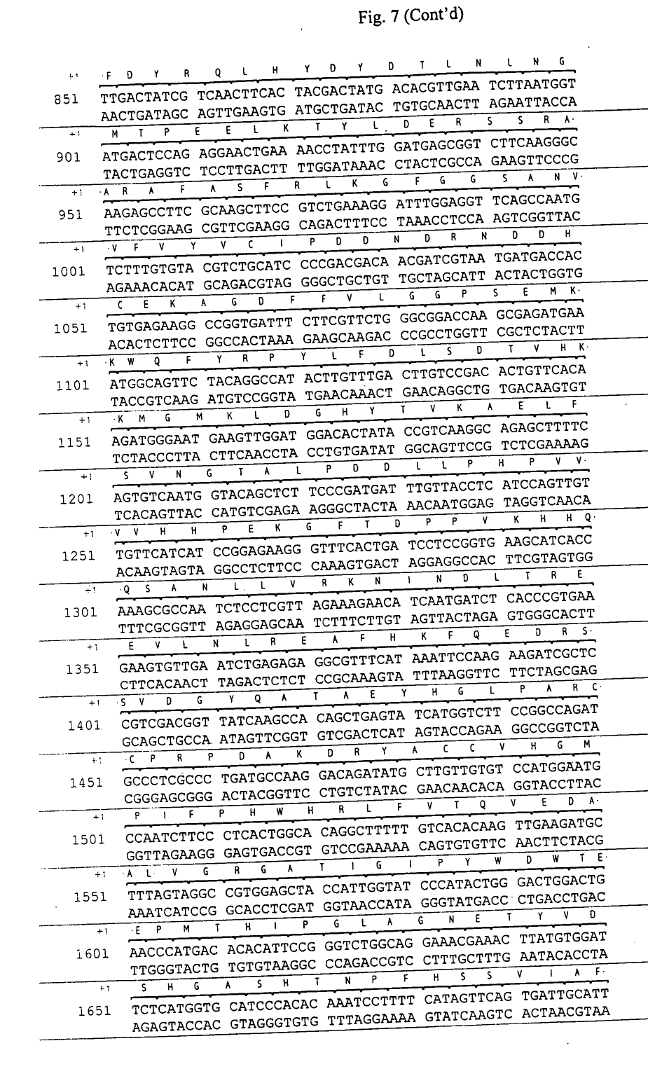

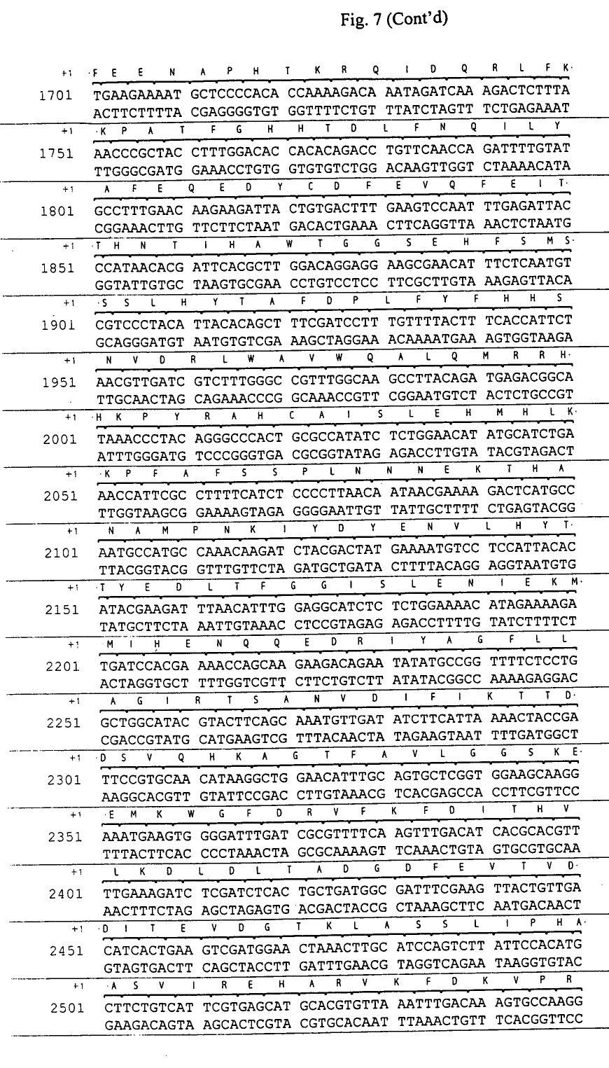

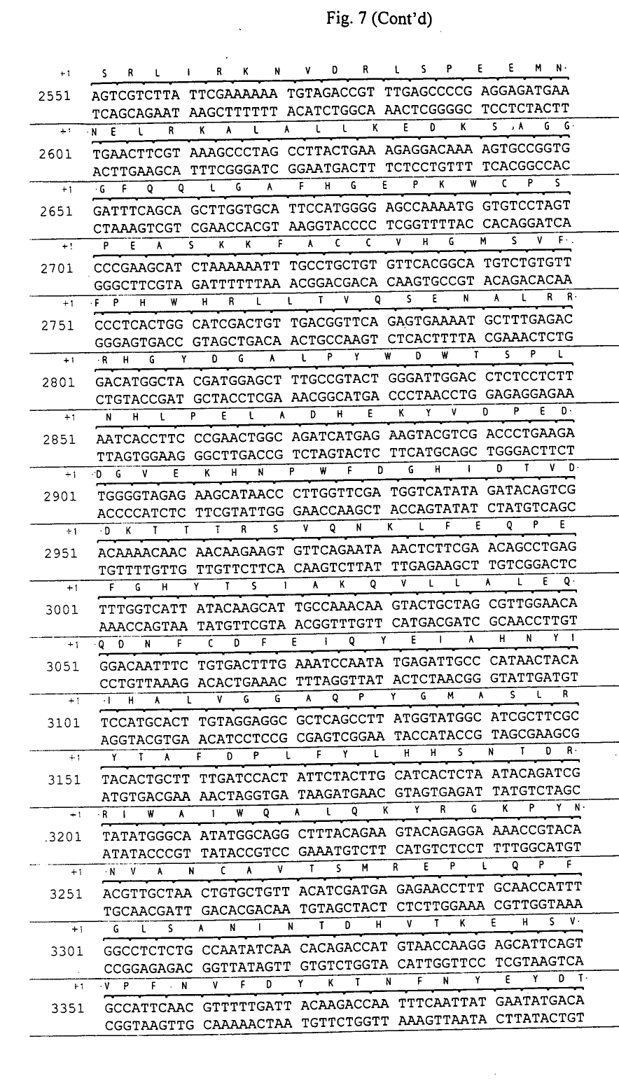

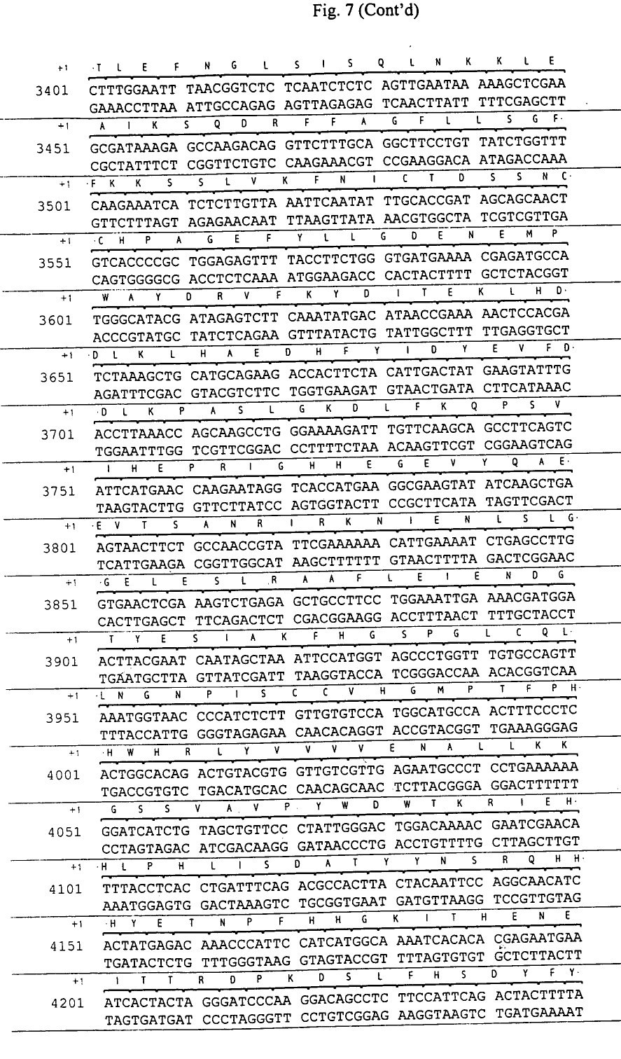

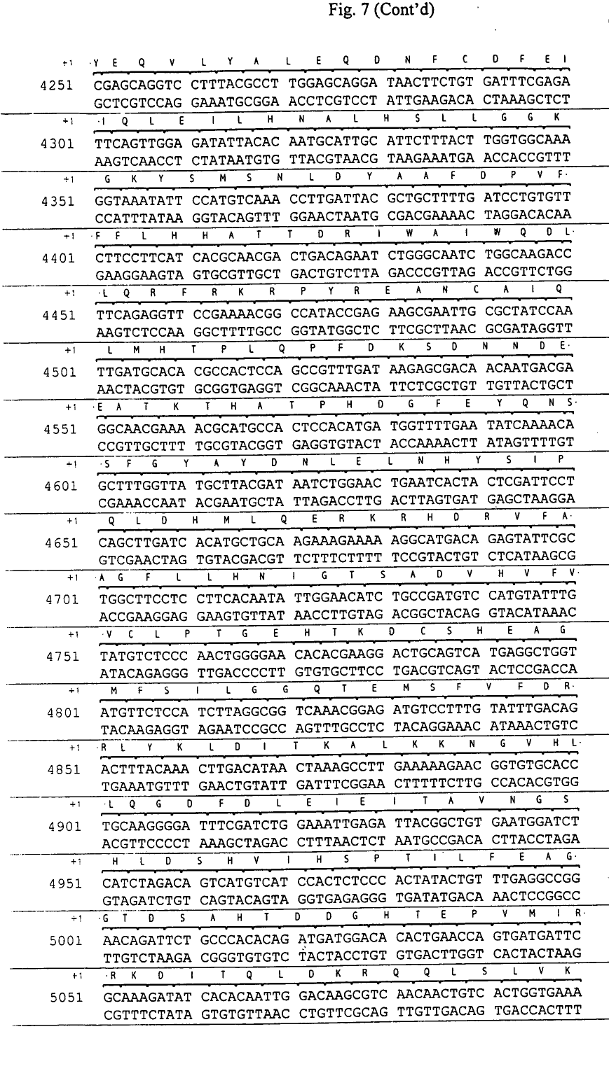

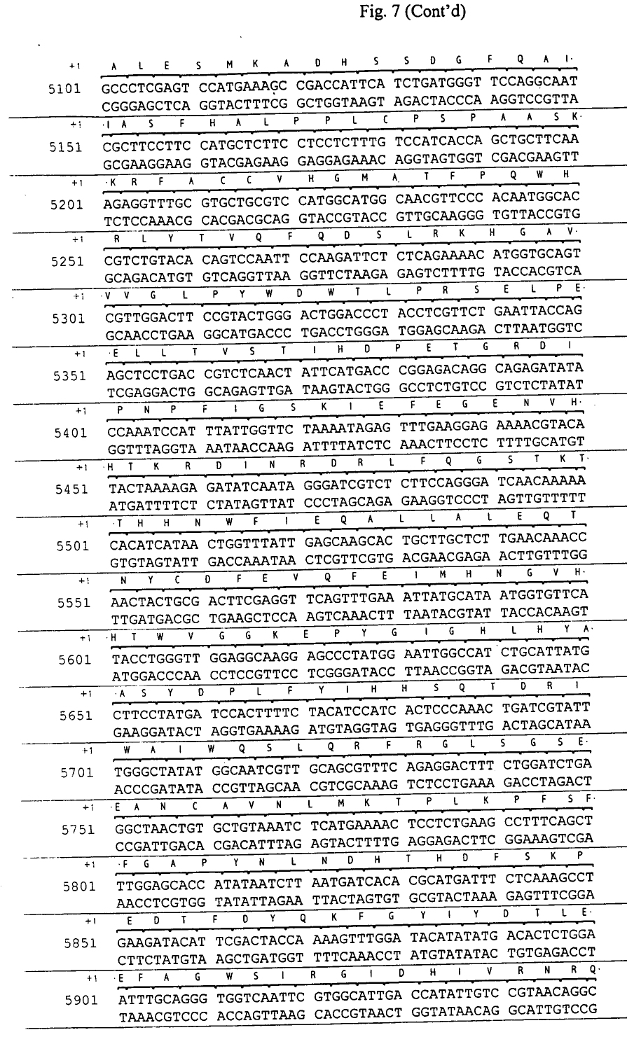

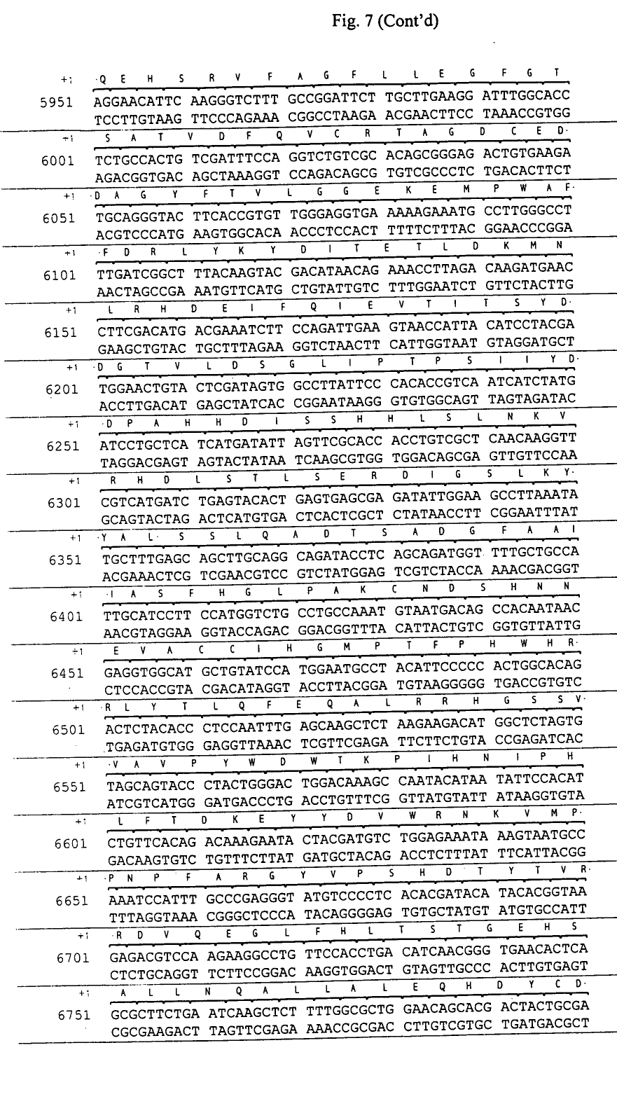

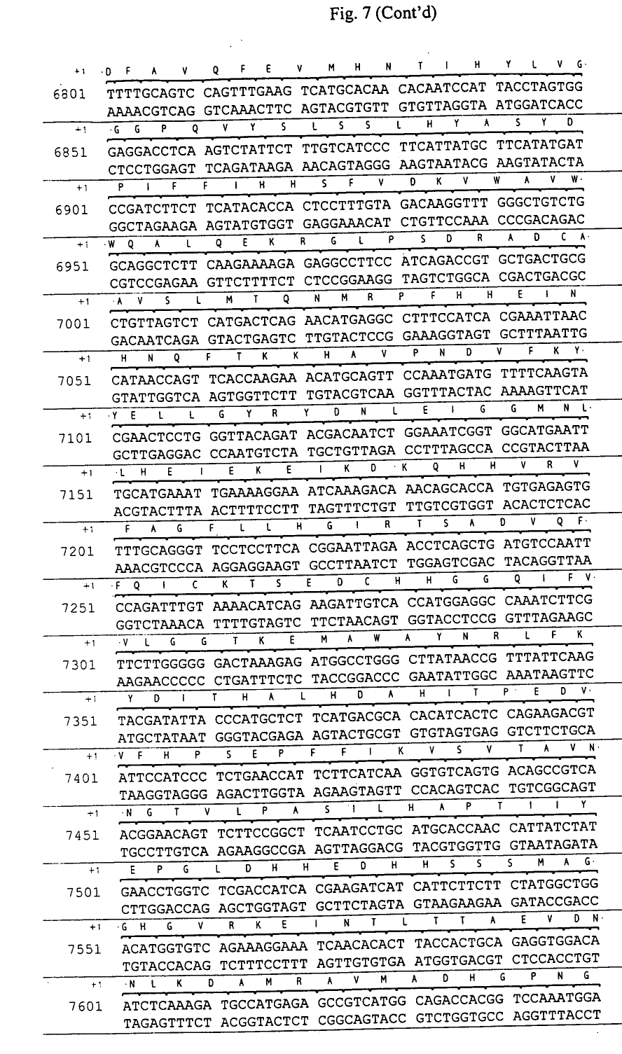

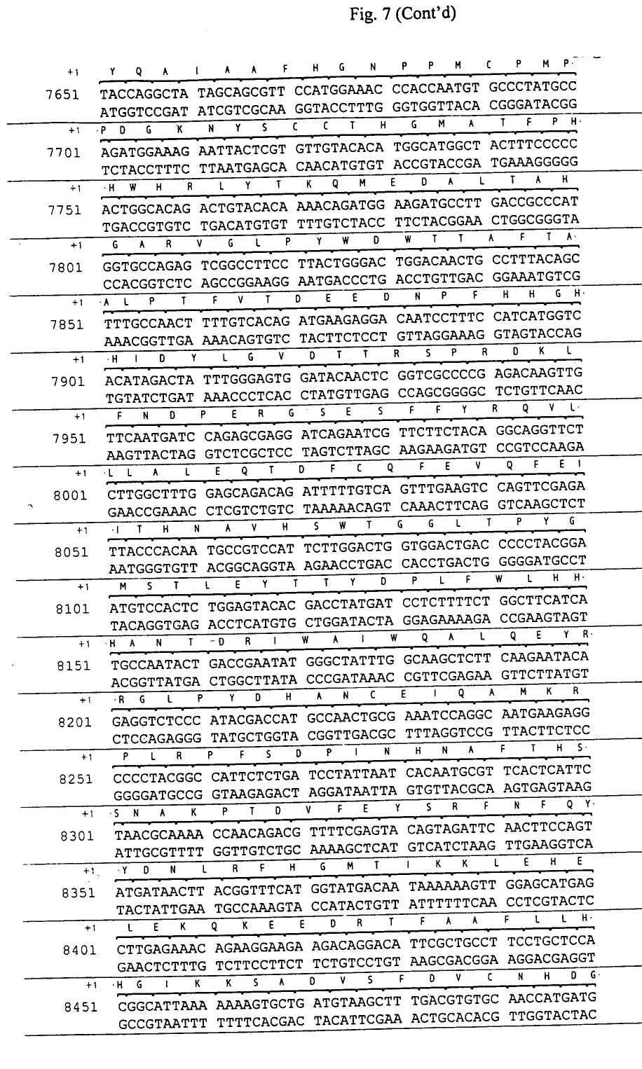

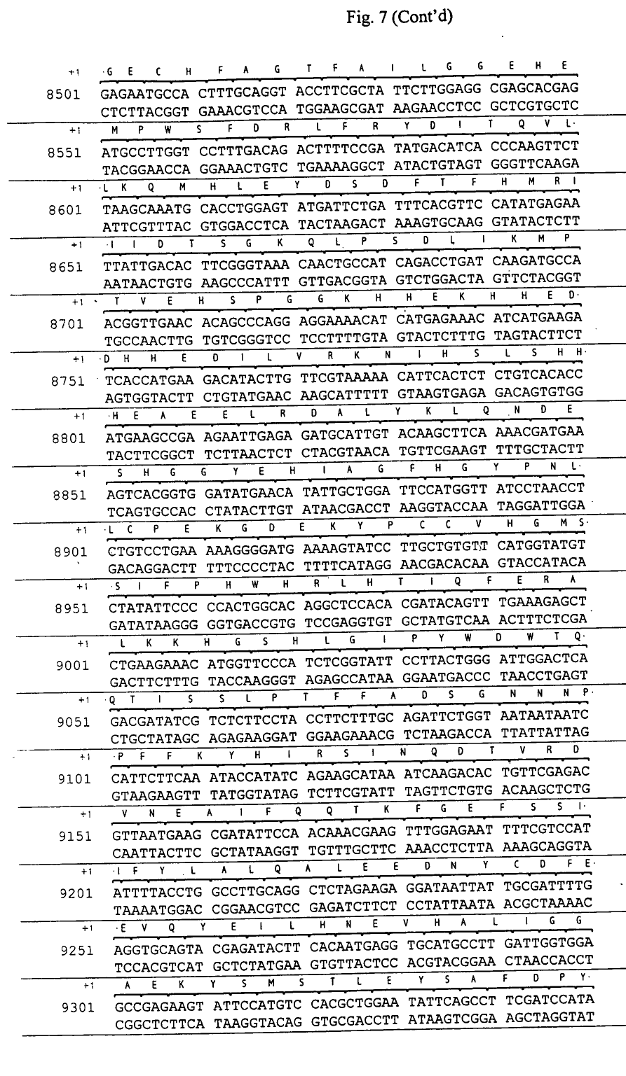

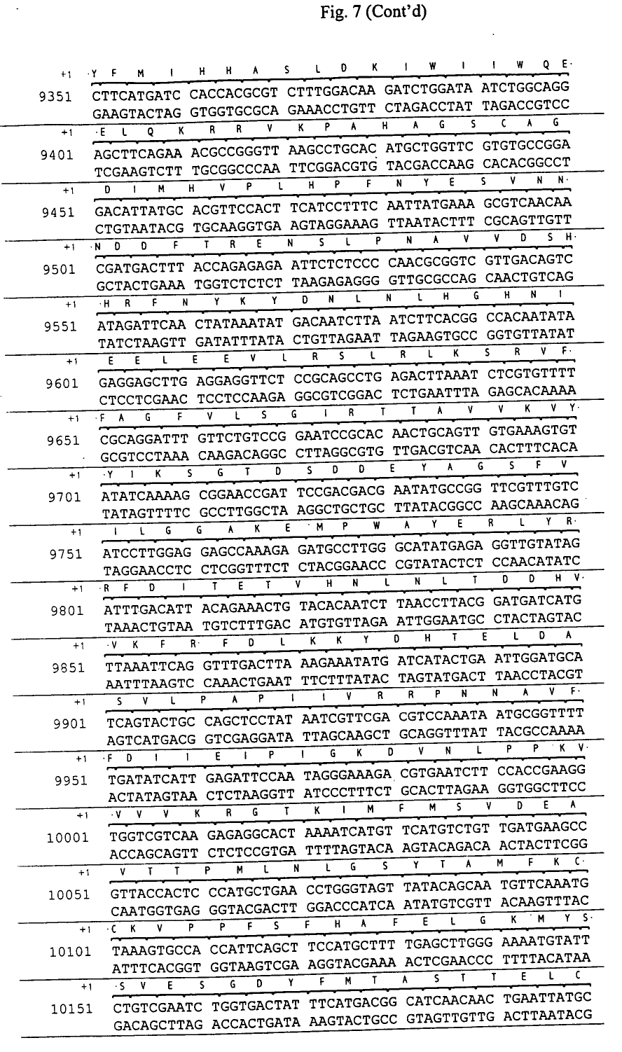

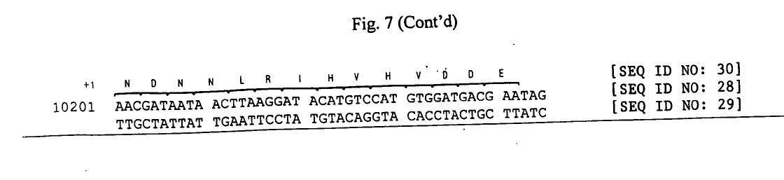

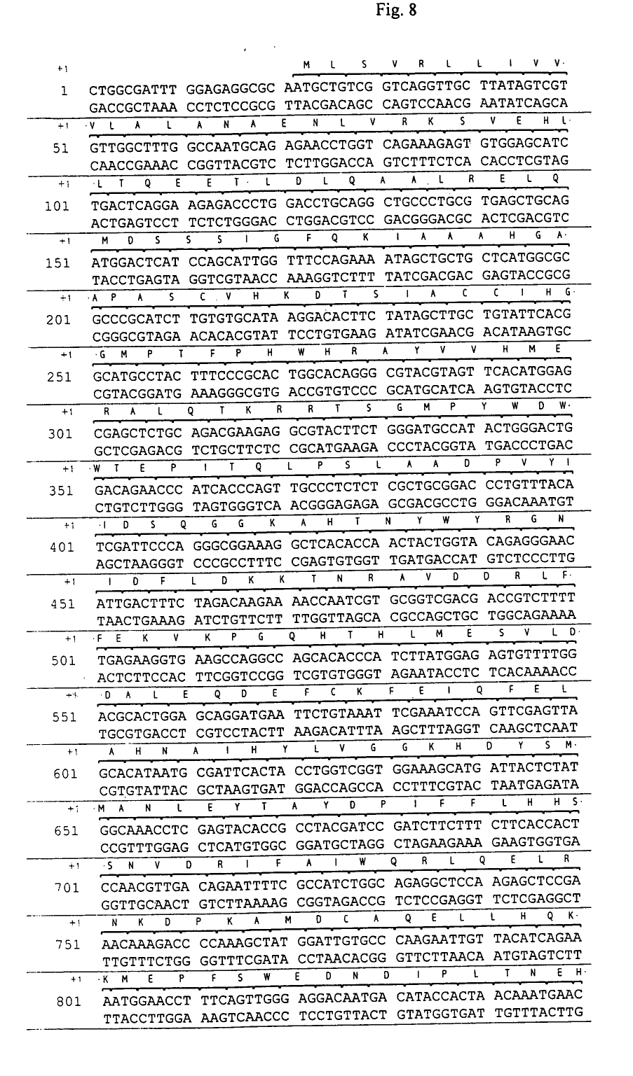

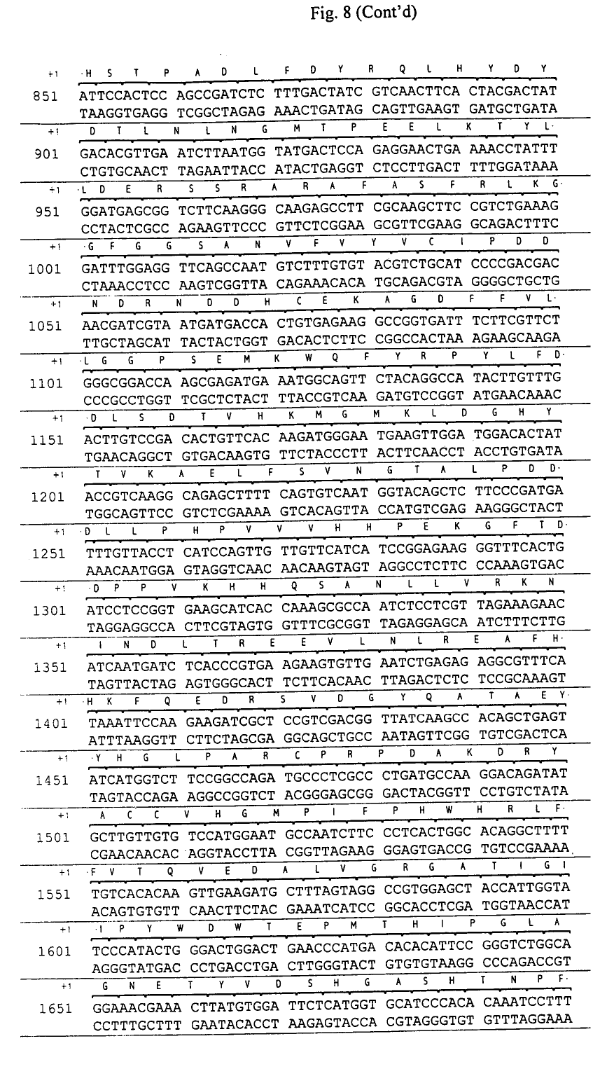

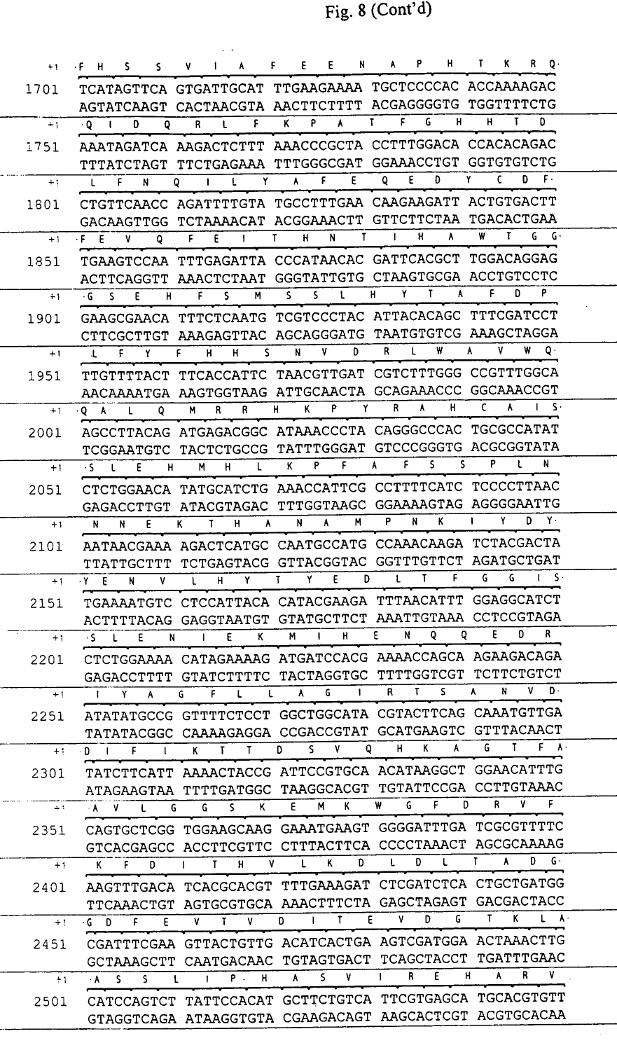

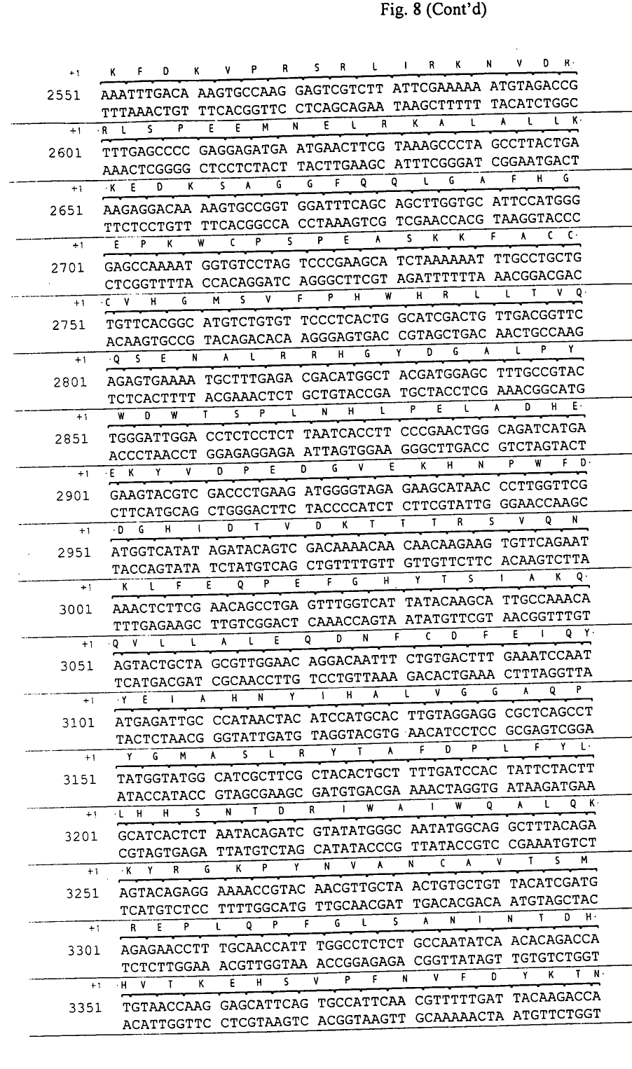

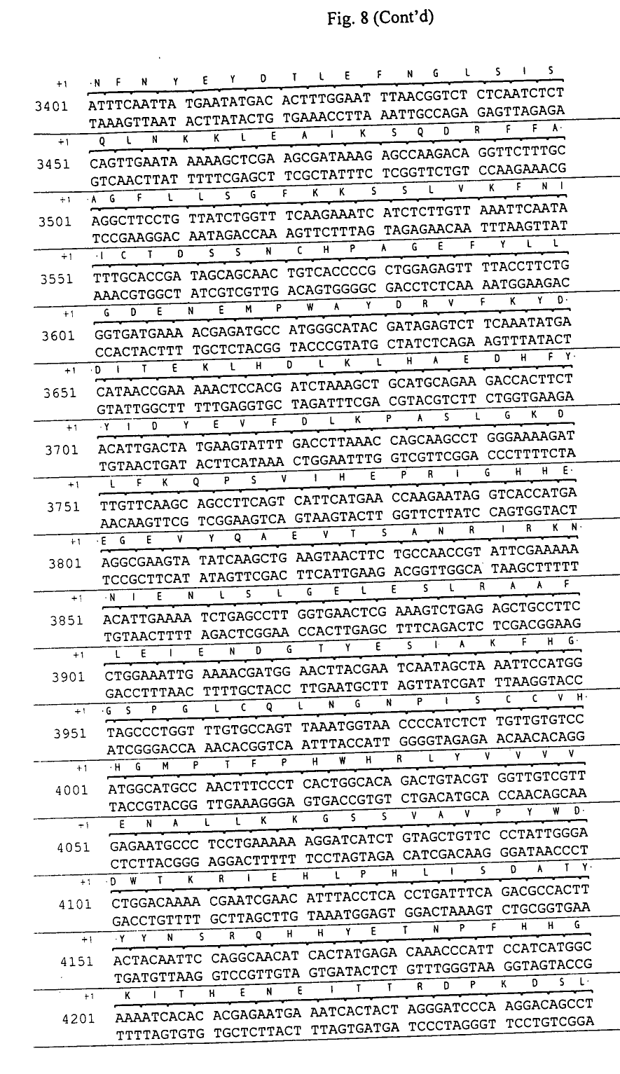

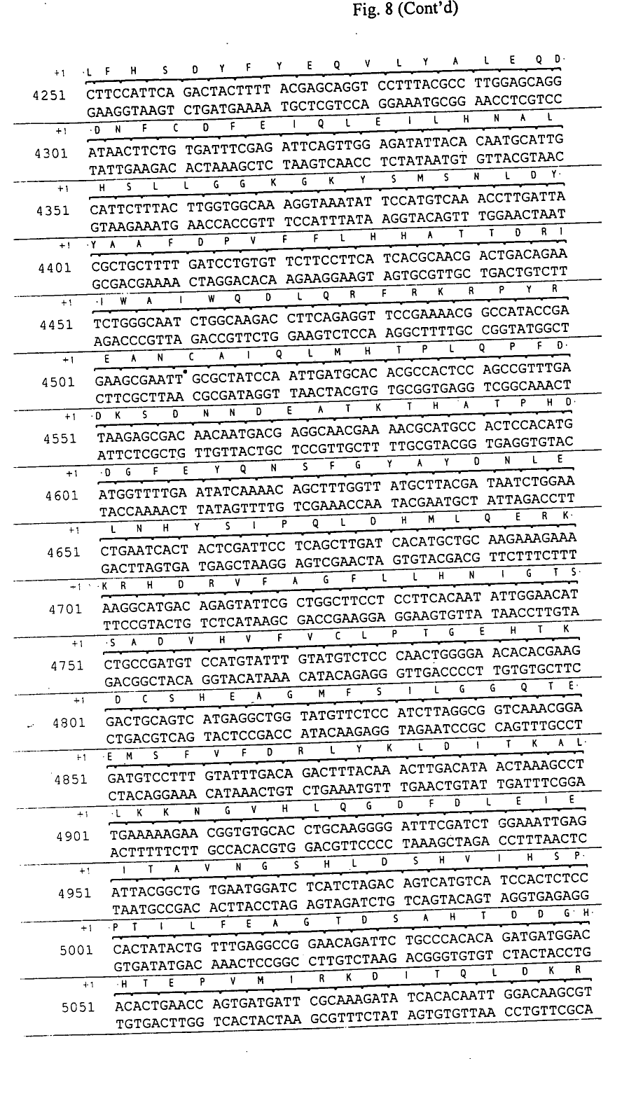

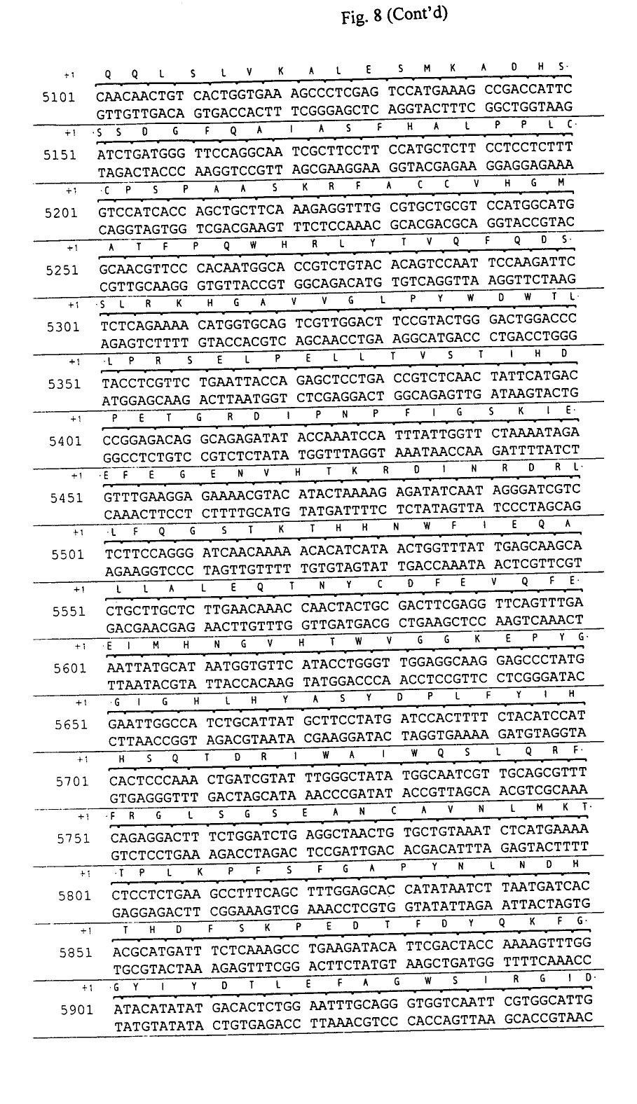

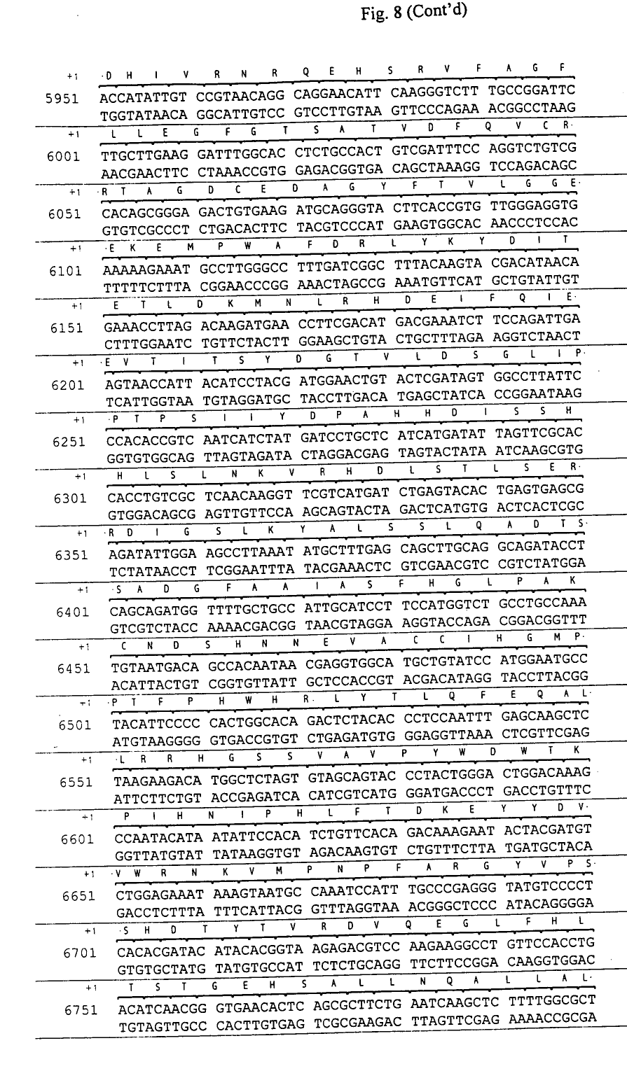

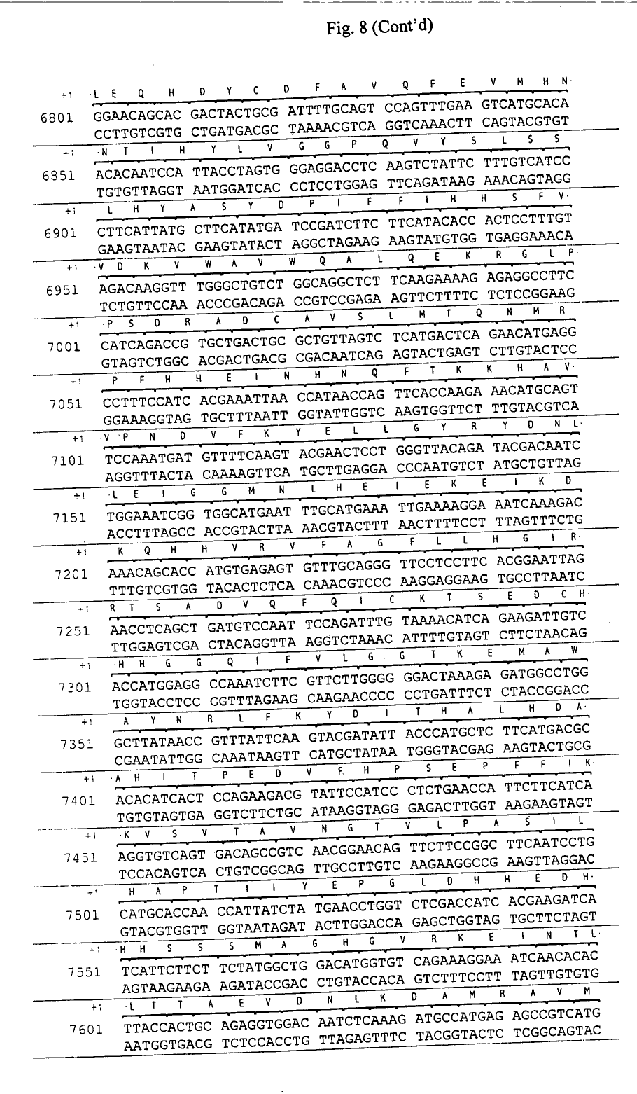

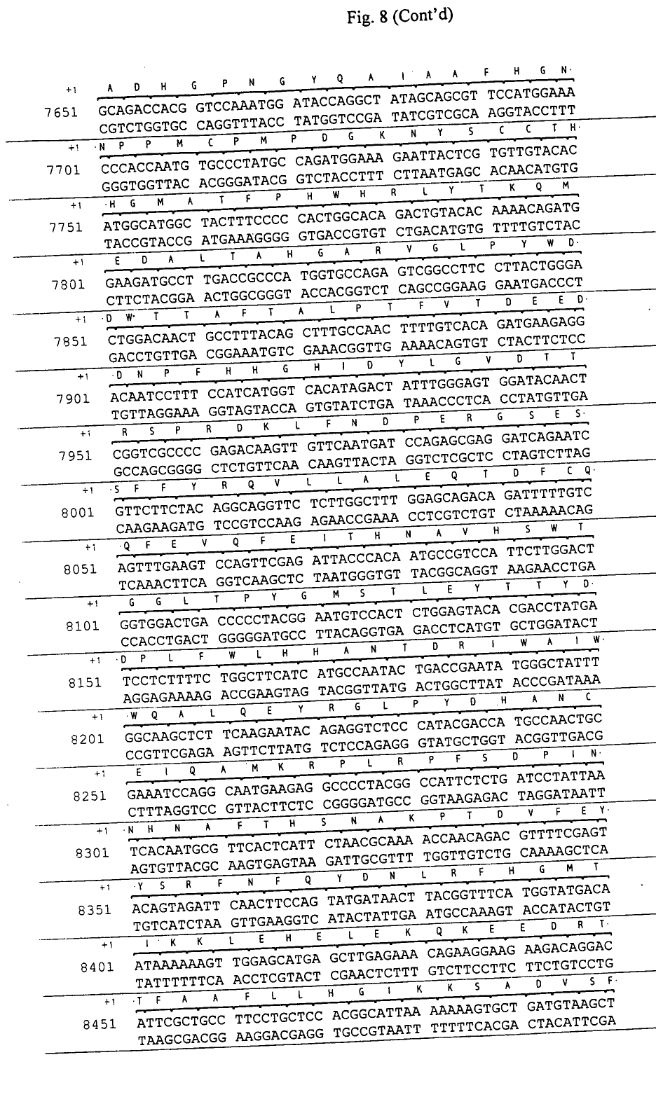

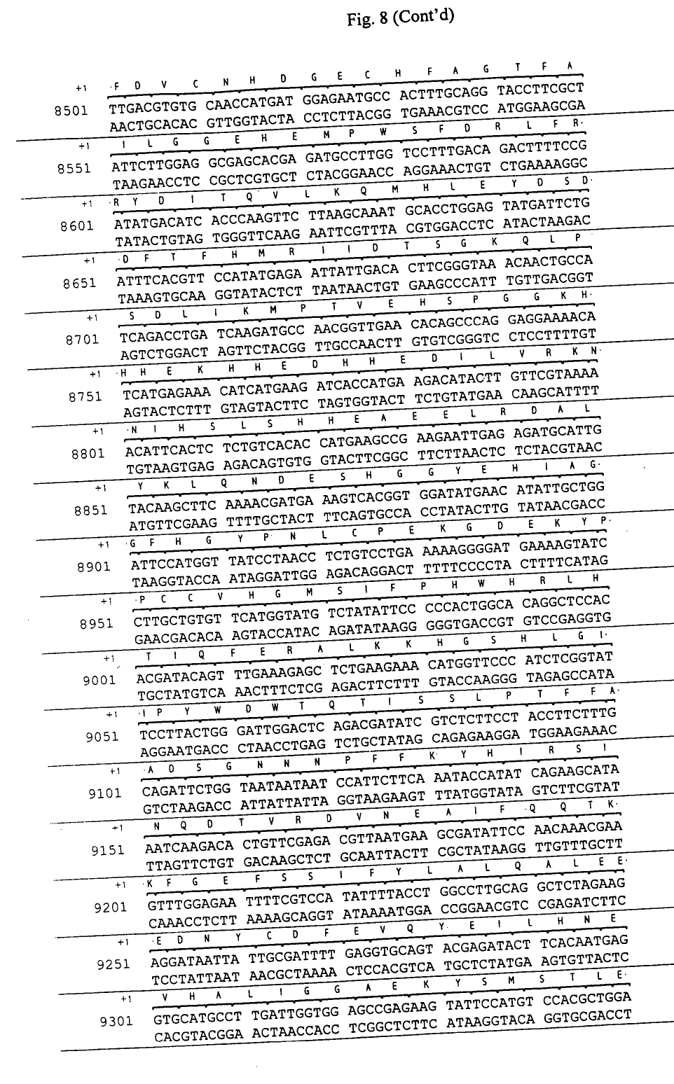

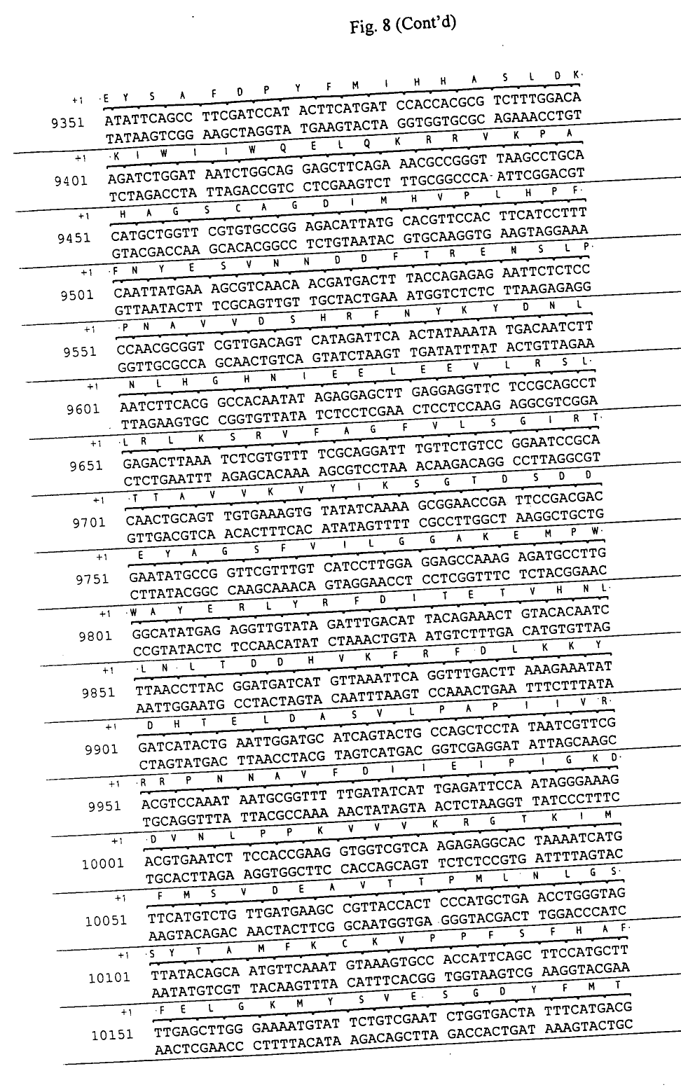

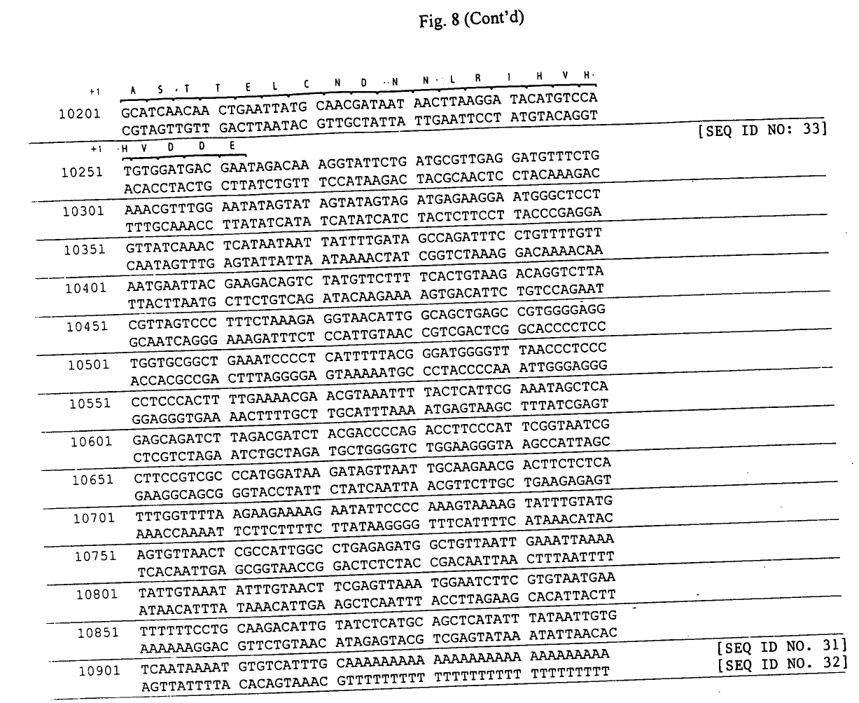

### DETAILED DESCRIPTION OF THE INVENTION

The present invention provides an isolated or purified nucleic acid molecule consisting essentially of a nucleotide sequence encoding KLH-2 or a fragment thereof comprising at least 1,080 (or at least 1,100, 1,125, 1,150, 1,175 or 1,200 or more) contiguous nucleotides. The present invention further provides an isolated or purified nucleic acid molecule consisting essentially of a nucleotide sequence encoding KLH-1 or a fragment thereof comprising at least 2,300 (or at least 2,325, 2,350, 2,375 or 2,400 or more) contiguous nucleotides. By “isolated” is meant the removal of a nucleic acid from its natural environment. By “purified” is meant that a given nucleic acid, whether one that has been removed from nature (including genomic DNA and mRNA) or synthesized (including cDNA) and/or amplified under laboratory conditions, has been increased in purity, wherein “purity” is a relative term, not “absolute purity.” “Nucleic acid molecule” is intended to encompass a polymer of DNA or RNA, i.e., a polynucleotide, which can be single-stranded or double-stranded and which can contain non-natural or altered nucleotides. Desirably, the isolated or purified nucleic acid molecule does not contain any introns or portions thereof.

Preferably, the isolated or purified nucleic acid molecule that consists essentially of a nucleotide sequence encoding KLH-2 or a fragment thereof (i) encodes the amino acid sequence of SEQ ID NO: 3 or at least 360 (or at least 375, 400, 425 or 450 or more) contiguous amino acids of SEQ ID NO: 3, (ii) consists essentially of the nucleotide sequence of SEQ ID NO: 1 or a fragment thereof comprising at least 1,080 contiguous nucleotides, (iii) hybridizes under moderately stringent conditions to an isolated or purified nucleic acid molecule consisting essentially of the nucleotide sequence that is complementary to SEQ ID NO: 1 or a fragment thereof comprising at least 1,080 (or at least 1,100, 1,125, 1,150, 1,175, 1,200 or more) contiguous nucleotides and is characterized by no more than 40% mismatch, or (iv) shares 60% (or 65%, 70%, 75, 80%, 85%, 90% or 95%) or more identity with SEQ ID NO: 1, such as naturally occurring and artificially generated variants as described herein. An isolated or purified nucleic acid molecule that hybridizes under highly stringent conditions to an isolated or purified nucleic acid molecule consisting essentially of the nucleotide sequence that is complementary to SEQ ID NO: 1 or a fragment thereof comprising at least 1,080 contiguous nucleotides preferably is characterized by no more than 20% mismatch.

When the fragment is obtained from nucleotides 1-1,530 or nucleotides 9,012-10,206 of SEQ ID NO: 1 or from nucleotides 1-1,527 or nucleotides 7,975-10,197 of SEQ ID NO: 22, the fragment can be substantially smaller than 1,080 contiguous nucleotides (e.g., a fragment encoding a single epitope, such as an immunogenic epitope (e.g., an epitope that induces an immune response in a mammal)), and all fragments, irrespective of length (e.g., 1,050, 1,025, 1,000, 900, 800, 700, 600, 500, 475, 450, 425, 400, 375, 350, 325, 300, 275, 250, 225, 200, 175, 150, 125, 100, 75, 50, 25, 20, and 12-15) and unique to nucleotides 1-1,530 or nucleotides 9,012-10,206 of SEQ ID NO: 1 or nucleotides 1-1,527 or nucleotides 7,975-10,197 of SEQ ID NO: 22, are contemplated by the present invention. Another fragment that is unique to SEQ ID NO: 1 is that which spans nucleotides 7,472-7,495 of SEQ ID NO: 1, and all fragments, irrespective of length and unique to nucleotides 7,472-7,495 of SEQ ID NO: 1, are also contemplated. One of ordinary skill in the art will readily appreciate that fragments unique to KLH-2 can overlap the aforementioned regions in whole or in part and extend into adjoining regions of SEQ ID NO: 1. Likewise, fragments unique to KLH-1 can overlap the aforementioned regions in whole or in part and extend into adjoining regions of SEQ ID NO: 22.

Preferably, the isolated or purified nucleic acid molecule that consists essentially of a nucleotide sequence encoding KLH-1 or a fragment thereof (i) encodes the amino acid sequence of SEQ ID NO: 24 or at least 770 (or at least 800, 825, 850, 875 or 900 or more) contiguous amino acids of SEQ ID NO: 24, (ii) consists essentially of the nucleotide sequence of SEQ ID NO: 22 or a fragment thereof comprising at least 2,300 contiguous nucleotides, (iii) hybridizes under moderately stringent conditions to an isolated or purified nucleic acid molecule consisting essentially of the nucleotide sequence that is complementary to SEQ ID NO: 22 or a fragment thereof comprising at least 2,300 (or at least 2,325, 2,350, 2,375, 2,400 or more) contiguous nucleotides, or (iv) shares 65% (or 70%, 75, 80%, 85%, 90% or 95%) or more identity with SEQ ID NO: 22, such as naturally occurring and artificially generated variants as described herein.

N-terminal sequence analysis of proteolytic cleavage products (Sohngen et al., Eur. J. Biochem. 248: 602-614 (1997)), along with analysis of amino acid sequence homology and conserved domains, enabled the determination of functional domains. Functional unit A of KLH-1 comprises amino acids 1-420 (SEQ ID NO: 24), nucleotides 1-1,260 (SEQ ID NO: 22), whereas functional unit A of KLH-2 comprises amino acids 1-422 (SEQ ID NO: 3), nucleotides 1-1,266 (SEQ ID NO: 1). Functional unit H of KLH-1 comprises amino acids 2,899-3,398 (SEQ ID NO: 24), nucleotides 8,695-10,197 (SEQ ID NO: 22), whereas functional unit H of KLH-2 comprises amino acids 2,905-3,401 (SEQ ID NO: 3), nucleotides 8,713-10,206 (SEQ ID NO: 1). Functional unit B of KLH-1 comprises amino acids 421-509 (SEQ ID NO: 24), nucleotides 1,261-1,527 (SEQ ID NO: 22), whereas functional unit B of KLH-2 comprises amino acids 423-510 (SEQ ID NO: 3), nucleotides 1,267-1,530 (SEQ ID NO: 1). Functional unit G of KLH-1 comprises amino acids 2,659-2,898 (SEQ ID NO: 24), nucleotides 7,975-8,694 (SEQ ID NO: 22).

An isolated or purified nucleic acid molecule consisting essentially of a nucleotide sequence encoding a variant KLH-2, or a fragment thereof comprising at least 1,080 (or at least 1,100, 1,125, 1,150, 1,175, or 1,200 or more) contiguous nucleotides, comprises one or more insertions, deletions, substitutions and/or inversions. Desirably, the variant KLH-2 does not differ functionally from the corresponding unmodified KLH-2, such as that comprising SEQ ID NO: 3. Preferably, the variant KLH-2 functions as an immunogen at least about 50%, more preferably at least about 75%, most preferably at least about 90% as well as the unmodified KLH-2 comprising SEQ ID NO: 3 as determined by an in vitro immunogenic assay. The manner in which the assay is carried out is not critical and can be conducted in accordance with methods known in the art. Preferably, the one or more substitution(s) results in the substitution of an amino acid of the encoded KLH-2 with another amino acid of approximately equivalent mass, structure and/or charge. A preferred variant KLH-2 comprises a deletion of all or part of the leader sequence, i.e., SEQ ID NO: 4, such that the encoded variant KLH-2 cannot be secreted by a cell.

An isolated or purified nucleic acid molecule consisting essentially of a nucleotide sequence encoding a variant KLH-1, or a fragment thereof comprising at least 2,300 (or at least 2,325, 2,350, 2,375, 2,400 or more) contiguous nucleotides, comprises one or more insertions, deletions, substitutions and/or inversions. Desirably, the variant KLH-1 does not differ functionally from the corresponding unmodified KLH-1, such as that comprising SEQ ID NO: 24. Preferably, the variant KLH-1 functions as an immunogen at least about 50%, more preferably at least about 75%, most preferably at least about 90% as well as the unmodified KLH-1 comprising SEQ ID NO: 24 as determined by an in vitro immunogenic assay. The manner in which the assay is carried out is not critical and can be conducted in accordance with methods known in the art. Preferably, the one or more substitution(s) results in the substitution of an amino acid of the encoded KLH-1 with another amino acid of approximately equivalent mass, structure and/or charge. A preferred variant KLH-1 comprises a deletion of all or part of the leader sequence, i.e., SEQ ID NO: 25, such that the encoded variant KLH-2 cannot be secreted by a cell.

The present invention also provides an isolated or purified nucleic acid molecule consisting essentially of a nucleotide sequence that is complementary to a nucleotide sequence encoding KLH-2 or a fragment thereof comprising at least 1,080 (or at least 1,100, 1,125, 1,150, 1,175, or 1,200 or more) contiguous nucleotides. Such an isolated or purified nucleic acid molecule preferably (i) is complementary to a nucleotide sequence encoding the amino acid sequence of SEQ ID NO: 3 or at least 360 (or at least 375, 400, 425 or 450 or more) contiguous amino acids of SEQ ID NO: 3, (ii) is complementary to the nucleotide sequence of SEQ ID NO: 1 or a fragment thereof comprising at least 1,080 contiguous nucleotides, (iii) hybridizes under moderately stringent conditions to an isolated or purified nucleic acid molecule consisting essentially of SEQ ID NO: 1 or a fragment thereof comprising at least 1,080 contiguous nucleotides and is characterized by no more than 40% mismatch, or (iv) shares 60% (or 65%, 70%, 75, 80%, 85%, 90% or 95%) or more identity with the nucleotide sequence that is complementary to SEQ ID NO: 1.

Likewise, the present invention provides an isolated or purified nucleic acid molecule consisting essentially of a nucleotide sequence that is complementary to a nucleotide sequence encoding KLH-1 or a fragment thereof comprising at least 2,300 (or at least 2,325, 2,350, 2,375, 2,400 or more) contiguous nucleotides. Such an isolated or purified nucleic acid molecule preferably (i) is complementary to a nucleotide sequence encoding the amino acid sequence of SEQ ID NO: 24 or at least 770 (or at least 800, 825, 850, 875, 900 or more) contiguous amino acids of SEQ ID NO: 24, (ii) is complementary to the nucleotide sequence of SEQ ID NO: 22 or a fragment thereof comprising at least 2,300 contiguous nucleotides, (iii) hybridizes under moderately stringent conditions to an isolated or purified nucleic acid molecule consisting essentially of SEQ ID NO: 22 or a fragment thereof comprising at least 2,300 contiguous nucleotides and is characterized by no more than 35% mismatch, or (iv) shares 65% (or 70%, 75, 80%, 85%, 90% or 95%) or more identity with the nucleotide sequence that is complementary to SEQ ID NO: 22. An isolated or purified nucleic acid molecule that hybridizes under highly stringent conditions to an isolated or purified nucleic acid molecule consisting essentially of the nucleotide sequence that is complementary to SEQ ID NO: 22 or a fragment thereof comprising at least 2,300 contiguous nucleotides preferably is characterized by no more than 20% mismatch.

An isolated or purified nucleic acid molecule can consist essentially of a nucleotide sequence that is complementary to either of a nucleotide sequence encoding a variant KLH-2 or at least 360 (or at least 375, 400, 425 or 450 or more) contiguous amino acids of a variant KLH-2 as described above. Likewise, an isolated or purified nucleic acid molecule can consist essentially of a nucleotide sequence that is complementary to either of a nucleotide sequence encoding a variant KLH-1 or at least 770 (or at least 800, 825, 850, 875 or 900 or more) contiguous amino acids of a variant KLH-1 as described above.

With respect to the above, one of ordinary skill in the art knows how to generate insertions, deletions, substitutions and/or inversions, in a given nucleic acid molecule. See, for example, the references cited herein under “Example.” With respect to the above isolated or purified nucleic acid molecules, it is preferred that any such insertions, deletions, substitutions and/or inversions are introduced into the nucleotide sequence encoding the regions between the copper-binding domains of KLH or a variant thereof. It is also preferred that the one or more substitution(s) result(s) in the substitution of an amino acid with another amino acid of approximately equivalent size, shape and charge. Insertions, deletions, substitutions and/or inversions also can be introduced into one or more of the copper-binding domains. If such mutations are introduced into the nucleotide sequences encoding one or more of the copper-binding domains, desirably not all such domains are mutated and those copper-binding domains that are mutated are located at the N-terminal or C-terminal ends of KLH.

Also with respect to the above, “does not differ functionally from” is intended to mean that the variant KLH has activity characteristic of the unmodified KLH. In other words, it can induce an immune response in a mammal, resulting in the production of a specific antibody that can be measured by standard immunoassay techniques known in the art. However, the variant KLH can be more or less active than the unmodified KLH as desired in accordance with the present invention.

An indication that polynucleotide sequences are substantially identical is if two molecules selectively hybridize to each other under stringent conditions. The phrase “hybridizes to” refers to the selective binding of a single-stranded nucleic acid probe to a single-stranded target DNA or RNA sequence of complementary sequence when the target sequence is present in a preparation of heterogeneous DNA and/or RNA. “Stringent conditions” are sequence-dependent and will be different in different circumstances. Generally, stringent conditions are selected to be about 20° C. lower than the thermal melting point (Tm) for the specific sequence at a defined ionic strength and pH. The Tm is the temperature (under defined ionic strength and pH) at which 50% of the target sequence hybridizes to a perfectly matched probe.

For example, under stringent conditions, as that term is understood by one skilled in the art, hybridization is preferably carried out using a standard hybridization buffer at a temperature ranging from about 50° C. to about 75° C., from about 60° C. to about 70° C., or from about 65° C. to about 68° C. Alternately, formamide can be included in the hybridization reaction, and the temperature of hybridization can be reduced to from about 35° C. to about 45° C., from about 40° C. to about 45° C., or to about 42° C. Desirably, formamide is included in the hybridization reaction at a concentration of from about 30% to about 50%, preferably from about 35% to about 45%, and optimally at about 40%. Moreover, optionally, the hybridized sequences are washed (if necessary to reduce non-specific binding) under relatively highly stringent conditions, as that term is understood by those skilled in the art. For instance, desirably, the hybridized sequences are washed one or more times using a solution comprising salt and detergent, at a temperature of from about 50° C. to about 75° C., from about 60° C. to about 70° C., or from about 65° C. to about 68° C. Preferably, a salt (e.g., such as sodium chloride) is included in the wash solution at a concentration of from about 0.01 M to about 1.0 M. Optimally, a detergent (e.g., such as sodium dodecyl sulfate) is also included at a concentration of from about 0.01% to about 1.0%. The following are examples of highly stringent and moderately stringent conditions for a Southern hybridization in aqueous buffers (no formamide) (Sambrook and Russell, Molecular Cloning, 3rd Ed. SCHL Press (2001)):

| | |
|---|---|
| Highly stringent | Moderately Stringent |
| hybridization conditions: | hybridization conditions: |
| 6X SSC or 6X SSPE | 6X SSC or 6X SSPE |
| 5x Denhardt's Reagent | 5x Denhardt's Reagent |
| 1% SDS | 1% SDS |
| 100 μg/ml salmon sperm DNA | 10 μg/ml salmon sperm DNA |
| hybridization at 65-68° C. | hybridization at 58-64° C. |
| Highly stringent | Moderately stringent |
| washing conditions: | washing conditions: |
| 0.1X SSC/0.1% SDS | 2X SSC/0.1% SDS |
| washing at 65-68° C. | washing at 58-64° C. |

In view of the above, “highly stringent conditions” allow for up to about 20% mismatch, preferably up to about 15% mismatch, more preferably up to about 10% mismatch, and most preferably less than about 5% mismatch, such as 4%, 3%, 2% or 1% mismatch. “At least moderately stringent conditions” preferably allow for up to about 40% mismatch, more preferably up to about 30% mismatch, and most preferably up to about 20% mismatch. “Low stringency conditions” preferably allow for up to about 60% mismatch, more preferably up to about 50% mismatch, and most preferably up to about 40% mismatch. With respect to the preceding ranges of mismatch, 1% mismatch corresponds to one degree decrease in the melting temperature.

The above isolated or purified nucleic acid molecules also can be characterized in terms of “percentage of sequence identity.” In this regard, a given nucleic acid molecule as described above can be compared to a nucleic acid molecule encoding a corresponding gene (i.e., the reference sequence) by optimally aligning the nucleic acid sequences over a comparison window, wherein the portion of the polynucleotide sequence in the comparison window may comprise additions or deletions (i.e., gaps) as compared to the reference sequence, which does not comprise additions or deletions, for optimal alignment of the two sequences. The percentage of sequence identity is calculated by determining the number of positions at which the identical nucleic acid base occurs in both sequences, i.e., the number of matched positions, dividing the number of matched positions by the total number of positions in the window of comparison, and multiplying the result by 100 to yield the percentage of sequence identity. Optimal alignment of sequences for comparison may be conducted by computerized implementations of known algorithms (e.g., GAP, BESTFIT, FASTA, and TFASTA in the Wisconsin Genetics Software Package, Genetics Computer Group (GCG), 575 Science Dr., Madison, Wis., or BlastN and BlastX available from the National Center for Biotechnology Information, Bethesda, Md.), or by inspection. Sequences are typically compared using BESTFIT or BlastN with default parameters.

“Substantial sequence identity” means that at least 75%, preferably at least 80%, more preferably at least 90%, and most preferably at least 95% (such as 96%, 97%, 98% or 99%) of the sequence of a given nucleic acid molecule is identical to a given reference sequence. Typically, two polypeptides are considered to be substantially similar if at least 40%, preferably at least 60%, more preferably at least 90%, and most preferably at least 95% (such as 96%, 97%, 98% or 99%) of the amino acids of which the polypeptides are comprised are identical to or represent conservative substitutions of the amino acids of a given reference sequence.

One of ordinary skill in the art will appreciate, however, that two polynucleotide sequences can be substantially different at the nucleic acid level, yet encode substantially similar, if not identical, amino acid sequences, due to the degeneracy of the genetic code. The present invention is intended to encompass such polynucleotide sequences.

While the above-described nucleic acid molecules can be isolated or purified, alternatively they can be synthesized. Methods of nucleic acid synthesis are known in the art. See, e.g., the references cited herein under “Example.”

The above-described nucleic acid molecules can be used, in whole or in part (i.e., as fragments or primers), to identify and isolate corresponding genes from other organisms for use in the context of the present inventive methods using conventional means known in the art. See, for example, the references cited herein under “Examples.” Evolutionarily conserved portions of the sequence given in SEQ ID NO:1 or SEQ ID NO: 22, for example, can be used to identify highly related hemocyanin nucleic acids, such as in Rapana thomasiana and Megathura crenulata, among others. In addition, the above-described nucleic acid molecules (or fragments thereof) can be used as probes to identify limpet cells that produce KLH.

In view of the above, the present invention also provides a vector comprising an above-described isolated or purified nucleic acid molecule, optionally as part of an encoded fusion protein. A nucleic acid molecule as described above can be cloned into any suitable vector and can be used to transform or transfect any suitable host. The selection of vectors and methods to construct them are commonly known to persons of ordinary skill in the art and are described in general technical references (see, in general, “Recombinant DNA Part D,” Methods in Enzymology, Vol. 153, Wu and Grossman, eds., Academic Press (1987) and the references cited herein under “Example”). Desirably, the vector comprises regulatory sequences, such as transcription and translation initiation and termination codons, which are specific to the type of host (e.g., bacterium, fungus, plant or animal) into which the vector is to be introduced, as appropriate and taking into consideration whether the vector is DNA or RNA. Preferably, the vector comprises regulatory sequences that are specific to the genus of the host. Most preferably, the vector comprises regulatory sequences that are specific to the species of the host.

Constructs of vectors, which are circular or linear, can be prepared to contain an entire nucleic acid sequence as described above or a portion thereof ligated to a replication system functional in a prokaryotic or eukaryotic host cell. Replication systems can be derived from ColE1, 2 mμ plasmid, λ, SV40, bovine papilloma virus, and the like.

In addition to the replication system and the inserted nucleic acid, the construct can include one or more marker genes, which allow for selection of transformed or transfected hosts. Marker genes include biocide resistance, e.g., resistance to antibiotics, heavy metals, etc., complementation in an auxotrophic host to provide prototrophy, and the like.

Suitable vectors include those designed for propagation and expansion or for expression or both. A preferred cloning vector is selected from the group consisting of the pUC series the pBluescript series (Stratagene, LaJolla, Calif.), the pET series (Novagen, Madison, Wis.), the pGEX series (Pharmacia Biotech, Uppsala, Sweden), and the pEX series (Clontech, Palo Alto, Calif.). Bacteriophage vectors, such as λGT10, λGT11, αZapII (Stratagene), λ EMBL4, and λ NM1149, also can be used. Examples of plant expression vectors include pBI101, pBI101.2, pBI101.3, pBI121 and pBIN19 (Clontech). Examples of animal expression vectors include pEUK-C1, pMAM and pMAMneo (Clontech).

An expression vector can comprise a native or nonnative promoter operably linked to an isolated or purified nucleic acid molecule as described above. The selection of promoters, e.g., strong, weak, inducible, tissue-specific and developmental-specific, is within the skill in the art. Similarly, the combining of a nucleic acid molecule as described above with a promoter is also within the skill in the art.

Optionally, the isolated or purified nucleic acid molecule can be part of an encoded fusion protein. The generation of fusion proteins is within the ordinary skill in the art (see, e.g., references cited under “Example”) and can involve the use of restriction enzyme or recombinational cloning techniques (see, e.g., Gateway™ (Invitrogen, Carlsbad, Calif.). See, also, U.S. Pat. No. 5,314,995.

Also in view of the above, the present invention provides a host cell comprising and expressing an isolated or purified nucleic acid molecule, optionally in the form of a vector, as described above. Examples of host cells include, but are not limited to, a human cell, a human cell line, E. coli (e.g., E. coli TB-1, TG-2, DH5α, XL-Blue MRF' (Stratagene), SA2821 and Y1090), B. subtilis, P. aerugenosa, S. cerevisiae, N. crassa, insect cells (e.g., Sf9, Ea4) and others set forth herein below. Depending on the construct, it may be necessary to keep a culture of E. coli at low density for cloning purposes and, possibly, also for expression.

The present invention further provides an isolated or purified polypeptide molecule consisting essentially of an amino acid sequence encoding KLH-2 or at least 360 (or at least 375, 400, 425 or 450 or more) contiguous amino acids of KLH-2, which is optionally glycosylated, amidated, carboxylated, phosphorylated, esterified, N-acylated or converted into an acid addition salt and/or dimerized or polymerized, wherein the isolated or purified polypeptide molecule is essentially free from KLH-1. Also provided is an isolated or purified polypeptide molecule consisting essentially of an amino acid sequence encoding KLH-1 or at least 770 (or at least 800, 825, 850, 875 or 900 or more) contiguous amino acids of KLH-1, which is optionally glycosylated, amidated, carboxylated, phosphorylated, esterified, N-acylated or converted into an acid addition salt and/or dimerized or polymerized, wherein the isolated or purified polypeptide molecule is essentially free from KLH-2. By “polymerized” is meant at least trimerized. Especially preferred polymers include decamers and didecamers. Methods of protein modification (e.g., glycosylation, amidation, carboxylation, phosphorylation, esterification, N-acylation, and conversion into acid addition salts), dimerization and polymerization are known in the art.

When the fragment is obtained from amino acids 1-510 or amino acids 3,004-3,401 of SEQ ID NO: 3, the fragment can be substantially smaller than 360 contiguous amino acids (e.g., a fragment comprising a single epitope, such as an immunogenic epitope (e.g., an epitope that induces an immune response in a mammal)), and all fragments, irrespective of length (e.g., 350, 325, 300, 275, 250, 225, 200, 175, 150, 125, 100, 75, 50, 25, 20, 15, 10, and 4-5) and unique to amino acids 1-510 or amino acids 3,004-3,401 of SEQ ID NO: 3, are contemplated by the present invention. Other fragments that are unique to SEQ ID NO: 3 are those that span amino acids 2,491-2,498 and 2,892-2,907 of SEQ ID NO: 3. One of ordinary skill in the art will readily appreciate that fragments unique to KLH-2 can overlap the aforementioned regions in whole or in part and extend into adjoining regions of SEQ ID NO: 3.

When the fragment is obtained from amino acids 1-509 or amino acids 2,659-3,398 of SEQ ID NO: 24, the fragment can be substantially smaller than 770 continguous amino acids (e.g., a fragment comprising a single epitope, such as an immunogenic epitope (e.g., an epitope that induces an immune response in a mammal)), and all fragments, irrespective of length (e.g., 750, 725, 700, 675, 650, 625, 600, 575, 550, 525, 500, 475, 450, 425, 400, 375, 350, 325, 300, 275, 250, 225, 200, 175, 150, 125, 100, 75, 50, 25, 20, 15, 10, and 4-5) and unique to amino acids 1-509 or amino acids 2,659-3,398 of SEQ ID NO: 24 are contemplated by the present invention. One of ordinary skill in the art will readily appreciate that fragments unique to KLH-1 can overlap the aforementioned regions in whole or in part and extend into adjoining regions of SEQ ID NO: 24.

One of ordinary skill in the art will readily appreciate that one or more fragments of KLH-2, such as from amino acids 1-510 or amino acids 3,004-3,401 of SEQ ID NO: 3, can be combined with one or more fragments of KLH-1, such as from amino acids 1-509 or amino acids 2,659-3,398 of SEQ ID NO: 24. Such combinations of fragments can be generated at the nucleic acid or amino acid level in accordance with methods known in the art.

An isolated or purified polypeptide molecule can consist essentially of an amino acid sequence encoding a variant KLH-2 or at least 360 (or at least 375, 400, 425 or 450 or more) contiguous amino acids of a variant KLH-2, which is optionally glycosylated, amidated, carboxylated, phosphorylated, esterified, N-acylated, converted into an acid addition salt and/or dimerized or polymerized, wherein the isolated or purified polypeptide molecule is essentially free from KLH-1. Alternatively, an isolated or purified polypeptide molecule can consist essentially of an amino acid sequence encoding a variant KLH-1 or at least 770 (or at least 800, 825, 850, 875 or 900 or more) contiguous amino acids of a variant KLH-1, which is optionally glycosylated, amidated, carboxylated, phosphorylated, esterified, N-acylated, converted into an acid addition salt and/or dimerized or polymerized, wherein the isolated or purified polypeptide molecule is essentially free from KLH-2.

In view of the above isolated or purified polypeptide molecules and variants thereof, the present invention also provides fragments thereof comprising at least one epitope that can bind to a receptor on the surface of a cell that functions in cellular immunity, such as a T cell or a B cell. An example of such a receptor is the T cell receptor. Such fragments can consist essentially of from about 5 to about 8 amino acids, from about 10 to about 16 amino acids, from about 20 to about 30 amino acids, or can comprise substantially more amino acids. The fragments can be readily generated by the ordinarily skilled artisan in accordance with methods known in the art. The ability of such fragments to bind to a receptor on the surface of a cell that functions in cellular immunity also can be determined in accordance with methods known in the art. The fragments can comprise from about 5 to about 8 amino acids or more in the following regions of SEQ ID NO: 3: amino acids 1-510, 539-543, 823-827, 1,655-1,660, 2,491-2,498, 2,892-2,907, and 3,004-3,401 or in the following regions of SEQ ID NO: 24: amino acids 1-509 and 2,659-3,398. These regions are exemplary and are not limiting.

Functional units of KLH can be determined by N-terminal sequence analysis of proteolytic cleavage products. See, e.g., Harris et al., Micron 30: 597-623 (1999). Reverse immunogenetics can be used to determine peptides presented by the major histocompatibility complex (MHC) class I and II molecules to T-cell receptors upon stimulation with KLH in order to identify the most potent peptides and domains of KLH for antigen presentation. See, e.g., Immunobiology, 5th ed., Janeway et al., eds., Garland Publishing, New York and London (2001).

In view of the foregoing, the present invention provides a method of identifying an immunogenic portion of KLH. The method comprises:

(i) contacting antigen-presenting cells with KLH or a portion thereof,

(ii) harvesting the antigen-presenting cells that have been contacted with KLH or a portion thereof,

(iii) purifying complexes of major histocompatibility complex (MHC) and peptidic fragments derived from KLH from the harvested antigen-presenting cells,

(iv) isolating the peptidic fragments from the purified complexes, and

(v) analyzing the peptidic fragments to determine which fragments were bound to MHC, whereupon an immunogenic portion of KLH is identified. The method can be conducted in vitro or in vivo. If conducted in vivo, a preferred organism is a mammal. The complexes can be purified by any suitable purification method know in the art, such as immuno-affinity purification. The peptidic fragments can be isolated from the purified complexes by any suitable means, such as acetic acid elution. Preferably, the KLH is KLH-1 in the absence of KLH-2 or KLH-2 in the absence of KLH-1. The peptides can be analyzed in accordance with methods well-known in the art, such as by post-source decay and matrix-assisted laser desorption ionization time-of-flight mass spectrometry (PSD-MALDI-TOF-MS) (see, e.g., J. Immunol. Methods 246: 1-12 (2000)). Accordingly, the present invention provides an immunogenic portion of KLH identified in accordance with the method.

The polypeptide preferably comprises an amino end and a carboxyl end. The polypeptide can comprise D-amino acids, L-amino acids or a mixture of D- and L-amino acids. The D-form of the amino acids, however, is particularly preferred since a polypeptide comprised of D-amino acids is expected to have a greater retention of its biological activity in vivo, given that the D-amino acids are not recognized by naturally occurring proteases.

The polypeptide can be prepared by any of a number of conventional techniques. The polypeptide can be isolated or purified from a naturally occurring source or from a recombinant source. While the polypeptide can be isolated or purified from a naturally occurring source, such isolation is difficult and costly and does not readily or easily enable the separation of KLH polypeptides of isotypes 1 and 2. Thus, recombinant production is preferred. For instance, in the case of recombinant polypeptides, a DNA fragment encoding a desired peptide can be subcloned into an appropriate vector using well-known molecular genetic techniques (see, e.g., Maniatis et al., Molecular Cloning: A Laboratory Manual, 2nd ed. (Cold Spring Harbor Laboratory, 1982); Sambrook et al., Molecular Cloning: A Laboratory Manual, 2^{nd} ed. (Cold Spring Harbor Laboratory, 1989). The fragment can be transcribed and the polypeptide subsequently translated in vitro. Commercially available kits can also be employed (e.g., such as manufactured by Clontech, Palo Alto, Calif.; Amersham Pharmacia Biotech Inc., Piscataway, N.J.; InVitrogen, Carlsbad, Calif., and the like). The polymerase chain reaction optionally can be employed in the manipulation of nucleic acids.

Alterations of the native amino acid sequence to produce variant polypeptides can be done by a variety of means known to those skilled in the art. For instance, site-specific mutations can be introduced by ligating into an expression vector a synthesized oligonucleotide comprising the modified site. Alternately, oligonucleotide-directed site-specific mutagenesis procedures can be used such as disclosed in Walder et al., Gene 42: 133 (1986); Bauer et al., Gene 37: 73 (1985); Craik, Biotechniques, 12-19 (January 1995); and U.S. Pat. Nos. 4,518,584 and 4,737,462.

With respect to the above isolated or purified polypeptides, it is preferred that any such insertions, deletions and/or substitutions are introduced between copper-binding domains of the KLH or a variant thereof. It is also preferred that the one or more substitution(s) result(s) in the substitution of an amino acid with another amino acid of approximately equivalent mass, structure and charge. Insertions, deletions and/or substitutions also can be introduced into one or more of the copper-binding domains. If such mutations are introduced into one or more of the copper-binding domains, desirably not all copper-binding domains are mutated and domains that are mutated are located at the N-terminal or C-terminal ends of KLH.

Any appropriate expression vector (e.g., as described in Pouwels et al., Cloning Vectors: A Laboratory Manual (Elsevier, NY: 1985)) and corresponding suitable host can be employed for production of recombinant polypeptides. Expression hosts include, but are not limited to, bacterial species within the genera Escherichia, Bacillus, Pseudomonas, Salmonella, mammalian or insect host cell systems including baculovirus systems (e.g., as described by Luckow et al., Bio/Technology 6: 47 (1988)), and established cell lines such as the COS-7, C127, 3T3, CHO, HeLa, BHK cell line, and the like. The ordinarily skilled artisan is, of course, aware that the choice of expression host has ramifications for the type of polypeptide produced. For instance the glycosylation of polypeptides produced in yeast or mammalian cells (e.g., COS-7 cells) will differ from that of polypeptides produced in bacterial cells such as Escherichia coli.

Alternately, the polypeptide (including the variant polypeptides) can be synthesized using standard peptide synthesizing techniques well-known to those of skill in the art (e.g., as summarized in Bodanszky, Principles of Peptide Synthesis, (Springer-Verlag, Heidelberg: 1984)). In particular, the polypeptide can be synthesized using the procedure of solid-phase synthesis (see, e.g., Merrifield, J. Am. Chem. Soc. 85: 2149-54 (1963); Barany et al., Int. J. Peptide Protein Res. 30: 705-739 (1987); and U.S. Pat. No. 5,424,398). If desired, this can be done using an automated peptide synthesizer. Removal of the t-butyloxycarbonyl (t-BOC) or 9-fluorenylmethyloxycarbonyl (Fmoc) amino acid blocking groups and separation of the polypeptide from the resin can be accomplished by, for example, acid treatment at reduced temperature. The polypeptide-containing mixture can then be extracted, for instance, with dimethyl ether, to remove non-peptidic organic compounds, and the synthesized polypeptide can be extracted from the resin powder (e.g., with about 25% w/v acetic acid). Following the synthesis of the polypeptide, further purification (e.g., using high performance liquid chromatography (HPLC)) optionally can be done in order to eliminate any incomplete polypeptides or free amino acids. Amino acid and/or HPLC analysis can be performed on the synthesized polypeptide to validate its identity. For other applications according to the invention, it may be preferable to produce the polypeptide as part of a larger fusion protein, such as by the methods described herein or other genetic means, or as part of a larger conjugate, such as through physical or chemical conjugation, as known to those of ordinary skill in the art and described herein.

If desired, the polypeptides of the invention (including variant polypeptides) can be modified, for instance, by glycosylation, amidation, carboxylation, or phosphorylation, or by the creation of acid addition salts, amides, esters, in particular C-terminal esters, and N-acyl derivatives of the polypeptides of the invention. The polypeptides also can be modified to create polypeptide derivatives by forming covalent or noncovalent complexes with other moieties in accordance with methods known in the art. Covalently-bound complexes can be prepared by linking the chemical moieties to functional groups on the side chains of amino acids comprising the polypeptides, or at the N- or C-terminus.

Thus, in this regard, the present invention also provides a fusion protein and a conjugate comprising an above-described isolated or purified polypeptide molecule or fragment thereof and a therapeutically or prophylactically active agent. “Prophylactically” as used herein does not necessarily mean prevention, although prevention is encompassed by the term. Prophylactic activity also can include lesser effects, such as inhibition of the spread of cancer or a pathogenic infection; etc. Preferably, the active agent is a hapten, an allergen, a sperm coat protein (e.g., for use as a contraceptive), an immunogen, or hemocyanin β-adrenergic receptor peptide. Preferably, the immunogen is an antigen of a pathogen, e.g., bacterial, viral or parasitic pathogen, or an antigen of a cancer. Methods of conjugation are known in the art. In addition, conjugate kits are commercially available. For examples of methods of conjugation and conjugates see, e.g., Hermanson, G. T., Bioconjugate Techniques, 1996, Academic Press, San Diego, Calif.; U.S. Pat. Nos. 6,013,779; 6,274,552 and 6,080,725 and Ragupathi et al., Glycoconjugate Journal 15: 217-221 (1998).

The present invention also provides a composition comprising an above-described isolated or purified polypeptide molecule, optionally in the form of a conjugate or a fusion protein comprising a prophylactically or therapeutically active agent, and an excipient or an adjuvant. Excipients and adjuvants are well-known in the art, and are readily available. The choice of excipient/adjuvant will be determined in part by the particular route of administration and whether a nucleic acid molecule or a polypeptide molecule (or conjugate or fusion protein thereof) is being administered. Accordingly, there is a wide variety of suitable formulations for use in the context of the present invention, and the invention expressly provides a pharmaceutical composition that comprises an active agent of the invention and a pharmaceutically acceptable excipient/adjuvant. The following methods and excipients/adjuvants are merely exemplary and are in no way limiting.

Formulations suitable for oral administration can consist of (a) liquid solutions, such as an effective amount of the compound dissolved in diluent, such as water, saline, or orange juice; (b) capsules, sachets or tablets, each containing a predetermined amount of the active ingredient, as solids or granules; (c) suspensions in an appropriate liquid; and (d) suitable emulsions. Tablet forms can include one or more of lactose, mannitol, corn starch, potato starch, microcrystalline cellulose, acacia, gelatin, colloidal silicon dioxide, croscarmellose sodium, talc, magnesium stearate, stearic acid, and other excipients, colorants, diluents, buffering agents, moistening agents, preservatives, flavoring agents, and pharmacologically compatible excipients. Lozenge forms can comprise the active ingredient in a flavor, usually sucrose and acacia or tragacanth. Pastilles can comprise the active ingredient in an inert base, such as gelatin and glycerin, or sucrose and acacia, emulsions, gels, and the like containing, in addition to the active ingredient, such excipients/carriers as are known in the art.

An active agent of the present invention, either alone or in combination with other suitable components, can be made into aerosol formulations to be administered via inhalation. These aerosol formulations can be placed into pressurized acceptable propellants, such as dichlorodifluoromethane, propane, nitrogen, and the like. They also can be formulated as pharmaceuticals for non-pressured preparations such as in a nebulizer or an atomizer.

Formulations suitable for parenteral administration include aqueous and non-aqueous, isotonic sterile injection solutions, which can contain anti-oxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. The formulations can be presented in unit-dose or multi-dose sealed containers, such as ampules and vials, and can be stored in a freeze-dried (lyophilized) condition requiring only the addition of the sterile liquid excipient, for example, water, for injections, immediately prior to use. Extemporaneous injection solutions and suspensions can be prepared from sterile powders, granules, and tablets of the kind previously described.

Additionally, active agents of the present invention can be made into suppositories by mixing with a variety of bases such as emulsifying bases or water-soluble bases. Formulations suitable for vaginal administration can be presented as pessaries, tampons, creams, gels, pastes, foams, or spray formulas containing, in addition to the active ingredient, such carriers as are known in the art to be appropriate. Further suitable formulations are found in Remington's Pharmaceutical Sciences, 17th ed., (Mack Publishing Company, Philadelphia, Pa.: 1985), and methods of drug delivery are reviewed in, for example, Langer, Science 249: 1527-1533 (1990).

In view of the above, the present invention provides a method of treating cancer prophylactically or therapeutically in a mammal. The method comprises administering to the mammal an effective amount of:

(a) an isolated or purified nucleic acid molecule encoding a fusion protein comprising (i) KLH, a variant KLH or an immunogenic fragment of either of the foregoing, and (ii) an antigen of the cancer, wherein the isolated or purified nucleic acid molecule is optionally in the form of a vector,

(b) a fusion protein comprising (i) KLH, a variant KLH or an immunogenic fragment of either of the foregoing, and (ii) an antigen of the cancer,

(c) a conjugate comprising (i) (a) KLH-2, a variant KLH-2, or an immunogenic fragment of either of the foregoing comprising at least 360 contiguous amino acids, wherein the KLH-2 is essentially free from KLH-1, or (b) KLH-1, a variant KLH-1 or an immunogenic fragment of either of the foregoing comprising at least 770 amino acids, wherein the KLH-1 is essentially free from KLH-2, and (ii) an antigen of the cancer or an anti-cancer agent, or

(d) a composition comprising (i) (a) KLH-2, a variant KLH-2, or an immunogenic fragment of either of the foregoing comprising at least 360 contiguous amino acids, wherein the KLH-2 is essentially free from KLH-1, or (b) KLH-1, a variant KLH-1, or an immunogenic fragment of either of the foregoing comprising at least 770 amino acids, wherein the KLH-1 is essentially free from KLH-2, and (ii) an antigen of the cancer and/or an anti-cancer agent, whereupon the mammal is treated for the cancer prophylactically or therapeutically. Such a method can further comprise the co-administration, whether simultaneously or sequentially, in either order, of an anti-cancer agent by the same or different route.

Preferably, the cancer is adenocarcinoma, bladder cancer, colon cancer, breast cancer, lung cancer or skin cancer. The skin cancer is preferably melanoma.

The anti-cancer agent can be a chemotherapeutic agent, e.g., a polyamine or an analogue thereof. Examples of therapeutic polyamines include those set forth in U.S. Pat. Nos. 5,880,161, 5,541,230 and 5,962,533, Saab et al., J. Med. Chem. 36: 2998-3004 (1993), Bergeron et al., J. Med. Chem. 37(21): 3464-3476 (1994), Casero et al., Cancer Chemother. Pharmacol 36: 69-74 (1995), Bernacki et al., Clin. Cancer Res. 1: 847-857 (1995); Bergeron et al., J. Med. Chem. 40: 1475-1494 (1997); Gabrielson et al., Clinical Cancer Res. 5: 1638-1641 (1999), and Bergeron et al., J. Med. Chem. 43: 224-235 (2000), which can be administered alone or in combination with other active agents, such as anti-cancer agents, e.g., cis-diaminedichloroplatinum (II) and 1,3-bis(2-chloroethyl)-1-nitrosourea.

Preferred routes of administration in the first embodiment of the method of treating cancer include intratumoral and peritumoral routes of administration. A preferred manner of administering a separate anti-cancer agent is by targeting to a cancer cell. In this regard, examples of cancer-specific, cell-surface molecules include placental alkaline phosphatase (testicular and ovarian cancer), pan carcinoma (small cell lung cancer), polymorphic epithelial mucin (ovarian cancer), prostate-specific membrane antigen, α-fetoprotein, B-lymphocyte surface antigen (B-cell lymphoma), truncated EGFR (gliomas), idiotypes (B-cell lymphoma), gp95/gp97 (melanoma), N-CAM (small cell lung carcinoma), cluster w4 (small cell lung carcinoma), cluster 5A (small cell carcinoma), cluster 6 (small cell lung carcinoma), PLAP (seminomas, ovarian cancer, and non-small cell lung cancer), CA-125 (lung and ovarian cancers), ESA (carcinoma), CD19, 22 or 37 (B-cell lymphoma), 250 kD proteoglycan (melanoma), P55 (breast cancer), TCR-IgH fusion (childhood T-cell leukemia), blood group A antigen in B or O type individual (gastric and colon tumors), and the like. See, e.g., U.S. Pat. No. 6,080,725 for other examples.

Examples of cancer-specific, cell-surface receptors include erbB-2, erbB-3, erbB-4, IL-2 (lymphoma and leukemia), IL-4 (lymphoma and leukemia), IL-6 (lymphoma and leukemia), MSH (melanoma), transferrin (gliomas), tumor vasculature integrins, and the like. Preferred cancer-specific, cell-surface receptors include erbB-2 and tumor vasculature integrins, such as CD11a, CD11b, CD11c, CD18, CD29, CD51, CD61, CD66d, CD66e, CD106, and CDw145.

There are a number of antibodies to cancer-specific, cell-surface molecules and receptors that are known. C46 Ab (Amersham) and 85A12 Ab (Unipath) to carcino-embryonic antigen, H17E2 Ab (ICRF) to placental alkaline phosphatase, NR-LU-10 Ab (NeoRx Corp.) to pan carcinoma, HMFC1 Ab (ICRF) to polymorphic epithelial mucin, W14 Ab to B-human chorionic gonadotropin, RFB4 Ab (Royal Free Hospital) to B-lymphocyte surface antigen, A33 Ab (Genex) to human colon carcinoma, TA-99 Ab (Genex) to human melanoma, antibodies to c-erbB2 (JP 7309780, JP 8176200 and JP 7059588), and the like. ScAbs can be developed, based on such antibodies, using techniques known in the art (see for example, Bind et al., Science 242: 423-426 (1988), and Whitlow et al., Methods 2(2): 97-105 (1991)).

Also in view of the above, the present invention provides a method of treating a mammal prophylactically or therapeutically for a pathogenic infection. The method comprises administering to the mammal an effective amount of:

(a) an isolated or purified nucleic acid molecule encoding a fusion protein comprising (i) KLH, a variant KLH or an immunogenic fragment of either of the foregoing, and (ii) an antigen of the pathogen, wherein the isolated or purified nucleic acid molecule is optionally in the form of a vector,

(b) a fusion protein comprising (i) KLH, a variant KLH or an immunogenic fragment of either of the foregoing, and (ii) an antigen of the pathogen,

(c) a conjugate comprising (i) (a) KLH-2, a variant KLH-2, or an immunogenic fragment of either of the foregoing comprising at least 360 contiguous amino acids, wherein the KLH-2 is essentially free from KLH-1, or (b) KLH-1, a variant KLH-2, or an immunogenic fragment of either of the foregoing comprising at least 770 contiguous amino acids, wherein the KLH-1 is essentially free from KLH-2, and (ii) an antigen of the pathogen or an anti-pathogen agent, or

(d) a composition comprising (i) (a) KLH-2, a variant KLH-2, or an immunogenic fragment of either of the foregoing comprising at least 360 contiguous amino acids, wherein the KLH-2 is essentially free from KLH-1, or (b) KLH-1, a variant KLH-1, or an immunogenic fragment of either of the foregoing comprising at least 770 contiguous amino acids, wherein the KLH-1 is essentially free from KLH-2, and (ii) an antigen of the pathogen or an anti-pathogen agent, whereupon the mammal is treated for the viral infection prophylactically or therapeutically. The pathogen can be any pathogen, such as a virus, a bacterium or a fungus. For examples of bacterial and viral antigens, see, e.g., U.S. Pat. No. 6,080,725. Fungal antigens are known in the art. Anti-pathogen agents, i.e., active agents used to treat prophylactically or therapeutically infection of an animal, such as a mammal, in particular a human, with a virus, bacterium or fungus, are known in the art.

A method of stimulating an immune response (i.e., immune reactants, e.g., antibodies or specific effector T-cells, directed at an antigen) in a mammal is also provided. In one embodiment, the method comprises administering to the mammal an effective amount of:

(a) an isolated or purified nucleic acid molecule consisting essentially of a nucleotide sequence encoding KLH-2, a variant KLH-2 or an immunogenic fragment of either of the foregoing comprising at least 1,080 contiguous nucleotides, wherein the isolated or purified nucleic acid molecule is optionally in the form of a vector,

(b) an isolated or purified nucleic acid molecule consisting essentially of a nucleotide sequence encoding KLH-1, a variant KLH-1 or an immunogenic fragment of either of the foregoing comprising at least 2,300 contiguous nucleotides, wherein the isolated or purified nucleic acid molecule is optionally in the form of a vector,

(c) an isolated or purified polypeptide molecule consisting essentially of an amino acid sequence encoding KLH-2, a variant KLH-2 or an immunogenic fragment thereof comprising at least 360 contiguous amino acids, wherein the KLH-2 is essentially free from KLH-1, or

(d) an isolated or purified polypeptide molecule consisting essentially of an amino acid sequence encoding KLH-1, a variant KLH-1 or an immunogenic fragment thereof comprising at least 770 contiguous amino acids, wherein the KLH-1 is essentially free from KLH-2,

whereupon an immune response in the mammal is stimulated. Preferably, the stimulation of the immune response results in the inhibition of tyrosinase. In another embodiment, the method comprises stimulating an immune response to an antigen in a mammal by administering to the mammal an effective amount of dendritic cells which have been previously isolated from the mammal and treated in vitro with:

(a) a fusion protein comprising (i) KLH, a variant KLH or an immunogenic fragment of either of the foregoing, and (ii) an antigen,

(b) a conjugate comprising (i) (a) KLH-2, a variant KLH-2, or an immunogenic fragment of either of the foregoing comprising at least 360 contiguous amino acids, wherein the KLH-2 is essentially free from KLH-1, or (b) KLH-1, a variant KLH-1, or an immunogenic fragment of either of the foregoing comprising at least 770 contiguous amino acids, wherein the KLH-1 is essentially free from KLH-2, and (ii) an antigen, or

(c) a composition comprising (i) (a) KLH-2, a variant KLH-2, or an immunogenic fragment of either of the foregoing comprising at least 360 contiguous amino acids, wherein the KLH-2 is essentially free from KLH-1, or (b) KLH-1, a variant KLH-1, or an immunogenic fragment of either of the foregoing comprising at least 770 contiguous amino acids, wherein the KLH-1 is essentially free from KLH-2, and (ii) an antigen,

whereupon an immune response to the antigen is induced in the mammal.

Preferably, the antigen is an antigen of a pathogen or an antigen of a cancer as herein described. The method can further comprise the simultaneous or sequential co-administration of an active agent (e.g., an anti-cancer agent or an anti-pathogen agent) by the same or different route. Methods of isolating, treating ex vivo, and administering dendritic cells are known in the art. See, e.g., International Patent Application WO 00/73432.

Further provided is a method of treating high blood pressure in a mammal. The method comprises administering to the mammal an effective amount of:

(a) an isolated or purified nucleic acid molecule encoding a fusion protein comprising (i) KLH, a variant KLH or an immunogenic fragment of either of the foregoing, and (ii) hemocyanin β-adrenergic receptor peptide, wherein the isolated or purified nucleic acid molecule is optionally in the form of a vector,

(b) a fusion protein comprising (i) KLH, a variant KLH or an immunogenic fragment of either of the foregoing, and (ii) hemocyanin β-adrenergic receptor peptide,

(c) a conjugate comprising (i) (a) KLH-2, a variant KLH-2, or an immunogenic fragment of either of the foregoing comprising at least 360 contiguous amino acids, wherein the KLH-2 is essentially free from KLH-1, or (b) KLH-1, a variant KLH-1, or an immunogenic fragment of either of the foregoing comprising at least 770 contiguous amino acids, wherein the KLH-1 is essentially free from KLH-2, and (ii) hemocyanin β-adrenergic receptor peptide, or

(d) a composition comprising (i) (a) KLH-2, a variant KLH-2, or an immunogenic fragment of either of the foregoing comprising at least 360 contiguous amino acids, wherein the KLH-2 is essentially free from KLH-1, or (b) KLH-1, a variant KLH-1, or an immunogenic fragment of either of the foregoing comprising at least 770 contiguous amino acids, wherein the KLH-1 is essentially free from KLH-2, and (ii) hemocyanin β-adrenergic receptor peptide, whereupon the mammal is treated for high blood pressure prophylactically or therapeutically.

Generally, when KLH (or a conjugate or fusion protein thereof) is administered to an animal, such as a mammal, in particular a human, it is preferable that KLH be administered in a dose of from about 1 to about 1,000 μg/kg body weight/treatment when given parenterally. However, this dosage range is merely preferred, and higher or lower doses may be chosen in appropriate circumstances. For instance, the actual dose and schedule can vary depending on whether the composition is administered in combination with other pharmaceutical compositions, or depending on interindividual differences in pharmacokinetics, drug disposition, and metabolism. One skilled in the art easily can make any necessary adjustments in accordance with the necessities of the particular situation.

Those of ordinary skill in the art can easily make a determination of the amount of an above-described isolated and purified nucleic acid molecule to be administered to an animal, such as a mammal, in particular a human. The dosage will depend upon the particular method of administration, including any vector or promoter utilized. For purposes of considering the dose in terms of particle units (pu), also referred to as viral particles, it can be assumed that there are 100 particles/pfu (e.g., 1×10¹² pfu is equivalent to 1×10¹⁴ pu). An amount of recombinant virus, recombinant DNA vector or RNA genome sufficient to achieve a tissue concentration of about 10² to about 10¹² particles per ml is preferred, especially of about 10⁶ to about 10¹⁰ particles per ml. In certain applications, multiple daily doses are preferred. Moreover, the number of doses will vary depending on the means of delivery and the particular recombinant virus, recombinant DNA vector or RNA genome administered.

A targeting moiety also can be used in the contact of a cell with an above-described isolated or purified nucleic acid molecule. In this regard, any molecule that can be linked with the therapeutic nucleic acid directly or indirectly, such as through a suitable delivery vehicle, such that the targeting moiety binds to a cell-surface receptor, can be used. The targeting moiety can bind to a cell through a receptor, a substrate, an antigenic determinant or another binding site on the surface of the cell. Examples of a targeting moiety include an antibody (i.e., a polyclonal or a monoclonal antibody), an immunologically reactive fragment of an antibody, an engineered immunoprotein and the like, a protein (target is receptor, as substrate, or regulatory site on DNA or RNA), a polypeptide (target is receptor), a peptide (target is receptor), a nucleic acid, which is DNA or RNA (i.e., single-stranded or double-stranded, synthetic or isolated and purified from nature; target is complementary nucleic acid), a steroid (target is steroid receptor), and the like. Analogs of targeting moieties that retain the ability to bind to a defined target also can be used. In addition, synthetic targeting moieties can be designed, such as to fit a particular epitope. Alternatively, the therapeutic nucleic acid can be encapsulated in a liposome comprising on its surface the targeting moiety.

The targeting moiety includes any linking group that can be used to join a targeting moiety to, in the context of the present invention, an above-described nucleic acid molecule. It will be evident to one skilled in the art that a variety of linking groups, including bifunctional reagents, can be used. The targeting moiety can be linked to the therapeutic nucleic acid by covalent or non-covalent bonding. If bonding is non-covalent, the conjugation can be through hydrogen bonding, ionic bonding, hydrophobic or van der Waals interactions, or any other appropriate type of binding.

An isolated or purified nucleic acid molecule consisting essentially of SEQ ID NO: 4 or SEQ ID NO: 25 or encoding SEQ ID NO: 6 or SEQ ID NO: 27 is also provided. Additionally provided is an isolated or purified nucleic acid molecule consisting essentially of the aforementioned nucleic acid molecule coupled 5′ to an isolated or purified nucleic acid molecule encoding a polypeptide or protein, optionally in the form of a vector, which, upon expression in a eukaryotic cell, results in the secretion of the encoded polypeptide or protein by the eukaryotic cell. See, e.g., U.S. Pat. No. 6,169,172. Thus, a eukaryotic cell comprising and expressing such a nucleic acid molecule is also provided.

### EXAMPLES

The following examples serve to illustrate the present invention and is not intended to limit its scope in any way.

The following references, to the extent that they provide exemplary procedural or other details supplementary to those set forth herein, are specifically incorporated herein by reference:

Birren et al., Genome Analysis: A Laboratory Manual Series, Volume 1, Analyzing DNA, Cold Spring Harbor Laboratory Press, Cold Spring Harbor, N.Y. (1997),

Birren et al., Genome Analysis: A Laboratory Manual Series, Volume 2, Detecting Genes, Cold Spring Harbor Laboratory Press, Cold Spring Harbor, N.Y. (1998),

Birren et al., Genome Analysis: A Laboratory Manual Series, Volume 3, Cloning Systems, Cold Spring Harbor Laboratory Press, Cold Spring Harbor, N.Y. (1999),

Birren et al., Genome Analysis: A Laboratory Manual Series, Volume 4, Mapping Genomes, Cold Spring Harbor Laboratory Press, Cold Spring Harbor, N.Y. (1999),

Harlow et al., Antibodies: A Laboratory Manual, Cold Spring Harbor Laboratory Press, Cold Spring Harbor, N.Y. (1988),

Harlow et al., Using Antibodies: A Laboratory Manual, Cold Spring Harbor Laboratory Press, Cold Spring Harbor, N.Y. (1999),

Hoffman, Cancer and the Search for Selective Biochemical Inhibitors, CRC Press (1999),

Pratt, The Anticancer Drugs, 2nd edition, Oxford University Press, NY (1994), and

Sambrook et al., Molecular Cloning: A Laboratory Manual, 2nd edition, Cold Spring Harbor Laboratory Press, Cold Spring Harbor, N.Y. (1989).

### Example 1

This example describes the sequencing of the KLH-2 cDNA.

Fresh limpet tissue associated with the respiratory system and other organ tissues were dissected and immediately frozen on dry ice. The tissue was stored at −70° C.

Two hundred mg of tissue were homogenized with a PowerGen 125 (Fisher Scientific, Pittsburgh, Pa.) in 3.0 ml Trizol Reagent (Life Technologies, Gaithersburg, Md.). The sample was centrifuged at 15,000×g at 4° C. for 10 min. The sample was allowed to come to room temperature (15-30° C.) for two min. The aqueous layer was extracted twice with 0.3 ml of chloroform. The RNA was then precipitated with isopropanol. The RNA pellets were washed with 75% ethanol, dried and dissolved in distilled water. The RNA solution was stored at −70° C.

cDNA was prepared using the Thermoscript™ RT-PCR system (Life Technologies, Gaithersburg, Md.) following the manufacturer's recommended directions. The cDNA was primed with oligo dT.

Degenerate PCR primers were designed using the amino acid sequence of the KLH functional unit 2c (Sohngen et al., Eur. J. Biochem. 248: 602-614 (1997); and Stoeva et al., Biochim. Biophys. Acta 1435: 94-109 (1999)).

The design of the degenerate PCR primers took into consideration codon usage frequency. The Codon Usage Database is an extended WWW version of CUTG (Codon Usage Tabulated from GenBank) and was used to determine codon usage.

Codon frequency tables were also generated using the Countcodon program (version 4). Octopus dolfleini was used as a model hemocyanin and the codon usage correlated well with other standard codon usage tables. In addition, the degenerate PCR primers were designed to avoid, when possible, amino acids with numerous codons, e.g., leucine, arginine and serine.

IUB group codes were used to identify redundancies.

R=A+G

Y=C+T

M=A+C

K=G+T

S=G+C

W=A+T

H=A+T+C

B=G+T+C

D=G+A+T

N=A+G+T+C

V=G+A+C

| | | | |
|---|---|---|---|
| Primer 12 | k1h2cSns-lessdeg71 | TTYCCWCAYTGGCAY | [SEQ ID NO: 13] |
| Primer 11 | k1h2cSns-deg71 | TTYCCNCAYTGGCAY | [SEQ ID NO: 14] |
| Primer 30 | k1h2cAnti-lessdeg223 | TTGCCAAATDGGCCA | [SEQ ID NO: 15] |
| Primer 15 | k1h2cSns-deg96 | CNTAYTGGGAYTGGAC | [SEQ ID NO: 16] |
| Primer 53 | K1h2cAnti-deg354 | GGGATYTCNGCYTCRTC | [SEQ ID NO: 17] |

PCR reactions were done with the following conditions:

94° C., 15 sec

52° C., 30 sec

72° C., 1 min

35 cycles

72° C., 10 min

4° C. Hold

Primer 12+Primer 53 yielded PCR product of approximately 850 base pairs. Plasmid 3.1

Primer 11+Primer 30 yielded PCR product of approximately 460 base pairs. Plasmid 6.2

Primer 11+Primer 30 yielded PCR product of approximately 460 base pairs. Plasmid 6.1

Primer 12+Primer 30 yielded PCR product of approximately 460 base pairs. Plasmid 8.1

Primer 12+Primer 30 yielded PCR product of approximately 460 base pairs. Plasmid 8.2

Primer 15+Primer 30 yielded PCR product of approximately 380 base pairs. Plasmid 10.2

PCR products were separated on a 1% agarose gel. Bands of the expected size were excised with a razor blade.

The amplified DNA was extracted from the agarose using an Ultrafree-DA (Millipore, Bedford, Mass.) spin column. The gel-purified fragments were then cloned into pGEM-T Easy vector (Promega, Madison, Wis.) according to the manufacturer's recommended directions.

Cells were transformed with pGEM-T easy vector containing PCR product. Max efficiency DH5 alpha chemically competent cells (Life Technologies) were used according to the manufacturer's recommended directions.

After transformation, colonies were picked and cultured in LB broth containing 100 μg/ml of ampicillin. Plasmids were purified using the QIAprep Spin Miniprep Kit (Qiagen, Valencia, Calif.) according to the manufacturer's directions.

Plasmids were screened for inserts by digesting with Not I and subsequent gel electrophoresis. Plasmids containing inserts of the expected size were sequenced.

Sequence data were translated and clones of the open reading frames were compared with other hemocyanins using the BLAST sequence analysis program. Clones containing homology to hemocyanins found in the database were used to make primers for 5′ and 3′ RACE reactions.

5′ and 3′ RACE reactions were performed using the SMART™ RACE cDNA Amplification Kit (Catalog K 1811-1, Clontech) according to the manufacturer's recommended protocols.

5′ RACE primers:

| | | |
|---|---|---|
| Primer 1 | CCATAGCGTCAAACAGATGCGTGTGGTGTCC | [SEQ ID NO: 18] |
| Primer 2 | GCAAAGCTTGCCAAATGGCCCAAAGTCG | [SEQ ID NO: 19] |

5′RACE reaction products were separated on a 1% agarose gel. The fragments corresponding to the expected size were gel purified using an Ultrafree-DA spin column (Millipore Corporation, Bedford, Mass.) and TA cloned into TOPOXL (Invitrogen, Carlsbad, Calif.) and then used to transform TOP 10 (Invitrogen) electrocompetent cells. Colonies from the transformation reaction were used to inoculate 2 ml LB broth containing 50 μg/ml of kanamycin for plasmid minipreps (QIAprep Spin Miniprep Kit, Qiagen). Plasmids were digested with Eco RI to determine inserts of the expected size.

RACE Thermal cycling conditions:

1) 94° C., 30 seconds

2) 72° C., 3 minutes

3) Repeat steps 1 and 2, 5 times

4) 94° C., 30 seconds

5) 70° C., 30 seconds

6) 72° C., 30 seconds

7) repeat steps 4 through 6, 5 times

8) 94° C., 30 seconds

9) 68° C., 30 seconds

10) 72° C., 3 minutes

11) Repeat steps 8-10, 25 times

12) 4° C. hold

Plasmids

4-2 generated by 5′ RACE primer 2

7-4 generated by 5′ RACE primer 2

7-6 generated by 5′ RACE primer 2

6-3 generated by 5′ RACE primer 1

were sequenced. Plasmids containing overlapping sequence corresponding to the previously cloned portion of the KLH2 cDNA were sequenced.

3′ RACE primers:

| | | |
|---|---|---|
| 3′ RACE primer 1 | GAGCGCCCTTCCACATTTTGTTG | [SEQ ID NO: 20] |
| 3′ RACE primer 2 | AGCGCCCTTCCACATTTTGTTGC | [SEQ ID NO: 21] |

3′RACE reaction products were separated on a 1% agarose gel. The fragments corresponding to the expected size were gel-purified using an Ultrafree-DA spin column (Amicon, Millipore) and TA cloned into TOPOXL (Invitrogen) and then used to transform TOP10 (Invitrogen) electrocompetent cells. Colonies from the transformation reaction were used to inoculate 2 ml LB broth containing 50 μg/ml of kanamycin for plasmid minipreps (QIAprep Spin Miniprep Kit) (Qiagen). Plasmids were digested with Eco RI to determine inserts of the expected size.

3′ RACE plasmids 1-3, 1-4, and 2-5 were sequenced. The overlapping 5′ and 3′ RACE clones contained the complete cDNA sequence of KLH isotype 2 and, in addition, part of the 5′ untranslated region, including the N-terminal methionine, a leader peptide sequence for secretion of the KLH-2 protein, a 3′ untranslated region and the 3′ poly A tail.

### Example 2

This example describes the cloning and sequencing of the KLH-1 cDNA.

Fresh limpet tissue associated with the respiratory system and other organ tissues were dissected and immediately frozen on dry ice. The tissue was stored at −70° C.

Two hundred mg of tissue were homogenized with a PowerGen 125 (Fisher Scientific) in 3.0 ml Trizol Reagent (Life Technologies). The sample was centrifuged at 15,000×g at 4° C. for 10 min. The sample was allowed to come to room temperature (15-30° C.) for two min. The aqueous layer was extracted twice with 0.3 ml of chloroform. The RNA was then precipitated with isopropanol. The RNA pellets were washed with 75% ethanol, dried and dissolved in distilled water. The RNA solution was stored at −70° C.

Hemocyanin sequences for Megathura crenulata found on NCBI's GenBank database were used to make primers for 5′ and 3′ RACE reactions (International Patent Application WO 01/14536, SEQ ID NO: 210, 211, and 217). 5′ and 3′ RACE reactions were performed using the SMART™ RACE cDNA Amplification Kit (Catalog K 1811-1, CLONTECH) according to the manufacturer's recommended protocols.

| | | |
|---|---|---|
| 5′ RACE primer: | | |
| Primer 1 | CGGGGTGACAGTTGCTGCTATCGGTGCA | [SEQ ID NO: 34] |
| 3′ RACE primer: | | |
| Primer 2 | GCCTTGACCGCCCATGGTGCCAGA | [SEQ ID NO: 35] |
| PCR Primers: | | |
| Primer 3 | GGGGCGACCGAGTTGTATCCACTCCCA | [SEQ ID NO: 36] |
| Primer 4 | GGCTGGAACATTTGCAGTGCTCGGTGG | [SEQ ID NO: 37] |

PCR reactions were performed with 5′ RACE-ready cDNA with primers 3 and 4 under the following thermal cycling conditions:

1) 94° C., 30 seconds

2) 72° C., 10 minutes

3) Repeat steps 1 and 2, 5 times

4) 94° C., 30 seconds

5) 70° C., 30 seconds

6) 72° C., 10 minutes

7) repeat steps 4 through 6, 5 times

8) 94° C., 30 seconds

9) 68° C., 30 seconds

10) 72° C., 10 minutes

11) Repeat steps 8-10, 25 times

12) 72° C., 10 minutes

13) 4° C. hold

5′ RACE thermal cycling conditions for Primer 1:

1) 94° C., 15 seconds

2) Gradient (69.5-72° C.) 6 minutes

3) Repeat steps 1 and 2, 5 times

4) 94° C., 15 seconds

5) 70° C., 30 seconds

6) 72° C., 6 minutes

7) repeat steps 4 through 6, 5 times

8) 94° C., 15 seconds

9) 68° C., 30 seconds

10) 72° C., 6 minutes

11) Repeat steps 8-10, 29 times

12) 72° C., 10 minutes

13) 4° C. hold

3′ RACE Thermal cycling conditions for Primer 2:

1) 94° C., 30 seconds

2) 70° C., 10 minutes

3) Repeat steps 1 and 2, 5 times

4) 94° C., 30 seconds

5) 68° C., 30 seconds

6) 72° C., 10 minutes

7) repeat steps 4 through 6, 5 times

8) 94° C., 30 seconds

9) 66° C., 30 seconds

10) 72° C., 10 minutes

11) Repeat steps 8-10, 25 times

12) 72° C., 10 minutes

13) 4° C. hold

5′ RACE reaction yielded a product of approximately 3500 bp. 3′ RACE reaction yielded a product of approximately 2900 bp. PCR reactions with 316A and 302B primers yielded PCR product of approximately 5600 bp. Reaction products were separated on a 1% agarose gel. The fragments corresponding to the expected size were gel-purified using an Ultrafree-DA spin column (Amicon, Millipore, Bedford, Mass.) and TA cloned into TOPOXL (Invitrogen) and then used to transform TOP10 (Invitrogen) electrocompetent cells. Colonies from the transformation reaction were used to inoculate 2 ml LB broth containing 50 μg/ml of kanamycin for plasmid minipreps (QIAprep Spin Miniprep Kit)(Qiagen). Plasmids were digested with Eco RI and Not 1 (Promega, Madison, Wis.) to determine inserts of the expected size.

Plasmids 5-4, 16-1, and 12-3 were sequenced. The overlapping 5′ and 3′ RACE clones contained the complete cDNA sequence of KLH-1 and, in addition, part of the 5′ untranslated region, including the N-terminal methionine, a leader peptide sequence for secretion of the KLH-1 protein, a 3′ untranslated region and the 3′ poly A tail.

### Example 3

This example describes the construction of vectors containing full-length KLH1 and KLH2 cDNA.

RNA extraction was performed as described in Example 1.

cDNA was prepared using the SMART cDNA Synthesis Kit (Catalog K1052-1, CLONTECH) according to the manufacturer's recommended protocols.

| | | |
|---|---|---|
| KLH2 Primer 1 (forward): 5′ to 3′ | AGGCTACGCGTTCGAAGGAGATAGAACCATGTGGACCATCTTGGCTCTC | [SEQ ID NO: 38] |
| KLH2 Primer 2 (reverse): 5′ to 3′ | CTGGTGCGGCCGCCTTACTCGTCTTCAATGTGGATATG | [SEQ ID NO: 39] |
| KLH1 Primer 1 (forward): 5′ to 3′ | ATGCTGTCGGTCAGGTTGCTTATAGTCG | [SEQ ID NO: 40] |
| KLH1 Primer 2 (reverse): 5′ to 3′ | CTATTCGTCATCCACATGGACATGTATCC | [SEQ ID NO: 41] |

KLH1 and KLH2 thermal cycling conditions:

1) 95° C., 2 minutes

2) 94° C., 20 seconds

3) Gradient (58-70° C.) 30 seconds

4) 72° C., 10 minutes

5) Repeat steps 2 through 4, 29 times

6) 72° C., 10 minutes

7) 4° C. hold

KLH1 and KLH2 PCR reactions yielded products of approximately 10,000 bp. PCR products were separated on a 1% agarose gel. The fragments were gel-purified using an Ultrafree-DA spin column (Amicon, Millipore, Bedford, Mass.), TA cloned into pCR-XL-TOPO (Invitrogen) using the TOPOXL PCR Cloning Kit (Catalog K4700-10, Invitrogen), and used to transform TOP10 (Invitrogen) electrocompetent cells. Colonies from the transformation reaction were used to inoculate 2 mL LB broth containing 50 μg/mL of kanamycin for plasmid minipreps (QIAprep Spin Miniprep Kit, QIAGEN).

KLH1 plasmids were digested with Not1 restriction enzyme (New England Biolabs). KLH2 plasmids were digested with Mlu1 and Not1 restriction enzymes (New England Biolabs). Digests were separated on a 1% agarose gel to determine inserts of the expected size.

KLH2 plasmid 13 and KLH1 plasmid 5-1 were each sequenced. Two-three kilobases of both of the 5′ and 3′ ends of each insert were sequenced for confirmation. The plasmids were found to contain the coding regions of KLH2 and KLH1, respectively.

All of the references cited herein, including patents, patent applications, and publications, are hereby incorporated in their entireties by reference.

While this invention has been described with an emphasis upon preferred embodiments, it will be obvious to those of ordinary skill in the art that variations of the preferred embodiments may be used and that it is intended that the invention may be practiced otherwise than as specifically described herein. Accordingly, this invention includes all modifications encompassed within the spirit and scope of the invention as defined by the following claims.

1. An isolated or purified nucleic acid molecule consisting essentially of a nucleotide sequence encoding keyhole limpet hemocyanin (KLH)-2 or a fragment thereof comprising at least 1,080 contiguous nucleotides.

2. The isolated or purified nucleic acid molecule of claim 1, which (i) encodes the amino acid sequence of SEQ ID NO: 3 or at least 360 contiguous amino acids of SEQ ID NO: 3, (ii) consists essentially of the nucleotide sequence of SEQ ID NO: 1 or a fragment thereof comprising at least 1,080 contiguous nucleotides, (iii) hybridizes under moderately stringent conditions to an isolated or purified nucleic acid molecule consisting essentially of the nucleotide sequence that is complementary to SEQ ID NO: 1 or a fragment thereof comprising at least 1,080 contiguous nucleotides and is characterized by no more than 40% mismatch, or (iv) shares 60% or more identity with SEQ ID NO: 1.

3. The isolated or purified nucleic acid molecule of claim 2, which hybridizes under highly stringent conditions to an isolated or purified nucleic acid molecule consisting essentially of the nucleotide sequence that is complementary to SEQ ID NO: 1 or a fragment thereof comprising at least 1,080 contiguous nucleotides and is characterized by no more than 20% mismatch.

4. An isolated or purified nucleic acid molecule consisting essentially of a nucleotide sequence that is complementary to a nucleotide sequence encoding KLH-2 or a fragment thereof comprising at least 1,080 contiguous nucleotides.

5. The isolated or purified nucleic acid molecule of claim 4, which (i) is complementary to a nucleotide sequence encoding the amino acid sequence of SEQ ID NO: 3 or at least 360 contiguous amino acids of SEQ ID NO: 3, (ii) is complementary to the nucleotide sequence of SEQ ID NO: 1 or a fragment thereof comprising at least 1,080 contiguous nucleotides, (iii) hybridizes under moderately stringent conditions to an isolated or purified nucleic acid molecule consisting essentially of SEQ ID NO: 1 or a fragment thereof comprising at least 1,080 contiguous nucleotides and is characterized by no more than 40% mismatch, or (iv) shares 60% or more identity with the nucleotide sequence that is complementary to SEQ ID NO: 1.

6. An isolated or purified nucleic acid molecule consisting essentially of a nucleotide sequence encoding KLH-1 or a fragment thereof comprising at least 2,300 contiguous nucleotides.

7. The isolated or purified nucleic acid molecule of claim 6, which (i) encodes the amino acid sequence of SEQ ID NO: 24 or at least 770 contiguous amino acids of SEQ ID NO: 24, (ii) consists essentially of the nucleotide sequence of SEQ ID NO: 22 or a fragment thereof comprising at least 2,300 contiguous nucleotides, (iii) hybridizes under moderately stringent conditions to an isolated or purified nucleic acid molecule consisting essentially of the nucleotide sequence that is complementary to SEQ ID NO: 22 or a fragment thereof comprising at least 2,300 contiguous nucleotides and is characterized by no more than 35% mismatch, or (iv) shares 65% or more identity with SEQ ID NO: 22.

8. The isolated or purified nucleic acid of claim 7, which hybridizes under highly stringent conditions to an isolated or purified nucleic acid molecule consisting essentially of the nucleotide sequence that is complementary to SEQ ID NO: 22 or a fragment thereof comprising at least 2,300 contiguous nucleotides and is characterized by no more than 20% mismatch.

9. An isolated or purified nucleic acid molecule consisting essentially of a nucleotide sequence that is complementary to a nucleotide sequence encoding KLH-1 or a fragment thereof comprising at least 2,300 contiguous nucleotides.

10. The isolated or purified nucleic acid molecule of claim 9, which (i) is complementary to a nucleotide sequence encoding the amino acid sequence of SEQ ID NO: 24 or at least 770 contiguous amino acids of SEQ ID NO: 24, (ii) is complementary to the nucleotide sequence of SEQ ID NO: 22 or a fragment thereof comprising at least 2,300 contiguous nucleotides, (iii) hybridizes under moderately stringent conditions to an isolated or purified nucleic acid molecule consisting essentially of SEQ ID NO: 22 or a fragment thereof comprising at least 2,300 contiguous nucleotides and is characterized by no more than 35% mismatch, or (iv) shares 65% or more identity with the nucleotide sequence that is complementary to SEQ ID NO: 22.

11. A vector comprising the isolated or purified nucleic acid molecule of claim 1, optionally as part of an encoded fusion protein.

12. A vector comprising the isolated or purified nucleic acid molecule of claim 4.

13. A vector comprising the isolated or purified nucleic acid molecule of any of claim 6, optionally as part of an encoded fusion protein.

14. A vector comprising the isolated or purified nucleic acid molecule of claim 9.

15. A cell comprising and expressing the isolated or purified nucleic acid molecule of claim 1, optionally in the form of a vector.

16. A cell comprising and expressing the isolated or purified nucleic acid molecule of claim 4, optionally in the form of a vector.

17. A cell comprising and expressing the isolated or purified nucleic acid molecule of any of claim 6, optionally in the form of a vector.

18. A cell comprising and expressing the isolated or purified nucleic acid molecule of claim 9, optionally in the form of a vector.

19. An isolated or purified polypeptide molecule consisting essentially of an amino acid sequence of KLH-2 or at least 360 contiguous amino acids of KLH-2, which is optionally glycosylated, amidated, carboxylated, phosphorylated, esterified, N-acylated or converted into an acid addition salt and/or dimerized or polymerized, wherein the isolated or purified polypeptide molecule is essentially free from KLH-1.

20. A conjugate or fusion protein comprising the isolated or purified polypeptide molecule of claim 19 and a therapeutically or prophylactically active agent.

21. The conjugate or fusion protein of claim 20, wherein the active agent is an immunogen, a hapten, an allergen, an antigen of a pathogen, an antigen of a cancer, hemocyanin β-adrenergic receptor peptide, or a sperm coat protein.

22. A composition comprising the isolated or purified polypeptide molecule of claim 19, optionally in the form of a conjugate or a fusion protein comprising a therapeutically or prophylactically active agent, and an excipient or an adjuvant.

23. An isolated or purified polypeptide molecule consisting essentially of an amino acid sequence of KLH-1 or at least 770 contiguous amino acids of KLH-1, which is optionally glycosylated, amidated, carboxylated, phosphorylated, esterified, N-acylated or converted into an acid addition salt and/or dimerized or polymerized, wherein the isolated or purified polypeptide molecule is essentially free from KLH-2.

24. A conjugate or fusion protein comprising the isolated or purified polypeptide molecule of claim 23 and a therapeutically or prophylactically active agent.

25. The conjugate or fusion protein of claim 24, wherein the active agent is an immunogen, a hapten, an allergen, an antigen of a pathogen, an antigen of a cancer, hemocyanin β-adrenergic receptor peptide, or a sperm coat protein.

26. A composition comprising the isolated or purified polypeptide molecule of claim 23, optionally in the form of a conjugate or a fusion protein comprising a therapeutically or prophylactically active agent, and an excipient or an adjuvant.

27. An isolated or purified nucleic acid molecule consisting essentially of nucleotides 1-1,260 of SEQ ID NO: 22, nucleotides 1-1,266 of SEQ ID NO: 1, nucleotides 8,695-10,197 of SEQ ID NO: 22, nucleotides 8,713-10,206 of SEQ ID NO: 1, nucleotides 1,261-1,527 of SEQ ID NO: 22, nucleotides 1,267-1,530 of SEQ ID NO: 1, or nucleotides 7,975-8,694 of SEQ ID NO: 22.

28. An isolated or purified polypeptide molecule consisting essentially of amino acids 1-420 of SEQ ID NO: 24, amino acids 1-422 of SEQ ID NO: 3, amino acids 2,899-3,398 of SEQ ID NO: 24, amino acids 2,905-3,401 of SEQ ID NO: 3, amino acids 421-509 of SEQ ID NO: 24, amino acids 423-510 of SEQ ID NO: 3, or amino acids 2,659-2,898 of SEQ ID NO: 24.

29. A method of treating cancer prophylactically or therapeutically in a mammal, which method comprises administering to the mammal an effective amount of:
(a) an isolated or purified nucleic acid molecule encoding a fusion protein comprising (i) KLH or an immunogenic fragment thereof, and (ii) an antigen of the cancer, wherein the isolated or purified nucleic acid molecule is optionally in the form of a vector,
(b) a fusion protein comprising (i) KLH or an immunogenic fragment thereof, and (ii) an antigen of the cancer,
(c) a conjugate comprising (i) (a) KLH-2 or an immunogenic fragment thereof comprising at least 360 contiguous amino acids, wherein the KLH-2 is essentially free from KLH-1, or (b) KLH-1 or an immunogenic fragment thereof comprising at least 770 contiguous amino acids, wherein the KLH-1 is essentially free from KLH-2, and (ii) an antigen of the cancer or an anti-cancer agent, or
(d) a composition comprising (i) (a) KLH-2 or an immunogenic fragment thereof comprising at least 360 contiguous amino acids, wherein the KLH-2 is essentially free from KLH-1, or (b) KLH-1 or an immunogenic fragment thereof comprising at least 770 contiguous amino acids, wherein the KLH-1 is essentially free from KLH-2, and (ii) an antigen of the cancer and/or an anti-cancer agent, whereupon the mammal is treated for the cancer prophylactically or therapeutically.

30. The method of claim 29, wherein the cancer is adenocarcinoma, bladder cancer, colon cancer, breast cancer, lung cancer or skin cancer.

31. The method of claim 30, wherein the skin cancer is melanoma.

32. A method of treating a mammal prophylactically or therapeutically for a pathogenic infection, which method comprises administering to the mammal an effective amount of:
(a) an isolated or purified nucleic acid molecule encoding a fusion protein comprising (i) KLH or an immunogenic fragment thereof, and (ii) an antigen of the pathogen, wherein the isolated or purified nucleic acid molecule is optionally in the form of a vector,
(b) a fusion protein comprising (i) KLH or an immunogenic fragment thereof, and (ii) an antigen of the pathogen,
(c) a conjugate comprising (i) (a) KLH-2 or an immunogenic fragment thereof comprising at least 360 contiguous amino acids, wherein the KLH-2 is essentially free from KLH-1, or (b) KLH-1 or an immunogenic fragment thereof comprising at least 770 contiguous amino acids, wherein the KLH-1 is essentially free from KLH-2, and (ii) an antigen of the pathogen or an anti-pathogen agent, or
(d) a composition comprising (i) (a) KLH-2 or an immunogenic fragment thereof comprising at least 360 contiguous amino acids, wherein the KLH-2 is essentially free from KLH-1, or (b) KLH-1 or an immunogenic fragment thereof comprising at least 770 contiguous amino acids, wherein the KLH-1 is essentially free from KLH-2, and (ii) an antigen of the pathogen and/or an anti-pathogen agent, whereupon the mammal is treated for the pathogenic infection prophylactically or therapeutically.

33. A method of stimulating an immune response in a mammal, which method comprises administering to the mammal an effective amount of:
(a) an isolated or purified nucleic acid molecule consisting essentially of a nucleotide sequence encoding KLH-2 or an immunogenic fragment thereof comprising at least 1,080 contiguous nucleotides, wherein the isolated or purified nucleic acid molecule is optionally in the form of a vector,
(b) an isolated or purified nucleic acid molecule consisting essentially of a nucleotide sequence encoding KLH-1 or an immunogenic fragment thereof comprising at least 2,300 contiguous nucleotides, wherein the isolated or purified nucleic acid molecule is optionally in the form of a vector,
(c) an isolated or purified polypeptide molecule consisting essentially of an amino acid sequence encoding KLH-2 or an immunogenic fragment thereof comprising at least 360 contiguous amino acids, wherein the KLH-2 is essentially free from KLH-1, or
(d) an isolated or purified polypeptide molecule consisting essentially of an amino acid sequence encoding KLH-1 or an immunogenic fragment thereof comprising at least 770 contiguous amino acids, wherein the KLH-1 is essentially free from KLH-2,
whereupon an immune response in the mammal is stimulated.

34. The method of claim 33, in which the immune response in the mammal that is stimulated results in the inhibition of tyrosinase.

35. A method of stimulating an immune response to an antigen in a mammal, which method comprises administering to the mammal an effective amount of dendritic cells which have been previously isolated from the mammal and treated in vitro with:
(a) a fusion protein comprising (i) KLH or an immunogenic fragment thereof, and (ii) an antigen,
(b) a conjugate comprising (i) (a) KLH-2 or an immunogenic fragment thereof comprising at least 360 contiguous amino acids, wherein the KLH-2 is essentially free from KLH-1, or (b) KLH-1 or an immunogenic fragment thereof comprising at least 770 contiguous amino acids, wherein the KLH-1 is essentially free from KLH-2, and (ii) an antigen, or
(c) a composition comprising (i) (a) KLH-2 or an immunogenic fragment thereof comprising at least 360 contiguous amino acids, wherein the KLH-2 is essentially free from KLH-1, or (b) KLH-1 or an immunogenic fragment thereof comprising at least 770 contiguous amino acids, wherein the KLH-1 is essentially free from KLH-2, and (ii) an antigen,
whereupon an immune response to the antigen is induced in the mammal.

36. The method of claim 35, wherein the antigen is an antigen of a pathogen or an antigen of a cancer.

37. The method of claim 36, wherein the method further comprises the administration of an anti-pathogen agent or an anti-cancer agent.

38. A method of treating high blood pressure in a mammal, which method comprises administering to the mammal an effective amount of:
(a) an isolated or purified nucleic acid molecule encoding a fusion protein comprising (i) KLH or an immunogenic fragment thereof, and (ii) hemocyanin β-adrenergic receptor peptide, wherein the isolated or purified nucleic acid molecule is optionally in the form of a vector,
(b) a fusion protein comprising (i) KLH or an immunogenic fragment thereof, and (ii) hemocyanin β-adrenergic receptor peptide,
(c) a conjugate comprising (i) (a) KLH-2 or an immunogenic fragment thereof comprising at least 360 contiguous amino acids, wherein the KLH-2 is essentially free from KLH-1, or (b) KLH-1 or an immunogenic fragment thereof at least 770 contiguous amino acids, wherein the KLH-1 is essentially free from KLH-2, and (ii) hemocyanin β-adrenergic receptor peptide, or
(d) a composition comprising (i) (a) KLH-2 or an immunogenic fragment thereof comprising at least 360 contiguous amino acids, wherein the KLH-2 is essentially free from KLH-1, or (b) KLH-1 or an immunogenic fragment thereof comprising at least 770 contiguous amino acids, wherein the KLH-1 is essentially free from KLH-2, and (ii) hemocyanin β-adrenergic receptor peptide, whereupon the mammal is treated for high blood pressure prophylactically or therapeutically.

39. An isolated or purified nucleic acid molecule consisting essentially of SEQ ID NO: 4 or encoding SEQ ID NO: 6.

40. An isolated or purified nucleic acid molecule consisting essentially of the isolated or purified nucleic acid molecule of claim 39 coupled to an isolated or purified nucleic acid molecule encoding a polypeptide or protein, optionally in the form of a vector, which, upon expression in a eukaryotic cell, results in the secretion of the encoded polypeptide or protein by the eukaryotic cell.

41. A eukaryotic cell comprising and expressing the isolated or purified nucleic acid molecule of claim 40.

42. An isolated or purified nucleic acid molecule consisting essentially of SEQ ID NO: 25 or encoding SEQ ID NO: 27.

43. An isolated or purified nucleic acid molecule consisting essentially of the isolated or purified nucleic acid molecule of claim 42 coupled to an isolated or purified nucleic acid molecule encoding a polypeptide or protein, optionally in the form of a vector, which, upon expression in a eukaryotic cell, results in the secretion of the encoded polypeptide or protein by the eukaryotic cell.

44. A eukaryotic cell comprising and expressing the isolated or purified nucleic acid molecule of claim 43.

45. A method of identifying an immunogenic portion of KLH, wherein the KLH is KLH-1 in the absence of KLH-2 or wherein the KLH is KLH-2 in the absence of KLH-1, which method comprises:
(i) contacting antigen-presenting cells with KLH or a portion thereof,
(ii) harvesting the antigen-presenting cells that have been contacted with KLH or a portion thereof,
(iii) purifying complexes of major histocompatibility complex (MHC) and peptidic fragments derived from KLH from the harvested antigen-presenting cells,
(iv) isolating the peptidic fragments from the purified complexes, and
(v) analyzing the peptidic fragments to determine which fragments were bound to MHC,
whereupon an immunogenic portion of KLH is identified.

46-47. (Cancelled).

48. An immunogenic portion of KLH-1 identified in accordance with the method of claim 45.

49. An immunogenic portion of KLH-2 identified in accordance with the method of claim 45.
